(19) United States
(12) Patent Application Publication
(10) Pub. No.: US 2026/0117269 A1
(43) Pub. Date: Apr. 30, 2026

(54) CYP76AD1-BETA CLADE POLYNUCLEOTIDES, POLYPEPTIDES, AND USES THEREOF

(71) Applicant: YEDA RESEARCH AND DEVELOPMENT CO. LTD., Rehovot (IL)

(72) Inventors: Asaph AHARONI, Tel Aviv (IL); Guy POLTURAK, Rehovot (IL)

(73) Assignee: YEDA RESEARCH AND DEVELOPMENT CO. LTD., Rehovot (IL)

(21) Appl. No.: 19/428,740

(22) Filed: Dec. 22, 2025

Related U.S. Application Data
(62) Division of application No. 18/436,233, filed on Feb. 8, 2024, now Pat. No. 12,529,083.
Application No. 18/436,233 is a continuation of application No. 16/940,666, filed on Jul. 28, 2020, now Pat. No. 11,932,887.
Application No. 16/940,666 is a continuation of application No. 15/758,372, now Pat. No. 10,767,201.

(30) Foreign Application Priority Data
Sep. 10, 2015 (IL) 241462

(51) Int. Cl. C12P 13/22; A23B 2/729; A23B 2/762; A23B 2/771; A23L 5/46; C07K 14/415; C12N 9/02; C12N 9/12; C12P 13/04; C12P 17/12; C12P 19/02

(52) U.S. Cl. CPC C12P 13/225; A23B 2/729; A23B 2/762; A23B 2/771; A23L 5/46; C07K 14/415; C12N 9/0083; C12N 9/1205; C12P 13/04; C12P 17/12; C12P 19/02; C12Y 207/01095; A23V 2002/00

(57) ABSTRACT

The invention provides recombinant polynucleotides comprising a nucleic acid encoding a CYP76AD6 or related gene and their use for producing L-DOPA from tyrosine and treating dopamine-responsive disorders, such as Parkinson's Disease. The invention also provides recombinant polynucleotides comprising a nucleic acid encoding a CYP76AD1 and/or CYP76AD6, a nucleic acid encoding a DOPA 4,5-dioxygenase (DOD) enzyme, such as Beta vulgaris DODA1, and, in some cases, a nucleic acid encoding betalain related glucosyltransferase, such as M. jalapa gene cyclo-DOPA 5-O-glucosyltransferase (cDOPA5GT), and their use for producing betalains. Finally, the invention provides chimeric polypeptides, expression vectors, cells, compositions, and organisms, including plants, and their uses in various methods of the invention.

### CROSS REFERENCE TO RELATED APPLICATIONS

This application is a divisional of U.S. patent application Ser. No. 18/436,233 filed on Feb. 8, 2024, which is a continuation of U.S. patent application Ser. No. 16/940,666 filed on Jul. 28, 2020, which is a continuation of U.S. patent application Ser. No. 15/758,372 filed on Mar. 8, 2018, now U.S. Pat. No. 10,767,201, issued on Sep. 8, 2020, which is a National Phase of PCT Patent Application No. PCT/IL2016/051010 having an International filing date of Sep. 11, 2016, which claims the benefit of priority under 35 U.S.C. § 119(e) of Israel Patent Application No. 241462, filed on Sep. 10, 2015. The contents of the above applications are all incorporated by reference as if fully set forth herein in their entirety.

### REFERENCE TO AN ELECTRONIC SEQUENCE LISTING

The contents of the electronic sequence listing (PHYT-P-001-US2_corrected.xml; Size: 276,696 bytes; and Date of Creation: Apr. 1, 2024) is herein incorporated by reference in its entirety.

### FIELD OF THE INVENTION

The invention provides recombinant polynucleotides comprising a nucleic acid encoding a CYP76AD6 or related gene and their use for producing L-DOPA from tyrosine and treating dopamine-responsive disorders, such as Parkinson's Disease. The invention also provides recombinant polynucleotides comprising a nucleic acid encoding a CYP76AD1 and/or CYP76AD6, a nucleic acid encoding a DOPA 4,5-dioxygenase (DOD) enzyme, such as Beta vulgaris DODA1, and, in some cases, a nucleic acid encoding betalain related glucosyltransferase, such as M. jalapa gene cyclo-DOPA 5-O-glucosyltransferase (cDOPA5GT), and their use for producing betalains. Finally, the invention provides chimeric polypeptides, expression vectors, cells, compositions, and organisms, including plants, and their uses in various methods of the invention.

### BACKGROUND OF THE INVENTION

Betalains are tyrosine-derived, red-violet and yellow plant pigments found in only one group of angiosperms, the Caryophyllales order, in which they occur in a mutually exclusive fashion with the chemically distinct and widespread anthocyanin pigments. The betalain class contains a wide array of compounds, which are generally classified into two groups; the red-violet betacyanins, and the yellow betaxanthins.

A key enzyme in betalain biosynthesis, DOPA 4,5-dioxygenase (DOD) converts L-DOPA to betalamic acid, which constitutes the basic backbone of all betalains (FIG. 1). Spontaneous conjugation of betalamic acid with amines or with L-DOPA derivatives, results in the formation of yellow betaxanthins or red-violet betacyanins, respectively. Betalain related glucosyltransferase have also been characterized in several Caryophyllales plant species, catalyzing 5-O glucosylation of cyclo-DOPA or alternatively 5-O or 6-O glucosylation of betanidin. The enzyme catalyzing the formation of L-DOPA from tyrosine is unknown.

Due to their high stability, pH independence and antioxidative properties, betalains may be used as natural food colorants and dietary supplements.

While potential edible plant sources of anthocyanins are numerous, betalains are found in very few edible plants, with red beet being the only major source for betalain extraction in commercial use today. Despite its high betacyanin content, red beet extract has several drawbacks as a source of food colorants; it mainly produces betanin and thus has limited color variability, it carries adverse earthy flavors due to the occurrence of geosmin and various pyrazines, and it holds the risk of carry-over of soil-borne microbes. There are currently no natural sources in large-scale use for betaxanthins as food dyes. Yellow beet, for example, is not used likely due to co-occurring phenolics that are easily oxidized and mask the yellow hue of betaxanthins. Evidently, it is of interest to develop alternative sources for betalains, particularly betaxanthins, as there are currently no natural yellow, water-soluble pigments available for commercial use in the food industry. Heterologous production of betalains may provide numerous new viable sources for these pigments, such as plants, plant cell cultures, algae and yeast.

A betalain pathway intermediate, L-3,4-dihydroxyphenylalanine (L-DOPA), is also a commercially valuable metabolite that is widely used for treatment of Parkinson's disease.

Parkinson's disease is a progressive disorder of the nervous system primarily affecting the motor system of the body. It is the second most common neurodegenerative disorder and the most common movement disorder, affecting an estimate of 5 million people worldwide. A major feature of Parkinson's disease is the reduced levels of dopamine, an important signaling molecule in the nervous system. The most effective therapy for Parkinson's disease is the administration L-DOPA (3,4-dihydroxyphenylalanine), which is converted to dopamine in the brain. L-DOPA is the most effective drug for the treatment of Parkinson's disease, since dopamine fails to pass through the blood brain barrier.

L-DOPA is also widely marketed as a dietary supplement and is a precursor of additional high-value metabolites, which include among others catecholeamines (e.g. dopamine, epinephrine), benzilisoquinoline alkaloids (e.g. morphine and other opiates), betalain pigments and melanin.

Although L-DOPA is produced in many plants and animal species, it rarely accumulates in substantial quantities. This is partially due to the fact that L-DOPA is universally formed by the enzyme tyrosinase, which catalyzes the hydroxylation of tyrosine to L-DOPA but also immediately converts it to its oxidized form, dopaquinone.

L-DOPA for the pharma industry is currently produced using one of several methods, all of which have severe limitations, as chemical synthesis can only be achieved in a costly process that involves many chemical reactions and requires the use of expensive substrates and harsh production conditions. Biotechnological production of L-DOPA via the use of tyrosinase enzymes (also called polyphenol oxidase—PPO) has also been explored. The dual reaction of tyrosinase described above is highly problematic for the commercial production of L-DOPA, as the product of interest is directly metabolized to the commercially useless dopaquinone, and is therefore a major bottleneck for its use in enzymatic biosynthesis for large-scale production of L-DOPA from tyrosine. Thus, there is an unmet need for a more efficient and less expensive method for preparing L-DOPA.

Betalain biosynthesis has remained poorly understood in comparison to the other major classes of plant pigments, namely anthocyanins and carotenoids, especially in regard to the enzyme that catalyzes the conversion of tyrosine to L-DOPA in the betalain synthetic pathway in Caryophyllales.

### SUMMARY OF THE INVENTION

In one embodiment, the present invention provides a recombinant polynucleotide comprising a nucleic acid encoding a CYP76AD6 gene, a nucleic acid encoding CYP76AD15 gene, or a nucleic acid encoding a combination thereof under the control of a promoter. In one embodiment, the polynucleotide further comprises a nucleic acid encoding a DOPA 4,5-dioxygenase (DOD) enzyme.

In another embodiment, the present invention provides a recombinant polynucleotide comprising a nucleic acid encoding CYP76AD1, a nucleic acid encoding a DOPA 4,5-dioxygenase (DOD) enzyme, and a nucleic acid encoding betalain related glucosyltransferase, wherein said nucleic acids are inserted into the polynucleotide in frame. In one embodiment, the polynucleotide further comprises a nucleic acid encoding CYP76AD6.

In another embodiment, the present invention provides a composition comprising a recombinant polynucleotide as described hereinabove.

In another embodiment, the present invention provides an expression vector comprising a recombinant polynucleotide as described hereinabove.

In another embodiment, the present invention provides a cell comprising the expression vector as described hereinabove.

In another embodiment, the present invention provides a chimeric polypeptide encoded by a recombinant polynucleotide as described hereinabove.

In another embodiment, the present invention provides a method of producing L-DOPA in a cell comprising the step of contacting said cell with a recombinant polynucleotide comprising a nucleic acid encoding a CYP76AD6 gene, a nucleic acid encoding CYP76AD15 gene, or a nucleic acid encoding a combination thereof under the control of a promoter, under conditions sufficient to produce L-DOPA, thereby producing L-DOPA.

In another embodiment, the present invention provides a method of producing L-DOPA from tyrosine comprising the step of combining CYP76AD6, CYP76AD15, or a combination thereof, and tyrosine under conditions sufficient to produce L-DOPA, thereby producing L-DOPA.

In another embodiment, the present invention provides a use of a CYP76AD1-β clade gene or one or more cells from an organism comprising high levels of said CYP76AD1-β clade gene in the preparation of a composition for treating or suppressing a dopamine-responsive disorder in a subject.

In another embodiment, the present invention provides a method of betalain production comprising the step of contacting a recombinant polynucleotide comprising a nucleic acid encoding a CYP76AD6 gene, a nucleic acid encoding CYP76AD15 gene, or a nucleic acid encoding a combination thereof under the control of a promoter; a nucleic acid encoding a DOPA 4,5-dioxygenase (DOD) enzyme; and a nucleic acid encoding CYP76AD1, wherein said nucleic acids are inserted into the polynucleotide in frame, with one or more cells, under conditions sufficient to produce betalains, thereby producing betalains.

In another embodiment, the present invention provides a method of producing betaxanthins comprising the step of contacting a recombinant polynucleotide comprising a nucleic acid encoding a CYP76AD6 gene, a nucleic acid encoding CYP76AD15 gene, or a nucleic acid encoding a combination thereof under the control of a promoter; and a nucleic acid encoding a DOPA 4,5-dioxygenase (DOD) enzyme, wherein said nucleic acids are inserted into the polynucleotide in frame, with one or more cells, under conditions sufficient to produce betaxanthins, thereby producing betaxanthins.

In another embodiment, the present invention provides a method of increasing the resistance of a plant or plant part to one or more biotic or abiotic stress factors comprising the step of contacting one or more cells of said plant or plant part with a nucleic acid encoding CYP76AD6, a nucleic acid encoding CYP76AD15, a nucleic acid encoding CYP76AD1, or a combination thereof, a nucleic acid encoding a DOPA 4,5-dioxygenase (DOD) enzyme; and, optionally a nucleic acid encoding a betalain related glucosyltransferase, under conditions sufficient to produce betalains, thereby producing betalains and increasing the resistance of said plant or plant part to said one or more stress factors.

In another embodiment, the present invention provides a method of increasing the resistance of a plant or plant part to one or more fungal diseases comprising the step of contacting one or more cells of a plant or plant part with a nucleic acid encoding CYP76AD6, a nucleic acid encoding CYP76AD15, a nucleic acid encoding CYP76AD1, or a combination thereof, a nucleic acid encoding a DOPA 4,5-dioxygenase (DOD) enzyme; and, optionally a nucleic acid encoding a betalain related glucosyltransferase, under conditions sufficient to produce betalains, thereby producing betalains and increasing the resistance of said plant or plant part to said one or more fungal diseases.

In another embodiment, the present invention provides a method of increasing the levels of one or more betalains in an organism or in a part of an organism comprising causing or allowing within at least one part of the organism the expression of a DOPA 4,5-dioxygenase (DOD) enzyme and CYP76AD6, CYP76AD1, or a combination thereof, wherein if said organism expresses CYP76AD1 and DOD and not CYP76AD6 or CYP76AD15, said organism also expresses a betalain related glucosyltransferase, thereby increasing the levels of one or more betalains in said organism or in said part of an organism.

In another embodiment, the present invention provides an organism or part thereof comprising a recombinant nucleic acid sequence encoding a CYP76AD1-α clade gene, a nucleic acid sequence encoding a CYP76AD1-β clade gene, or a nucleic acid sequence encoding a combination thereof, and a nucleic acid sequence encoding a DOPA 4,5-dioxygenase (DOD) enzyme.

In another embodiment, the present invention provides a method of betalain production from tyrosine comprising the step of contacting a composition comprising CYP76AD1, a DOPA 4,5-dioxygenase (DOD) enzyme with tyrosine, and CYP76AD6, CYP76AD15, or a combination thereof, under conditions sufficient to produce betalains, thereby producing betalains.

In another embodiment, the present invention provides a method of producing betaxanthins from tyrosine comprising the step of contacting a composition comprising CYP76AD6, CYP76AD15, or a combination thereof, and a DOPA 4,5-dioxygenase (DOD) enzyme with tyrosine, under conditions sufficient to produce betaxanthins, thereby producing betaxanthins.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1. The betalain biosynthetic pathway. Genes taking part in the pathway are shown in magenta. Enzymatic reactions: EI, hydroxylation of tyrosine; EII, DOPA-4,5 dioxygenase; EIII, oxidation of DOPA; EIV, glucosylation of cyclo-DOPA; EV, glucosylation of betanidin; EVI, additional glucosylation of betacyanins; EVII, acylation of betacyanins. Predicted spontaneous reactions: SI, condensation (aldimine formation). Enzymatic reactions EII and EIII are followed by a spontaneous cyclization reaction and are therefore marked with an asterisk. EV can alternatively be catalyzed by a betanidin-6-O-glucosyltransferase, leading to formation of gomphrenin instead of betanin. Dashed lines designate reactions of an alternative pathway, which was shown to occur in Mirabilis jalapa, in which cyclo-DOPA is first glycosylated and then condensates with betalamic acid to form betanin.

FIGS. 2A-2G. Identification of MjCYP76 (SEQ ID NO: 1) as a betalain-related candidate gene. Gene expression in Mirabilis jalapa was analyzed in a transcriptome dataset derived from 24 tissues, including four floral organ types in five developmental stages, red or green stem epidermis, and red or green leaves. FIG. 2A: Petals. FIG. 2B: Stamen filaments. FIG. 2C: Stem epidermis from node or internode. FIG. 2D: Stigmas. FIG. 2E: Anthers. FIG. 2F: Red-young or mature-green leaf. FIG. 2G: Betalain-related genes cyclo-dopa-5-O-glucosyltransferase (cDOPA5GT; SEQ ID NO: 2), DOPA 4,5-dioxygenase (MjDOD; SEQ ID NO: 3) and the cytochrome P450 CYP76AD3 (Genbank Accession No. HQ656026.1; SEQ ID NO: 4) exhibit expression patterns which parallel pigment accumulation. The cytochrome P450 gene MjCYP76 (SEQ ID NO: 1) is highly co-expressed with cDOPA5GT (Pearson correlation r value=0.90) and to a lesser extent with MjDOD (r value=0.67).

FIG. 3. Gene expression patterns of putative polyphenol oxidase-encoding genes MjPPO1 (SEQ ID NO: 5); MjPPO2 (SEQ ID NO: 6); MjPPO3 (SEQ ID NO: 7); and MjPPO4 (SEQ ID NO: 8) in the Mirabilis jalapa RNA-seq dataset.

FIG. 4A. qRT-PCR analysis of four CYP76AD paralogs shows partial downregulation following infection with pTRV2:CYP76AD1 or pTRV2:CYP76AD1-CYP76AD6 vectors, suggesting off-target silencing. Upregulation of Bv20048 was observed in tissues infected with pTRV2:CYP76AD1-CYP76AD6. Relative quantification (RQ) values indicate means of three biological replicates±SE. Asterisks denote statistical significance of differential expression in comparison with non-silenced tissue. *, P<0.05; **, P<0.01.

FIG. 4B. Maximum-likelihood phylogenetic tree of CYP76AD paralogs in B. vulgaris. CYP76AD6 (GenBank accession KT962274; SEQ ID NO: 30); CYP76AD1 (accession AET43289.1; SEQ ID NO: 14); BvCYP76new (accession KU041699; SEQ ID NO: 9); Bv20048 (accession KU041700); Bv10427 (accession KU041701); Bv31911 (accession KU041702); Bv15899 (accession KU041703); Bv15885 (accession KU041704).

FIGS. 5A-5C. Co-silencing of CYP76AD1 and CYP76AD6 inhibits betalain production. Virus Induced Gene Silencing (VIGS) assays in red beet were used for silencing of BvDODA1 or CYP76AD1. CYP76AD1 was additionally co-silenced with each of the two new candidates BvCYP76new (SEQ ID NO: 9) and CYP76AD6. While silencing of CYP76AD1 alone or co-silencing with BvCYP76new blocks formation of betacyanins but not of betaxanthins, co-silencing of CYP76AD1 and CYP76AD6 prevents production of both types of pigments, resulting in a phenotype similar to the one obtained by silencing of BvDODA1. Reduction of betaxanthin levels is observed by a visible decrease in yellow color and lack of fluorescence under blue light, typical for betaxanthins. In FIG. 5A, whole-plant images, 3.5 weeks post infiltration are presented; in FIG. 5B, single-leaf images, bright field and in FIG. 5C, single-leaf images, blue light are shown.

FIG. 6A. Relative quantification of betaxanthin in VIGS-silenced beet leaves. Relative betaxanthin content was assessed spectrophotometrically by light absorption measurements at 475 nm and 600 nm. Values indicate the mean±SEM of four biological replicates, each consisting of gene-silenced tissue from two to three plants. Asterisks denote statistically significant difference from pTRV2:CYP76AD1. **, P value <0.01. RQ, relative quantification.

FIG. 6B. Quantitative real-time PCR (qRT-PCR) analysis of CYP76AD1 and CYP76AD6. CYP76AD1 is down-regulated in tissues infected with pTRV2:CYP76AD1 or pTRV2:CYP76AD1-CYP76AD6. CYP76AD6 is significantly down-regulated following infection with pTRV2: CYP76AD1-CYP76AD6. Relative quantification (RQ) values indicate means of three biological replicates±SE. Asterisks denote statistical significance of differential expression in comparison with noninfected plants. **, P-value <0.01.

FIGS. 7A-7F. Recombinant expression of CYP76AD1 or CYP76AD6 in Nicotiana benthamiana leaves enables L-DOPA and betalain production. FIG. 7A: Co-infiltration of agrobacteria harboring plasmids for expression of CYP76AD1 with cDOPA5GT (pAD1-GT) and BvDODA1 (pDODA) in Nicotiana benthamiana leaves causes red pigmentation. FIG. 7B: LC-MS analysis of red-pigmented tissue shows occurrence of betanin and iso-betanin. FIG. 7C: Co-infiltration of the CYP76AD6 expression vector (pAD6) and pDODA in N. benthamiana leaves causes yellow pigmentation. FIG. 7D: Infiltration of pAD1-GT in N. benthamiana leaves enables production of L-DOPA. L-DOPA was not detected in control experiment, where YFP is expressed (pYFP). FIG. 7E: Analysis of yellow-pigmented tissue allows the identification of several betaxanthin compounds, including indicaxanthin. FIG. 7F: Infiltration of pAD6 in N. benthamiana leaves enables production of L-DOPA. L-DOPA was not detected in control experiment (pDODA+pYFP). XIC, extracted ion chromatogram.

FIGS. 8A-8C. Cytochrome P450s CYP76AD1 and CYP76AD6 differ in activity and phylogeny.

FIG. 8A: Recombinant expression of CYP76AD1, CYP76AD6 and BvDODA1 in yeast cells. The image presented is of media in which yeast were grown following overnight galactose induction. Each yeast clone was transformed with three vectors, for expression of CYP76AD1, CYP76AD6, BvDODA1 and beta-glucoronidase (GUS) in different combinations, and grown in standard SD media without L-DOPA supplementation. Combined expression of BvDODA1 and CYP76AD1 resulted in red-violet pigmentation of the culture media, whereas expression of BvDODA1 together with CYP76AD6 led to formation of yellow pigmentation, indicative of the distinction in catalytic activity between CYP76AD1 and CYP76AD6. Expression of BvDODA1 with both cytochrome P450S resulted in orange-red pigmentation of the media. No pigmentation was observed when each of the cytochrome P450s or BvDODA1 was expressed alone.

FIG. 8B: Multiple sequence alignment of CYP76AD1-like protein sequences from betalain-producing Caryophyllales plants was processed into a maximum-likelihood phylogenetic tree, resulting in the formation of two separate clades, previously named alpha and beta clades (Brockington et al., 2015). CYP76AD6 falls in the β clade, while CYP76AD1 is positioned in the α clade. In the CYP76AD1-β clade: B. vulgaris, Beta vulgaris (CYP76AD6; GenBank accession KT962274; SEQ ID NO: 30); Bv. maritima, Beta vulgaris ssp. maritima (accession AKI33834.1; SEQ ID NO: 10); F. latifolia, Froelichia latifolia (accession AKI33838.1; SEQ ID NO: 11); A. caracasana, Alternanthera caracasana (accession AKI33835.1; SEQ ID NO: 12); A. ficoidea, Alternanthera ficoidea (accession AKI33831.1; SEQ ID NO: 13). In the CYP76AD1-a clade: B. vulgaris, Beta vulgaris (CYP76AD1; accession AET43289.1; SEQ ID NO: 14); A. cruentus, Amaranthus cruentus (CYP76AD2; accession AET43291.1; SEQ ID NO: 15); M. jalapa, Mirabilis jalapa (CYP76AD3; accession AET43292.1; SEQ ID NO: 16); C. cristata, Celosia cristata (CYP76AD4; accession AGI78466.1; SEQ ID NO: 17).

FIG. 8C: Liquid chromatography-mass spectrometry (LC-MS) analysis of pigmented yeast media revealed the occurrence of the betalamic acid chromophore when either CYP76AD1, CYP76AD6 or both were expressed with BvDODA1, in addition to several betaxanthins, including glutamine-betaxanthin and valinebetaxanthin (Table 2; not shown in chromatogram). Occurrence of the betacyanins betanidin and iso-betanidin was observed only where CYP76AD1 was expressed. No peaks corresponding to betalain compounds were found where BvDODA1 was not expressed. The y-axes (peak intensity) of all six chromatograms are linked. XIC, extracted ion chromatograms of masses corresponding to betalamic acid [M+H=212.05] and betanidin isomers [M+H=389.10]. Time, retention time (min).

FIG. 9. Alignment of the CYP76AD1 (SEQ ID NO: 14) and CYP76AD6 (SEQ ID NO: 18) protein sequences shows a 72 percent identity between them.

FIG. 10. Schematic of the pX11 overexpression vector (SEQ ID NO: 19). KanR, kanamycin resistance-conferring gene nptII; DODA1, B. vulgaris DOPA 4,5-dioxygenase; CYP76AD1, B. vulgaris cytochrome P450; cDOPA5GT, M. jalapa cyclo-DOPA-5-O-glucosyltransferase; nos, nopaline synthase promoter/terminator; 35S, CaMV 35S promoter/terminator; Act2, Arabidopsis actin 2 terminator; Ub10, Arabidopsis ubiquitin 10 promoter;

FIGS. 11A-11B. Production of betalains in multiple naturally-non-producing plant species.

FIG. 11A: Agrobacteria-mediated transformation of the pX11 vector, overexpressing cytochrome P450 CYP76AD1, DOPA 4,5-dioxygenase (BvDODA1) and cyclo-dopa 5-O-glucosyltransferase (cDOPA5GT), causes development of red-violet calli or shoots in a variety of Solanaceous plant species, typically observed within 1-2 wk of cultivation in tissue culture: Solanum lycopersicum (tomato), Solanum tuberosum (potato), Solanum melongena (eggplant), Nicotiana tabacum (tobacco), Nicotiana glauca (tree tobacco), Solanum nigrum (European black nightshade), Petunia x hybrida (Petunia), Nicotiana benthamiana.

FIGS. 11B1-11B2: Nicotiana glauca (FIG. 11B1) and Nicotiana benthamiana (FIG. 11B2) calli were sampled and analyzed with liquid chromatography-mass spectrometry (LC-MS), allowing the identification of the betacyanins betanin and isobetaninin in both species, in addition to a novel unidentified betacyanin compound, found only in the N. glauca tissue (betacyanin III; Table 2). XIC, extracted ion chromatograms of masses corresponding to the novel betacyanin [M+H=593.10] and betanin isomers [M+H=551.10]. Time, retention time (min).

FIGS. 12A-12C. Betacyanin identification and quantification in engineered and naturally-producing species.

FIG. 12A1-12A2: LC-MS analysis of Nicotiana benthamiana leaves agroinfiltrated with the pX11 vector enabled the identification of betanin, which exhibits the same accurate mass, retention time, fragmentation patterns and UV-VIS absorption spectra as betanin from red beet (Beta vulgaris) leaf extract.

FIG. 12B: Agroinfiltration of agrobacteria harbouring the pX11 vector into N. benthamiana leaves leads to intense red pigmentation in infiltrated area, typically observed within 2-3 days.

FIG. 12C: Betacyanin content of pX11-agroinfiltrated N. benthamiana and pX11-transformed Nicotiana tabacum was assessed in comparison to several betalain-producing species. Betacyanin content was assessed spectrophotometrically by light absorption measurements at 535 nm and 600 nm. Values indicate the mean±SE of the mean of four biological replicates, each consisting of 100 mg tissue (fresh weight).

FIGS. 13A-13E. Pigmentation phenotypes observed in transgenic tobacco plants engineered for betalain production. Three betalain pathway genes, namely, CYP76AD1, BvDODA1 and cDOPA5GT (in the pX11 binary vector) were heterologously expressed in Nicotiana tabacum, resulting in red pigmentation in different plant organs, including leaves, stems, roots and flowers. FIG. 13A: Left, wild-type tobacco; right, pX11-transformed tobacco. FIG. 13B: Top row, wild-type tobacco flower; bottom row, pX11 flower. Pigment accumulation was also evident in pX11 roots (FIG. 13C), stem epidermis section, ×20 magnified (FIG. 13D) and leaf glandular trichomes, ×20 magnified (FIG. 13E).

FIGS. 14A-14B. Betalain production in food crops tomato, potato and eggplant.

FIG. 14A: Transformation of the pX11 vector results in formation of red-pigmented plants in potato (Solanum tuberosum var. Desiree—top left panels), eggplant (Solanum melongena line DR2—bottom left panels) and tomato (Solanum lycopersicum var. MicroTom—right panels).

FIG. 14B: Betanin and isobetanin were identified as the major betacyanins in potato tuber, tomato fruit and eggplant fruit by LC-MS analysis. Extracted ion chromatogram (XIC) of betanin/isobetanin-corresponding mass [M+H=551.1] and UV-VIS absorption of betanin peak is shown for all three tissues.

FIG. 15. Schematic of the pX11 (SEQ ID NO: 19), pX11(E8) (SEQ ID NO: 20), pX11(CHS) (SEQ ID NO: 21), pX12 (SEQ ID NO: 22), and pX13 (SEQ ID NO: 23) overexpression vectors. KanR, kanamycin resistance-conferring gene nptII; DODA1, B. vulgaris DOPA 4,5-dioxygenase; CYP76AD1/CYP76AD6, B. vulgaris cytochrome P450; cDOPA5GT, M. jalapa cyclo-DOPA-5-O-glucosyltransferase; nos, nopaline synthase promoter/terminator; 35S, CaMV 35S promoter/terminator; Act2, Arabidopsis actin 2 terminator; Ub10, Arabidopsis ubiquitin 10 promoter; E8, S. lycopersicum E8 promoter; CHS, Petunia x hybrida chalcone synthase promoter.

FIGS. 16A-16B. Fruit-specific accumulation of betalains in tomato.

FIG. 16A: Introduction of the pX11(E8) vector into tomato (Solanum lycopersicum var. M-82) results in betalain pigmentation that is restricted to ripening and ripe fruit.

FIG. 16B: Whole fruit and cross-section of wildtype and pX11(E8) tomato fruit.

FIGS. 17A-17B. Color variation in flowers of transgenic betalain-producing tobacco is determined by varying betacyanin/betaxanthin ratios.

FIG. 17A: Introduction of pX11, pX12 or pX13 vectors into tobacco results in formation of different colored flowers. Viewed from top (top row) or magnified and viewed under bright-field (middle row) or blue light (bottom row), in which betaxanthins are typically fluorescent.

FIG. 17B: LC-MS analysis of pX11, pX12 and pX13 tobacco petals. Extracted ion chromatograms (XIC) of masses [M+H=309.1, 331.1, 340.1, 551.1], respectively corresponding to proline-betaxanthin, tyramine-betaxanthin, glutamine-betaxanthin, and betanin/isobetanin. Vertical axes are linked.

FIGS. 18A-18B. Production of betalains in tobacco BY2 cells.

FIG. 18A: Introduction of pX11 or pX13 into cells of tobacco BY2 line results in red-violet or yellow-orange pigmentation, respectively.

FIG. 18B: LC-MS analysis of pX11 and pX13 BY2 cells. Extracted ion chromatograms (XIC) of masses [M+H=283.1, 340.1, 551.1], respectively corresponding to alanine-betaxanthin, glutamine-betaxanthin, and betanin/isobetanin. Vertical axes are linked.

FIG. 19. Schematic of the pDOPA1-pDOPA4 (SEQ ID NOs: 24-27) overexpression vectors. nptII, kanamycin resistance-conferring gene neomycin phosphotransferase H; CYP76AD6, B. vulgaris cytochrome P450; AroG, E. coli AroG175 (DAHPS) mutant; AAH, Physcomitrella patens aromatic amino acid hydroxylase; 35S, CaMV 35S promoter/terminator; Ub10, Solanum lycopersicum ubiquitin 10 promoter.

FIGS. 20A-20C. Production of L-DOPA in tobacco plants and BY2 cells. FIG. 20A: Identification of L-DOPA in leaf extracts of pDOPA1 and pDOPA3-expressing tobacco plants by LC-MS analysis. FIG. 20B: pDOPA2 and pDOPA4 expression in BY2 cells causes blackening of calli in varying degrees, after several weeks of cultivation. FIG. 20C: L-DOPA relative quantification in pDOPA2, pDOPA4 and wildtype BY2 cells by LC-MS analysis.

FIGS. 21A-21B. Seed germination assays of betalain-producing tobacco under osmotic and salinity stress conditions. FIG. 21A: Wildtype and pX11 tobacco seedlings grown under high salinity stress conditions (150 mM NaCl) and control conditions (MS) were photographed and scored for seed germination rates. FIG. 21B: Wildtype and pX11 tobacco seedlings grown under osmotic stress conditions (400 mM mannitol) and control conditions (MS) were photographed and scored for seed germination rates.

FIGS. 22A-22C. Botrytis cinerea resistance assays of betalain-producing tobacco plants. FIG. 22A: Leaves of wildtype and pX11 tobacco plants were infected with droplets of B. cinerea suspension and photographed 5 days post infection. FIG. 22B: Wildtype and pX11 plants infected with a total of approximately 500 B. cinerea spores per plant were scored for lesion size. Average lesion areas are shown of approximately 30 plants per genotype. FIG. 22C: Infected leaves of wildtype tobacco plants showed increased signs of necrosis versus leaves of pX11 plants.

FIGS. 23A-23D. Recombinant expression of CYP76AD15 in Nicotiana benthamiana enables betaxanthin and L-DOPA production. FIG. 23A: Co-infiltration of agrobacteria harboring plasmids for expression of CYP76AD15 and BvDODA1 in Nicotiana benthamiana leaves causes yellow pigmentation in infiltrated area. FIG. 23B: LC-MS analysis of yellow-pigmented tissue shows occurrence of several betaxanthins, including dopamine-betaxanthin [M+H=347.1] and valine-betaxanthin [M+H=311.1]. XIC, extracted ion chromatogram. FIG. 23C: UV-VIS absorption of peaks representing dopamine-betaxanthin and valine-betaxanthin in analyzed tissue. FIG. 23D: Mass-spectra of L-DOPA peak identified in leaf tissue of N. benthamiana expressing CYP76AD15 shows typical L-DOPA fragments [M+H=181.05, 152.07] and molecular ion [M+H=198.07].

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In one embodiment, the present invention provides compositions comprising an enzyme that catalyzes the first step in betalain synthesis in Caryophyllales and uses of the enzyme in genetic engineering of plants and for the production of L-DOPA from tyrosine. In general, tyrosinases are substrate promiscuous oxygenases that exhibit unwanted catechol oxidase activity. A prior study demonstrated that the plant cytochrome P450 CYP76AD1 exhibited this characteristic promiscuity and despite a screening strategy to identify mutations that increased the enzyme's substrate specificity and activity, still produced undesired side products (DeLoache et al., 2015; Nat Chem Biol. 2015 July; 11(7):465-71, incorporated herein by reference in its entirety). It was therefore not expected that a different plant cytochrome P450 would be specific to tyrosine and would catalyze only the reaction to L-DOPA, thereby avoiding the accumulation of undesired end products.

### CYP76AD6

The present invention provides a novel gene (belonging to the Cytochrome P450 gene family) which catalyzes tyrosine hydroxylation, to form L-DOPA. This enzyme potentially holds a significant advantage over the tyrosinase enzyme which is being used today for production of L-DOPA; while tyrosinase catalyzes the formation of L-DOPA from tyrosine, and immediately oxidizes it to the unusable metobolite dopaquinone, the novel P450 enzyme only catalyzes the formation of L-DOPA without its oxidation. This P450 enzyme can therefore be used for a more efficient method for L-DOPA production than the use of a tyrosinase enzyme.

Thus, in one embodiment, the present invention provides a novel enzyme which can be used for the biosynthetic production of L-DOPA, a pharmaceutical used for treatment of Parkinson's disease, and a precursor for the economically important drugs benzylisoquinoline alkaloids (e.g. morphine). This enzyme can potentially be used for the commercial production of L-DOPA in several ways; its encoding gene can be expressed in plants or microorganisms to induce production of L-DOPA in vivo, or alternatively it can be recombinantly expressed and used for in vitro enzymatic catalysis.

### Methods for L-DOPA and Betalain Production

Production from Proteins

In one embodiment, the methods of the present invention are performed in vitro, such that the tyrosine and enzymes required to produce L-DOPA or betalains are provided in vitro.

In one embodiment, the present invention provides a method of producing L-DOPA from tyrosine comprising the step of combining a CYP76AD1-β clade protein and tyrosine under conditions sufficient to produce L-DOPA, thereby producing L-DOPA.

In one embodiment, the present invention provides a method of producing L-DOPA from tyrosine comprising the step of combining CYP76AD6, CYP76AD15, or a combination thereof, and tyrosine under conditions sufficient to produce L-DOPA, thereby producing L-DOPA.

In another embodiment, the present invention provides a method of producing L-DOPA from tyrosine in vitro comprising the step of combining CYP76AD6, CYP76AD15, or a combination thereof, and tyrosine under conditions sufficient to produce L-DOPA, thereby producing L-DOPA.

In another embodiment, the present invention provides a method of betalain production from tyrosine comprising the step of contacting tyrosine with CYP76AD6, CYP76AD1, or a combination thereof, under conditions sufficient to produce betalains, thereby producing betalains. In one embodiment, the method further comprises the step of contacting the tyrosine and CYP76AD6, CYP76AD15, or a combination thereof, CYP76AD1, or a combination thereof with a DOPA 4,5-dioxygenase (DOD) enzyme.

In another embodiment, the present invention provides a method of betalain production from tyrosine comprising the step of contacting tyrosine with a CYP76AD1-β clade protein and a DOPA 4,5-dioxygenase (DOD) enzyme and, optionally, CYP76AD1, under conditions sufficient to produce betalains, thereby producing betalains.

In another embodiment, the present invention provides a method of betalain production from tyrosine comprising the step of contacting tyrosine with CYP76AD6, CYP76AD15, or a combination thereof, and a DOPA 4,5-dioxygenase (DOD) enzyme and, optionally, CYP76AD1, under conditions sufficient to produce betalains, thereby producing betalains.

In another embodiment, the present invention provides a method of producing a mixture of betalains comprising the step of contacting tyrosine with CYP76AD6, CYP76AD15, or a combination thereof, CYP76AD1, a DOPA 4,5-dioxygenase (DOD) enzyme and a betalain related glucosyltransferase, under conditions sufficient to produce a combination of betacyanin and betaxanthin, thereby producing a mixture of betalains.

In one embodiment, the betalain production described in the present invention is performed without provision of L-DOPA, which had never before been accomplished. In one embodiment, betalains comprise betacyanin and betaxanthin. In one embodiment, betalains, betacyanin, and betaxanthin may be used as food coloring or other dyes.

In one embodiment, betacyanins produce a red-violet color. In one embodiment, betaxanthins produce a yellow color. In one embodiment, a combination of betacyanin and betaxanthin produce an orange color. In one embodiment, a combination of betacyanin and betaxanthin produce an orange-pink color.

In another embodiment, the present invention provides a method of producing an orange dye comprising the step of contacting tyrosine with CYP76AD6, CYP76AD15, or a combination thereof, CYP76AD1, a DOPA 4,5-dioxygenase (DOD) enzyme and a betalain related glucosyltransferase, under conditions sufficient to produce a combination of betacyanin and betaxanthin, thereby producing an orange dye.

In another embodiment, the present invention provides a method of producing betaxanthins from tyrosine comprising the step of contacting tyrosine with CYP76AD6, CYP76AD15, or a combination thereof, and a DOPA 4,5-dioxygenase (DOD) enzyme, under conditions sufficient to produce betaxanthins, thereby producing betaxanthins.

In another embodiment, the present invention provides a method of producing a yellow dye comprising the step of contacting tyrosine with CYP76AD6, CYP76AD15, or a combination thereof and a DOPA 4,5-dioxygenase (DOD) enzyme under conditions sufficient to produce betaxanthins, thereby producing a yellow dye. In one embodiment, said yellow dye is a food coloring.

In one embodiment, the methods of the present invention produce one or more betaxanthins. In one embodiment, the betaxanthin comprises Vulgaxanthin I (glutamine-betaxanthin). In another embodiment, the betaxanthin comprises Indicaxanthin (proline-betaxanthin). In another embodiment, the betaxanthin comprises Dopaxanthin-hexoside. In another embodiment, the betaxanthin comprises Betaxanthin I (unknown), as described in Table 2 herein below. In another embodiment, the betaxanthin comprises histamine-betaxanthin. In another embodiment, the betaxanthin comprises alanine-betaxanthin. In another embodiment, the betaxanthin comprises dopamine-betaxanthin. In another embodiment, the betaxanthin comprises valine-betaxanthin.

In another embodiment, the present invention provides a method of producing betacyanins from tyrosine comprising the step of contacting tyrosine with CYP76AD1, a DOPA 4,5-dioxygenase (DOD) enzyme, and a betalain related glucosyltransferase under conditions sufficient to produce betacyanins, thereby producing betacyanins.

In another embodiment, the present invention provides a method of producing red-violet dye comprising the step of contacting tyrosine with CYP76AD1, a DOPA 4,5-dioxygenase (DOD) enzyme, and a betalain related glucosyltransferase under conditions sufficient to produce betacyanins, thereby producing a red-violet dye. In one embodiment, said red-violet dye is a food coloring.

In one embodiment, the method as described herein provides a glycosylated betalain (betanin) rather than an aglycone (betanidin). In one embodiment, there is a substantial advantage in that betanin is more stable than betanidin. Betanidin is a highly labile compound and ascorbic acid must be added to prevent its degradation. Betanidin is much more susceptible to both enzymatic and non-enzymatic oxidative degradation. The betalain extract used for food coloration comprises betanin. Betanidin would not be usable for food coloration, because of its instability.

In another embodiment, the methods of the present invention produce one or more betacyanins. In one embodiment, the betacyanins comprise betanin. In another embodiment, the betacyanins comprise iso-betanin. In another embodiment, the betacyanins comprise betacyanin I (unknown) or betacyanin II (unknown), as described in Table 2 herein below. In another embodiment, the methods of the present invention produce one or more betacyanin fragments, where, in one embodiment, the betacyanin fragment is betanidin.

In another embodiment, the present invention provides a method of producing a pigment or dye comprising the step of contacting tyrosine with CYP76AD6, CYP76AD15, or a combination thereof, and a DOPA 4,5-dioxygenase (DOD) enzyme and, optionally, CYP76AD1, under conditions sufficient to produce betalains, thereby producing betalains, thereby producing a pigment or dye.

In another embodiment, the present invention provides a method of increasing the levels of one or more betalains in an organism, plant or in a plant part comprising causing or allowing the expression of a DOPA 4,5-dioxygenase (DOD) enzyme, a betalain related glucosyltransferase, and CYP76AD6, CYP76AD15, or a combination thereof, CYP76AD1, or a combination thereof, within said organism, plant or plant part, thereby increasing the levels of one or more betalains in said organism, plant or plant part. In one embodiment, said plant is an ornamental plant. In another embodiment, said plant is a food crop. In one embodiment, the organism or plant does not naturally produce detectable levels of betalains.

Production from Nucleic Acids

In another embodiment, the methods of the present invention are performed in vivo. In one embodiment, the tyrosine is endogenous to the cell. In another embodiment, the tyrosine is provided to the cell. In another embodiment, the cell is transformed with a gene that enhances tyrosine availability. In one embodiment, the gene is AroG175 (Tzin et al., 2012 New Phytologist 194: 430-439; Genbank Accession No. JC233128.1, SE) ID NO: 28), aromatic amino acid hydroxylase (AAH) (Pribat et al., 2010 Plant Cell 22: 3410-3422; Genbank Accession No. HQ003815.1, SEQ ID NO: 29), or a combination thereof. In one embodiment, the enzymes required to produce betalains are provided to a cell via transfer of a polynucleotide encoding the enzymes.

In one embodiment, the present invention provides a method of L-DOPA production comprising the step of contacting an organism with a nucleic acid encoding a CYP76AD1-β clade gene under conditions sufficient to produce L-DOPA, thereby producing L-DOPA.

In another embodiment, the present invention provides a method of L-DOPA production comprising the step of contacting an organism with a nucleic acid encoding CYP76AD6, CYP76AD15, or a combination thereof, under conditions sufficient to produce L-DOPA, thereby producing L-DOPA.

In another embodiment, the present invention provides a method of L-DOPA production comprising the step of contacting an organism with a nucleic acid encoding CYP76AD6, CYP76AD15, or a combination thereof, under conditions sufficient to produce L-DOPA, thereby producing L-DOPA.

In one embodiment, the organism is a plant. In one embodiment, the plant is a tobacco plant.

In another embodiment, the present invention provides a method of betalain production comprising the step of contacting an organism with a nucleic acid encoding a CYP76AD1-β clade gene, a nucleic acid encoding a CYP76AD1-α clade gene, or a combination thereof, under conditions sufficient to produce betalains, thereby producing betalains.

In another embodiment, the present invention provides a method of betalain production comprising the step of contacting an organism with a nucleic acid encoding CYP76AD6, CYP76AD15, or a combination thereof, a nucleic acid encoding CYP76AD1, or a combination thereof, under conditions sufficient to produce betalains, thereby producing betalains.

In one embodiment, the method further comprises the step of contacting the organism comprising tyrosine with a nucleic acid encoding a DOPA 4,5-dioxygenase (DOD) enzyme.

In one embodiment, the nucleic acid encoding genes disclosed in the present invention, including, inter alia, CYP76AD6, CYP76AD15, or a combination thereof, CYP76AD1, and DOD are present together on a single recombinant polynucleotide, as described in more detail herein below.

In another embodiment, the present invention provides a method of betalain production comprising the step of contacting an organism with a nucleic acid encoding CYP76AD6, CYP76AD15, or a combination thereof, a nucleic acid encoding CYP76AD1, a nucleic acid encoding a DOPA 4,5-dioxygenase (DOD) enzyme, and a nucleic acid encoding a betalain related glucosyltransferase under conditions sufficient to produce betalains, thereby producing betalains.

In another embodiment, the present invention provides a method of producing orange dye comprising the step of contacting an organism comprising tyrosine with a nucleic acid encoding CYP76AD6, CYP76AD15, or a combination thereof, a nucleic acid encoding CYP76AD1, a nucleic acid encoding a DOPA 4,5-dioxygenase (DOD) enzyme, and a nucleic acid encoding a betalain related glucosyltransferase, under conditions sufficient to produce betalains, thereby producing orange dye.

In another embodiment, the betalain production described in the present invention is performed without substrate feeding.

In another embodiment, the present invention provides a method of producing betaxanthins comprising the step of contacting an organism comprising tyrosine with a nucleic acid encoding CYP76AD6, CYP76AD15, or a combination thereof, a nucleic acid encoding a DOPA 4,5-dioxygenase (DOD) enzyme, and a nucleic acid encoding a betalain related glucosyltransferase under conditions sufficient to produce betaxanthins, thereby producing betaxanthins.

In another embodiment, the present invention provides a method of producing yellow dye comprising the step of contacting an organism comprising tyrosine with a nucleic acid encoding CYP76AD6, CYP76AD15, or a combination thereof, a nucleic acid encoding a DOPA 4,5-dioxygenase (DOD) enzyme under conditions sufficient to produce betaxanthins, thereby producing yellow dye.

In another embodiment, the present invention provides a method of producing betacyanins comprising the step of contacting an organism comprising tyrosine with a nucleic acid encoding CYP76AD1, a nucleic acid encoding a DOPA 4,5-dioxygenase (DOD) enzyme, and a nucleic acid encoding a betalain related glucosyltransferase under conditions sufficient to produce betacyanins, thereby producing betacyanins. In one embodiment, the method of producing betacyanins is a method of producing predominantly betacyanins.

In another embodiment, the present invention provides a method of producing red-violet dye comprising the step of contacting an organism comprising tyrosine with a nucleic acid encoding CYP76AD1, a nucleic acid encoding a DOPA 4,5-dioxygenase (DOD) enzyme, and a nucleic acid encoding a betalain related glucosyltransferase under conditions sufficient to produce betacyanins, thereby producing red-violet dye.

In one embodiment, the methods described hereinabove provide methods of producing a natural red-violet dye. In another embodiment, the methods described hereinabove provide methods of producing a natural yellow dye. In another embodiment, the methods described hereinabove provide methods of producing a natural orange dye. In one embodiment, the dyes may be used for textiles.

In one embodiment, the organism is a plant. In another embodiment, the organism is yeast. In another embodiment, the organism is a bacterium. In another embodiment, the organism is an alga.

In one embodiment, the methods of the present invention are conducted in planta. In one embodiment, the color of the pigment in the plant is dependent upon other natural or engineered pigments expressed in the plant. Thus, in one embodiment, the method of the present invention produce a red pigment, a yellow pigment, an orange pigment, a brown pigment, and the like, or any of the pigment colors that are known in the art.

It is also to be understood that by varying the ratio of CYP76AD6, CYP76AD15, or a combination thereof, and/or CYP76AD1 expression, it is possible to change the pigment color as well.

In another embodiment, the present invention provides a method of betalain production from tyrosine comprising the step of contacting tyrosine with a nucleic acid encoding CYP76AD6, CYP76AD15, or a combination thereof, a nucleic acid encoding a DOPA 4,5-dioxygenase (DOD) enzyme, and, optionally, a nucleic acid encoding CYP76AD1, under conditions sufficient to produce betalains, thereby producing betalains.

In another embodiment, the present invention provides a method of producing orange dye comprising the step of contacting tyrosine with a nucleic acid encoding CYP76AD1, a nucleic acid encoding CYP76AD6, CYP76AD15, or a combination thereof, and a nucleic acid encoding a DOPA 4,5-dioxygenase (DOD) enzyme, under conditions sufficient to produce betalains, thereby producing orange dye.

In another embodiment, the present invention provides a method of producing betaxanthins from tyrosine comprising the step of contacting tyrosine with a nucleic acid encoding CYP76AD6, CYP76AD15, or a combination thereof, and a nucleic acid encoding a DOPA 4,5-dioxygenase (DOD) enzyme, under conditions sufficient to produce betaxanthins, thereby producing betaxanthins.

In another embodiment, the present invention provides a method of producing yellow dye comprising the step of contacting tyrosine with a nucleic acid encoding CYP76AD6, CYP76AD15, or a combination thereof, and a nucleic acid encoding a DOPA 4,5-dioxygenase (DOD) enzyme under conditions sufficient to produce betaxanthins, thereby producing yellow dye.

In another embodiment, the present invention provides a method of producing betacyanins from tyrosine comprising the step of contacting tyrosine with a nucleic acid encoding CYP76AD1 and a nucleic acid encoding a DOPA 4,5-dioxygenase (DOD) enzyme, under conditions sufficient to produce betacyanins, thereby producing betacyanins.

In another embodiment, the present invention provides a method of producing red-violet dye comprising the step of contacting tyrosine with a nucleic acid encoding CYP76AD1, a nucleic acid encoding a DOPA 4,5-dioxygenase (DOD) enzyme, and a nucleic acid encoding a betalain related glucosyltransferase, under conditions sufficient to produce betacyanins, thereby producing red-violet dye.

In one embodiment, a dye of the present invention is food coloring. In one embodiment, methods of the present invention for producing dyes may be applied to producing pigments, and the like.

In one embodiment, the present invention provides a food coloring, dye, or pigment produced by the methods of the present invention or comprising the compositions of the present invention.

In another embodiment, the present invention provides a method of producing L-DOPA in a cell comprising the step of contacting said cell with a polynucleotide comprising a nucleic acid encoding CYP76AD6, CYP76AD15, or a combination thereof, under conditions sufficient to produce L-DOPA, thereby producing L-DOPA.

In another embodiment, the present invention provides a method of producing betalains, orange dye, or a combination thereof in a cell comprising the step of contacting said cell with a nucleic acid encoding CYP76AD6, CYP76AD15, or a combination thereof, a nucleic acid encoding CYP76AD1, or a combination thereof, under conditions sufficient to produce betalains, thereby producing betalains, orange dye, or a combination thereof.

In another embodiment, the present invention provides a method of producing betaxanthins, yellow dye, or a combination thereof in a cell comprising the step of contacting said cell with a nucleic acid encoding CYP76AD6, CYP76AD15, or a combination thereof, and a nucleic acid encoding a DOPA 4,5-dioxygenase (DOD) enzyme, under conditions sufficient to produce betaxanthins, thereby producing betaxanthins, yellow dye, or a combination thereof.

In another embodiment, the present invention provides a method of producing betacyanins, red-violet dye, or a combination thereof in a cell comprising the step of contacting said cell with a nucleic acid encoding CYP76AD1, a nucleic acid encoding a DOPA 4,5-dioxygenase (DOD) enzyme, and a nucleic acid encoding a betalain related glucosyltransferase, under conditions sufficient to produce betacyanins, thereby producing betacyanins, red-violet dye, or a combination thereof.

In one embodiment, the cell described hereinabove is part of a cell line.

In one embodiment, betalains obtained by the methods of the present invention, including betaxanthins, betacyanins, or a combination thereof, are produced in a cell line. In one embodiment, the cell line is a plant cell line. In one embodiment, the plant cell line is tobacco BY2 or arabidopsis T87.

In another embodiment, L-DOPA obtained by the methods of the present invention is produced in a cell line. In one embodiment, the cell line is a tobacco cell line. In one embodiment, the tobacco cell line is BY2.

In another embodiment, the present invention provides a method of producing a metabolite of L-DOPA comprising the step of producing L-DOPA by contacting a cell with a nucleic acid encoding CYP76AD6, CYP76AD15, or a combination thereof, under conditions sufficient to produce L-DOPA, and either allowing formation of the L-DOPA metabolite or further contacting said cell with a nucleic acid encoding an enzyme that metabolizes L-DOPA, thereby producing a metabolite of L-DOPA. In one embodiment, the L-DOPA metabolite is a catecholeamine, benzilisoquinoline alkaloid, betalain, melanin, or a combination thereof. In one embodiment, the catecholeamine is dopamine, norepinephrine, or a combination thereof. In one embodiment, the benzilisoquinoline alkaloid is an opiate, which in one embodiment is morphine.

In one embodiment, the enzyme that metabolizes L-DOPA is a DOPA 4,5-dioxygenase (DOD). In another embodiment, the enzyme is CYP76AD1. In another embodiment, the enzyme is a glucosyltransferase. In one embodiment, the glucosyltransferase is cyclo-DOPA 5-O-glucosyltransferase or betanidin-5-O-glucosyltransferase. In one embodiment, the cyclo-DOPA 5-O-glucosyltransferase is M. jalapa gene cyclo-DOPA 5-O-glucosyltransferase (cDOPA5GT).

In another embodiment, the enzyme that metabolizes L-DOPA is DOPA decarboxylase. In another embodiment, the enzyme that metabolizes L-DOPA is dopamine beta-hydroxylase. In another embodiment, the enzyme that metabolizes L-DOPA is aromatic L-amino acid decarboxylase. In another embodiment, the enzyme that metabolizes L-DOPA is catechol-O-methyl transferase. In another embodiment, the enzyme that metabolizes L-DOPA is phenylethanolamine N-methyltransferase.

In another embodiment, the enzyme that metabolizes L-DOPA is norcoclaurine synthetase (NCS). In another embodiment, the enzyme that metabolizes L-DOPA is norcoclaurine 6-O-methyltransferase (6OMT). In another embodiment, the enzyme that metabolizes L-DOPA is coclaurine-N-methyltransferase (CNMT). In another embodiment, the enzyme that metabolizes L-DOPA is 3′-hydroxy-N-methylcoclaurine 4′-O-methyltransferase (4′OMT).

### Methods of Genetic Transformation

In one embodiment, the present invention provides methods comprising the step of “contacting” a cell with a polynucleotide or expression vector as described herein.

In one embodiment, contacting comprises transforming a cell with the nucleic acid molecule or construct of the invention. Methods for transforming a plant cell with nucleic acids sequences are well known in the art. As used herein the term “transformation” or “transforming” may refer to a process by which a foreign DNA, such as a DNA construct, including expression vector, enters and changes a recipient cell into a transformed, genetically modified or transgenic cell. Transformation may be stable, wherein the nucleic acid sequence is integrated into the plant genome and as such represents a stable and inherited trait, or transient, wherein the nucleic acid sequence is expressed by the cell transformed but is not integrated into the genome, and as such represents a transient trait.

In one embodiment, the present invention provides a transgenic plant. In one embodiment, a transgenic plant of the present invention is genetically modified with foreign or heterologous genes. In one embodiment, transgenic plants of the present invention are used for biofuel. In another embodiment, transgenic plants of the present invention are food crop plants.

In another embodiment, the present invention provides a cisgenic plant. In one embodiment, a cisgenic plant of the present invention is genetically modified, but contains no foreign or heterologous genes. According to this aspect and in one embodiment, betalain enzymes may be overexpressed in plants already comprising betalain enzymes, thereby changing the ratio between betacyanins and betaxanthins. In one embodiment, food crops of the present invention are cisgenic.

Any method or delivery system may be used for the delivery and/or transformation (plant cells)/transfection (algae cells) of the nucleic acid vectors encoding CYP76AD6 and homologs, paralogs, etc. in the host cell, e.g., plant protoplast. The vectors may be delivered to the host cell either alone, or in combination with other agents. Transient expression systems may also be used. Homologous recombination may also be used.

In one embodiment, polynucleotides as described herein are provided to a cell of the present invention via transformation. Transformation may be accomplished by a wide variety of means, as is known to those of ordinary skill in the art. Such methods include, but are not limited to, particle bombardment mediated transformation (e.g., Finer et al., 1999, Curr. Top. Microbiol. Immunol., 240:59), protoplast electroporation (e.g., Bates, 1999, Methods Mol. Biol., 111:359), viral infection (e.g., Porta and Lomonossoff, 1996, Mol. Biotechnol. 5:209), microinjection, and liposome injection. Other exemplary delivery systems that can be used to facilitate uptake by a cell of the nucleic acid include calcium phosphate and other chemical mediators of intracellular transport, microinjection compositions, and homologous recombination compositions (e.g., for integrating a gene into a preselected location within the chromosome of the cell). Alternative methods may involve, for example, the use of liposomes, electroporation, or chemicals that increase free (or “naked”) DNA uptake, transformation using viruses or pollen and the use of microprojection. Standard molecular biology techniques are common in the art (e.g., Sambrook et al., 1989, Molecular Cloning: A Laboratory Manual, 2nd ed., Cold Spring Harbor Laboratory Press, New York).

### Plant Transformation

There are various methods of introducing foreign genes into both monocotyledonous and dicotyledonous plants (See Potrykus I 1991. Annu Rev Plant Physiol Plant Mol Biol 42, 205-225; Shimamoto K. et al., 1989. Nature 338, 274-276). Transformation methods may include, for example, but are not limited to, the use of liposomes, electroporation, chemicals that increase free DNA uptake, injection of the DNA directly into the plant, particle gun bombardment, transformation using viruses and microprojection.

In one embodiment, polynucleotides as described herein are provided to a cell via agroinfiltration, in one embodiment, via Agrobacterium-mediated transformation (e.g., Komari et al., 1998, Curr. Opin. Plant Biol., 1:161), including floral dip transformation. In one embodiment agroinfiltration induces transient expression of genes in a plant to produce a desired protein, by injected a suspension of Agrobacterium tumefaciens containing the desired gene or genes into a plant leaf. In one embodiment, the transformation can be performed by an Agrobacterium-mediated gene transfer. The Agrobacterium-mediated system includes the use of plasmid vectors that contain defined DNA segments which integrate into the plant genomic DNA. Methods of inoculation of the plant tissue vary depending upon the plant species and the Agrobacterium delivery system. The transformation can be performed with any suitable tissue explant that provides a good source for initiation of whole-plant differentiation (See Horsch et al., 1988. Plant Molecular Biology Manual A5, 1-9, Kluwer Academic Publishers, Dordrecht).

Plant transformation methods are fully described in U.S. Patent Application Publications US 20110209247; US 20110113514; US 20100199371; US 20070079396; US 20080307541; US 20030028913; and US20030196219; and U.S. Pat. Nos. 5,015,580; 5,550,318; 5,538,880; 6,160,208; 6,399,861; 6,403,865; 5,635,055; 5,824,877; 5,591,616; 5,981,840 and 6,384,301, which are incorporated by reference herein in their entirety.

For the Agrobacterium tumefaciens-based plant transformation system, additional elements present on transformation constructs will, in one embodiment, include T-DNA left and right border sequences to facilitate incorporation of the recombinant polynucleotide into the plant genome.

In one embodiment, the transformation can be performed by a direct DNA uptake. There are various methods of direct DNA transfer into plant cells. In electroporation, the protoplasts are briefly exposed to a strong electric field, opening up mini-pores to allow DNA to enter. In microinjection, the DNA is mechanically injected directly into the cells using micropipettes. In microparticle bombardment, the DNA is adsorbed on microprojectiles such as magnesium sulfate crystals or tungsten particles, and the microprojectiles are physically accelerated into cells or plant tissues.

In another embodiment, polynucleotides as described herein are provided to a cell via viral transformation (transduction) using a suitable plant virus, using gene gun techniques or electroporation.

In one embodiment, the heterologous genes of the polynucleotide of the present invention are integrated into the plant chromosome. In another embodiment, the heterologous genes of the polynucleotide of the present invention remain extrachromosomal. In one embodiment, the heterologous genes are in a plasmid within the cell. Plasmids useful for either application are known to one skilled in the art.

In one embodiment, DNA is inserted randomly, i.e. at a non-specific location, in the genome of a target plant line. In another embodiment, DNA insertion is targeted in order to achieve site-specific integration, e.g. to replace an existing gene in the genome, to use an existing promoter in the plant genome, or to insert a recombinant polynucleotide at a predetermined site known to be active for gene expression. Several site specific recombination systems exist which are known to function in plants including cre-lox and FLP-FRT.

In one embodiment, transformation methods of this invention are practiced in tissue culture on media and in a controlled environment. “Media” refers to the numerous nutrient mixtures that are used to grow cells in vitro, that is, outside of the intact living organism. Recipient cell targets include, but are not limited to, meristem cells, callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Cells capable of proliferating as callus are also recipient cells for genetic transformation. Practical transformation methods and materials for making genetically modified plants of this invention, e.g. various media and recipient target cells, transformation of immature embryos and subsequent regeneration of fertile genetically modified plants are known in the art.

In another embodiment in which polynucleotides are introduced into plant cells, the betalains may be expressed or detected in any plant organ, including leaves, stems, roots, flowers, seeds, tuber, or fruit, or a combination thereof.

In another embodiment, the methods of the present invention may be performed in a whole plant, such that the whole plant expresses the betalains as described herein. In addition and in one embodiment, the compositions of the present invention may describe a whole plant that has been genetically modified to express a polynucleotide or polypeptide of the present invention.

### Algae Transformation

In one embodiment, the method of transformation of algae comprises any of the methods as described hereinabove. In one embodiment, transformation of algae is accomplished using glass bead-assisted transformation, particle gun-mediated (biolistic) transformation, treatment with cellulolytic enzymes to weaken their cell walls, or homologous recombination.

In another aspect, the nucleic acids of the present invention can be transformed into algae. In one embodiment, the alga is a single cell alga. In another embodiment, the alga is a multi-cellular alga. In one embodiment, the alga is a cyanobacterium, diatom, chlamydomonas, Dunaliella, or hematococus. The genes can be over expressed in algae. Betalain, DOPA, or a combination thereof can be produced through such transgenic algae. Method for algae transformation are well known in the art and fully described in U.S. Patent Application Publications US 20150011008; US 20150004704; US 20130130389; US 20120094385; US 20120034698; US 20110300633; and US 20040133937, which are incorporated by reference herein in their entirety.

In one embodiment, the genetically modified algae of the present invention may be used in biofuel production.

### Yeast Transformation

In another aspect, the nucleic acids of the present invention can be transformed into yeast. The genes can be over expressed in yeast. Betalain, DOPA, or a combination thereof can be produced through such transgenic yeast. Method for yeast transformation are described herein below and are well known in the art and fully described in U.S. Patent Application Publications US 20090264320; US 20010031724; US 20030049785; US 20050158861; US 20070264716; US 20090325247; and US 20100190223, which are incorporated by reference herein in their entirety.

### Viral Transformation

In another embodiment, the nucleic acids of the present invention can be transformed into a virus. In another embodiment, the nucleic acids may be over-expressed in a virus. Betalain, dyes, pigments, DOPA, or a combination thereof can be produced through such viral over expression.

### Markers of Genetic Transformation

In one embodiment, DNA is introduced into only a small percentage of target cells in any one experiment.

In one embodiment, cells comprising the transferred exogenous DNA may be identified by color, as described in Example 6 for yeast. In one embodiment, cells expressing CYP76AD1 and DODA are red-violet. In one embodiment, cells expressing CYP76AD6, CYP76AD15, or a combination thereof, and DODA are yellow. In one embodiment, cells expressing CYP76AD1, CYP76AD6, CYP76AD15, or a combination thereof, and DODA are orange or orange-red.

In another embodiment, marker genes are used to provide an efficient system for identification of those cells that are stably transformed by receiving and integrating a genetically modified DNA construct into their genomes.

In another embodiment, selection genes provide selective markers that confer resistance to a selective agent, such as an antibiotic or herbicide. Potentially transformed cells are exposed to the selective agent. In the population of surviving cells will be those cells where, generally, the resistance-conferring gene has been integrated and expressed at sufficient levels to permit cell survival. Cells may be tested further to confirm stable integration of the exogenous DNA.

Useful selective marker genes include those conferring resistance to antibiotics such as kanamycin (nptII), hygromycin B (aph IV), nptII, hpt, aadA and gentamycin (aac3 and aacC4) or resistance to herbicides such as glufosinate (bar or pat) and glyphosate (EPSPS). In another embodiment, the selection gene is an anti metabolite. In one embodiment, the antimetabolite is dhf.

Screenable markers which provide an ability to visually identify transformants can also be employed, e.g., a gene expressing a colored or fluorescent protein such as a cat, hacZ, uidA, luciferase or green fluorescent protein (GFP) or a gene expressing a beta-glucuronidase or uidA gene (GUS) for which various chromogenic substrates are known. It is also contemplated that combinations of screenable and selectable markers will be useful for identification of transformed cells.

In one embodiment, the selection gene is a positive selectable marker gene that is conditional on non-toxic agents that may be substrates for growth or that induce growth and differentiation of the transformed tissues.

In one embodiment, cells that survive exposure to the selective agent, or cells that have been scored positive in a screening assay, may be cultured in regeneration media and allowed to mature into plants. Developing plantlets can be transferred to soil less plant growth mix, and hardened off, e.g., in an environmentally controlled chamber at about 85% relative humidity, 600 ppm CO₂, and 25-250 microeinsteins m⁻²s⁻¹ of light, prior to transfer to a greenhouse or growth chamber for maturation. Plants are preferably matured either in a growth chamber or greenhouse. Plants are regenerated from about 6 weeks to 10 months after a transformant is identified, depending on the initial tissue. During regeneration, cells are grown to plants on solid media at about 19 to 28° C. After regenerating plants have reached the stage of shoot and root development, they may be transferred to a greenhouse for further growth and testing. Plants may be pollinated using conventional plant breeding methods known to those of skill in the art and seed produced.

Progeny may be recovered from transformed plants and tested for expression of the exogenous recombinant polynucleotide. Useful assays include, for example, “molecular biological” assays, such as Southern and Northern blotting and PCR; “biochemical” assays, such as detecting the presence of RNA, e.g. double stranded RNA, or a protein product, e.g., by immunological means (ELISAs and Western blots) or by enzymatic function; plant part assays, such as leaf or root assays; and also, by analyzing the phenotype of the whole regenerated plant.

One of skill in the art will be able to select an appropriate vector for introducing the encoding nucleic acid sequence in a relatively intact state. Thus, any vector which will produce a host cell, e.g., plant protoplast, carrying the introduced encoding nucleic acid should be sufficient. The selection of the vector, or whether to use a vector, is typically guided by the method of transformation selected.

### Promoters

In another aspect, cell, tissue, or organ specific promoters can be used to transform the genes of the invention and make them express in a specific cell, tissue, or organ. For example, fruit specific promoter can be used to transform the genes of the invention into a plant (e.g., tomato) or plant cell in order to produce betalains in fruits (e.g., tomato fruits).

In one embodiment, a recombinant polynucleotide of the present invention comprises at least one promoter. In one embodiment, a recombinant polynucleotide of the present invention comprises a single promoter controlling the expression of the tandem nucleic acids that are in the same reading frame. In another embodiment, each nucleic acid in the recombinant polynucleotide of the present invention is under the control of a separate promoter.

In one embodiment, the promoter is a constitutive promoter. In one embodiment, the constitutive promoter is the CaMV 35S promoter. In another embodiment, the constitutive promoter is an opine promoter. In another embodiment, the constitutive promoter is a monocot promoter. In another embodiment, the constitutive promoter is a Plant ubiquitin promoter (Ubi). In another embodiment, the constitutive promoter is the Arabidopsis ubiquitin-10 promoter. In another embodiment, the constitutive promoter is the Solanum lycopersicum ubiquitin 10 promoter (SlUb10). In another embodiment, the constitutive promoter is Rice actin 1 promoter (Act-1). In another embodiment, the constitutive promoter is Maize alcohol dehydrogenase 1 promoter (Adh-1).

In another embodiment, the promoter is an inducible promoter. In one embodiment, the inducible promoter is a galactose-inducible promoter. In another embodiment, the inducible promoter is selected from AlcR/AlcA (ethanol inducible); GR fusions, GVG, and pOp/LhGR (dexamethasone inducible); XVE/OlexA (beta-estradiol inducible); and heat shock induction. In another embodiment, the inducible promoter is selected from tetracycline, dexamethasone, copper, salicyclic acid and herbicide inducible promoters. In another embodiment, mGal4:VP16/UAS or pOp/LhG4 may be used for transactivation, and the alc-switch, GVE/VGE, and XVE systems may be used for chemical induction. These methods are known in the art.

In another embodiment, the promoter is a tissue-specific or a development-stage-specific promoter. In another embodiment, the promoter is a synthetic promoter, which is produced by bringing together the primary elements of a promoter region from diverse origins.

In some embodiments, a fruit specific promoter can be used to transform the genes of the invention in combination with one or more other genes (e.g. anthocyanin genes) into a plant (e.g., tomato) or plant cell in order to produce betalains in fruits (e.g., tomato fruits). In one embodiment, the genes of the invention can be transformed into a transgenic plant (e.g., transgenic tomato plant already transformed with recombinant anthocyanin genes) or plant cell in order to produce betalains in fruits (e.g., tomato fruits).

Thus, in one embodiment, a promoter for any of the polynucleotides described herein, may be a fruit-specific promoter. In one embodiment, the fruit-specific promoter is the E8 promoter. In another embodiment, the promoter may be a flower-specific promoter. In one embodiment, the flower-specific promoter is a chalcone synthase (CHS) promoter. In one embodiment, the CHS promoter is a Petunia x hybrida CHS promoter. In another embodiment, the promoter may be a root-specific promoter. In another embodiment, the promoter may be a stem-specific promoter. In another embodiment, the promoter may be a leaf-specific promoter. In another embodiment, the promoter may be a seed-specific promoter. In another embodiment, the promoter may be a tuber-specific promoter.

In one embodiment, the promoter may be specific to a part of the tissue. For example and in one embodiment, a promoter may be specific to the petal, stamen, anther, stigma, or a combination thereof.

In another embodiment, the same nucleic acid may be expressed under different non-constitutive promoter sequences to engineer an organism that exhibits two colors of pigmentation and/or expresses both betacyanins and betaxanthins in different organs or in different parts of the same organ. For example, in one embodiment, CYP76AD6 or CYP76AD15 or a homolog thereof may be expressed under a flower-specific promoter and CYP76AD1 may be expressed under a fruit-specific promoter so that the fruit is red-purple due to the presence of both betacyanins and betaxanthins, and the flower is yellow due to the presence of betaxanthins only. In addition, inducible promoters may be used to engineer an organism that exhibits two colors of pigmentation and/or expresses both betacyanins and betaxanthins in different organs or in different parts of the same organ.

In another embodiment, the promoter may be specific to a developmental stage. For example and in one embodiment, a promoter may be specific to Stage 1, which, in one embodiment, is when the flower length is approximately 1 cm. In another embodiment, the developmental stage is Stage 2, which, in one embodiment, is when the flower length is approximately 2 cm. In another embodiment, the developmental stage is Stage 3, which, in one embodiment, is when the flower length is approximately 3 cm. In another embodiment, the developmental stage is Stage 4, which, in one embodiment, is when the flower length is approximately 4 cm. In another embodiment, the developmental stage is Stage 5, which, in one embodiment, is when the flower length is approximately 5-6 cm.

In one embodiment, the promoter may be specific to a tissue and a developmental stage. In one embodiment, the fruit specific E8 promoter is expressed in ripening and ripe fruit. In another embodiment, the flower-specific CHS promoter is expressed in petals during flower maturation.

### Gene Silencing Applications

In one embodiment, the present invention provides a method of treating or suppressing a dopamine-responsive disorder in a subject comprising the step of administering a food crop, cell, or cell line comprising high levels of a CYP76AD1-β clade polypeptide, thereby providing said subject with L-DOPA, thereby treating or inhibiting said dopamine-responsive disorder in said subject. In one embodiment, said food crop, cell, or cell line endogenously produces betalains. In one embodiment, the expression of DOPA 4,5-dioxygenase, cyclo-DOPA 5-O-glucosyltransferase, and a CYP76AD1-alpha clade gene in said food crop, cell, or cell line has been suppressed.

In one embodiment, the present invention provides a method of producing yellow plant parts in a plant comprising betalains, comprising silencing CYP76AD1 gene expression.

In one embodiment, the present invention provides a method of producing green plant parts in a plant comprising betalains, comprising silencing CYP76AD1 and CYP76AD6, CYP76AD15, or a combination thereof, gene expression.

In one embodiment, the plant parts comprise leaves, stems, roots, flowers, tuber, fruit, seeds, or a combination thereof.

In one embodiment, gene silencing is gene knockdown, which in one embodiment, is a reduced expression of the gene. In one embodiment, virus induced gene silencing is used to silence the genes encoding CYP76AD1 and/or CYP76AD6. In one embodiment, virus-induced gene silencing (VIGS) is a technology that exploits an RNA-mediated antiviral defense mechanism. In plants infected with unmodified viruses the mechanism is specifically targeted against the viral genome. However, with virus vectors carrying inserts derived from host genes the process can be additionally targeted against the corresponding mRNAs. Such methods are exemplified herein below in Examples 1 and 3.

Other methods of gene silencing that may be used in the present invention include gene silencing with antisense oligonucleotides, ribozymes, RNA interference, three prime untranslated regions/microRNAs. Such methods are well known in the art as well as being described in Example 1 herein below.

In another embodiment, the genes encoding CYP76AD1 and/or CYP76AD6, CYP76AD15, or a combination thereof, are knocked out using methods known in the art.

In one embodiment, the plant comprises betalains, which in one embodiment, is a betacyanin, and in another embodiment, is a betaxanthin.

In one embodiment, methods of gene silencing are performed in plants comprising betalains. In one embodiment, plants comprising betalains are from the Caryophyllales plant order.

In one embodiment, methods of gene silencing are performed in Caryophyllales which comprises cacti, carnations, amaranths, ice plants, beets, and many carnivorous plants. In another embodiment, the plant is from the Caryophyllineae suborder. In another embodiment, the plant is from the Polygonineae suborder. In another embodiment, the plant is from a family selected from one of the following families of the Caryophyllales plant order: Achatocarpaceae; Aizoaceae; Amaranthaceae; Anacampserotaceae; Ancistrocladaceae; Asteropeiaceae; Barbeuiaceae; Basellaceae; Cactaceae; Caryophyllaceae; Didiereaceae; Dioncophyllaceae; Droseraceae; Drosophyllaceae; Frankeniaceae; Gisekiaceae; Halophytaceae; Kewaceae; Limeaceae; Lophiocarpaceae; Macarthuriaceae; Microteaceae; Molluginaceae; Montiaceae; Nepenthaceae; Nyctaginaceae; Physenaceae; Phytolaccaceae; Plumbaginaceae; Polygonaceae; Portulacaceae; Rhabdodendraceae; Sarcobataceae; Simmondsiaceae; Stegnospermataceae; Talinaceae; and Tamaricaceae.

In some embodiments, polynucleotides of the present invention are prepared using PCR techniques using procedures and methods known to one skilled in the art. In some embodiments, the procedure involves the legation of two different DNA sequences (See, for example, “Current Protocols in Molecular Biology”, eds. Ausubel et al., John Wiley & Sons, 1992).

### Polynucleotides

In one embodiment, the present invention provides a recombinant polynucleotide comprising a nucleic acid encoding CYP76AD6. In one embodiment, the nucleic acid sequence encoding CYP76AD6 comprises:

(SEQ ID NO: 30)
tcgatgtgttttcaagaagagcttgagcctacattctacaacaatatatt
gttcctctcttgcgcaattaagcccttaaacctgcaattatcttagaatt
tttgggtttctaatttattactcctttaattagctagatatagattattc
atatttttccagcattttcaaacaatggataacgcaacacttgctgtgat
cattccattttgtttgtgttttaccacattttcaaatcctattcaccaat
tatcatacgtaggatcctcctggtcccaaacccgtgccaatttttggcaa
cattttcgatcttggcgaaaagcctcatcgatctgccaatctatctaaaa
ttcacggccattgattagcctaaagttaggaagtgtaacaactattgttg
tttcctcggcctagtggccgaggaaatgttccttaaaaatgaccaagcac
ttgctaaccgaaccattcctgactcggttagggctggtgaccacgacaaa
ttatccatgtcgtggttgcctgtttcccaaaaatggagaaatatgagaaa
aataccgctgtccaattactaccaaccaaaaacttgatgctagtcaacct
cttagacaagctaaggtgaaacaacttttatcatacgtacaagtttgttc
cgaaaaaatgcaacccgtcgatattggacgggccgcatttacaacgtcac
ttaatttattatcaaacacatttttctcaatcgaattagcaagtcatgaa
tctagtgatcccaagagtttaaacaactcatgtggaatattatggaggaa
attggaaggcctaattatgctgattttttccctattcttggttacattga
tccattggtataagacgtcgtttggctggttactttgataaactcattga
tgattccaagacattattcgtgaaagacaaaagatcgatcttctaattct
tccggcgcaaaacaaacaaatgacattcttgatactatcttaaactccat
gaagataatgagttgagtatgcctgaaattaatcaccttctcgtggatat
ctttgacgccggaacagacacaacagcaagcacattagaatgggcgatgg
ccgaacttgtgaaaaacccggaaatgatgactaaagttcaaattgaaatc
gaacaagctcttggaaaagattgcttagacatacaagaatccgacatctc
aaaactaccttatttacaagccattataaaagaaacgttacgtttacacc
ctcctactgtgtttttgctgcctcgaaaggcagacaatgacgtagagtta
tatggctacgttgtaccaaagaatgctcaagtccttgtcaatctttgggc
aattggtcgtgatccaaaggtatggaaaaatccggaagtattttacctga
aaggtttttagattgcaatatcgattataaaggacgagatttcgaacttt
taccattggtgctggtagaaggatatgccaggacttactttggcatatag
aatgttgaacttgatgttggctactatcttcaaaactacaattggaaact
tgaagatggtatcaatcctaaggatttagacatggatgagaaatttggga
ttacattgcaaaaggttaaacctcttcaagttattccagttcccagaaac
tagctagtgggttgtgccgttgtgggctgtgggctgattgtgcactgttt
cctctaattgttgttgcaaacttctctagaagggtattatcttttgtata
aataaagcgaaagctacatgtcctattattaaattagtgtatactatatt
aagtaagtatgagcatatagtatccattattgatttggttgaatcgtatt
aaactgataaatcaagtaaatgtctccaacctaagcattcaattattctg
ttagtcataacatatgtagagggtaagtatgaagaattaataacggcgtg
atcatcctta.

In another embodiment, the coding sequence of CYP76AD6 comprises the following sequence:

(SEQ ID NO: 31)
atggataacgcaacacttgctgtgatcattccattttgtttgtgttttac
cacattttcaaatcctttttcaccaattcttcatctcgtaggcttcctcc
tggtcccaaacccgtgccaatttttggcaacattttcgatatggcgaaaa
gcctcatcgatatttgccaatctatctaaaattcacggccctttgattag
cctaaagttaggaagtgtaacaactattgttgtttcctcggcctagtggc
cgaggaaatgttccttaaaaatgaccaagcacttgctaaccgaaccattc
ctgactcggttagggctggtgaccacgacaaattatccatgtcgtggttg
cctgtttcccaaaaatggagaaatatgagaaaaatctccgctgtccaatt
actaccaaccaaaaacttgatgctagtcaacctatagacaagctaaggtg
aaacaacttttatcatacgtacaagtttgttccgaaaaaatgcaacccgt
cgatattggacgggccgcatttacaacgtcacttaatttattatcaaaca
catttttctcaatcgaattagcaagtcatgaatctagtgatcccaagagt
ttaaacaactcatgtggaatattatggaggaaattggaaggcctaattat
gctgattttttccctattatggttacattgatccattggtataagacgtc
gtttggctggttactttgataaactcattgatgttttccaagacattatt
cgtgaaagacaaaagatcgatcttctaattcttccggcgcaaaacaaaca
aatgacattcttgatactcttcttaaactccatgaagataatgagttgag
tatgcctgaaattaatcaccttctcgtggatatctttgacgccggaacag
acacaacagcaagcacattagaatgggcaatggccgaacttgtgaaaaac
ccggaaatgatgactaaagttcaaattgaaatcgaacaagctatggaaaa
gattgatagacatacaagaatccgacatacaaaactaccttatttacaag
ccattataaaagaaacgttacgtttacaccctcctactgtgtttttgctg
cctcgaaaggcagacaatgacgtagagttatatggctacgttgtaccaaa
gaatgctcaagtccttgtcaatattgggcaattggtcgtgatccaaaggt
atggaaaaatccggaagtattttacctgaaaggtttttagattgcaatat
cgattataaaggacgagatttcgaacttttaccattggtgctggtagaag
gatatgccaggacttactttggcatatagaatgttgaacttgatgttggc
tactatcttcaaaactacaattggaaacttgaagatggtatcaatcctaa
ggatttagacatggatgagaaatttgggattacattgcaaaaggttaaac
ctatcaagttattccagttcccagaaactag.

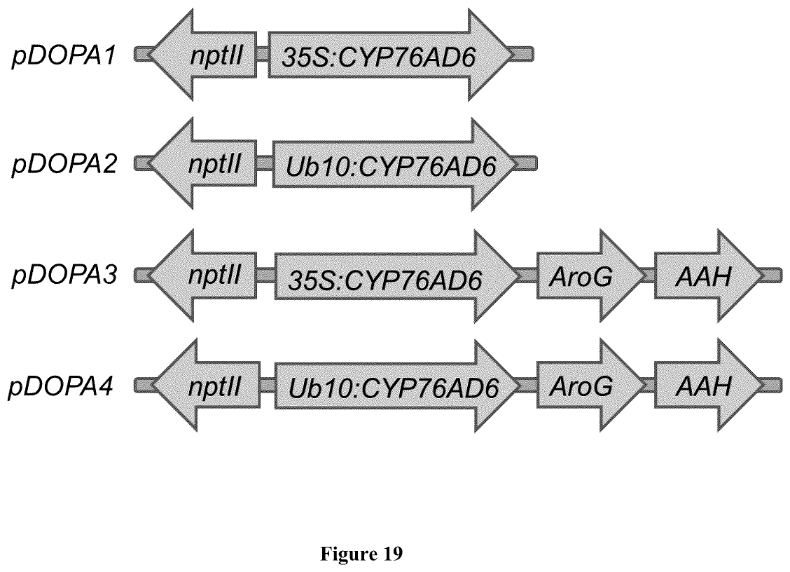

In one embodiment, the nucleic acid encoding CYP76AD6 is driven by the CaMV 35S promoter (pDOPA1; FIG. 19). In another embodiment, the nucleic acid encoding CYP76AD6 is driven by the Solanum lycopersicum ubiquitin 10 promoter (SlUb10) (pDOPA2; FIG. 19).

In one embodiment, the nucleic acid encoding CYP76AD6, CYP76AD15, or a combination thereof, is expressed together with AroG175 and aromatic amino acid hydroxylase (AAI).

In one embodiment, the polynucleotide comprising the nucleic acid encoding CYP76AD6, CYP76AD15, or a combination thereof, also comprises the neomycin phosphotransferase II (nptII) gene, which in one embodiment, confers kanamycin resistance.

In another embodiment, the present invention provides a recombinant polynucleotide comprising a nucleic acid encoding a CYP76AD1-α clade gene. In one embodiment, the CYP76AD1-α clade gene comprises CYP76AD1. In one embodiment, the nucleic acid sequence encoding CYP76AD1 comprises:

(SEQ ID NO: 32)
tactaatgtgcaaatgcataaatttaatttcagatactcttattataaat
attattatactgtgcattatttaacccaaaaaaaaaactgtcttaataca
ccatttaattcctttctctaggatccactatcacatgtagtatataaata
taatcctatacagttcacgttatcaaacaccaaagcatcaaaagccttcc
acacttgtattattttggggtagttgatttgttagcgtgttatttgtgag
atcatcatggatcatgcaacattagcaatgatactagccatttggttcat
ttcttttcatttcataaaattactttttagccaacaaactaccaaacttc
ttcctcctggtccaaaaccattgccaataattggtaacatcttagaagtt
ggtaaaaaaccccatcgttcatttgctaatcttgctaaaattcacggccc
tttaatatcgttacgtctaggaagtgtaacaactattgttgtatcatcag
cagatgtagctaaagaaatgttcttaaaaaaagaccaccctattctaacc
gtactattcctaattctgtcacggccggtgaccaccataaactcaccatg
tcgtggttgcccgtttcgccgaaatggcggaattttcgtaagattacagc
cgtccatttgattctcctcaaagacttgatgatgccaaacctttcgccat
gccaaggtgcaacagctttatgaatatgtacaagaatgtgcacaaaaagg
ccaagctgttgatattggcaaagctgcatttactacctcccttaatttgt
tatctaaactattcttttcagtggaattagcccaccataaatcacacact
tacaagagtttaaggaactaatatggaacattatggaagatattgggaaa
cctaattatgctgattattttcctattttaggctgtgttgatccatcagg
tattcgtcgaagattagcatgtagttttgacaagttgattgcagtttttc
agggtataatatgtgaaaggcttgcgcctgattatcaactacaacaacaa
cgacgactgatgatgtgctagacgttatcttcagacttcaaacaaaatga
gacactatgggcgagattaaccatttgacgtcgacatttttgatgctgga
acagacactacatcaagtacttttgagtgggtcatgacagagttaattag
gaatcctgaaatgatggaaaaggctcaagaagaaattaagcaagtattgg
gcaaagataaacaaattcaggaatcagacattattaacctaccttactta
caagccattatcaaagaaactttgcgactacatccaccaactgtatttct
tttgcctcgtaaagccgacactgatgttgaactatatggttatattgtgc
ctaaagatgcacaaatacttgttaacttatgggctattggaagagatcct
aatgcatggcaaaatgctgatattttttcgcccgaaagatttatagggtg
tgaaattgatgtcaaaggaagagattttggactcttaccttttggagccg
gaagaaggatatgtcctgggatgaatttggccattagaatgttaactttg
atgctagctactttacttcaattcttcaattggaagcttgaaggagacat
aagtccaaaagacttagacatggatgagaaatttgggattgcgttacaaa
agacaaagcctttaaaacttattccaatacctaggtattgaaatttgtta
gacttacgtacaacaattattattgtttgtggttggttttgggttagctt
cctgttcatgtttgtttgatgtgacgattgaatttactacaaatcaacga
actccaattatatcttgtcaacttggagtaatggcttgggtttctcatct
gtcatctcttttggttggctgtgcaagggagtgagcttgcagcttagcat
ccttcaagaagatttaattaataatatgtgtatgtgtatgtgtatgtgtg
gattgtatacgtcattcattttgcttgttgtaatgattttagttaattat
tgtaattataaaggatttgatcctcattgtgttgaaattatatatataat
gtgtagcacgacaataaagtgacagataacttatataatcttattttcta
tgaaatgttacagacttgattgtgttttaaaaaaa.

In another embodiment, the CYP76AD1-α clade gene comprises A. cruentus, Amaranthus cruentus (CYP76AD2, accession AET43291.1; SEQ ID NO: 15); M. jalapa, Mirabilis jalapa (CYP76AD3, accession AET43292.1; SEQ ID NO: 16); C. cristata, Celosia cristata (CYP76AD4, accession AGI78466.1; SEQ ID NO: 17); or a combination thereof.

In one embodiment, the present invention provides a recombinant polynucleotide comprising a nucleic acid encoding a DOPA 4,5-dioxygenase (DOD) enzyme. In one embodiment, the DOD is Beta vulgaris DODA1 (BvDODA1). In one embodiment, the nucleic acid sequence encoding BvDODA1 comprises:

(SEQ ID NO: 33)
aaaacaagaagaaaacaaaacaaccttttatataactagaaagcaacaaa
aaaaaaagaatgaaaatgatgaatggtgaagatgctaatgatcaaatgat
caaagaaagcttcttcataacacatggaaacccaatattaacagtagaag
acacacatccattaagacctttctttgaaacttggagagagaaaatcttt
tctaagaaacctaaggcaattcttattatttctggtcattgggaaactgt
taaacctactgttaatgctgtccatatcaatgatactatccatgattttg
atgactatcctgctgctatgtaccagttcaagtatccagctcctggggaa
ccagaattggcaagaaaagtagaggaaattctgaaaaaatcgggtttcga
aacggcggaaactgatcaaaaacgtgggcttgatcatggtgcatgggtac
ctctaatgctaatgtatcctgaggctgatataccagtatgtcagctctca
gttcagccgcatttagatggaacataccattacaacttaggacgagcatt
ggctcccttgaaaaacgacggcgtattaatcattggttcaggaagtgcaa
ctcaccctttggatgaaactcctcattattttgatggagttgcaccttgg
gcagctgcctttgattcatggcttcgtaaagctctcattaatggaaggtt
tgaagaagtgaatatatatgaaagcaaagcaccaaattggaaattagcac
atcctttcccagaacatttttatccattgcatgttgttcttggcgctgct
ggtgaaaaatggaaggcagagcttattcatagcagttgggatcatggcac
cttgtgtcatggctcctacaagttcacttcagcctagttttacttttaaa
cactatgtctacagtctatgttattgtatttggtaatttggtatgtggtg
ttgtttttgttttattcttttgagttattgtacttggtatttggtgttgt
tttcatttggtgcataattcttttagtctatatattgctattcttttgaa
tgaggaataaatcgcgagctcgatgtaatagtttgttttattgtttcaaa
tctatcattatatatatatatatattattaaaaaaaaattattgtgttac
tcgcaaaaaaa.

In one embodiment, the DOD is not Mirabilis jalapa DOD.

In one embodiment, the present invention provides a recombinant polynucleotide comprising a nucleic acid encoding a betalain related glucosyltransferase. In one embodiment, the nucleic acid sequence encoding the betalain related glucosyltransferase is the nucleic acid sequence encoding cyclo-DOPA 5-O-glucosyltransferase (cDOPA5GT). In one embodiment, cDOPA5GT is from Mirabilis Jalapa. In one embodiment, the nucleic acid sequence encoding cDOPA5GT comprises:

(SEQ ID NO: 2)
cctcacctccataacaaagaaatgaccgccattaaaatgaacaccaatgg
tgaaggtgaaacacaacatatactaatgatacctttcatggcgcaagggc
atttacgtcctttccttgagcttgctatgtttctatataaacgaagtcat
gttatcattactcttcttactaccccgctcaatgcgggtttcctacgaca
tctccttcaccaccatagctattctagctcggggatcagaattgtcgagt
tacctttcaactcaaccaatcatggtcttccacctggcattgaaaacact
gataaacttacactcccacttgtagtatcactattcattcaaccatttct
cttgaccctcaccttagagattatatttcccgccatttctcccctgcgcg
ccctcctctgtgtgtcatacatgatgtgttccttggttgggttgatcaag
ttgctaaagacgtgggctcaactggtgttgtttttactacgggtggcgcg
tatggtacaagcgcatatgtgtccatttggaatgatctgcctcaccagaa
ttactctgatgatcaagagtttccgcttcctggtttcccggagaatcata
aattccgacgttctcaacttcatcggtttctgaggtatgccgatggatca
gatgattggtcgaaatatttccaaccgcaattgaggcaatcaatgaagag
ttttggatggctatgtaattcagttgaggaaatcgaaacacttgggttta
gtatcctcaggaactacactaaactacccatttggggtattggaccgttg
atagcttcacctgtacaacattcatcatctgataataacagtactggtgc
cgagtttgttcaatggttgagcttgaaagaaccagattctgtattataca
tctcatttggatcacagaacacaatttcaccaacccagatgatggaacta
gcagccggtttggagtcaagtgagaagccgtattgtgggtgattcgagca
ccatttgggttcgatatcaatgaggaaatgagaccagaatggctaccaga
gggattcgaggagcgaatgaaggtgaaaaaacaaggaaagttggtgtata
agttgggaccacagttggagatacttaaccatgagtcaatcggagggttc
ttaactcattgtgggtggaattcgatccttgagtcacttcgagaaggtgt
gcctatgttagggtggccattggcagccgaacaagcttataatttgaagt
atttggaggacgaaatgggggttgcagtcgagttagcgaggggattggaa
ggagagataagtaaagagaaagtgaagagaattgtggagatgattttaga
gagaaatgaaggaagtaaaggatgggaaatgaaaaatagagcagtagaaa
tggggaagaaacttaaagacgctgtcaatgaggagaaggaactgaagggt
tcttctgttaaggcaatagatgatttcttagatgcggtcatgcaagctaa
gttggaaccttctcttcaataataagatcgatagtttatctcgtctcaga
gactcaaacaattgactactaggatgatcagaaagagagttatgttcagt
gttctgttattatgcaagacttcaataataacagaaaaattcgtgattag
attcataactt.

In one embodiment, several nucleic acids are combined into a single recombinant polynucleotide as described herein. In one embodiment, the present invention provides a recombinant polynucleotide comprising a nucleic acid encoding CYP76AD6, CYP76AD15, or a combination thereof, and a nucleic acid encoding DOD. In another embodiment, the present invention provides a recombinant polynucleotide comprising a nucleic acid encoding CYP76AD1 and a nucleic acid encoding DOD. In another embodiment, the present invention provides a recombinant polynucleotide comprising a nucleic acid encoding CYP76AD1 and a nucleic acid encoding CYP76AD6. In another embodiment, the present invention provides a recombinant polynucleotide comprising a nucleic acid encoding CYP76AD1, a nucleic acid encoding CYP76AD6, CYP76AD15, or a combination thereof, and a nucleic acid encoding DOD.

In another embodiment, several nucleic acids are incorporated into a cell, but each nucleic acid is a separate polynucleotide as described herein.

In one embodiment, a polynucleotide of the present invention comprises a nucleic acid sequence encoding CYP76AD6, CYP76AD15, or a combination thereof, and a nucleic acid sequence encoding CYP76AD1. In another embodiment, a polynucleotide of the present invention comprises a nucleic acid sequence encoding CYP76AD6, CYP76AD15, or a combination thereof, and a nucleic acid sequence encoding a DOD enzyme. In another embodiment, a polynucleotide of the present invention comprises a nucleic acid sequence encoding CYP76AD1 and a nucleic acid sequence encoding a DOD enzyme. In another embodiment, a polynucleotide of the present invention comprises a nucleic acid sequence encoding CYP76AD6, CYP76AD15, or a combination thereof, a nucleic acid sequence encoding CYP76AD1, and a nucleic acid sequence encoding a DOD enzyme. In another embodiment, a polynucleotide of the present invention comprises a nucleic acid sequence encoding CYP76AD6, CYP76AD15, or a combination thereof, a nucleic acid sequence encoding CYP76AD1, and a nucleic acid sequence encoding a betalain related glucosyltransferase. In another embodiment, a polynucleotide of the present invention comprises a nucleic acid sequence encoding CYP76AD6, CYP76AD15, or a combination thereof, a nucleic acid sequence encoding a DOD enzyme and a nucleic acid sequence encoding a betalain related glucosyltransferase. In another embodiment, a polynucleotide of the present invention comprises a nucleic acid sequence encoding CYP76AD1, a nucleic acid sequence encoding a DOD enzyme and a nucleic acid sequence encoding a betalain related glucosyltransferase. In another embodiment, a polynucleotide of the present invention comprises a nucleic acid sequence encoding CYP76AD6, CYP76AD15, or a combination thereof, a nucleic acid sequence encoding CYP76AD1, a nucleic acid sequence encoding a DOD enzyme and a nucleic acid sequence encoding a betalain related glucosyltransferase.

In another embodiment, any of the recombinant polynucleotides described hereinabove further comprises a betalain related glucosyltransferase. In one embodiment, the betalain related glucosyltransferase is cyclo-DOPA 5-O-glucosyltransferase or betanidin-5-O-glucosyltransferase. In one embodiment, the cyclo-DOPA 5-O-glucosyltransferase is M. jalapa gene cyclo-DOPA 5-O-glucosyltransferase (cDOPA5GT) (SEQ ID NO: 2).

In another embodiment, the present invention provides a recombinant polynucleotide comprising a nucleic acid encoding CYP76AD1, a nucleic acid encoding CYP76AD6, CYP76AD15, or a combination thereof, a nucleic acid encoding a DOPA 4,5-dioxygenase (DOD) enzyme, and a nucleic acid sequence encoding a betalain related glucosyltransferase, wherein said nucleic acids are in the same reading frame.

In another embodiment, the present invention provides a recombinant polynucleotide comprising a nucleic acid encoding CYP76AD1, a nucleic acid encoding a DOPA 4,5-dioxygenase (DOD) enzyme, and a nucleic acid sequence encoding a betalain related glucosyltransferase, wherein said nucleic acids are in the same reading frame.

In another embodiment, the present invention provides a recombinant polynucleotide comprising a nucleic acid encoding CYP76AD6, CYP76AD15, or a combination thereof and a nucleic acid encoding a DOPA 4,5-dioxygenase (DOD) enzyme expressed in tandem.

In another embodiment, the present invention provides a recombinant polynucleotide comprising a nucleic acid encoding CYP76AD1, CYP76AD6, CYP76AD15, or a combination thereof, or a combination thereof, a nucleic acid encoding a DOPA 4,5-dioxygenase (DOD) enzyme, and a nucleic acid sequence encoding a betalain related glucosyltransferase, expressed in tandem.

In one embodiment, the polynucleotides as described herein comprise multiple nucleic acid sequences that are in the same reading frame. In one embodiment, the polynucleotides as described herein comprise multiple nucleic acid sequences that are expressed in tandem.

In one embodiment, a polynucleotide of the present invention comprises a nucleic acid encoding CYP76AD1, a nucleic acid encoding a DOPA 4,5-dioxygenase (DOD) enzyme, and a nucleic acid sequence encoding a betalain related glucosyltransferase, wherein said nucleic acids are in the same reading frame. In one embodiment, the DOD gene is under a CaMV 35S promoter. In one embodiment, the CYP76AD1 gene is under a CaMV 35S promoter. In one embodiment, the betalain related glucosyltransferase gene is under an Arabidopsis Ubiquitin-10 promoter. In one embodiment, the polynucleotide further comprises a kanamycin resistance gene. In one embodiment, the polynucleotide is the pX11 vector, as described in Example 8, hereinbelow. In one embodiment, the nucleic acid sequence of the pX11 vector comprises (SEQ ID NO: 19).

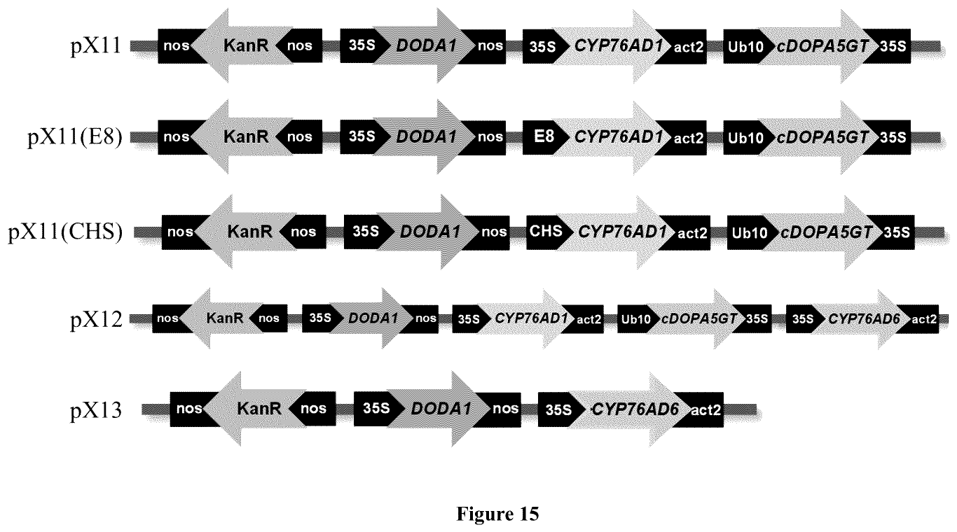

In another embodiment, the CYP76AD1 gene is under an E8 promoter (pX11(E8), SEQ ID NO: 20; FIG. 15). In another embodiment, the CYP76AD1 gene is under a CHS promoter (pX11(CHS) SEQ ID NO: 21; FIG. 15).

In another embodiment, a polynucleotide of the present invention comprises a nucleic acid encoding CYP76AD6, CYP76AD15, or a combination thereof, a nucleic acid encoding a DOPA 4,5-dioxygenase (DOD) enzyme, and a nucleic acid sequence encoding a betalain related glucosyltransferase, wherein said nucleic acids are in the same reading frame. In one embodiment, the DOD gene is under a CaMV 35S promoter. In one embodiment, the CYP76AD6, CYP76AD15, or a combination thereof, gene is under a CaMV 35S promoter. In one embodiment, the betalain related glucosyltransferase gene is under an Arabidopsis Ubiquitin-10 promoter. In one embodiment, the polynucleotide further comprises a kanamycin resistance gene. In one embodiment, the polynucleotide is the pX13 vector, as described in Example 14, hereinbelow.

In another embodiment, the CYP76AD6, CYP76AD15, or a combination thereof, gene is under an E8 promoter. In another embodiment, the CYP76AD6, CYP76AD15, or a combination thereof, gene is under a CHS promoter.

In another embodiment, a polynucleotide of the present invention comprises a nucleic acid encoding CYP76AD6, CYP76AD15, or a combination thereof, a nucleic acid encoding CYP76AD1, a nucleic acid encoding a DOPA 4,5-dioxygenase (DOD) enzyme, and a nucleic acid sequence encoding a betalain related glucosyltransferase, wherein said nucleic acids are in the same reading frame. In one embodiment, the DOD gene is under a CaMV 35S promoter. In one embodiment, the CYP76AD6, CYP76AD15, or a combination thereof, gene is under a CaMV 35S promoter. In one embodiment, the CYP76AD1 gene is under a CaMV 35S promoter. In one embodiment, the betalain related glucosyltransferase gene is under an Arabidopsis Ubiquitin-10 promoter. In one embodiment, the polynucleotide further comprises a kanamycin resistance gene. In one embodiment, the polynucleotide is the pX12 vector, as described in Example 14, hereinbelow.

In another embodiment, the CYP76AD6, CYP76AD15, or a combination thereof, gene is under an E8 promoter. In another embodiment, the CYP76AD6, CYP76AD15, or a combination thereof, gene is under a CHS promoter. In another embodiment, the CYP76AD1 gene is under an E8 promoter. In another embodiment, the CYP76AD1 gene is under a CHS promoter.

In one embodiment, the present invention provides a comprising a nucleic acid encoding a CYP76AD1-β clade gene under the control of a promoter. In one embodiment, the CYP76AD1-clade gene is CYP76AD6. In another embodiment, the CYP76AD1-β clade gene is CYP76AD15.

In one embodiment, the present invention provides a recombinant nucleic acid comprising a CYP76AD1-β clade gene from red beet. In one embodiment, the present invention provides a recombinant nucleic acid comprising a CYP76AD1-β clade gene that is not from sugar beet. In one embodiment, the present invention provides a recombinant nucleic acid comprising a CYP76AD1-β clade gene but excluding the nucleic acid sequence of the CYP76AD1 paralogs from sugar beet (DeLoache et al., 2015), which do not produce betaxanthin when transformed into yeast with DOD. In one embodiment, the present invention provides a recombinant nucleic acid comprising a CYP76AD1-β clade gene but excluding the nucleic acid encoding SEQ ID NO: 34.

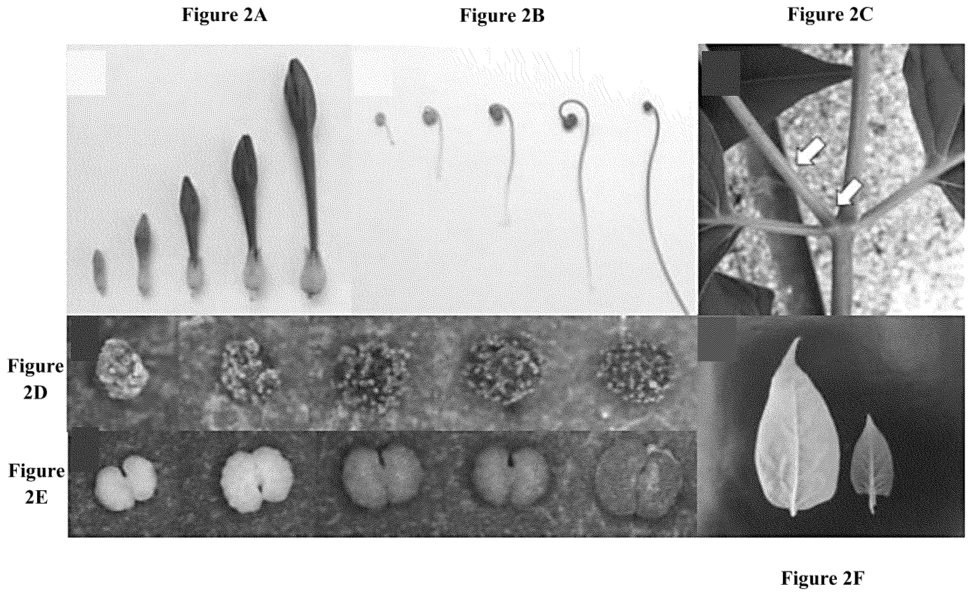

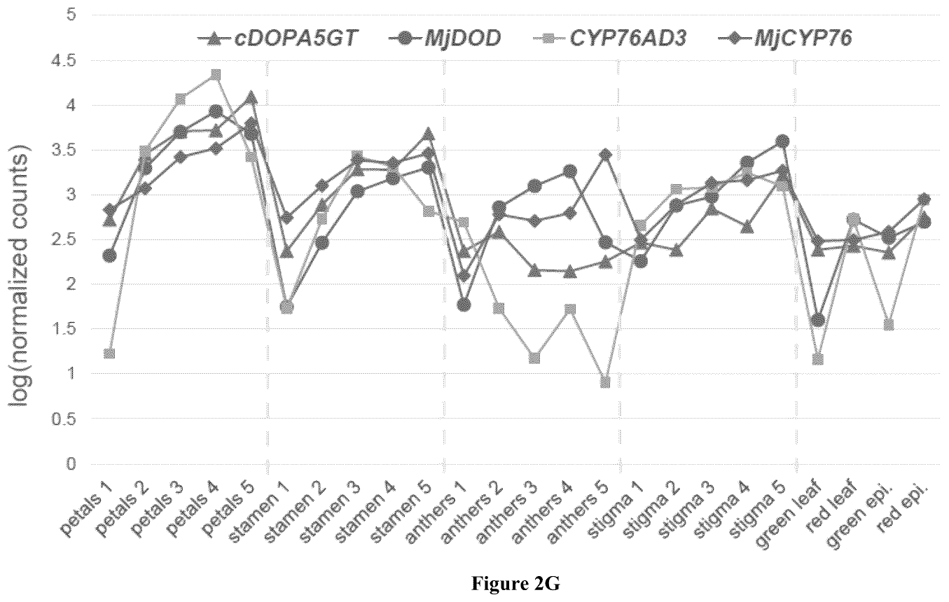

In another embodiment, the CYP76AD1-β clade gene is a CYP76AD1-β clade gene as set forth in Brockington et al. 2015 (New Phytol. 2015 September; 207(4):1170-80), which is incorporated herein by reference in its entirety). In one embodiment, the CYP76AD1-β clade gene is a CYP76AD1-β clade gene as set forth in Supplemental FIG. 2 of Brockington et al. 2015.

In one embodiment, the present invention provides a recombinant polynucleotide comprising a nucleic acid encoding a CYP76AD6 gene, a CYP76AD15 gene, or a combination thereof under the control of a promoter.

In one embodiment, the present invention provides a recombinant polynucleotide comprising a nucleic acid encoding a CYP76AD1-β clade gene and a nucleic acid encoding a DOPA 4,5-dioxygenase (DOD) enzyme, wherein said nucleic acids are inserted into the polynucleotide in frame. In one embodiment, the polynucleotide further comprises a nucleic acid encoding betalain related glucosyltransferase.

In one embodiment, the nucleic acid encoding a CYP76AD1-β clade gene is Bv. maritima, Beta vulgaris ssp. maritima (GenBank accession AKI33834.1; SEQ ID NO: 10); F. latifolia, Froelichia latifolia (accession AKI33838.1; SEQ ID NO: 11); A. caracasana, Alternanthera caracasana (accession AKI33835.1; SEQ ID NO: 12); A. ficoidea, Alternanthera ficoidea (accession AKI33831.1; SEQ ID NO: 13), or a combination thereof.

In one embodiment, the present invention provides a recombinant polynucleotide comprising a nucleic acid encoding a CYP76AD6 gene, a CYP76AD15 gene, or a combination thereof and a nucleic acid encoding a DOPA 4,5-dioxygenase (DOD) enzyme, wherein said nucleic acids are inserted into the polynucleotide in frame. In one embodiment, the polynucleotide further comprises a nucleic acid encoding betalain related glucosyltransferase.

In another embodiment, the present invention comprises additional nucleic acids encoding polypeptides that modify betalain structure. In one embodiment, the polypeptide is a glycosylating enzyme. In another embodiment, the polypeptide is an acylating enzyme. In another embodiment, additional nucleic acids encode polypeptides involved in subcellular transport. In another embodiment, additional nucleic acids encode polypeptides involved in detoxification.

### Homologs and Variants

In another embodiment, a recombinant polynucleotide or polypeptide of the present invention is homologous to a sequence set forth herein, either expressly or by reference to a GenBank entry. The terms “homology,” “homologous,” etc, when in reference to any amino acid or nucleic acid sequence, refer, in one embodiment, to a percentage of amino acids or nucleotides, respectively, in the candidate sequence that are identical with the residues of a corresponding native polypeptide or polynucleotide, after aligning the sequences and introducing gaps, if necessary, to achieve the maximum percent homology, and not considering any conservative substitutions as part of the sequence identity. Methods and computer programs for the alignment are well known in the art such as, for example, the BLAST, DOMAIN, BEAUTY (BLAST Enhanced Alignment Utility), GENPEPT and TREMBL packages.

In one embodiment, a homolog of CYP76AD1 may be used instead of CYP76AD1 in accordance with the compositions and methods of the present invention.

In another embodiment, a homolog of CYP76AD6 may be used instead of CYP76AD6, in accordance with the compositions and methods of the present invention.

In another embodiment, a homolog of DOPA 4,5-dioxygenase (DOD) enzyme may be used instead of DOPA 4,5-dioxygenase (DOD) enzyme, in accordance with the compositions and methods of the present invention.

Homology may be at the nucleotide sequence and/or encoded amino acid sequence level. In one embodiment, the nucleic acid and/or amino acid sequence shares at least about 50%, or 60%, or 70%, or 80% homology. In another embodiment, the nucleic acid and/or amino acid sequence shares at least about 90%, 95%, 96%, 97%, 98% or 99% homology with a sequence of the present invention. Homology may be at the nucleotide sequence and/or encoded amino acid sequence level. In one embodiment, the nucleic acid and/or amino acid sequence shares at least about 72% homology. In one embodiment, the nucleic acid and/or amino acid sequence shares at least about 75% homology.

In another embodiment, homology is determined via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y).

In one embodiment, the homolog is isolated from another Caryophyllales plant. In one embodiment, the homolog is isolated from a cactus, a carnation, an amaranth, an ice plant, a beet, a carnivorous plant, or a combination thereof. In one embodiment, the carnivorous plant is a Dionocophyllaceae such as Triphyophyllum, a Droseraceae, such as Aldrovanda (Waterwheel Plant), Dionaea (Venus Flytrap), Drosera (Sundew), a Drosophyllaceae such as a Drosophyllum (Dewy Pine or Portugese Sundew), or a Nepenthaceae such as Tropical Pitcher Plant or Monkey Cup.

In one embodiment, the homolog is isolated based on temporal expression. In one embodiment, the homolog is isolated based on developmental stage. In one embodiment, the developmental stage is Stage 1, which, in one embodiment, is when the flower length is approximately 1 cm. In another embodiment, the developmental stage is Stage 2, which, in one embodiment, is when the flower length is approximately 2 cm. In another embodiment, the developmental stage is Stage 3, which, in one embodiment, is when the flower length is approximately 3 cm. In another embodiment, the developmental stage is Stage 4, which, in one embodiment, is when the flower length is approximately 4 cm. In another embodiment, the developmental stage is Stage 5, which, in one embodiment, is when the flower length is approximately 5-6 cm.

In another embodiment, the homolog is isolated based on tissue expression. In one embodiment, the tissue expression of a CYP76AD1, CYP76AD6, CYP76AD15, or a combination thereof, or DOD homolog is in a floral tissue. In one embodiment, the floral tissue is a petal. In another embodiment, the floral tissue is a stamen. In another embodiment, the floral tissue is an anther. In another embodiment, the floral tissue is a stigma.

In another embodiment, a recombinant polynucleotide or polypeptide of the present invention is a variant of a sequence set forth herein. In another embodiment, a recombinant polynucleotide or polypeptide of the present invention is an isoform of a sequence set forth herein. In another embodiment, a recombinant polynucleotide or polypeptide of the present invention is a fragment of a sequence set forth herein. In one embodiment, a recombinant polynucleotide or polypeptide of the present invention is a functional variant of a sequence set forth herein. In another embodiment, a recombinant polynucleotide or polypeptide of the present invention is a functional isoform of a sequence set forth herein. In another embodiment, a recombinant polynucleotide or polypeptide of the present invention is a functional fragment of a sequence set forth herein. In another embodiment, a recombinant polynucleotide or polypeptide of the present invention is a functional homologue of a sequence set forth herein.

In another embodiment, the present invention provides a recombinant polynucleotide comprising a nucleic acid encoding a polypeptide as described herein.

In one embodiment, the nucleic acid sequence encoding CYP76AD6 is as set forth in SEQ ID NO: 30. In another embodiment a polynucleotide of the present invention optionally comprises a nucleic acid sequence encoding CYP76AD6. In one embodiment, the nucleic acid sequence of CYP76AD6 is as set forth in SEQ ID NO: 30. In another embodiment, the CYP76AD6 utilized in methods and compositions of the present invention is a homologue of SEQ ID NO: 30. In another embodiment, the CYP76AD6 utilized in methods and compositions of the present invention is a variant of SEQ ID NO: 30. In another embodiment, the CYP76AD6 utilized in methods and compositions of the present invention is a fragment of SEQ ID NO: 30. In another embodiment, the CYP76AD6 utilized in methods and compositions of the present invention is an isoform of SEQ ID NO: 30. In another embodiment, the CYP76AD6 utilized in methods and compositions of the present invention is a functional homologue, functional variant, functional fragment, or functional isoform of SEQ ID NO: 30.

As demonstrated in Example 19, CYP76AD15, an ortholog of CYP76AD6 in Mirabilis jalapa, had the same activity as CYP76AD6 during transient expression in Nicotiana benthamiana. In another embodiment, CYP76AD15 or a homologue, isoform, or variant thereof, may be used in place of CYP76AD6 in the compositions and methods of the present invention. In one embodiment, the CYP76AD15 gene product from Mirabilis jalapa has a similar function as the CYP76AD6 gene product from Beta vulgaris. In one embodiment, CYP76AD15 from Mirabilis jalapa is the gene encoding an enzyme involved in converting tyrosine to L-DOPA.

In one embodiment, the present invention provides a recombinant polynucleotide comprising a nucleic acid encoding CYP76AD15. In one embodiment, the nucleic acid sequence encoding CYP76AD15 comprises:

(SEQ ID NO: 35)
tttgtctattattggctcaaaatccactactatctttttgtaaaagaaaa
tagttgttcaacttagggaattattgatatttcattatggaaaacacaat
gttaggtgttatcctagcaaccattttcctcacttttcacataatgaaga
tgttatttagtccttccaaggttaaactacccccgggtccgagaccattg
ccaattattggtaatattctcgagcttggggataaaccacatcgttattt
gcaaaccttgcgaaaattcacggtcctttagttactttgaaactcgggag
tgtaaccactattgtggtttcctcttctgaagttgctaaagaaatgtttt
tgaaaaatgaccaacctttggcaaatcgtaccatacctgactcagtaaga
gcaggtaaccatgacaaactatcaatgtcgtggttgcctgtatcacccaa
atggcgaaatcttagaaagatttcagccgtccaattgactcaactcaacg
acttgatgcaagtcaagctcatagacaagctaaaatcaaacaacttattg
agtacgtaaaaaaatgcagtaaaatcggccaatacgtcgatattggccaa
gttgcattcactacatcacttaatttactatcaaacacattcttttcaaa
agaactagcatcatttgattcagataatgcacaagagttcaaacaactaa
tgtggtgcattatggaagaaattggtaggcctaattatgccgattatttt
cctatcttgggttatgtcgatccattcggtgctagacgtcgactttacgt
tacttcgatcaactaattgaagtatttcaagtgattattcgtgagagact
tacacatgataataatattgtgggtaataacaatgatgttttagctacgt
tgctcgatattataaacaaaacgagttaactatggatgaaatcaaccatt
tactagtggacatttttgatgctggtacggatacaacagcaagtacacta
gaatgggcaatgtcagagctcataaaaaatccacacataatggccaaagc
tcaagaggaggtccggcgagccaccatgtctcacggcggagctacggtgg
cggaaatacaagaatcggatatcaataatcttccatacatacaatctatt
attaaagaaacacttcgtttacacccaccaactgtgtttttacttcctag
aaaagctgacgtggatgtccaattattcggctatgtggtccccaaaaatg
ctcaagtcctagtcaatttatgggccattggtcgtgacccaaatgtgtgg
cccgacccggaagtttttagtcccgaaagatttatggattgtgagattga
tgtcaagggtcgtgattttgagctattgcctttcggggcgggtcgtcgga
tttgtccgggattgtattggcttatcggatgcttaatttgatgttggcta
atatggtacattatttgattggaaattacccggtgttgaaaatggatccg
ggtcggaaatggatagtttggatatggatgagaaatttggtatcactttg
caaaaggttcaaccccttaaggttattcctgtctcaaggaaatagatgga
agatcgatggttgggagataatcatattgtttttaattattgttggtgta
ttttattgttattattggggacaattttataataaaagatataaataagt
tcctaattgtgatttatattgtgaattttctagttaatataaatacatat
gaatatttatgatgaaaaaaaaaaaaaaaaaaaaagg.

In another embodiment, a polynucleotide of the present invention comprises a nucleic acid sequence encoding BvCYP76new, as described in Example 3. In another embodiment, a polynucleotide of the present invention comprises a nucleic acid sequence encoding MjCYP76 from M. jalapa (SEQ ID NO: 1). In one embodiment, CYP76AD15, BvCYP76new or MjCYP76 may be used in the compositions and methods of the present invention. In another embodiment, other cytochrome P450-encoding genes co-expressed with one or more of the known betalain related genes used as baits may be used in the compositions and methods of the present invention. In one embodiment, CYP82G1-like (SEQ ID NO: 36), CYP78A9-like (SEQ ID NO: 37), CYP86B1-like (SEQ ID NO: 38), or a combination thereof may be used in the compositions and methods of the present invention. In one embodiment, may be used in place of CYP76AD6.

In one embodiment a polynucleotide of the present invention comprises a nucleic acid sequence encoding CYP76AD1. In one embodiment a polynucleotide of the present invention optionally comprises a nucleic acid sequence encoding CYP76AD1. In one embodiment, the nucleic acid sequence of CYP76AD1 is as set forth in SEQ ID NO: 32. In another embodiment, the CYP76AD1 utilized in methods and compositions of the present invention is a homologue of SEQ ID NO: 32. In another embodiment, the CYP76AD1 utilized in methods and compositions of the present invention is a variant of SEQ ID NO: 32. In another embodiment, the CYP76AD1 utilized in methods and compositions of the present invention is a fragment of SEQ ID NO: 32. In another embodiment, the CYP76AD1 utilized in methods and compositions of the present invention is an isoform of SEQ ID NO: 32. In another embodiment, the CYP76AD1 utilized in methods and compositions of the present invention is a functional homologue, functional variant, functional fragment, or functional isoform of SEQ ID NO: 32.

In another embodiment, a polynucleotide of the present invention comprises a nucleic acid sequence encoding CYP76AD3 (SEQ ID NO: 4), as described in Example 9. In one embodiment, CYP76AD3 may be used instead of CYP76AD1 in the compositions and methods of the present invention.

In one embodiment a polynucleotide of the present invention comprises a nucleic acid sequence encoding Beta vulgaris DODA1 (BvDODA1). In one embodiment a polynucleotide of the present invention optionally comprises a nucleic acid sequence encoding BvDODA1. In one embodiment, the nucleic acid sequence of BvDODA1 is as set forth in SEQ ID NO: 33. In another embodiment, the BVDODA1 utilized in methods and compositions of the present invention is a homologue of SEQ ID NO: 33. In another embodiment, the BVDODA1 utilized in methods and compositions of the present invention is a variant of SEQ ID NO: 33. In another embodiment, the BVDODA1 utilized in methods and compositions of the present invention is a fragment of SEQ ID NO: 33. In another embodiment, the BVDODA1 utilized in methods and compositions of the present invention is an isoform of SEQ ID NO: 33. In another embodiment, the BVDODA1 utilized in methods and compositions of the present invention is a functional homologue, functional variant, functional fragment, or functional isoform of SEQ ID NO: 33.

In another embodiment, the DOD enzyme is PgDOD from Portulaca grandiflora (Accession No. AJ580598; SEQ ID NO: 39), MjDOD from Mirabilis jalapa (Accession No. AB435372; SEQ ID NO: 3), BgDOD from Bougainvillea glabra (Accession No. AB435373; SEQ ID NO: 40), or AmDOD from Amanita muscaria (Accession No. P87064; SEQ ID NO: 41).

In one embodiment, a polynucleotide of the present invention comprises a nucleic acid sequence encoding cyclo-DOPA 5-O-glucosyltransferase (cDOPA5GT). In one embodiment, a polynucleotide of the present invention optionally comprises a nucleic acid sequence encoding cDOPA5GT. In one embodiment, the nucleic acid sequence of cDOPA5GT is as set forth in SEQ ID NO: 2. In another embodiment, the cDOPA5GT utilized in methods and compositions of the present invention is a homologue of SEQ ID NO: 2. In another embodiment, the cDOPA5GT utilized in methods and compositions of the present invention is a variant of SEQ ID NO: 2. In another embodiment, the cDOPA5GT utilized in methods and compositions of the present invention is a fragment of SEQ ID NO: 2. In another embodiment, the cDOPA5GT utilized in methods and compositions of the present invention is an isoform of SEQ ID NO: 2. In another embodiment, the cDOPA5GT utilized in methods and compositions of the present invention is a functional homologue, functional variant, functional fragment, or functional isoform of SEQ ID NO: 2.

In one embodiment, “variant” refers to an amino acid or nucleic acid sequence (or in other embodiments, an organism or tissue) that is different from the majority of the population but is still sufficiently similar to the common mode to be considered to be one of them, for example splice variants. In one embodiment, the variant may a sequence conservative variant, while in another embodiment, the variant may be a functional conservative variant. In one embodiment, a variant may comprise an addition, deletion or substitution of 1 amino acid. In one embodiment, a variant may comprise an addition, deletion, substitution, or combination thereof of 2 amino acids. In one embodiment, a variant may comprise an addition, deletion or substitution, or combination thereof of 3 amino acids. In one embodiment, a variant may comprise an addition, deletion or substitution, or combination thereof of 4 amino acids. In one embodiment, a variant may comprise an addition, deletion or substitution, or combination thereof of 5 amino acids. In one embodiment, a variant may comprise an addition, deletion or substitution, or combination thereof of 7 amino acids. In one embodiment, a variant may comprise an addition, deletion or substitution, or combination thereof of 10 amino acids. In one embodiment, a variant may comprise an addition, deletion or substitution, or combination thereof of 2-15 amino acids. In one embodiment, a variant may comprise an addition, deletion or substitution, or combination thereof of 3-20 amino acids. In one embodiment, a variant may comprise an addition, deletion or substitution, or combination thereof of 4-25 amino acids.

In one embodiment, the term “fragment” is used herein to refer to a protein or polypeptide that is shorter or comprises fewer amino acids than the full length protein or polypeptide. In another embodiment, fragment refers to a nucleic acid that is shorter or comprises fewer nucleotides than the full length nucleic acid. In another embodiment, the fragment is an N-terminal fragment. In another embodiment, the fragment is a C-terminal fragment. In one embodiment, the fragment is an intrasequential section of the protein, peptide, or nucleic acid. In another embodiment, the fragment is a functional section within the protein, peptide or nucleic acid.

In one embodiment, “isoform” refers to a protein isoform or a gene isoform. In one embodiment, a protein isoform is a different form of a protein coded from the same gene, or a protein from a different gene with amino acid sequence and functional similarities. In one embodiment, a gene isoform is an mRNA that is produced from the same locus but has different transcription start sites (TSSs), protein coding DNA sequences (CDSs) and/or untranslated regions (UTRs). In one embodiment, the gene isoform has altered activity, while in another embodiment, the gene isoform does not have altered activity. Thus, in one embodiment, an isoform is an alternate version of a molecule, for example, a protein, that has the same function as the first molecule but which may have small differences in its structure or sequence. In one embodiment, isoforms may be produced from different but related genes, or in another embodiment, may arise from the same gene by alternative splicing. In another embodiment, isoforms are caused by single nucleotide polymorphisms.

In one embodiment, “functional” within the meaning of the invention, is used herein to refer to the innate ability of a protein, peptide, nucleic acid, fragment or a variant thereof to exhibit a biological activity or function. In one embodiment, such a biological function is its binding property to an interaction partner, e.g., a membrane-associated receptor, and in another embodiment, its trimerization property. In the case of functional fragments and the functional variants of the invention, these biological functions may in fact be changed, e.g., with respect to their specificity or selectivity, but with retention of the basic biological function.

Numerous methods for measuring the biological activity of a protein, polypeptide, or molecule are known from the related art, for example, protein assays, which use labeled substrates, substrate analyses by chromatographic methods, such as HPLC or thin-layer chromatography, spectrophotometric methods, etc. (see, e.g., Maniatis et al. (2001) Molecular Cloning: A Laboratory Manual, Cold Spring Harbor Laboratory Press, Cold Spring Harbor, N.Y.).

### Expression Vectors

In another embodiment, the present invention provides an expression vector comprising any of the polynucleotides of the present invention as described herein.

In another embodiment, the present invention provides an expression vector comprising a recombinant polynucleotide comprising a nucleic acid encoding CYP76AD1, a nucleic acid encoding a DOPA 4,5-dioxygenase (DOD) enzyme, and, optionally, a nucleic acid sequence encoding a betalain related glucosyltransferase, wherein said nucleic acids are in the same reading frame.

In another embodiment, the present invention provides an expression vector comprising a recombinant polynucleotide comprising a nucleic acid encoding CYP76AD6, CYP76AD15, or a combination thereof, and a nucleic acid encoding a DOPA 4,5-dioxygenase (DOD) enzyme expressed in tandem.

In another embodiment, the present invention provides an expression vector comprising a recombinant polynucleotide comprising a nucleic acid encoding CYP76AD1, CYP76AD6, CYP76AD15, or a combination thereof, a nucleic acid encoding a DOPA 4,5-dioxygenase (DOD) enzyme, and, optionally, a nucleic acid sequence encoding a betalain related glucosyltransferase, expressed in tandem.

In one embodiment, polynucleotides of the present invention are inserted into expression vectors (i.e., a nucleic acid construct) to enable expression of the recombinant polypeptide. In one embodiment, the expression vector of the present invention includes additional sequences which render this vector suitable for replication and integration in prokaryotes. In one embodiment, the expression vector of the present invention includes additional sequences which render this vector suitable for replication and integration in eukaryotes. In one embodiment, the expression vector of the present invention includes a shuttle vector which renders this vector suitable for replication and integration in both prokaryotes and eukaryotes. In some embodiments, cloning vectors comprise transcription and translation initiation sequences (e.g., promoters, enhancer) and transcription and translation terminators (e.g., polyadenylation signals).

In one embodiment, a variety of prokaryotic or eukaryotic cells can be used as host-expression systems to express the polypeptides of the present invention. In some embodiments, these include, but are not limited to, microorganisms, such as bacteria transformed with a recombinant bacteriophage DNA, plasmid DNA or cosmid DNA expression vector containing the polypeptide coding sequence; yeast transformed with recombinant yeast expression vectors containing the polypeptide coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the polypeptide coding sequence.

In one embodiment, viral vectors of the present invention overexpress the pathway genes described herein. In another embodiment, viral infection of cells expressing the pathway genes described herein induces the cells to overexpress the pathway genes.

In some embodiments, non-bacterial expression systems are used (e.g. mammalian expression systems such as CHO cells) to express the polypeptide of the present invention. In one embodiment, the expression vector used to express polynucleotides of the present invention in mammalian cells is pCI-DHFR vector comprising a CMV promoter and a neomycin resistance gene.

In some embodiments, in bacterial systems of the present invention, a number of expression vectors can be advantageously selected depending upon the use intended for the polypeptide expressed. In one embodiment, large quantities of polypeptide are desired. In one embodiment, vectors that direct the expression of high levels of the protein product, possibly as a fusion with a hydrophobic signal sequence, which directs the expressed product into the periplasm of the bacteria or the culture medium where the protein product is readily purified are desired. In one embodiment, certain fusion protein engineered with a specific cleavage site to aid in recovery of the polypeptide. In one embodiment, vectors adaptable to such manipulation include, but are not limited to, the pET series of E. coli expression vectors (Studier et al., Methods in Enzymol. 185:60-89 1990).

In one embodiment, yeast expression systems are used. In one embodiment, a number of vectors containing constitutive or inducible promoters can be used in yeast as disclosed in U.S. Pat. No. 5,932,447. In another embodiment, vectors which promote integration of foreign DNA sequences into the yeast chromosome are used.

In one embodiment, the expression vector of the present invention can further include additional polynucleotide sequences that allow, for example, the translation of several proteins from a single mRNA such as an internal ribosome entry site (IRES) and sequences for genomic integration of the promoter-chimeric polypeptide.

In some embodiments, mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors are useful for in vivo expression of the polypeptides of the present invention since they offer advantages such as lateral infection and targeting specificity. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

In one embodiment, various methods can be used to introduce the expression vector of the present invention into cells. Such methods include, for example, stable or transient transfection, lipofection, electroporation and infection with recombinant viral vectors.

In some embodiments, introduction of nucleic acid by viral infection offers several advantages over other methods such as lipofection and electroporation, since higher transfection efficiency can be obtained due to the infectious nature of viruses.

In one embodiment, it will be appreciated that the polypeptides of the present invention can also be expressed from a nucleic acid construct administered to the individual employing any suitable mode of administration, described hereinabove (i.e., in-vivo gene therapy). In one embodiment, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex-vivo gene therapy).

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

Various methods, in some embodiments, can be used to introduce the expression vector of the present invention into the host cell system. In some embodiments, such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In some embodiments, transformed cells are cultured under effective conditions, which allow for the expression of high amounts of recombinant polypeptide. In some embodiments, effective culture conditions include, but are not limited to, effective media, bioreactor, temperature, pH and oxygen conditions that permit protein production. In one embodiment, an effective medium refers to any medium in which a cell is cultured to produce the recombinant polypeptide of the present invention. In some embodiments, a medium typically includes an aqueous solution having assimilable carbon, nitrogen and phosphate sources, and appropriate salts, minerals, metals and other nutrients, such as vitamins. In some embodiments, cells of the present invention can be cultured in conventional fermentation bioreactors, shake flasks, test tubes, microtiter dishes and petri plates. In some embodiments, culturing is carried out at a temperature, pH and oxygen content appropriate for a recombinant cell. In some embodiments, culturing conditions are within the expertise of one of ordinary skill in the art.

In some embodiments, depending on the vector and host system used for production, resultant polypeptides of the present invention either remain within the recombinant cell, secreted into the fermentation medium, secreted into a space between two cellular membranes, such as the periplasmic space in E. coli; or retained on the outer surface of a cell or viral membrane.

### Compositions

In another embodiment, the present invention provides a composition comprising one or more recombinant polynucleotides as described herein. In another embodiment, the present invention provides a composition comprising recombinant nucleic acids as described herein.

In one embodiment, the present invention provides a recombinant polynucleotide comprising a nucleic acid encoding CYP76AD1, a nucleic acid encoding a DOPA 4,5-dioxygenase (DOD) enzyme, and, optionally, a nucleic acid sequence encoding a betalain related glucosyltransferase, wherein said nucleic acids are in the same reading frame, wherein said nucleic acids are in the same reading frame.

In another embodiment, the present invention provides a composition comprising a recombinant polynucleotide comprising a nucleic acid encoding CYP76AD6, CYP76AD15, or a combination thereof, and a nucleic acid encoding a DOPA 4,5-dioxygenase (DOD) enzyme expressed in tandem.

In another embodiment, the present invention provides an expression vector comprising a recombinant polynucleotide as described herein.

In another embodiment, the present invention provides a composition comprising an expression vector comprising a recombinant polynucleotide comprising a nucleic acid encoding CYP76AD1, a nucleic acid encoding a DOPA 4,5-dioxygenase (DOD) enzyme, and, optionally, a nucleic acid sequence encoding a betalain related glucosyltransferase, wherein said nucleic acids are in the same reading frame, wherein said nucleic acids are in the same reading frame.

In another embodiment, the present invention provides a composition comprising an expression vector comprising a recombinant polynucleotide comprising a nucleic acid encoding CYP76AD6, CYP76AD15, or a combination thereof, and a nucleic acid encoding a DOPA 4,5-dioxygenase (DOD) enzyme expressed in tandem.

In one embodiment, provides a composition comprising a recombinant polynucleotide or a chimeric polypeptide of the present invention. In one embodiment, the polypeptides and polynucleotides of the present invention can be provided to the individual per se.

In another embodiment, the present invention provides a cell comprising an expression vector or a recombinant polynucleotide as described herein. In another embodiment, the present invention provides a composition comprising a cell as described herein.

### Cells

In another embodiment, the present invention provides a cell comprising a recombinant polynucleotide as described herein. In another embodiment, the present invention provides a cell comprising an expression vector as described herein.

In another embodiment, the present invention provides a cell comprising a recombinant polynucleotide comprising a nucleic acid encoding CYP76AD1, a nucleic acid encoding a DOPA 4,5-dioxygenase (DOD) enzyme, and, optionally, a nucleic acid sequence encoding a betalain related glucosyltransferase, wherein said nucleic acids are in the same reading frame.

In another embodiment, the present invention provides a cell comprising a recombinant polynucleotide comprising a nucleic acid encoding CYP76AD6, CYP76AD15, or a combination thereof, and a nucleic acid encoding a DOPA 4,5-dioxygenase (DOD) enzyme expressed in tandem.

In another embodiment, the present invention provides a cell comprising a recombinant polynucleotide comprising a nucleic acid encoding CYP76AD1, CYP76AD6, CYP76AD15, or a combination thereof, a nucleic acid encoding a DOPA 4,5-dioxygenase (DOD) enzyme, and, optionally, a nucleic acid sequence encoding a betalain related glucosyltransferase, expressed in tandem.

In another embodiment, the present invention provides a cell comprising an expression vector comprising a recombinant polynucleotide comprising a nucleic acid encoding CYP76AD1, a nucleic acid encoding a DOPA 4,5-dioxygenase (DOD) enzyme, and, optionally, a nucleic acid sequence encoding a betalain related glucosyltransferase, wherein said nucleic acids are in the same reading frame.

In another embodiment, the present invention provides a cell comprising an expression vector comprising a recombinant polynucleotide comprising a nucleic acid encoding CYP76AD6, CYP76AD15, or a combination thereof, and a nucleic acid encoding a DOPA 4,5-dioxygenase (DOD) enzyme expressed in tandem.

In another embodiment, the present invention provides a cell comprising an expression vector comprising a recombinant polynucleotide comprising a nucleic acid encoding CYP76AD1, CYP76AD6, CYP76AD15, or a combination thereof, a nucleic acid encoding a DOPA 4,5-dioxygenase (DOD) enzyme, and, optionally, a nucleic acid sequence encoding a betalain related glucosyltransferase, expressed in tandem.

In one embodiment, when the genes of the invention can be transformed into a plant cell, a plant cell suspension culture may be produced. In one embodiment, the plant cell is a plant stem cell. Betalain, L-DOPA, or a combination thereof can be isolated from plant suspension culture. Method for making plant cell suspension culture are well known in the art and fully described in U.S. Patent Application Publications US 2014/0051135; US 2011/0251408; US 2010/0159545; and US 2007/0026506, which are incorporated by reference herein in their entirety.

In one embodiment, the plant cells are selected from tobacco (Nicotiana tabacum), tomato (Solanum lycopersicum), potato (Solanum tuberosum), eggplant (Solanum melongena), tree tobacco (Nicotiana glauca), European black nightshade (Solanum nigrum), Petunia (Petunia x hybrida) and Nicotiana benthamiana cells. In another embodiment, the plant cells are from any of the plants described hereinbelow.

In one embodiment in which polynucleotides are introduced into plant cells, the plant cells may be derived from any plant organ of interest, as described hereinbelow. In one embodiment, the polynucleotide is introduced into a portion of a plant organ. In one embodiment, the polynucleotide is introduced into a portion of a flower. In one embodiment, the portion of the flower is the petal, stamen, anther, stigma, or a combination thereof.

This invention also provides methods for manufacturing transgenic seed that can be used to produce a crop of transgenic plants with an enhanced trait resulting from expression of a stably-integrated recombinant DNA construct.

In another embodiment, cells of the present invention are cells from a micro-organism. In another embodiment, the cell is a yeast cell. In one embodiment, the yeast cell is a Saccharomyces cerevisiae cell. In another embodiment, the cell of the present invention is a bacterial cell. In one embodiment, the bacterial cell is an Escherichia coli cell. In another embodiment, the cell is an Acremonium rutilum, Aspergillus orvzae, Yarrowia lipolytica, Bacillus sp. JPJ, Brevundimonas sp. SGJ, E. herbicola, Citrobacter freundii, Symbiobacterium, or Pseudomonas aeruginosa cell. In another embodiment, the bacterial cell is from a bacterium involved in fermentation of dairy products. In one embodiment, the bacterium is Streptococcus lactis. In another embodiment, the bacterium is a Lactobacillus. In one embodiment, the Lactobacillus is Lactobacillus bulgaricus. In another embodiment, the bacterium is a Lactococcus or a Leuconostoc.

### Plants and Plant Parts

In another embodiment, the present invention provides a plant or part thereof produced by a method of increasing the levels of one or more betalains or L-DOPA in a plant or in a plant part comprising causing or allowing the expression of a DOPA 4,5-dioxygenase (DOD) enzyme, CYP76AD6, CYP76AD15, or a combination thereof, CYP76AD1, or a combination thereof, within said plant or plant part. In one embodiment, a plant expressing DOD and CYP76AD1 and not CYP76AD6 or CYP76AD15, also expresses a betalain related glucosyltransferase.

In one embodiment, the plant does not naturally produce betalains. In another embodiment, the plant produces low levels of betalains. In another embodiment, the plant does not naturally produce detectable levels of betalains.

In another embodiment, plants or plant parts as described herein naturally produce betalains; however, the genetically modified plant or plant parts have an altered level of betalains or of a particular betalain, thereby altering the color and/or properties of the genetically modified plant. In one embodiment, altered levels of betalains are increased levels. In another embodiment, altered levels of betalains are decreased levels.

In another embodiment, the present invention provides a plant or part thereof comprising a nucleic acid sequence encoding CYP76AD1, a nucleic acid sequence encoding CYP76AD6, CYP76AD15, or a combination thereof, a nucleic acid sequence encoding DOPA 4,5-dioxygenase (DOD) enzyme, or a combination thereof.

In another embodiment, betalains or L-DOPA are produced as described herein in a whole plant. In one embodiment, a seed is genetically modified such that betalains or L-DOPA are produced in a whole plant. In another embodiment, betalains or L-DOPA are produced in a particular organ of a plant. In another embodiment, betalains or L-DOPA are produced in a section of an organ of a plant. For example, betalains or L-DOPA may be produced in some plant cells in the leaf or other organ of a plant.

In another embodiment, the present invention provides an ornamental plant produced by a method of increasing the levels of one or more betalains in a plant or in a plant part thereof comprising causing or allowing the expression of a DOPA 4,5-dioxygenase (DOD) enzyme and CYP76AD6, CYP76AD15, or a combination thereof, CYP76AD1, or a combination thereof, within said plant or plant part. In one embodiment, if the ornamental plant expresses CYP76AD1 and DOD and not CYP76AD6 or CYP76AD15, it further expresses a betalain related glucosyltransferase.

In another embodiment, the present invention provides an ornamental plant comprising a nucleic acid sequence encoding CYP76AD1, CYP76AD6, CYP76AD15, or a combination thereof, and a nucleic acid sequence encoding DOPA 4,5-dioxygenase (DOD) enzyme.

In one embodiment, different parts or organs of an organism of the present invention comprise different pigmentation due to the expression of multiple cytochrome P450s (e.g., CYP76AD6, CYP76AD1) under different, non-constitutive promoter sequences (e.g. fruit-specific, flower-specific or inducible promoters). In one embodiment, the organism is a plant and the different pigmentation are in different parts of the plant. In one embodiment, the plant is an ornamental plant.

In another embodiment, the present invention provides a food crop produced by a method of increasing the levels of one or more betalains in a plant or part thereof that serves as a food crop comprising causing or allowing the expression of a DOPA 4,5-dioxygenase (DOD) enzyme and CYP76AD6, CYP76AD15, or a combination thereof, CYP76AD1, or a combination thereof, within said plant or part thereof. In one embodiment, if the food crop expresses CYP76AD1 and DOD and not CYP76AD6 or CYP76AD15, it further expresses a betalain related glucosyltransferase.

In another embodiment, the present invention provides plant or part thereof that serves as a food crop comprising a nucleic acid sequence encoding CYP76AD1, CYP76AD6, CYP76AD15, or a combination thereof, and a nucleic acid sequence encoding DOPA 4,5-dioxygenase (DOD) enzyme.

In one embodiment, the compositions and method of the present invention comprise one or more plant parts. In one embodiment, a plant part as described herein is a plant organ. In one embodiment, the plant part is a leaf, stem, root, flower, seed, tuber, or fruit. In one embodiment, the plant part is the edible portion of the plant. In one embodiment, the compositions and method of the present invention comprise one or more portions of one or more plant parts. In one embodiment, the portion of the plant part is a portion of a flower. In one embodiment, the portion of the flower is the petal, stamen, anther, or stigma.

In one embodiment, CYP76AD6, CYP76AD15, or a combination thereof, and other genes of the invention described herein can be transformed into a plant or a plant cell. The term “plant,” as used herein may relate to any monocot or dicot plant. Examples of monocot plants include, but are not limited to, corn, wheat, rice, sugar cane, and banana. Examples of dicot plants include, but are not limited to, soybean, beans, peas, lentils, peanuts, tomatoes, potatoes, cotton, and perennial fruit trees including grapes, apple, and orange.

In one embodiment, the plant is tobacco (Nicotiana tabacum), tomato (Solanum lycopersicum), potato (Solanum tuberosum), eggplant (Solanum melongena), tree tobacco (Nicotiana glauca), European black nightshade (Solanum nigrum), Petunia (Petunia x hybrida) or Nicotiana benthamiana. In another embodiment, the plant is B. vulgaris. In another embodiment, the plant is Mirabilis jalapa.

In one embodiment, a plant of the present invention is a crop plant. In one embodiment, the crop plant is Solanum tuberosum (Potato). In another embodiment, the crop plant is Zea mays (Maize). In another embodiment, the crop plant is Oryza sativa (Rice). In another embodiment, the crop plant is Manihot esculenta (Cassava). In another embodiment, the crop plant is Hordeum vulgare (Barley). In another embodiment, the crop plant is Triticum aestivum (Wheat). In another embodiment, the crop plant is Sorghum bicolor. In another embodiment, the crop plant is Brassica napus (Rapeseed). In another embodiment, the crop plant is Ricinus communis (Castor). In another embodiment, the crop plant is Phaseolus vulgaris (Bean). In another embodiment, the crop plant is Gossypium histrum (Cotton). In another embodiment, the crop plant is Glycine max (Soybean). In another embodiment, the crop plant is Beta vulgaris (Beet). In another embodiment, the crop plant is Musa acuminate (Banana). In another embodiment, the crop plant is Capsicum annuum (Sweet and Chili Peppers). In another embodiment, the crop plant is Cicer arietinum (Chick pea). In another embodiment, the crop plant is Solanum lycopersicum (Tomato). In another embodiment, the crop plant is Elaeis guineensis (African oilpalm). In another embodiment, the crop plant is Setaria italic (Foxtail millet).

In another embodiment, a plant of the present invention is a bamboo, which in one embodiment is river cane (Arundinaria gigantea) and switch cane (Arundinaria tecta). In another embodiment, the plant is a Lemna.

In another embodiment, a plant of the present invention is a moss. In one embodiment, the moss is a Sphagnum. In one embodiment, the Sphagnum species is cristatum or subnitens. In one embodiment, the moss is used for peat. In one embodiment, peat is used for fuel, as a horticultural soil additive, and in smoking malt in the production of Scotch whisky. In another embodiment, the moss is used for decorative purposes, such as in gardens and in the florist trade. In another embodiment, the moss is used as insulation. In another embodiment, the moss is used as an absorber of liquids. In another embodiment, moss is used for first-aid dressings, for diapers or napkins. In another embodiment, the moss is a Physcomitrella patens. In another embodiment, the moss is a Fontinalis antipyretica which, in one embodiment, is used to subdue fires.

Plants included in the invention are any plants amenable to transformation techniques, including gymnosperms and angiosperms, both monocotyledons and dicotyledons.

Examples of monocotyledonous angiosperms include, but are not limited to, asparagus, field and sweet corn, barley, wheat, rice, sorghum, onion, pearl millet, rye and oats and other cereal grains.

Examples of dicotyledonous angiosperms include, but are not limited to tomato, tobacco, cotton, rapeseed, field beans, soybeans, peppers, lettuce, peas, alfalfa, clover, cole crops or Brassica oleracea (e.g., cabbage, broccoli, cauliflower, brussel sprouts), radish, carrot, beets, eggplant, spinach, cucumber, squash, melons, cantaloupe, sunflowers and various ornamentals.

In another embodiment, any species of woody, ornamental or decorative, crop or cereal, fruit or vegetable plant, and algae (e.g., Chlamydomonas reinhardtii) may be used in the compositions and methods provided herein. Non-limiting examples of plants include plants from the genus Arabidopsis or the genus Oryza. Other examples include plants from the genuses Acorus, Aegilops, Allium, Amborella, Antirrhinum, Apium, Arachis, Beta, Betula, Brassica, Capsicum, Ceratopteris, Citrus, Cryptomeria, Cycas, Descurainia, Eschscholzia, Eucalyptus, Glycine, Gossypium, Hedyotis, Helianthus, Hordeum, Ipomoea, Lactuca, Linum, Liriodendron, Lotus, Lupinus, Lycopersicon, Medicago, Mesembryanthemum, Nicotiana, Nuphar, Pennisetum, Peryea, Phaseolus, Physcomitrella, Picea, Pinus, Poncirus, Populus, Prunus, Robinia, Rosa, Saccharum, Schedonorus, Secale, Sesamum, Solanum, Sorghum, Stevia, Thellungiella, Theobroma, Triphysaria, Triticum, Vitis, Zea, or Zinnia.

Examples of woody species include poplar, pine, sequoia, cedar, oak, etc.

In certain embodiments, plants of the present invention are crop plants (for example, cereals and pulses, maize, wheat, potatoes, tapioca, rice, sorghum, millet, cassaya, barley, pea, and other root, tuber, or seed crops). Exemplary cereal crops used in the compositions and methods of the invention include, but are not limited to, any species of grass, or grain plant (e.g., barley, corn, oats, rice, wild rice, rye, wheat, millet, sorghum, triticale, etc.), non-grass plants (e.g., buckwheat flax, legumes or soybeans, etc.). Grain plants that provide seeds of interest include oil-seed plants and leguminous plants. Other seeds of interest include grain seeds, such as corn, wheat, barley, rice, sorghum, rye, etc. Oil seed plants include cotton, soybean, safflower, sunflower, Brassica, maize, alfalfa, palm, coconut, etc. Other important seed crops are oil-seed rape, sugar beet, maize, sunflower, soybean, and sorghum. Leguminous plants include beans and peas. Beans include guar, locust bean, fenugreek, soybean, garden beans, cowpea, mungbean, lima bean, fava bean, lentils, chickpea, etc.

Horticultural plants to which the present invention may be applied may include lettuce, endive, and vegetable brassicas including cabbage, broccoli, and cauliflower, and carnations and geraniums. The present invention may also be applied to tobacco, cucurbits, carrot, strawberry, sunflower, tomato, pepper, chrysanthemum, poplar, eucalyptus, and pine.

The present invention may be used for transformation of other plant species, including, but not limited to, canola (Brassica napus, Brassica rapa ssp.), alfalfa (Medicago sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum, Nicotiana benthamiana), tree tobacco (Nicotiana glauca), peanuts (Arachis hypogaea), cotton (Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), tomato (Solanum lycopersicum), eggplant (Solanum melongena), European black nightshade (Solanum nigrum), Petunia (Petunia x hybrida), oats, barley, millet, fruit, vegetables, ornamentals, turfgrass, and conifers.

In addition to a plant, the present invention provides any clone of such a plant, seed, selfed or hybrid progeny and descendants, and any part of any of these, such as cuttings, seed. The invention provides any plant propagule, that is any part which may be used in reproduction or propagation, sexual or asexual, including cuttings, seed and so on. Also encompassed by the invention is a plant which is a sexually or asexually propagated off-spring, clone or descendant of such a plant, or any part or propagule of said plant, off-spring, clone or descendant. Plant extracts and derivatives are also provided.

Throughout this application a plant, plant part, seed or plant cell transformed with—or interchangeably transformed by—a construct or transformed with or by a nucleic acid is to be understood as meaning a plant, plant part, seed or plant cell that carries said construct or said nucleic acid as a transgene due the result of an introduction of said construct or said nucleic acid by biotechnological means. The plant, plant part, seed or plant cell therefore comprises said recombinant construct or said recombinant nucleic acid. Any plant, plant part, seed or plant cell that no longer contains said recombinant construct or said recombinant nucleic acid after introduction in the past, is termed null-segregant, nullizygote or null control, but is not considered a plant, plant part, seed or plant cell transformed with said construct or with said nucleic acid within the meaning of this application.

In one embodiment, tyrosine is endogenous to an organism or part of an organism of the present invention. In another embodiment, an organism or part of an organism of the present invention synthesizes tyrosine. In another embodiment, an organism or part of an organism of the present invention is genetically modified to produce tyrosine. In another embodiment, an organism or part of an organism of the present invention is provided or fed with tyrosine. In another embodiment, an organism or part of an organism that comprises endogenous tyrosine is genetically modified as described herein in order to produce larger amounts of tyrosine, thereby increasing L-DOPA or betalain production. In another embodiment, an organism or part of an organism that comprises endogenous tyrosine is fed tyrosine in order to have larger amounts of available tyrosine, thereby increasing L-DOPA or betalain production.

### Methods of Extracting L-DOPA and Betalains

In one embodiment, methods of the present invention further provide methods of extracting L-DOPA or betalains as described hereinbelow in Example 1 as well as using other L-DOPA or betalain extraction methods known in the art, such as, for example, Misra and Wagner (Indian J Biochem Biophys. 2007 February; 44(1):56-60), which is incorporated by reference herein in its entirety.

In another embodiment, L-DOPA or betalains are extracted from algae, yeast or bacterial culture using methods known in the art.

In another embodiment, the present invention provides a method of harvesting L-DOPA or betalains comprising the steps of producing L-DOPA or betalains as described herein and further comprising the step of extracting said L-DOPA or said betalain from the plant, plant part, colony, organ, tissue or cells that produce said L-DOPA or said betalain. In one embodiment, the plant part is a leaf, stem, root, flower, seed, tuber, or fruit.

In one embodiment, the L-DOPA or betalain is extracted from the root of a plant. In one embodiment, L-DOPA or betalain is secreted through the roots of a plant to a liquid or other medium and then harvested from the liquid or other medium. In another embodiment, the L-DOPA or betalain is extracted from the medium of a plant cell suspension, such as BY2 tobacco.

In another embodiment, L-DOPA or betalain is extracted from a water plant. In one embodiment, the water plant is a Lemna. In one embodiment, the Lemna is Lemna aequinoctialis; Lemna perpusilla; Lemna; Lemna gibba; Lemna minor; Lemna trisulcaUninerves; Lemna minuta; Lemna valdiviana; Lemna japonica; Lemna obscura; Lemna tenera; Lemna turionifera; Lemna yungensis, or a combination thereof.

### Polypeptides

In one embodiment, the present invention provides a chimeric polypeptide encoded by a recombinant polynucleotide comprising a nucleic acid encoding CYP76AD6. In one embodiment, the amino acid sequence encoding CYP76AD6 comprises:

(SEQ ID NO: 18)
MDNATLAVILSILFVFYHIFKSFFTNSSSRRLPPGPKPVPIFGNIFDLGE
KPHRSFANLSKIHGPLISLKLGSVTTIVVSSASVAEEMFLKNDQALANRT
IPDSVRAGDHDKLSMSWLPVSQKWRNMRKISAVQLLSNQKLDASQPLRQA
KVKQLLSYVQVCSEKMQPVDIGRAAFTTSLNLLSNTFFSIELASHESSAS
QEFKQLMWNIMEEIGRPNYADFFPILGYIDPFGIRRRLAGYFDKLIDVFQ
DIIRERQKLRSSNSSGAKQTNDILDTLLKLHEDNELSMPEINHLLVDIFD
AGTDTTASTLEWAMAELVKNPEMMTKVQIEIEQALGKDCLDIQESDISKL
PYLQAIIKETLRLHPPTVFLLPRKADNDVELYGYVVPKNAQVLVNLWAIG
RDPKVWKNPEVFSPERFLDCNIDYKGRDFELLPFGAGRRICPGLTLAYRM
LNLMLATLLQNYNWKLEDGINPKDLDMDEKFGITLQKVKPLQVIPVPRN.

In another embodiment, the present invention provides a chimeric polypeptide encoded by a recombinant polynucleotide comprising a nucleic acid encoding CYP76ADL. In one embodiment, the amino acid sequence encoding CYP76AD1 comprises:

(SEQ ID NO: 14)
MDHATLAMILAIWFISFHFIKLLFSQQTTKLLPPGPKPLPIIGNILEVGK
KPHRSFANLAKIHGPLISLRLGSVTTIVVSSADVAKEMFLKKDHPLSNRT
IPNSVTAGDHHKLTMSWLPVSPKWRNFRKITAVHLLSPQRLDACQTFRHA
KVQQLYEYVQECAQKGQAVDIGKAAFTTSLNLLSKLFFSVELAHHKSHTS
QEFKELIWNEVIEDIGKPNYADYFPILGCVDPSGIRRRLACSFDKLIAVF
QGIICERLAPDSSTTTTTTTDDVLDVLLQLFKQNELTMGEINHLLVDIFD
AGTDTTSSTFEWVMTELIRNPEMMEKAQEEIKQVLGKDKQIQESDIINLP
YLQAIIKETLRLHPPTVFLLPRKADTDVELYGYIVPKDAQILVNLWAIGR
DPNAWQNADIFSPERFIGCEIDVKGRDFGLLPFGAGRRICPGMNLAIRML
TLMLATLLQFFNWKLEGDISPKDLDMDEKFGIALQKTKPLKLIPIPRY.

In another embodiment, the present invention provides a chimeric polypeptide encoded by a recombinant polynucleotide comprising a nucleic acid encoding a DOPA 4,5-dioxygenase (DOD) enzyme. In one embodiment, the DOD enzyme is Beta vulgaris DODA1 (BvDODA1). In one embodiment, the amino acid sequence encoding BvDODA1 comprises:

(SEQ ID NO: 42)
MKMMNGEDANDQMIKESFFITHGNPILTVEDTHPLRPFFETWREKIFSKK
PKAILIISGHWETVKPTVNAVHINDTIHDFDDYPAAMYQFKYPAPGEPEL
ARKVEEILKKSGFETAETDQKRGLDHGAWVPLMLMYPEADIPVCQLSVQP
HLDGTYHYNLGRALAPLKNDGVLIIGSGSATHPLDETPHYFDGVAPWAAA
FDSWLRKALINGRFEEVNIYESKAPNWKLAHPFPEHFYPLHVVLGAAGEK
WKAELIHSSWDHGTLCHGSYKFTSA.

In one embodiment, the present invention provides a chimeric polypeptide encoded by a recombinant polynucleotide comprising a nucleic acid encoding cyclo-DOPA 5-O-glucosyltransferase (cDOPA5GT). In one embodiment, the amino acid sequence encoding cDOPA5GT comprises:

(SEQ ID NO: 43)
MTAIKMNTNGEGETQHILMIPFMAQGHLRPFLELAMFLYKRSHVIITLLT
TPLNAGFLRHLLHHHSYSSSGIRIVELPFNSTNHGLPPGIENTDKLTLPL
VVSLFHSTISLDPHLRDYISRHFSPARPPLCVIHDVFLGWVDQVAKDVGS
TGVVFTTGGAYGTSAYVSIWNDLPHQNYSDDQEFPLPGFPENHKFRRSQL
HRFLRYADGSDDWSKYFQPQLRQSMKSFGWLCNSVEEIETLGFSILRNYT
KLPIWGIGPLIASPVQHSSSDNNSTGAEFVQWLSLKEPDSVLYISFGSQN
TISPTQMMELAAGLESSEKPFLWVIRAPFGFDINEEMRPEWLPEGFEERM
KVKKQGKLVYKLGPQLEILNHESIGGFLTHCGWNSILESLREGVPMLGWP
LAAEQAYNLKYLEDEMGVAVELARGLEGEISKEKVKRIVEMILERNEGSK
GWEMKNRAVEMGKKLKDAVNEEKELKGSSVKAIDDFLDAVMQAKLEPSL
Q.

In one embodiment, a polypeptide of the present invention comprises an amino acid sequence encoding CYP76AD6, CYP76AD15, or a combination thereof, and an amino acid sequence encoding CYP76AD1. In another embodiment, a polypeptide of the present invention comprises an amino acid sequence encoding CYP76AD6, CYP76AD15, or a combination thereof, and an amino acid sequence encoding a DOD enzyme. In another embodiment, a polypeptide of the present invention comprises an amino acid sequence encoding CYP76AD1 and an amino acid sequence encoding a DOD enzyme. In another embodiment, a polypeptide of the present invention comprises an amino acid sequence encoding CYP76AD6, CYP76AD15, or a combination thereof, an amino acid sequence encoding CYP76AD1, and an amino acid sequence encoding a DOD enzyme. In another embodiment, a polypeptide of the present invention comprises an amino acid sequence encoding CYP76AD6, CYP76AD15, or a combination thereof, an amino acid sequence encoding CYP76AD1, and an amino acid sequence encoding a betalain related glucosyltransferase. In another embodiment, a polypeptide of the present invention comprises an amino acid sequence encoding CYP76AD6, CYP76AD15, or a combination thereof, an amino acid sequence encoding a DOD enzyme and an amino acid sequence encoding a betalain related glucosyltransferase. In another embodiment, a polypeptide of the present invention comprises an amino acid sequence encoding CYP76AD1, an amino acid sequence encoding a DOD enzyme and an amino acid sequence encoding a betalain related glucosyltransferase. In another embodiment, a polypeptide of the present invention comprises an amino acid sequence encoding CYP76AD6, CYP76AD15, or a combination thereof, an amino acid sequence encoding CYP76AD1, an amino acid sequence encoding a DOD enzyme and an amino acid sequence encoding a betalain related glucosyltransferase.

In another embodiment, any of the polypeptides described hereinabove further comprises a betalain related glucosyltransferase. In one embodiment, the betalain related glucosyltransferase is cyclo-DOPA 5-O-glucosyltransferase or betanidin-5-O-glucosyltransferase. In one embodiment, the cyclo-DOPA 5-O-glucosyltransferase is M. jalapa gene cyclo-DOPA 5-O-glucosyltransferase (cDOPA5GT) (SEQ ID NO: 43).

In one embodiment, the present invention provides a chimeric polypeptide encoded by a recombinant polynucleotide as described herein.

In another embodiment, the present invention provides a chimeric polypeptide comprising CYP76AD6, CYP76AD15, or a combination thereof, CYP76AD1, a DOPA 4,5-dioxygenase (DOD) enzyme, a betalain related glucosyltransferase, or a combination thereof, wherein said proteins are expressed in tandem.

In another embodiment, the present invention provides a chimeric polypeptide comprising CYP76AD1, a DOPA 4,5-dioxygenase (DOD) enzyme, and, optionally, a betalain related glucosyltransferase, wherein said proteins are expressed in tandem.

In another embodiment, the present invention provides a chimeric polypeptide comprising CYP76AD6, CYP76AD15, or a combination thereof, and a DOPA 4,5-dioxygenase (DOD) enzyme, wherein said proteins are expressed in tandem.

In another embodiment, the present invention provides a chimeric polypeptide comprising CYP76AD6, CYP76AD15, or a combination thereof, CYP76AD1, or a combination thereof, a DOPA 4,5-dioxygenase (DOD) enzyme, and, optionally a betalain related glucosyltransferase, wherein said proteins are expressed in tandem.

In one embodiment, a “chimeric” or “fusion” polypeptide or protein is a polypeptide or protein created through the joining of two or more genes that originally coded for separate proteins.

It is to be understood that according to the present invention, CYP76AD6 and CYP76AD6-like polypeptides from plants other than Beta vulgaris can be used in place of CYP76AD6 from Beta vulgaris in the compositions and methods described herein. In one embodiment, the CYP76AD6-like polypeptide is from Caryophyllales. In one embodiment, the CYP76AD6-like polypeptide is a homologue within the CYP76AD1-beta clade, as described in Brockington et al. (2015) (New Phytol. 2015 September; 207(4):1170-80), which is incorporated herein by reference in its entirety.

In one embodiment, CYP76AD15 or a homologue, isoform, or variant thereof, may be used in place of CYP76AD6 in the compositions and methods of the present invention. CYP76AD15 from Mirabilis jalapa has a similar function as CYP76AD6 from Beta vulgaris. In one embodiment, CYP76AD15 is an enzyme involved in converting tyrosine to L-DOPA. In one embodiment the amino acid sequence of CYP76AD15 comprises:

(SEQ ID NO: 44)
MENTMLGVILATIFLTFHIMKMLFSPSKVKLPPGPRPLPIIGNILELG
DKPHRSFANLAKIHGPLVTLKLGSVTTIVVSSSEVAKEMFLKNDQPLA
NRTIPDSVRAGNHDKLSMSWLPVSPKWRNLRKISAVQLLSTQRLDASQ
AHRQAKIKQLIEYVKKCSKIGQYVDIGQVAFTTSLNLLSNTFFSKELA
SFDSDNAQEFKQLMWCIIVIEEIGRPNYADYFPILGYVDPFGARRRLS
RYFDQLIEVFQVIIRERLTHDNNIVGNNNDVLATLLDLYKQNELTMDE
INHLLVDIFDAGTDTTASTLEWAMSELIKNPHIMAKAQEEVRRATMSH
GGATVAEIQESDINNLPYIQSIIKETLRLHPPTVFLLPRKADVDVQLF
GYVVPKNAQVLVNLWAIGRDPNVWPDPEVFSPERFMDCEIDVKGRDFE
LLPFGAGRRICPGLSLAYRMLNLMLANMVHSFDWKLPGVENGSGSEMD
SLDMDEKFGITLQKVQPLKVIPVSRK.

In one embodiment, the present invention provides an isolated polypeptide comprising an amino acid sequence encoding CYP76AD6, CYP76AD15, or a combination thereof, or another polypeptide described herein. In another embodiment, the present invention provides a non-naturally occurring polypeptide comprising an amino acid sequence encoding CYP76AD6, CYP76AD15, or a combination thereof, or another polypeptide described herein. In one embodiment, the CYP76AD6, CYP76AD15, or a combination thereof, sequence is a modified sequence. In one embodiment, it is an enhanced sequence. In one embodiment, it is an optimized sequence.

In one embodiment a polypeptide of the present invention optionally comprises CYP76AD6. In one embodiment, the amino acid sequence of CYP76AD6 is as set forth in SEQ ID NO: 18. In another embodiment, the CYP76AD6 utilized in methods and compositions of the present invention is a homologue of SEQ ID NO: 18. In another embodiment, the CYP76AD6 utilized in methods and compositions of the present invention is a variant of SEQ ID NO: 18. In another embodiment, the CYP76AD6 utilized in methods and compositions of the present invention is a fragment of SEQ ID NO: 18. In another embodiment, the CYP76AD6 utilized in methods and compositions of the present invention is an isoform of SEQ ID NO: 18. In another embodiment, the CYP76AD6 utilized in methods and compositions of the present invention is a functional homologue, functional variant, functional fragment, or functional isoform of SEQ ID NO: 18.

In one embodiment a polypeptide of the present invention optionally comprises CYP76AD15. In one embodiment, the amino acid sequence of CYP76AD15 is as set forth in SEQ ID NO: 44. In another embodiment, the CYP76AD15 utilized in methods and compositions of the present invention is a homologue of SEQ ID NO: 44. In another embodiment, the CYP76AD15 utilized in methods and compositions of the present invention is a variant of SEQ ID NO: 44. In another embodiment, the CYP76AD15 utilized in methods and compositions of the present invention is a fragment of SEQ ID NO: 44. In another embodiment, the CYP76AD15 utilized in methods and compositions of the present invention is an isoform of SEQ ID NO: 44.

In another embodiment, the CYP76AD15 utilized in methods and compositions of the present invention is a functional homologue, functional variant, functional fragment, or functional isoform SEQ ID NO: 44.

In one embodiment a polypeptide of the present invention optionally comprises CYP76AD1. In one embodiment, the amino acid sequence of CYP76AD1 is as set forth in SEQ ID NO: 14. In another embodiment, the CYP76AD1 utilized in methods and compositions of the present invention is a homologue of SEQ ID NO: 14. In another embodiment, the CYP76AD1 utilized in methods and compositions of the present invention is a variant of SEQ ID NO: 14. In another embodiment, the CYP76AD1 utilized in methods and compositions of the present invention is a fragment of SEQ ID NO: 14. In another embodiment, the CYP76AD1 utilized in methods and compositions of the present invention is an isoform of SEQ ID NO: 14.

In another embodiment, the CYP76AD1 utilized in methods and compositions of the present invention is a functional homologue, functional variant, functional fragment, or functional isoform of SEQ ID NO: 14.

In one embodiment a polypeptide of the present invention optionally comprises BvDODA1. In one embodiment, the amino acid sequence of BvDODA1 is as set forth in SEQ ID NO: 42. In another embodiment, the BVDODA1 utilized in methods and compositions of the present invention is a homologue of SEQ ID NO: 42. In another embodiment, the BVDODA1 utilized in methods and compositions of the present invention is a variant of SEQ ID NO: 42. In another embodiment, the BVDODA1 utilized in methods and compositions of the present invention is a fragment of SEQ ID NO: 42. In another embodiment, the BVDODA1 utilized in methods and compositions of the present invention is an isoform of SEQ ID NO: 42.

In another embodiment, the BVDODA1 utilized in methods and compositions of the present invention is a functional homologue, functional variant, functional fragment, or functional isoform of SEQ ID NO: 42.

In one embodiment, a polypeptide of the present invention optionally comprises cDOPA5GT. In one embodiment, the amino acid sequence of cDOPA5GT is as set forth in SEQ ID NO: 43. In another embodiment, the cDOPA5GT utilized in methods and compositions of the present invention is a homologue of SEQ ID NO: 43. In another embodiment, the cDOPA5GT utilized in methods and compositions of the present invention is a variant of SEQ ID NO: 43. In another embodiment, the cDOPA5GT utilized in methods and compositions of the present invention is a fragment of SEQ ID NO: 43. In another embodiment, the cDOPA5GT utilized in methods and compositions of the present invention is an isoform of SEQ ID NO: 43.

In another embodiment, the cDOPA5GT utilized in methods and compositions of the present invention is a functional homologue, functional variant, functional fragment, or functional isoform of SEQ ID NO: 43.

In some embodiments, “polypeptide”, “engineered polypeptide”, or “protein” as used herein encompasses native polypeptides (either degradation products, synthetically synthesized polypeptides or recombinant polypeptides) and peptidomimetics (typically, synthetically synthesized polypeptides), as well as peptoids and semipeptoids which are polypeptide analogs, which have, in some embodiments, modifications rendering the polypeptides even more stable while in a body or more capable of penetrating into cells.

In some embodiments, modifications include, but are limited to C terminus modification, polypeptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

In some embodiments, polypeptide bonds (—CO—NH—) within the polypeptide are substituted. In some embodiments, the polypeptide bonds are substituted by N-methylated bonds (—N(CH3)-CO—). In some embodiments, the polypeptide bonds are substituted by ester bonds (—C(R)H—C—O—O—C(R)—N—). In some embodiments, the polypeptide bonds are substituted by ketomethylen bonds (—CO—CH2-). In some embodiments, the polypeptide bonds are substituted by α-aza bonds (—NH—N(R)—CO—), wherein R is any alkyl, e.g., methyl, carba bonds (—CH2-NH—). In some embodiments, the polypeptide bonds are substituted by hydroxyethylene bonds (—CH(OH)—CH2-). In some embodiments, the polypeptide bonds are substituted by thioamide bonds (—CS—NH—). In some embodiments, the polypeptide bonds are substituted by olefinic double bonds (—CH═CH—). In some embodiments, the polypeptide bonds are substituted by retro amide bonds (—NH—CO—). In some embodiments, the polypeptide bonds are substituted by polypeptide derivatives (—N(R)—CH2-CO—), wherein R is the “normal” side chain, naturally presented on the carbon atom. In some embodiments, these modifications occur at any of the bonds along the polypeptide chain and in one embodiment at several (2-3 bonds) at the same time.

In some embodiments, natural aromatic amino acids of the polypeptide such as Trp, Tyr and Phe, are substituted for synthetic non-natural acid such as Phenylglycine, TIC, naphthylelanine (Nol), ring-methylated derivatives of Phe, halogenated derivatives of Phe or o-methyl-Tyr. In some embodiments, the polypeptides of the present invention include one or more modified amino acid or one or more non-amino acid monomers (e.g. fatty acid, complex carbohydrates etc).

In one embodiment, “amino acid” or “amino acid sequence” is understood to include the 20 naturally occurring amino acid; those amino acid often modified post-translationally in vivo, including, for example, hydroxyproline, phosphoserine and phosphothreonine; and other unusual amino acid including, but not limited to, 2-aminoadipic acid, hydroxylysine, isodesmosine, nor-valine, nor-leucine and ornithine. In one embodiment, “amino acid” includes both D- and L-amino acids.

In some embodiments, the polypeptides of the present invention are utilized in therapeutics which requires the polypeptides to be in a soluble form. In some embodiments, the polypeptides of the present invention include one or more non-natural or natural polar amino acid, including but not limited to serine and threonine which are capable of increasing polypeptide solubility due to their hydroxyl-containing side chain.

In some embodiments, the engineered polypeptide of the present invention is utilized in a linear form, although it will be appreciated by one skilled in the art that in cases where cyclicization does not severely interfere with engineered polypeptides characteristics, cyclic forms of the engineered polypeptides can also be utilized.

In some embodiments, the engineered polypeptides of the present invention are biochemically synthesized such as by using standard solid phase techniques. In some embodiments, these biochemical methods include exclusive solid phase synthesis, partial solid phase synthesis, fragment condensation, or classical solution synthesis.

In some embodiments, recombinant protein techniques are used to generate the engineered polypeptides of the present invention. In some embodiments, recombinant protein techniques are used for the generation of relatively long polypeptides (e.g., longer than 18-25 amino acids). In some embodiments, recombinant protein techniques are used for the generation of large amounts of the engineered polypeptides of the present invention. In some embodiments, recombinant techniques are described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463, which are incorporated herein by reference in their entirety.

In one embodiment, the present invention provides a chimeric polypeptide comprising a CYP76AD1-β clade polypeptide. In one embodiment, the CYP76AD1-β clade polypeptide is CYP76AD6. In another embodiment, the CYP76AD1-β clade polypeptide is CYP76AD15. In one embodiment, the present invention provides a chimeric polypeptide comprising a CYP76AD1-β clade polypeptide but excluding the amino acid sequence of the CYP76AD1 paralogs from sugar beet known as Bv9_228610 vgeq or Bv9_228860_ickx, which do not produce betaxanthin when transformed into yeast with DOD. In one embodiment, the present invention provides a chimeric polypeptide comprising a CYP76AD1-β clade polypeptide but excluding the following amino acid sequence:

(SEQ ID NO: 34)
MDNATLAVILSILFVFYHIFKSFFTNSSSRRLPPGPKPVPIFGNIFDL
GEKPHRSFANLSKIHGPLISLKLGSVTTIVVSSASVAEEMFLKNDQAL
ANRTIPDSVRAGDHDKLSMSWLPVSQKWRNMRKISAVQLLSNQKLDAS
QPLRQTKVKQLLSYVQDCSKKMQPVDIGRAAFTTSLNLLSNTFFSIEL
ASHESSASQEFKQLMWNIMEEIGRPNYADFFPILGYIDPFGIRRRLAG
YFDKLIDVFQDIIRERQKLRSSNSSGAKQTNDILDTLLKLHEDNELSM
PEINHLLVDIFDAGTDTTASTLEWAMAELVKNPEMMTKVQIEIEQALG
KDCLDIQESDISKLPYLQGIIKETLRLHPPTVFLLPRKADNDVELYGY
VVPKNAQVLVNLWAIGRDPKVWKNPEVFSPERFLDCNIDYKGRDFELL
PFGAGRRICPGLTLAYRMLNLMLATLLQNYNWKLEDGINPKDLDMDEK
FGITLQKVKPLQVIPVPRN.

In one embodiment, the present invention provides methods comprising the step of “contacting” tyrosine with one or more polypeptides as described herein. In one embodiment, said contacting step is conducted in vitro under conditions which allow the production of L-DOPA or betalains, as described herein.

### Polypeptide Recovery

In one embodiment, methods of the present invention further provide methods of extracting betalains or L-DOPA from plants as described hereinbelow in Example 1 as well as using other betalain extraction methods known in the art. In another embodiment, betalains are extracted from algae, yeast or bacterial culture using methods known in the art. In one embodiment, the algae, yeast or bacterial culture are genetically modified to express betalains or L-DOPA.

In one embodiment, following a predetermined time in culture, recovery of a polypeptide is affected.

In one embodiment, the phrase “recovering the polypeptide” used herein refers to collecting the whole fermentation medium containing the polypeptide and need not imply additional steps of separation or purification.

In one embodiment, polypeptides of the present invention are purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

In one embodiment, to facilitate recovery, the expressed coding sequence can be engineered to encode the polypeptide of the present invention and fused cleavable moiety. In one embodiment, a fusion protein can be designed so that the polypeptide can be readily isolated by affinity chromatography; e.g., by immobilization on a column specific for the cleavable moiety. In one embodiment, a cleavage site is engineered between the polypeptide and the cleavable moiety and the polypeptide can be released from the chromatographic column by treatment with an appropriate enzyme or agent that specifically cleaves the fusion protein at this site [e.g., see Booth et al., Immunol. Lett. 19:65-70 (1988); and Gardella et al., J. Biol. Chem. 265:15854-15859 (1990)].

In one embodiment, the polypeptide of the present invention is retrieved in “substantially pure” form.

In one embodiment, the phrase “substantially pure” refers to a purity that allows for the effective use of the protein in the applications described herein.

In one embodiment, the polypeptide of the present invention can also be synthesized using in vitro expression systems. In one embodiment, in vitro synthesis methods are well known in the art and the components of the system are commercially available.

Methods of producing betaxanthins in vitro using a DOD enzyme with its substrate L-DOPA and adding amino acids was described in Sekiguchi et al. (2010) (Journal of Agricultural and Food Chemistry 58, 12504-12509), which is incorporated herein by reference in its entirety. In one embodiment, the present method of in vitro synthesis of betalains is as described in Sekiguchi et al. but with the added step of synthesizing L-DOPA from tyrosine rather than providing L-DOPA as described in Sekiguchi et al.

In one embodiment, in vitro production of red-violet betalains (betacyanins) is performed using the same or similar conditions as for the production of betaxanthins, except that the CYP76AD1 enzyme is provided together with the DOD enzyme, and the substrate could be either tyrosine or L-DOPA. In one embodiment, the co-factor NADPH may be added to activate the CYP76AD1 enzyme.

In some embodiments, the recombinant polypeptides are synthesized and purified; their therapeutic efficacy can be assayed in vivo or in vitro.

### Pharmaceutical Compositions

In another embodiment, provides a pharmaceutical composition comprising a recombinant polynucleotide or a chimeric polypeptide of the present invention. In one embodiment, the polypeptides and polynucleotides of the present invention can be provided to the individual as part of a pharmaceutical composition where it is mixed with a pharmaceutically acceptable carrier.

In one embodiment, a “pharmaceutical composition” refers to a preparation of one or more of the active ingredients described herein with other chemical components such as physiologically suitable carriers and excipients. The purpose of a pharmaceutical composition is to facilitate administration of a compound to an organism.

In one embodiment, “active ingredient” refers to the recombinant polynucleotide or the chimeric polypeptide, which is accountable for the biological or biochemical effect.

In other embodiments, the composition comprises additional components. In one embodiment, such additional components may comprise carriers or diluents including, but not limited to, a gum, a starch (e.g. corn starch, pregeletanized starch), a sugar (e.g. lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

In other embodiments, pharmaceutically acceptable carriers for liquid formulations are aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs.

In another embodiment, parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

In other embodiments, the compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g. Tris-HCI., acetate, phosphate) of various pH and ionic strength, additives such as albumin or gelatin to prevent absorption to surfaces, detergents (e.g. Tween 20, Tween 80, Pluronic F68, bile acid salts), protease inhibitors, surfactants (e.g. sodium lauryl sulfate), permeation enhancers, solubilizing agents (e.g. glycerol, polyethylene glycerol), anti-oxidants (e.g. ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g. Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g. poloxamers or poloxamines), coating and film forming agents (e.g. ethyl cellulose, acrylates, polymethacrylates) and/or adjuvants. Each of the above excipients represents a separate embodiment of the present invention.

In another embodiment, the pharmaceutical compositions provided herein are controlled-release compositions, i.e., compositions in which the antigen is released over a period of time after administration. Controlled- or sustained-release compositions include formulation in lipophilic depots (e.g. fatty acids, waxes, oils). In another embodiment, the composition is an immediate-release composition, i.e., a composition in which all the antigen is released immediately after administration.

In another embodiment, the pharmaceutical composition is delivered in a controlled release system. In another embodiment, the agent is administered using intravenous infusion, an implantable osmotic pump, a transdermal patch, liposomes, or other modes of administration. In another embodiment, a pump is used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials are used; e.g. in microspheres in or an implant.

The compositions also include, in another embodiment, incorporation of the active material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

Also included in the present invention are particulate compositions coated with polymers (e.g. poloxamers or poloxamines) and the compound coupled to antibodies directed against tissue-specific receptors, ligands or antigens or coupled to ligands of tissue-specific receptors.

Also comprehended by the invention are compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds (Abuchowski et al., 1981; Newmark et al., 1982; and Katre et al., 1987). Such modifications also increase, in another embodiment, the compound's solubility in aqueous solution, eliminate aggregation, enhance the physical and chemical stability of the compound, and greatly reduce the immunogenicity and reactivity of the compound. In another embodiment, the desired in vivo biological activity is achieved by the administration of such polymer-compound abducts less frequently or in lower doses than with the unmodified compound.

The preparation of pharmaceutical compositions that contain an active component, for example by mixing, granulating, or tablet-forming processes, is well understood in the art. An active component is, in another embodiment, formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Each of the above additives, excipients, formulations and methods of administration represents a separate embodiment of the present invention.

In one embodiment, methods of the present invention comprise administering a polynucleotide or polypeptide of the present invention in a pharmaceutically acceptable carrier.

The pharmaceutical compositions containing the polypeptide can be, in another embodiment, administered to a subject by any method known to a person skilled in the art, such as parenterally, transmucosally, transdermally, intramuscularly, intravenously, intra-dermally, intra-nasally, subcutaneously, intra-peritonealy, intra-ventricularly, intra-cranially, or intra-vaginally. In another embodiment, compositions of the instant invention are administered via epidermal injection, in another embodiment, intramuscular injection, in another embodiment, subcutaneous injection, and in another embodiment, intra-respiratory mucosal injection.

In another embodiment, of methods and compositions of the present invention, the pharmaceutical compositions are administered orally, and are thus formulated in a form suitable for oral administration, i.e., as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment, of the present invention, the composition is formulated in a capsule. In another embodiment, compositions of the present invention comprise a hard gelating capsule.

In another embodiment, the pharmaceutical compositions are administered by intravenous, intra-arterial, or intra-muscular injection of a liquid preparation. Suitable liquid formulations include solutions, suspensions, dispersions, emulsions, oils and the like. In another embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In another embodiment, the pharmaceutical compositions are administered topically to body surfaces and are thus formulated in a form suitable for topical administration. Suitable topical formulations include gels, ointments, creams, lotions, drops and the like.

In another embodiment, the pharmaceutical composition is administered as a suppository, for example a rectal suppository or a urethral suppository. In another embodiment, the pharmaceutical composition is administered by subcutaneous implantation of a pellet. In another embodiment, the pellet provides for controlled release of antigen agent over a period of time.

In another embodiment, the pharmaceutical composition is delivered in a vesicle, e.g. a liposome.

### Plant Regeneration

In one embodiment, a transgenic plant of the present invention is grown under conditions suitable for the expression of the recombinant DNA construct or constructs. The regeneration, development and cultivation of plants from single plant protoplast transformants or from various transformed explants is well known in the art (See Weissbach and Weissbach, In.: Methods for Plant Molecular Biology, (Eds.), 1988 Academic Press, Inc., San Diego, Calif). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

Following transformation, plant cells transformed with a plant expression vector may be regenerated, e.g., from single cells, callus tissue or leaf discs according to standard plant tissue culture techniques. Almost any plant can be entirely regenerated from cells, tissues, and organs of the plant using methods that are known in the art.

The transformed plants may then be grown, and either pollinated with the same transformed strain or different strains, and the resulting hybrid having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved.

In one embodiment, a plant cell is regenerated to obtain a whole plant from the transformation process. The term “growing” or “regeneration” as used herein means growing a whole plant from a plant cell, a group of plant cells, a plant part (including seeds), or a plant piece (e.g., from a protoplast, callus, or tissue part).

Regeneration from protoplasts varies from species to species of plants, but generally a suspension of protoplasts is first made. In certain species, embryo formation can then be induced from the protoplast suspension. In one embodiment, the culture media will contain various amino acids and hormones, necessary for growth and regeneration. Examples of hormones utilized include auxins and cytokinins. Efficient regeneration will depend on the medium, on the genotype, and on the history of the culture. If these variables are controlled, regeneration is reproducible. Regeneration also occurs from plant callus, explants, organs or parts.

In vegetatively propagated crops, the mature genetically modified plants are propagated by utilizing cuttings or tissue culture techniques to produce multiple identical plants. Selection of desirable genetically modified plants is made and new varieties are obtained and propagated vegetatively for commercial use.

In seed propagated crops, mature genetically modified plants can be self-crossed to produce a homozygous inbred plant. The resulting inbred plant produces seed containing the genetic mutation. These seeds can be grown to produce plants that would produce the selected phenotype, e.g., increased lateral root growth, uptake of nutrients, overall plant growth and/or vegetative or reproductive yields.

Parts obtained from the regenerated plant, such as flowers, seeds, leaves, branches, fruit, and the like are included in the invention, provided that these parts comprise cells comprising the isolated nucleic acid of the present invention. Progeny and variants, and mutants of the regenerated plants are also included within the scope of the invention, provided that these parts comprise the introduced nucleic acid sequences. In one embodiment, genetically modified plants expressing a selectable marker can be screened for transmission of the nucleic acid of the present invention by, for example, standard immunoblot and DNA detection techniques. In another embodiment, genetically modified plant cells may be evaluated on levels of expression of the genetically modified nucleic acid. Expression at the RNA level can be determined initially to identify and quantitate expression-positive plants. Standard techniques for RNA analysis can be employed and include PCR amplification assays using oligonucleotide primers designed to amplify only the genetically modified RNA templates and solution hybridization assays using genetically modified nucleic acid-specific probes. The RNA-positive plants can then be analyzed for protein expression by Western immunoblot analysis using the specifically reactive antibodies of the present invention. In addition, in situ hybridization and immunocytochemistry according to standard protocols can be performed using genetically modified nucleic acid specific polynucleotide probes and antibodies, respectively, to localize sites of expression within genetically modified tissue. In one embodiment, a number of genetically modified lines are screened for the incorporated nucleic acid to identify and select plants with the most appropriate expression profiles.

In one embodiment, the present invention provides a genetically modified plant that is homozygous for the introduced nucleic acid; i.e., a genetically modified plant that contains two added nucleic acid sequences, one gene at the same locus on each chromosome of a chromosome pair. A homozygous genetically modified plant can be obtained by sexually mating (selfing) a heterozygous genetically modified plant that contains a single added genetically modified nucleic acid, germinating some of the seed produced and analyzing the resulting plants produced for altered expression of a polynucleotide of the present invention relative to a control plant (i.e., native, non-genetically modified). Back-crossing to a parental plant and out-crossing with a non-genetically modified plant are also contemplated.

Transformed plant cells which are derived by any of the above transformation techniques can be cultured to regenerate a whole plant which possesses the transformed genotype. In one embodiment, such regeneration techniques rely on manipulation of certain phytohormones in a tissue culture growth medium.

The regenerated plants containing the foreign, exogenous gene that encodes a protein of interest can then be further propagated as is well known in the art. The particular method of propagation will depend on the starting plant tissue and the particular plant species to be propagated.

In one embodiment, the generated transformed plants are clonally propagated. In another embodiment, the generated transformed plants are propagated by classical breeding techniques. In a particular embodiment, the regenerated plants are self-pollinated to provide homozygous transgenic plants. Otherwise, pollen obtained from the regenerated plants is crossed to seed-grown plants of agronomically important lines, or pollen from plants of these important lines is used to pollinate regenerated plants. A transgenic plant of the present invention containing a desired polypeptide is cultivated using methods well known to one of skill in the art.

### Products of Genetically Modified Plants

In one embodiment, the present invention provides a food crop comprising betalains. In one embodiment, the food crop does not endogenously comprise betalains. In another embodiment, the food crop does endogenously comprise betalains and the methods of the present invention serve to increase or enhance the amount of betalains present in the food crop.

In one embodiment, a food crop comprising betalains or enhanced amounts of betalains is nutritionally enhanced. In one embodiment, a food crop comprising betalains or enhanced amounts of betalains comprises anti-oxidative properties. In one embodiment, consumption of a food crop comprising betalains or enhanced amounts of betalains protects a subject from degenerative diseases or conditions. In one embodiment, consumption of betalains or food crops containing betalains may protect a subject from cancer (e.g. skin, lung, cervical, ovarian and bladder), cardiopathy, or neurodegenerative diseases. Betalains may also inhibit lipid peroxidation and heme decomposition; prevent oxidative hemolysis of red blood cells; and bind to human low-density lipoproteins increasing their resistance to oxidation.

In one embodiment, the present invention provides fruit comprising betalains of the present invention. In another embodiment, the present invention provides vegetables comprising betalains of the present invention. In another embodiment, the present invention provides underground organs, such as potatoes, comprising betalains of the present invention. In another embodiment, the present invention provides juices derived from fruits or vegetables comprising betalains of the present invention.

In one embodiment, the plant organs or derivatives thereof (such as juice) comprise, in addition to betalains of the present invention, natural or engineered anthocyanins, carotenes, or a combination thereof.

In another embodiment, the present invention provides a dietary supplement comprising a betalain produced by the methods as described herein or a plant or plant part as described herein.

In another embodiment, the present invention provides an allelochemical comprising L-DOPA produced by the methods as described herein or a plant or plant part as described herein.

### Additional Uses

The heterologous production of betalains enables the biofortification and enhancement of nutritional qualities of essential foods, as well as the development of new varieties of ornamental plants. The simple biosynthetic pathway of these pigments, which starts from the ubiquitous precursor tyrosine and requires expression of two to three genes, allows for the production of these pigments in numerous plant species or other species.

In another embodiment, the present invention provides a method of producing an ornamental plant, comprising the step of contacting a plant cell with a nucleic acid sequence encoding CYP76AD1, CYP76AD6, CYP76AD15, or a combination thereof, and a nucleic acid sequence encoding DOPA 4,5-dioxygenase (DOD) enzyme and under conditions sufficient to produce betalains, wherein if said cell is contacted with a nucleic acid sequence encoding CYP76AD1 and DOD and not a nucleic acid sequence encoding CYP76AD6 or CYP76AD15, then said cell is contacted with a nucleic acid sequence encoding a betalain related glucosyltransferase, thereby producing an ornamental plant. In one embodiment, the plant is not a naturally betalain-expressing plant. In one embodiment, the plant is not a Caiyophyllales plant. In one embodiment, the species of plant is: Syngonium Podophyllum, Nephrolepis exaltata ‘bostoniensis’, Phoenix Canariensis, Aglaonema, Asplenium nidus, Rhapis excelsa, Aspidistra elatior, Nertera granadensis, Pteris cretica, Dieffenbachia amoena, Chamaerops humilis, Epipremnum aureum, Codiaeum variegatum, Alocasia amazonica, Fiddle Leaf Fig—Ficus lyrata, Alocasia micholitziana, Philodendron scandens, Dracaena braunii, Adiantum raddianum, Chamaedorea elegans, Howea forsteriana, Dracaena marginata, Pachira aquatica, Calathea makoyana, Davallia fejeensis, Calathea roseopicta, Cycas Revoluta, Dracaena reflexa, Calathea lancifolia, Ficus elastica, Howea belmoreana, Spider Plant—Chlorophytum comosum, Dionaea muscipula, Peperomia argyela, Aphelandra squarrosa, Zamioculcas zamiifolia, Tradescantia, Ficus benjamina, Calathea zebrina, or a Cactus.

In one embodiment, changing the color of a plant, and in particular the color of a flower of a plant, will affect the pollination of the plant. In one embodiment, methods of the present invention will increase or enhance the pollination of a plant. In another embodiment, methods of the present invention will decrease the pollination of a plant. In another embodiment, methods of the present invention will change the organisms that are attracted to the plant, thereby changing the pollination of a plant. In one embodiment, the present invention provides a method of increasing the pollination of a plant comprising the step of inserting a recombinant polynucleotide or one or more nucleic acids of the present invention into at least one cell of said plant.

In another embodiment, the present invention provides a method of increasing the pollination of a plant, comprising the step of contacting a plant with a nucleic acid sequence encoding CYP76AD1, CYP76AD6, CYP76AD15, or a combination thereof, and, optionally, a nucleic acid sequence encoding DOPA 4,5-dioxygenase (DOD) enzyme and under conditions sufficient to produce betalains, thereby changing the color of the plant, thereby increasing the pollination of a plant. In one embodiment, the plant is not a naturally betalain-expressing plant. In one embodiment, the plant is not a Caryophyllales' plant.

In another embodiment, the methods of the present invention provide methods of changing the color of an organism. In one embodiment, the organism is a fish.

In another embodiment, the methods of the present invention provide a method of producing an organism in which one or more parts of said organism is red-violet comprising the step of contacting one or more cells of said organism with a recombinant polynucleotide comprising a nucleic acid encoding a CYP76AD1, a nucleic acid encoding a DOPA 4,5-dioxygenase (DOD) enzyme, and a nucleic acid encoding betalain related glucosyltransferase, wherein said nucleic acids are inserted into the polynucleotide in frame.

In another embodiment, the methods of the present invention provide a method of producing an organism in which one or more parts of said organism is yellow comprising the step of contacting one or more cells of said organism with a recombinant polynucleotide comprising a nucleic acid encoding a CYP76AD6 and a nucleic acid encoding a DOPA 4,5-dioxygenase (DOD) enzyme, wherein said nucleic acids are inserted into the polynucleotide in frame.

In another embodiment, the methods of the present invention provide a method of producing an organism in which one or more parts of said organism is orange, comprising the step of contacting one or more cells of said organism with a recombinant polynucleotide comprising a nucleic acid encoding a CYP76AD6, a nucleic acid encoding CYP76AD1, a nucleic acid encoding a DOPA 4,5-dioxygenase (DOD) enzyme, and a nucleic acid encoding betalain related glucosyltransferase, wherein said nucleic acids are inserted into the polynucleotide in frame.

In one embodiment, the organism is a fish. In one embodiment, the fish is an ornamental fish.

In one embodiment, the present invention provides a method of producing an ornamental fish comprising contacting a cell of a fish with a polynucleotide encoding a nucleic acid encoding a DOPA 4,5-dioxygenase (DOD) enzyme and a nucleic acid encoding CYP76AD6, a nucleic acid encoding CYP76AD15, a nucleic acid encoding CYP76AD1, or a combination thereof, wherein if said cell is contacted with a nucleic acid encoding CYP76AD1 and a nucleic acid encoding DOD and not a nucleic acid encoding CYP76AD6 or a nucleic acid encoding CYP76AD15, said cell is also contacted with a nucleic acid encoding a betalain related glucosyltransferase.

In another embodiment, the present invention provides a method of producing a plant expressing a betalain, comprising the step of contacting one or more cells of said plant with a nucleic acid sequence encoding CYP76AD1, CYP76AD6, CYP76AD15, or a combination thereof, and a nucleic acid sequence encoding DOPA 4,5-dioxygenase (DOD) enzyme, screening said one or more cells to identify cells comprising said nucleic acid sequences, and regenerating plants or plant parts from said cells, thereby producing a plant expressing a betalain.

In one embodiment, the plant does not naturally express a betalain. In another embodiment, the plant expresses a betalain and the methods of the present invention are used to increase the amount of one or more betalains that are expressed by the plant.

In another embodiment, the present invention provides a method of producing a food crop expressing a betalain, comprising the step of: contacting one or more cells of a food crop plant with a nucleic acid sequence encoding CYP76AD1, CYP76AD6, CYP76AD15, or a combination thereof, and a nucleic acid sequence encoding DOPA 4,5-dioxygenase (DOD) enzyme, screening said one or more cells to identify cells comprising said nucleic acid sequences, and regenerating plants or plant parts from said cells, thereby producing a food crop expressing a betalain.

In one embodiment, a “food crop” or an “agricultural crop” is a plant intentionally grown with the primary purpose of being eaten by humans or animals. In one embodiment, the food crop is plantains, yams, sorghum, sweet potatos, soybeans, cassava, rice, wheat, or corn. In another embodiment, the food crop is aubergines, peppers, broccoli, calabrese, buckwheat, maize, barley, grapes, berries, tomatoes, cucumbers, artichoke, onion, rhubarb, blackberries, blueberries, zucchini, radishes, carrots, brussel sprouts, lettuce, melons, beans, peas, grains, peanuts, sugarcane, watermelon, papaya, apple, pear, peach, cherry, strawberry or squash.

In one embodiment, the strong antioxidant activity of betalains is beneficial for human health.

They have been extensively studied for their potential health-promoting properties, including anti-cancer, hypolipidemic, anti-inflammatory, hepatoprotective and anti-diabetic activities. Thus, in one embodiment, the present invention provides a method of treating or suppressing cancer, hyperlipidemia, inflammation, liver disease, and diabetes comprising the step of feeding to a subject in need a plant or plant part of the present invention, which expresses betalains, betacyanins, and/or betaxanthins, thereby treating or suppressing cancer, hyperlipidemia, inflammation, liver disease, or diabetes in said subject.

In one embodiment, ingestion by a subject of a food crop of the present invention comprising betalains or enhanced amounts of betalains protect subjects against Inflammatory-Immune Injury (including, but not limited to glomerulonephritis, vasculitis, autoimmune disease, adult respiratory distress syndrome, rheumatoid arthritis, inflammatory bowel disease, pancreatitis); cancer (including, but not limited to radiation induced cancer, cervical carcinoma, hepatocellular carcinoma, promotors of carcinogenesis, cancer in inflammatory bowel disease); ischemia/reoxygenation (including, but not limited to stroke, myocardial infraction, organ transplantation (heart, lung, skin, cornea, kidney), organ preservation, reattachment of severed limbs, frostbite, Dupuytren's contracture, hemorrhagic shock, endotoxic shock, crush injury); metal overload (including, but not limited to hemochromatosis, thalassemia, kwashlorkor, chemotherapy for leukemias, fulminant hepatic failure, Wilson's disease, alcohol induced iron overload, nickel induced carcinogenesis, lead poisoning); toxins (including, but not limited to hemolytic drugs, lead, halogenated hydrocarbons, ozone, oxides of nitrogen, asbestos, other mineral dusts, sulfur dioxid, paraquat, aluminum, cigarette smoke, diabetogenic drugs, fava beans (hemolytic agents), anthracyclines (cardiotoxicity), heavy metals (nephrotoxicity), photosensitizing drugs, contact dermatitis); eye disorders (including, but not limited to cataract development, deterioration after ocular hemorrhage, photochemical retinal damage, retinopathy of prematurity (retrolental fibroplasia)); inborn disease (including, but not limited to porphyrias, sickle cell anemia, Fanconi's anemia, neuronal ceroid lipofuscinoses, thalassemia); insufficient antioxidant protection (including, but not limited to Keshan disease (severe selenuim deficiency), hemolytic disease of prematurity, retinopathy of prematurity, bronchopulmonary dysplasia, intracranial hemorrhage, neurological degeneration due to severe vitamin E deficiency (in inborn errors affecting intestinal fat absorption), acquired immunodeficiency syndrome); brain and central nervous system disorders (including, but not limited to stroke, trauma, neurotoxicities (e.g., of aluminum), effects of hyperbaric oxygen, Parkinson's disease, potentiation of traumatic injury, cerebral malaria).

In another embodiment, the present invention provides new ways of producing betalains in non-naturally betalain-expressing species, in which betalains may be used as a natural food colorant. Therefore, the present invention also provides a natural food colorant comprising betalains produced as described herein. In one embodiment, betalains are extracted from plants expressing betalain as described herein. In another embodiment, betalains are produced in vitro in a cell line. The heterologous production of betalains enables the biofortification and enhancement of nutritional qualities of essential foods.

In another embodiment, the methods of present invention may be used to enhance the aesthetic quality of a food crop or produce by changing its color by genetically engineering a plant to express one or more betalains.

Thus, in one embodiment, the present invention provides methods of treating or suppressing the diseases, disorders, and conditions mentioned hereinabove comprising the step of providing a subject with a food crop that has been genetically modified to express betalains, as described herein. In another embodiment, the present invention provides uses of polynucleotides, nucleic acids and other compositions described herein in the preparation of a composition for treating or suppressing the diseases, disorders, and conditions mentioned hereinabove. In one embodiment, the composition is a pharmaceutical composition. In another embodiment, the present invention provides polynucleotides, nucleic acids and other compositions described herein for use in treating or suppressing the diseases, disorders, and conditions mentioned hereinabove.

In another embodiment, a food crop comprising betalains or enhanced amounts of betalains has an increased shelf life compared to the same food crop lacking betalains or having lower level of betalains. In another embodiment, a food crop comprising betalains or enhanced amounts of betalains has increased resistance to fungal diseases compared to the same food crop lacking betalains or having lower level of betalains.

In another embodiment, betalains produced by the methods described herein may be used for food preservation.

Therefore, in one embodiment, the present invention provides methods of increasing the shelf life of a food crop comprising the step of contacting one or more cells of a food crop plant comprising tyrosine with a nucleic acid encoding CYP76AD6, CYP76AD15, or a combination thereof, a nucleic acid encoding CYP76AD1, or a combination thereof, under conditions sufficient to produce betalains, thereby producing betalains and increasing the shelf life of said food crop.

Within the plant kingdom, betalains are found in only one group of angiosperms, the Caryophyllales. In this order, betalains and anthocyanins occur in a mutually exclusive fashion, i.e. no plant species produces both types of pigments. One of the most prominent features of the Caryophyllales order is its dominance in arid and semi-arid regions, and habitation of saline and alkaline soils. While some families within the Caryophyllales are distributed worldwide in a variety of habitats, members of several families are particularly adapted to arid or saline regions, including the Aizoaceae (ice-plant family), Portulacaceae (purslane family) and most notably, the Cactaceae (cactus family; www.britannica.com/plant/Caryophyllales). Heterologous betalain production in plants was used hereinbelow for studying the roles for these pigments in conferring tolerance to different a-biotic stress cues that are associated with arid regions such as drought, high UV radiation, excess light and high salinity and as plant defense compounds, acting against pathogenic fungi.

In another embodiment, the present invention provides methods of increasing the resistance of an organism or part of an organism to one or more stress factors comprising the step of contacting one or more cells of said organism with a nucleic acid encoding CYP76AD6, CYP76AD15, or a combination thereof, a nucleic acid encoding CYP76AD1, or a combination thereof, under conditions sufficient to produce betalains, thereby producing betalains and increasing the resistance of said organism or said part of an organism to said one or more stress factors. In one embodiment, the organism is a plant. In one embodiment, the organism comprises tyrosine. In one embodiment, the stress factor is an abiotic stress factor. In another embodiment, the stress factor is high osmotic pressure, high salinity condition, or a combination thereof. In another embodiment, the stress factor is drought. In another embodiment, the stress factor is excess light. In one embodiment, the plant part is a seed. In one embodiment, the plant is a food crop.

In one embodiment, the method comprises the step of contacting one or more cells of a plant or plant part with a nucleic acid encoding CYP76AD1, a nucleic acid encoding a DOPA 4,5-dioxygenase (DOD) enzyme, and a nucleic acid encoding a betalain related glucosyltransferase.

In another embodiment, the present invention provides methods of increasing the resistance of a plant or plant part to high osmotic pressure comprising the step of contacting one or more cells of a plant or plant part comprising tyrosine with a nucleic acid encoding CYP76AD6, CYP76AD15, or a combination thereof, a nucleic acid encoding CYP76AD1, or a combination thereof, under conditions sufficient to produce betalains, thereby producing betalains and increasing the resistance of said plant or plant part to high osmotic pressure.

In another embodiment, the present invention provides methods of increasing the resistance of a plant or plant part to high salinity conditions comprising the step of contacting one or more cells of a plant or plant part comprising tyrosine with a nucleic acid encoding CYP76AD6, CYP76AD15, or a combination thereof, a nucleic acid encoding CYP76AD1, or a combination thereof, under conditions sufficient to produce betalains, thereby producing betalains and increasing the resistance of said plant or plant part to high salinity conditions.

In another embodiment, the present invention provides a method of increasing seed germination rates in a plant comprising the step of contacting one or more cells of a plant or plant part with a polynucleotide comprising a nucleic acid encoding CYP76AD1, a nucleic acid encoding DODA1 and a nucleic acid encoding cDOPA5GT, under conditions sufficient to produce betalains, thereby increasing seed germination rates in said plant.

In another embodiment, the present invention provides methods of increasing the resistance of an organism or part of an organism to one or more fungal diseases comprising the step of contacting one or more cells of said organism with a nucleic acid encoding CYP76AD6, CYP76AD15, or a combination thereof, a nucleic acid encoding CYP76AD1, or a combination thereof, under conditions sufficient to produce betalains, thereby producing betalains and increasing the resistance of said organism or said part of an organism to said one or more fungal diseases. In one embodiment, the organism comprises tyrosine. In one embodiment, the organism is a plant. In one embodiment, the plant is a food crop.

In one embodiment, the fungus causing said fungal disease is a phytopathogenic fungus. In one embodiment, the fungus causing said fungal disease is Botrytis cinerea. In one embodiment, the plant is a tobacco plant. In one embodiment, the plant part is the leaf.

In another embodiment, the present invention provides methods of increasing the resistance of a plant or plant part to one or more fungal diseases comprising the step of contacting one or more cells of a plant or plant part with a nucleic acid encoding CYP76AD1, a nucleic acid encoding a DOPA 4,5-dioxygenase (DOD) enzyme, and a nucleic acid encoding a betalain related glucosyltransferase, under conditions sufficient to produce betalains, thereby producing betalains and increasing the resistance of said plant or plant part to one or more fungal diseases. In one embodiment, the plant is a food crop.

In another embodiment, the present invention provides methods of decreasing the lesion area due to a fungal disease in a plant or plant part comprising the step of contacting one or more cells of said plant or plant part with a nucleic acid encoding CYP76AD1, a nucleic acid encoding a DOPA 4,5-dioxygenase (DOD) enzyme, and a nucleic acid encoding a betalain related glucosyltransferase, under conditions sufficient to produce betalains, thereby producing betalains and thereby decreasing said lesion area due to a fungal disease in said plant or plant part.

In another embodiment, the present invention provides methods of decreasing necrosis due to a fungal disease in a plant or plant part comprising the step of contacting one or more cells of said plant or plant part with a nucleic acid encoding CYP76AD1, a nucleic acid encoding a DOPA 4,5-dioxygenase (DOD) enzyme, and a nucleic acid encoding a betalain related glucosyltransferase, under conditions sufficient to produce betalains, thereby producing betalains and thereby decreasing necrosis due to a fungal disease in said plant or plant part.

In another embodiment, the present invention provides methods of inhibiting growth of plant species near to a first plant or plant part comprising the step of contacting one or more cells of said first plant or plant part with a nucleic acid encoding a CYP76AD1-β clade gene, under conditions sufficient to produce and secrete L-DOPA, thereby inhibiting growth of plants species near to said first plant or plant part. In one embodiment, the plant species is a heterologous plant species to the first plant.

In another embodiment, the present invention provides methods of allelopathic growth of an organism comprising the step of contacting one or more cells of an organism or part thereof with a nucleic acid encoding a CYP76AD1-β clade gene, under conditions sufficient for said organism or part thereof to produce and secrete L-DOPA, thereby allowing allelopathic growth of said organism.

In one embodiment, allelopathy is a biological phenomenon by which an organism produces one or more biochemicals that influence the germination, growth, survival, and reproduction of other organisms.

In another embodiment, the present invention provides methods of weed control comprising the step of contacting one or more cells of a plant or plant part with a nucleic acid encoding a CYP76AD1-β clade gene, under conditions sufficient for said plant or plant part to produce and secrete L-DOPA, thereby inhibiting the growth of weeds near to said plant or plant part.

In one embodiment, the step of contacting one or more cells of a plant or plant part with a nucleic acid encoding CYP76AD6, CYP76AD15, or a combination thereof, a nucleic acid encoding CYP76AD1, or a combination thereof, under conditions sufficient to produce betalains comprises contacting said one or more cells with a nucleic acid encoding CYP76AD1, a nucleic acid encoding a a DOPA 4,5-dioxygenase (DOD) enzyme, and a nucleic acid encoding a betalain related glucosyltransferase. In one embodiment, the DOD is Beta vulgaris DODA1. In one embodiment, the betalain related glucosyltransferase is cyclo-DOPA 5-O-glucosyltransferase or betanidin-5-O-glucosyltransferase. In one embodiment, the cyclo-DOPA 5-O-glucosyltransferase is M. jalapa gene cyclo-DOPA 5-O-glucosyltransferase (cDOPA5GT).

In one embodiment, a polynucleotide comprises the nucleic acids. In one embodiment, the polynucleotide is pX11. In another embodiment, the polynucleotide is pX12. In another embodiment, the polynucleotide is pX13.

In one embodiment, pX11-expressing species exhibited a predominantly red-violet color due to the production of higher amounts of betacyanins than betaxanthins.

The invention described herein has numerous applications in various diverse industries, including, for example, but not limited to, agriculture, food and beverages, pharmaceuticals, cosmetics, textiles, and consumer products.

In one embodiment, applications of the present invention include production of betalains and/or L-DOPA for fish feed. In another embodiment, production of betalains and/or L-DOPA is for inclusion in a food supplement. In one embodiment, the food supplement is for humans. In another embodiment, the food supplement is for non-human animals. In another embodiment, the food supplement is for veterinary use. In another embodiment, the food supplement is for aquaculture. In another embodiment, the food supplement is in the form of a vitamin. In another embodiment, the food supplement is added to milk or other dairy products.

In another embodiment, the present invention provides a method of increasing one or more betalains in a dairy product comprising the steps of a) contacting a bacterial cell with a polynucleotide encoding a nucleic acid encoding a DOPA 4,5-dioxygenase (DOD) enzyme and a nucleic acid encoding CYP76AD6, a nucleic acid encoding CYP76AD15, a nucleic acid encoding CYP76AD1, or a combination thereof, wherein if said cell is contacted with a nucleic acid encoding CYP76AD1 and a nucleic acid encoding DOD and not a nucleic acid encoding CYP76AD6 or a nucleic acid encoding CYP76AD15, said cell is also contacted with a nucleic acid encoding a betalain related glucosyltransferase, and b) fermenting a dairy product with bacteria grown from said cell, thereby increasing one or more betalains in said dairy product. In one embodiment, the bacteria is a bacteria used in fermentation for preparation of dairy products. In one embodiment, the bacteria is a Lactobacillus.

In another aspect, the genes of the invention can be transformed into a plant or a plant cell in order to protect against one or more biotic stresses such as, for example, but not limited to, fungi, bacteria, and virus and/or one or more abiotic stresses such as, for example, but not limited to, heat, cold, salt, drought, and ultraviolet exposure.

In one embodiment, a plant organ expressing a polynucleotide or polypeptide of the present invention may serve as a food coloring, dye, or pigment. In another embodiment, a derivative product of a plant organ expressing a polynucleotide or polypeptide of the present invention may serve as a food coloring, dye, or pigment.

In another embodiment, juice from transformed fruit can be extracted from such fruit for making beverages. In another embodiment, the juice or other extract from a plant organ expressing a polynucleotide or polypeptide of the present invention may serve as a food coloring, dye, or pigment. In one embodiment, such juice may be used to dye yogurt, sweet, chewing gum, ice cream and the like. Such dye or coloring agent can also be used in textile industry to dye clothes.

In another embodiment, an extract of a transgenic plant or plant cell as described herein may be sprayed on vulnerable plants to protect them from biotic and abiotic stresses, as described hereinabove. Thus, the present invention includes both an extract of a plant or food crop as described herein as well as a method of protecting a plant from biotic stress, abiotic stress, or a combination thereof comprising contacting said plant with a plant or plant extract produced by the methods of the present invention as described herein.

In another embodiment, production of betalains and/or L-DOPA is for cosmetic uses. In one embodiment, production of betalains and/or L-DOPA is for inclusion in sunscreens. In another embodiment, betalains and/or L-DOPA for use in sunscreens is produced in yeast.

In another embodiment, the present invention provides a method of treating, inhibiting, suppressing, or preventing a dopamine-responsive disorder in a subject comprising the step of administering a food crop, cell, or cell line comprising high levels of a CYP76AD1-β clade polypeptide, thereby providing said subject with L-DOPA, thereby treating, inhibiting, suppressing, or preventing dopamine-responsive disorder in said subject.

In another embodiment, the present invention provides a use of a CYP76AD1-β clade gene or a food crop, cell, or cell line comprising high levels of said CYP76AD1-β clade gene in the preparation of a composition for treating or inhibiting a dopamine-responsive disorder in a subject.

In another embodiment, the present invention provides a CYP76AD1-β clade gene or a food crop, cell, or cell line comprising high levels of said CYP76AD1-β clade gene for treating or inhibiting a dopamine-responsive disorder in a subject.

In one embodiment, the CYP76AD1-β clade polypeptide is CYP76AD6, CYP76AD15, or a combination thereof. In one embodiment, said dopamine-responsive disorder is Parkinson's Disease, Dopamine-responsive Dystonia, or a combination thereof.

In another embodiment, the present invention provides a method of treating, inhibiting, suppressing, or preventing Parkinson's Disease or Dopamine-responsive Dystonia in a subject, comprising providing said subject with a tyrosine-expressing plant or plant part genetically modified to express CYP76AD6, CYP76AD15, or a combination thereof, thereby providing said subject with L-DOPA, thereby treating, inhibiting, suppressing, or preventing Parkinson's Disease or Dopamine-responsive Dystonia in said subject.

In another embodiment, the present invention provides a method of treating, inhibiting, suppressing, or preventing Parkinson's Disease or Dopamine-responsive Dystonia in a subject, comprising administering a CYP76AD6, CYP76AD15, or a combination thereof, enzyme to said subject, optionally with phenylalanine or tyrosine, thereby producing L-DOPA from tyrosine, thereby treating, inhibiting, suppressing, or preventing Parkinson's Disease or Dopamine-responsive Dystonia in said subject.

In another embodiment, the present invention provides a method of suppressing the symptoms, stabilizing symptoms, delaying the onset, and/or slowing the progression, Parkinson's Disease or Dopamine-responsive Dystonia in a subject, comprising providing said subject with a tyrosine-expressing plant or plant part genetically modified to express CYP76AD6, CYP76AD15, or a combination thereof, thereby providing said subject with L-DOPA, thereby suppressing the symptoms, stabilizing symptoms, delaying the onset, and/or slowing the progression, Parkinson's Disease or Dopamine-responsive Dystonia in said subject.

In another embodiment, the present invention provides a method of suppressing the symptoms, stabilizing symptoms, delaying the onset, and/or slowing the progression, Parkinson's Disease or Dopamine-responsive Dystonia in a subject, comprising administering a CYP76AD6, CYP76AD15, or a combination thereof, enzyme to said subject, optionally with phenylalanine or tyrosine, thereby producing L-DOPA from tyrosine, thereby suppressing the symptoms, stabilizing symptoms, delaying the onset, and/or slowing the progression, Parkinson's Disease or Dopamine-responsive Dystonia in said subject.

In one embodiment, the Parkinson's Disease is idiopathic Parkinson's disease, postencephalitic parkinsonism, or symptomatic parkinsonism.

In one embodiment, the subject is additionally administered a peripheral DOPA decarboxylase inhibitor (DDCI). In one embodiment, the DDCI is carbidopa, a benserazide, or a combination thereof.

In another embodiment, the subject is additionally administered pyridoxine.

In one embodiment, administration of CYP76AD6, CYP76AD15, or a combination thereof, enzyme and/or the L-DOPA produced by the CYP76AD6, CYP76AD15, or a combination thereof, enzyme as described herein is via oral administration. In another embodiment, administration is via a catheter. In another embodiment, the enzyme is administered epidurally, intracerebrally, or intracerebroventricularly. Other suitable methods of administration are described hereinbelow.

According to any of the methods of the present invention and in one embodiment, the subject is human. In another embodiment, the subject is murine, which in one embodiment, is a mouse, and, in another embodiment, is a rat. In another embodiment, the subject is canine, feline, bovine, ovine, or porcine. In another embodiment, the subject is mammalian. In another embodiment, the subject is any organism susceptible to Parkinson's Disease.

In one embodiment, the methods of the present invention are employed in veterinary medicine. In one embodiment, the present invention provides treatment of domesticated mammals which are maintained as human companions (e.g., dogs, cats, horses), which have significant commercial value (e.g., dairy cows. beef cattle, sporting animals), which have significant scientific value (e.g., captive or free specimens of endangered species), or which otherwise have value.

In one embodiment, methods of the present invention include use of the compositions for treating and/or preventing a disease, disorder, or condition. In one embodiment, methods of the present invention include both therapeutic treatment or prophylactic or preventative measures, wherein the object is to prevent or lessen the targeted pathologic condition or disorder as described hereinabove. Thus, in one embodiment, methods of the present invention may include directly affecting or curing, suppressing, inhibiting, preventing, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment, symptoms are primary, while in another embodiment, symptoms are secondary. In one embodiment, “primary” refers to a symptom that is a direct result of the disease, disorder, or condition, while in one embodiment, “secondary” refers to a symptom that is derived from or consequent to a primary cause. In one embodiment, the polynucleotides, polypeptides, compositions, cells and their use in the present invention treat or inhibit primary or secondary symptoms or secondary complications related to Parkinson's Disease.

In another embodiment, “symptoms” may be any manifestation of Parkinson's Disease, comprising tremors, bradykinesia, rigid muscles, impaired posture and balance, loss of automatic movements, speech changes, writing changes, or a combination thereof.

In another embodiment, the present invention provides a method of marking a genetic transformation comprising the step of contacting a cell with a polynucleotide comprising a nucleic acid sequence encoding a) CYP76AD1, CYP76AD6, CYP76AD15, or a combination thereof, b) a nucleic acid sequence encoding DOPA 4,5-dioxygenase (DOD) enzyme; and c) an additional nucleic acid sequence of interest, under conditions sufficient to produce betalains in said cell, wherein the color produced by betalain production in said cell thereby marks said genetic transformation in said cell. In one embodiment, the contacting step further comprises contacting a betalain related glucosyltransferase.

In one embodiment, the method further comprises contacting a growth medium with ascorbic acid as a reducing agent to prevent spontaneous betanidin oxidation, which causes the pigment to polymerize and lose its violet color.

In another embodiment, the present invention provides a method of identifying betalain related genes in an organism comprising the step of searching a plant genome database for genes with expression patterns that highly correlate to those of the betalain related genes described herein. In one embodiment, the gene is MjDOD, cDOPA5GT CYP76AD3, or a combination thereof. In another embodiment, the gene is CYP76AD1, CYP76AD6, CYP76AD15, or a combination thereof.

### Combinations

In one embodiment, betalains produced using the compositions and methods of the present invention may be administered or provided by themselves. In another embodiment, betalains produced using the compositions and methods of the present invention may be administered or provided together with an additional dietary supplement. In another embodiment, betalains produced using the compositions and methods of the present invention may be administered or provided together with a terpene, which in one embodiment, is an astaxanthin. In another embodiment, betalains produced using the compositions and methods of the present invention may be administered or provided together with anthocyanins, carotenoids, or a combination thereof.

In another embodiment, betalains produced using the compositions and methods of the present invention may be produced together with an additional component, which in one embodiment, may serve as a dietary supplement. In another embodiment, betalains produced using the compositions and methods of the present invention may be produced together with a terpene, which in one embodiment, is an astaxanthin. In another embodiment, betalains produced using the compositions and methods of the present invention may be produced together with anthocyanins, carotenoids, or a combination thereof.

In another embodiment, the present invention provides a food crop comprising a polynucleotide of the present invention. In one embodiment, the food crop comprising a polynucleotide of the present invention further comprises anthocyanin, carotene, or a combination thereof. In one embodiment, the food crop comprising a polynucleotide of the present invention is genetically modified to comprise anthocyanin, carotene, or a combination thereof. In one embodiment, the food crop is purple anthocyanin tomatoes.

Any reference including patents, patent applications, or scientific publications, cited herein, are incorporated by reference in their entirety.

The following examples are presented in order to more fully illustrate the preferred embodiments of the invention. They should in no way be construed, however, as limiting the broad scope of the invention.

### EXAMPLES

### Example 1

### Materials and Methods (1)

### Plant Material and Growth Conditions

Mirabilis jalapa plants were soil-grown in a greenhouse with long-day light conditions (25° C.). Beta vulgaris and Nicotiana benthamiana plants were soil-grown in climate rooms (22° C.; 70% humidity; 18/6 hours of light/dark). Plant material for M. jalapa RNA-sequencing was collected from red flowers in five developmental stages (stage 1—flower length approx. 1 cm; stage 2-2 cm; stage 3-3 cm; stage 4-4 cm; stage 5-5-6 cm), red-young or green-mature leaves, and epidermis from stem node (red) or internode (green) areas. Plant material for B. vulgaris RNA-sequencing was collected from hypocotyls of 10 day old seedlings of three varieties (Red beet, Burpees Golden beet and Chioggia ‘striped’ beet). Collected tissue was immediately frozen in liquid nitrogen and maintained at −80° C. until processing.

### Transcriptome Sequencing, Assembly and Analysis

M. jalapa and B. vulgaris libraries for Illumina high-throughput strand-specific RNA-Seq were prepared as follows: total RNA was extracted from sampled tissues with the TRIzol method based on the TRI reagent user manual (Sigma-Aldrich). Five μg of total RNA from each sample was used for preparation of RNA-seq libraries using methods known in the art with minor modifications. Briefly: poly(A) RNA was isolated from total RNA using Dynabeads Oligo (dT)25 (Invitrogen), fragmented at 94° C. for 5 minutes and eluted. First-strand cDNA was synthesized using reverse transcriptase SuperScript III (Invitrogen) with random primers and dNTPs, and second-strand cDNA was generated using DNA polymerase I (Enzymatics) and dUTPs. After end-repair (Enzymatics), dA-tailing with Klenow 3′-5′ (Enzymatics) and adapter ligation (Quick T4 DNA Ligase, NEB), the dUTP-containing second-strand was digested by uracil DNA glycosylase (Enzymatics). The resulting first-strand adaptor-ligated cDNA was used for 13-15 cycles of PCR enrichment with NEBNext High-Fidelity PCR Master Mix (NEB). Indexed libraries were pooled and sequenced with an Illumina HiSeq2000 instrument. De-novo assembly and calculation of normalized count values for the M. jalapa dataset were done with Trinity software version Trinityrnaseq_r2013-02-25, following standard procedure. Contig annotations were assigned using Blast2GO. De-novo, genome-guided assembly of the B. vulgaris dataset was done with Trinity version Trinityrnaseq_r2014-07-17, using the B. vulgaris (sugar beet) genome resource as reference (http://bvseq.molgen.mpg.de). Gene annotation was obtained with the inclusive Trinotate module.

### Generation of DNA Constructs

Gene sequences used in this study; cDOPA5GT (GenBank accession AB182643.1; SEQ ID NO: 2), DODA1 (accession HQ656027.1; SEQ ID NO: 33), CYP76AD1 (accession HQ656023.1; SEQ ID NO: 32). Other sequences are provided in Materials and Methods. CYP76AD6 was named by Dr. David R. Nelson, University of Tennessee, Memphis USA, to maintain consistency in nomenclature. M. jalapa cDOPA5GT and B. vulgaris CYP76AD1, BvDODA1, BvCYP76new and CYP76AD6 transcripts were amplified from M. jalapa red petal and B. vulgaris red hypocotyl cDNA libraries, which were prepared using a High-Capacity cDNA Reverse Transcription Kit (Life technologies). PCR amplification was done using ‘Phusion’ DNA polymerase (Finnzyme) and oligonucleotides specified in Table 1. For VIGS assay, all gene fragments were cloned into a pTRV2 vector using XhoI and SacI restriction. Vectors for co-silencing were constructed by cloning a CYP76AD1 fragment into pTRV2: BvCYP76new and pTRV2:CYP76AD6 using EcoRI and BamI restriction. DNA constructs used for N. benthamiana agroinfiltration and for agrobacteria-mediated plant transformation were constructed with Goldenbraid cloning. CYP76AD1, BvDODA1, CYP76AD6 and cDOPA5GT were cloned into a pUPD vector using oligonucleotides specified in Table 1. pDODA, pAD1-GT, pYFP, pAD6 and pX11 are respectively 2α2, 2Ω2, 2α1, 2α1 and 3α1 vectors, all of which are based on a pCAMBIA backbone.

TABLE 1
Oligonucleotides used in this study

| | | |
|---|---|---|
| SEQ | | |
| ID | | |
| NO: | Oligonucleotide name | Sequence (5′ to 3′) |

Quantitative real-time PCR

| | | |
|---|---|---|
| 45 | CYP76AD1 qPCR Fwd | TGTGCTAGACGTTCTTCTTCAGCT |
| 46 | CYP76AD1 qPCR Rev | AAAATGTCGACGAGCAAATG |
| 47 | CYP76AD6 qPCR Fwd | CTTCATCTCGTAGGCTTCCTCC |
| 48 | CYP76AD6 qPCR Rev | GATGAGGCTTTTCGCCAAGA |
| 49 | Bv15885 qPCR Fwd | CCTGTATCGGCTAAATGGCG |
| 50 | Bv15885 qPCR Rev | CAATTGCACAGCGGAGATTTT |
| 51 | Bv20048 qPCR Fwd | TTGATGCTGTTTGCGCAGTC |
| 52 | Bv20048 qPCR Rev | TGCGAAGATTTCGCCATTTT |
| 53 | BvGAPDH qPCR Fwd | TGGTGCTGATTTTCGTCGTAGAG |
| 54 | BvGAPDH qPCR Rev | TGGCACCACCCTTCAAGTG |
| 55 | Bv10427 qPCR Fwd | TAAAGCTCCCTCCTGGTCCA |
| 56 | Bv10427 qPCR Rev | CCGCAGAACGATGAGGTTTG |
| 57 | BvCYP76new qPCR Fwd | TCAAACTCGGAAGCATCACTACA |
| 58 | BvCYP76new qPCR Rev | GGGCTAAGTCGTGCTCGAGG |

pTRV2 cloning

| | | |
|---|---|---|
| 59 | BvDODA1 Fwd + XhoI | AAAAACTCGAGGTTAAACCTACTGTTA |
| | | ATGCTGTC |
| 60 | BvDODA1 Rev + SacI | AAAAAGAGCTCGCTGCCCAAGGTGCAA |
| | | CTCC |
| 61 | CYP76AD1 Fwd + XhoI | AAAAACTCGAGGCTAATCTTGCTAAAA |
| | | TTCACGG |
| 62 | CYP76AD1 Rev + SacI | AAAAAGAGCTCTTATGGTGGGCTAATT |
| | | CCACTG |
| 63 | CYP76AD1 Fwd + | AAAAAGGATCCGCTAATCTTGCTAAAA |
| | BamHI | TTCACGG |
| 64 | CYP76AD1 Rev + EcoRI | AAAAAGAATTCTTATGGTGGGCTAATT |
| | | CCACTG |
| 65 | BvCYP76new Fwd + | AAAAACTCGAGCTGCAAGAGTCAATGA |
| | XhoI | TCTCA |
| 66 | BvCYP76new Rev + SacI | AAAAAGAGCTCTCATCTGGGAATTGGA |
| | | ATTGCT |
| 67 | CYP76AD6 Fwd + XhoI | AAAAACTCGAGCACAACAGCAAGCACA |
| | | TTAGA |
| 68 | CYP76AD6 Rev + SacI | AAAAAGAGCTCAGTCCAGGGCATATCC |
| | | TTCTAC |

Goldenbraid cloning

| | | |
|---|---|---|
| 69 | BvDODA1 Fwd | GCGCCGTCTCGCTCGAATGAAAATGATG |
| | | AATGGTGAAGATG |
| 70 | BvDODA1 Rev | GCGCCGTCTCGCTCGAAGCCTAGGCTGA |
| | | AGTGAACTTGTA |
| 71 | CYP76AD1 Fwd | GCGCCGTCTCGCTCGAATGGATCATGCA |
| | | ACATTAGCAATG |
| 72 | CYP76AD1 Rev | GCGCCGTCTCGCTCGAAGCTCAATACCT |
| | | AGGTATTGGAATAAG |
| 73 | CYP76AD6 Fwd1 | GCGCCGTCTCGCTCGAATGGATAACGCA |
| | | ACACTTGCT |
| 74 | CYP76AD6 Rev1 | GCGCCGTCTCGTTGCCCATTCTAATGTG |
| | | CTTG |
| 75 | CYP76AD6 Fwd2 | GCGCCGTCTCGGCAATGGCCGAACTTGT |
| | | GAA |
| 76 | CYP76AD6 Rev2 | GCGCCGTCTCGCTCGAAGCCTAGTTTCT |
| | | GGGAACTGGAAT |
| 77 | cDOPA5GT Fwd1 | GCGCCGTCTCGCTCGAATGACCGCCATT |
| | | AAAATGAACAC |
| 78 | cDOPA5GT Rev1 | GCGCCGTCTCGGCCTCATTTCCTCATTG |
| | | ATATC |
| 79 | cDOPA5GT Fwd2 | GCGCCGTCTCGAGGCCAGAATGGCTACC |
| | | AGA |
| 80 | cDOPA5GT Rev2 | GCGCCGTCTCGCTCGAAGCTTATTGAAG |
| | | AGAAGGTTCCAACTT |

Gateway cloning for yeast expression

| | | |
|---|---|---|
| 81 | BvDODA1 Fwd | ggggacaagtttgtacaaaaaagcaggatcATGAAAAT |
| | | GATGAATGGTGAAGAT |
| 82 | BvDODA1 Rev | ggggaccactttgtacaagaaagctgggtcCTAGGCTGA |
| | | AGTGAACTTGTAGGAG |
| 83 | CYP76AD1 Fwd | ggggacaagtttgtacaaaaaagcaggatcATGGATCA |
| | | TGCAACATTAGCAA |
| 84 | CYP76AD1 Rev | ggggaccactttgtacaagaaagctgggtcTCAATACCT |
| | | AGGTATTGGAATAAGTTT |
| 85 | CYP76AD6 Fwd | ggggacaagtttgtacaaaaaagcaggatcATGGATAA |
| | | CGCAACACTTGCTGTGATCC |
| 86 | CYP76AD6 Rev | ggggaccactttgtacaagaaagagggtcCTAGTTTCT |
| | | GGGAACTGGAATAACTTGAAGAG |

Virus-Induced Gene Silencing in Beta vulgaris

Fragments of BvDODA1 (407 bp), CYP76AD1 (418 bp), BvCYP76new (471 bp), and CYP76AD6 (420 bp) were cloned into a pTRV2 vector, transformed to agrobacteria strain GV3101 and introduced to B. vulgaris ‘Bull's Blood’ variety 10 day old seedlings using the previously described vacuum infiltration method. Agrobacteria were brought to O.D.600 2.0 in VIGS infiltration buffer (10 mM MES, 10 mM MgCl2, 200 μM acetosyringone), incubated at room temperature for 3 hours and mixed in a 1:1 ratio with pTRV1-carrying agrobacteria before infiltration. For each experiment, 48 seedlings were infected. In experiments resulting in visible phenotypes, depigmentation patches were typically observed in over half of the infected seedlings within 2-3 weeks. Tissues for spectrophotometric analysis were sampled 3.5 weeks post infection.

Quantitative Real-Time PCR (qRT-PCR) Analysis

qRT-PCR analysis was carried out on VIGS-infected red beet plants. Three biological replicates from each experiment were analyzed, each replicate consisting of leaf tissue from two to three plants, sampled 3.5 wk post infection. RNA was extracted with the TRIzol method (according to the Sigma-Aldrich user manual for the TRI reagent), DNase-treated and reverse-transcribed to cDNA with a High Capacity cDNA Reverse Transcription kit (Applied Biosystems, Foster City, CA, USA), according to the manufacturer's instructions. All oligonucleotides were designed using PRIMEREXPRESS software (Applied Biosystems) (Table 1). qRT-PCR was performed with Fast SYBR Green reagent and a StepOnePlus instrument (Applied Biosystems) in the following conditions: initial step in the thermal cycler for 20 s at 95° C., followed by PCR amplification for 40 cycles of 3 s at 95° C. and 30 s at 59° C., and finally dissociation analysis to confirm the specificity of PCR products. Each reaction consisted of 10 μl total volume, containing 2.5 μM of each primer. Relative transcript levels were calculated according to the ΔΔCt method (Livak & Schmittgen, 2001), using the ‘housekeeping’ gene GAPDH for reference.

Transient Expression in Nicotiana benthamiana

Transient gene expression assays in N. benthamiana with the pDODA, pAD1-GT, pYFP, pAD6 and pX11 vectors were based on a previously described agroinfiltration method. All constructs were transformed to Agrobacterium tumefaciens GV3101 strain, excluding pX11 which was transformed to A. tumefaciens EHA105 strain. In all cases, agrobacteria were grown overnight in LB media and brought to a final O.D.600 0.2 in infiltration buffer. When co-infiltrated, agrobacteria carrying separate constructs were each brought to O.D.600 0.2 and mixed in a 1:1 ratio before infiltration. Tissues used for subsequent LC-MS analysis were sampled from leaves 7 days post infiltration. For LC-MS analysis of L-DOPA and betalains, 3-4 biological replicates for each experiment were sampled, each consisting of infiltrated tissues from 2-3 different leaves.

### Metabolite Extraction and Analysis

For betalain analysis, extraction solution (80% ethanol and 0.1% formic acid in DDW) was added to frozen, ground plant tissue (0.1-0.25 g) in a ratio of 200 μL 0.1 g-1 tissue. Samples were incubated at room temperature for 30 min. followed by 5 min. sonication, 5 min. centrifugation and filtration through 0.22 μm PVDF filters (Millipore). Samples diluted 3 fold in DDW were analyzed using a high resolution UPLC/PDA-qTOF system comprised of a UPLC (Waters Acquity) connected on-line to an Acquity PDA detector (200-700 nm) and a qTOF detector (tandem quadrupole/time-of-flight mass spectrometer, XEVO, Waters) equipped with an electrospray ionization (ESI) source. ESI was used in positive ionization mode at the m/z range from 50 to 1600 Da. The following settings were used: capillary—1 kV, cone—27V, collision energy—6 eV. For MS/MS or MSE runs, collision energy ramp from 15 to 40 eV was used. Separation of compounds was performed on a UPLC HSS T3 column (Waters Acquity, 1.8 μm, 2.1×100 mm) using eluents A (1% acetonitrile, 0.1% formic acid) and B (100% acetonitrile, 0.1% formic acid) as follows: the first 3 min.—isocratic elution at 100% A; 3.0-22.0 min—a linear gradient to 75% A, 22.0-22.5 min—a linear gradient to 100% B, 22.5-25.5 min—washing at 100% B, 25.5-26.0 min—return to 100% A and 26.0-28.0 min—equilibration at 100% A. Column temperature was set to 35° C., flow—0.3 ml/min. Betanin and iso-betanin were assigned using red beet leaf extract as reference. Other betalain compounds were putatively identified based on accurate mass, UV-VIS spectra, and MS/MS or MSE fragmentation (detailed in Table 2).

TABLE 2
Betalain compounds identified by LC-MS analysis.

| | | | | | | | |
|---|---|---|---|---|---|---|---|
| | | | | Mass | | | |
| | | Elemental | [M + H]+, | error, | Rt, | MS | UV/VIS, |
| No. | Name | composition | Da | ppm | min | fragments | nm |

Betacyanins

| | | | | | | | |
|---|---|---|---|---|---|---|---|
| 1 | Betanin^{a,c,d,e,f} | C24H26N2O13 | 551.1513 | 0 | 8.31 | 389.10 [M + H—Hex] | 534 |
| | | | | | | 345.11 | |
| | | | | | | [M + H—Hex—COO]— | |
| | | | | | | 343.0 [M + H—Hex—H2COO] | |
| | | | | | | 297.09 [M + H—Hex—2H2COO] | |
| | | | | | | 194.05 | |
| | | | | | | 150.1 [M + H—Hex—H2COO—C9H7NO4] | |
| 2 | Iso- | C24H26N2O13 | 551.1502 | 2 | 9.06 | 389.10 [M + H—Hex] | 534 |
| | betanin^{a,c,d,e,f} | | | | | 343.0 [M + H—Hex—H2COO] | |
| | | | | | | 150.1 [M + H—Hex—H2COO—C9H7NO4] | |
| 3 | Betacyanin | C31H31N3O14 | 670.189 | 0.9 | 12.67 | 626.20 [M + H—COO] | 532 |
| | I^{c} | | | | | 582.21 [M + H-2 × COO] | |
| | (unknown) | | | | | 538.22 [M + H-3 × COO] | |
| | | | | | | 389.10 [M + H-281.0902 | |
| | | | | | | (C13H15NO6)] | |
| | | | | | | 345.11 [M + H-281-COO] | |
| | | | | | | 301.11 [M + H-281-2 × COO] | |
| | | | | | | 282.09 [M + H-389.10 | |
| | | | | | | (Betanidin)] | |
| | | | | | | 264.09 [M + H-389.1 | |
| | | | | | | (Betanidin)-H2O] | |
| | | | | | | 257.13 [M + H-281-3 × COO] | |
| | | | | | | 247.08 | |
| | | | | | | 180.10 | |
| | | | | | | 138.06 | |
| 4 | Betacyanin | C31H30N2O14 | 655.1771 | 0.8 | 15.68 | 389.10 [M + H-266.0790 | 533 |
| | II^{c} | | | | | (C13H14O6)] | |
| | (unknown) | | | | | 345.10 [M + H-266-COO] | |
| | | | | | | 343.10 [M + H-266-H2COO] | |
| | | | | | | 301.12 [M + H-266-2 × COO] | |
| | | | | | | 299.10 [M + H-266-H2COO—COO] | |
| | | | | | | 196.06 [M + H—C9H7NO4] | |
| | | | | | | 194.04 [M + H—C9H9NO4] | |
| | | | | | | 150.05 [M + H- | |
| | | | | | | 266.0790-H2COO—C9H7NO4] | |
| 5 | Betanidin^{g,i} | C18H16N2O8 | 389.0991 | 1.5 | 9.89 | 343.09 [M + H—H2CO2] | 539 |
| | | | | | | 297.09 [M + H—2H2CO2] | |
| | | | | | | 255.1 [M + H—2H2CO2—CO2] | |
| | | | | | | 253.08 [M + H—3H2CO2] | |
| | | | | | | 150.05 [M + H—Hex—H2CO2—C9H7NO4] | |
| 6 | Iso- | C18H16N2O8 | 389.0988 | 0.8 | 10.83 | 343.09 [M + H—H2CO2] | 539 |
| | betanidin^{g,i} | | | | | 297.09 [M + H—2H2CO2] | |
| | | | | | | 255.1 [M + H—2H2CO2—CO2] | |
| | | | | | | 253.08 [M + H—3H2CO2] | |
| | | | | | | 150.05 [M + H—Hex—H2CO2—C9H7NO4] | |
| 7 | Betacyanin | C26H28N2O14 | 593.1618 | 0.2 | 10.72 | 547.11 [M + H—H2COO] | 534 |
| | III^{e} | | | | | 511.17 [M + H-2 × H2O] | |
| | (unknown) | | | | | 389.09 [M + H—C8H12O6] | |
| | | | | | | 345.11 [M + H—C8H12O6—COO] | |
| | | | | | | 325.07 [M + H—C8H12O6—H2COO—H2O] | |
| | | | | | | 301.11 [M + H—C8H12O6-2-COO] | |
| | | | | | | 299.10 [M + H—C8H12O6—H2COO—COO] | |
| | | | | | | 297.08 [M + H—C8H12O6-2 × H2COO] | |
| | | | | | | 194.04 [M + H—C8H12O6—C9H9NO4] | |
| | | | | | | 178.05 [M + H—C8H12O6—C9H7NO4—H2O] | |
| | | | | | | 176.03 [M + H—C8H12O6—C9H9NO4—H2O] | |
| | | | | | | 150.05 [M + H—C8H12O6—H2CO2—C9H7NO4] | |

Betaxanthins

| | | | | | | | |
|---|---|---|---|---|---|---|---|
| 1 | Vulgaxanthin | C14H17N3O7 | 340.1147 | 0.6 | 2.21 | 323.08 [M + H—NH3] | 476 |
| | I^{f, g,h,i} | | | | | 277.08 [M + H—NH3—H2CO2] | |
| | (glutamine- | | | | | 231.07 [M + H—NH3-2 × H2CO2] | |
| | betaxanthin) | | | | | 194.04 | |
| | | | | | | 185.07 [M + H—3H2CO2—NH3] | |
| | | | | | | 157.06 [M + H—3H2CO2—NH3—CO] | |
| | | | | | | 150.05 [M + H—COO—C5H10N2O3] | |
| | | | | | | 148.05 [M + H—H2CO2—C5H10N2O3] | |
| | | | | | | 132.10 | |
| | | | | | | 86.1 | |
| 2 | Indicaxanthin^{b,c} | C14H16N2O6 | 309.1090 | 1.0 | 7.69 | 263.10 [M + H—H2COO] | 478 |
| | (proline- | | | | | 217.10 [M + H-2 × H2COO] | |
| | betaxanthin) | | | | | 219.11 [M + H—COO—H2COO] | |
| | | | | | | 189.10 | |
| | | | | | | 175.12 [M + H—H2COO—2COO] | |
| | | | | | | 173.11 [M + H-2 × H2COO—COO] | |
| | | | | | | 150.05 [M + H—COO—C5H9NO2] | |
| | | | | | | 145.07 | |
| | | | | | | 106.07 [M + H-2COO—C5H9NO2] | |
| 3 | Dopaxanthin- | C24H28N2O13 | 553.1667 | 0.5 | 7.97 | 391.11 | 478 |
| | hexoside^{b} | | | | | (C18H19N2O8) [M + H—Hex] | |
| | | | | | | 357.13 [M + H—Hex—2 × OH] | |
| | | | | | | 347.13 [M + H—Hex—COO] | |
| | | | | | | 345.11 [M + H—Hex—H2COO] | |
| | | | | | | 303.13 [M + H—Hex—2 × COO] | |
| | | | | | | 299.10 [M + H—Hex—2 × H2COO] | |
| | | | | | | 255.11 [M + H—Hex—COO-2 × H2COO] | |
| | | | | | | 211.07 [M + H—Hex—C9H8O4] | |
| | | | | | | 208.05 | |
| | | | | | | 194.04 | |
| | | | | | | 181.05 [M + H—C9H10N2O4] | |
| | | | | | | 165.05 [M + H—Hex—C9H8O4—H2COO] | |
| | | | | | | 150.05 [M + H—Hex—H2COO—C9H9NO4] | |
| | | | | | | 106.06 [M + H—Hex—H2COO—COO—C9H9NO4] | |
| 4 | Betaxanthin | C18H19N3O4 | 342.1445 | 2.6 | 6.67 | 298.15 [M + H—COO] | 476 |
| | I^{b} | | | | | 254.16 [M + H-2 × COO] | |
| | (unknown) | | | | | 252.15 [M + H—H2COO—COO] | |
| | | | | | | 237.14 [M + H-2 × COO—NH3] | |
| | | | | | | 225.14 | |
| | | | | | | 196.10 | |
| | | | | | | 159.09 | |
| | | | | | | 132.08 | |
| | | | | | | 117.05 | |
| | | | | | | 106.06 | |
| 5 | Valine- | C14H18N2O6 | 311.1245 | 0.6 | 10.69 | 267.11 [M + H—COO] | 469 |
| | betaxanthin^{g,h,i} | | | | | 265.11 [M + H—H2COO] | |
| | | | | | | 221.13 [M + H—COO—H2COO] | |
| | | | | | | 219.13 [M + H-2 × H2COO] | |
| | | | | | | 193.13 | |
| | | | | | | 175.12 [M + H—COO-2 × H2COO] | |
| | | | | | | 166.05 | |
| | | | | | | 150.05 [M + H—H2CO2—C5H9NO2] | |
| | | | | | | 132.05 [M + H—H2CO2—C5H9NO2—H2O] | |
| | | | | | | 119.06 | |
| | | | | | | 104.05 [M + H-2 × H2CO2—C5H9NO2] | |
| | | | | | | 94.05 | |
| 6 | Betalamic | C9H9NO5 | 212.0558 | 0.5 | 8.79 | 166.05 [M + H—H2COO] | 408 |
| | acid^{g,h,i} | | | | | 148.04 [M + H—H2COO—H2O] | |
| | | | | | | 138.05 [M + H—H2COO—CO] | |
| | | | | | | 120.05 [M + H-2 × (H2COO)] | |
| | | | | | | 92.05 [M + H-2 × (H2COO)—CO] | |
| | | | | | | 65.04 | |

Betalain compounds identified by LC-MS analysis in N. benthamiana agroinfiltration experiments of ^{a}pAD1-GT+pDODA1, ^{b}pAD6+pDODA1 and ^{c}pX11, ^{d}N. benthamiana callus, ^{e}N. glauca callus, ^{f}pX11-transformed N. tabacum, yeast expression experiments of ^{g}CYP76AD1+DODA1, ^{h}CYP76AD6+DODA1 and ⁱCYP76AD1/AD6+DODA1. All fragments were obtained in the MS/MS mode excluding betanidin and iso-betanidin, for which fragments were determined in the MS^{(E)} mode. Rt, retention time; MS fragments, masses of fragments obtained in positive ionization mode; UV/VIS, absorption maxima at UV/visible range; Hex, Hexose.

For L-DOPA analysis, samples were extracted as described above and analyzed using a UPLC/PDA-MSMS instrument, where a UPLC separation module (Waters Acquity) was connected to a photodiode array detector (Waters Acquity 2996, 190-800 nm), and (on-line) to a triple quadrupole MS detector (Waters Xevo TQ MS), equipped with an ESI source. Compounds were separated with a Phenomenex Luna column (2×150 mm, 3 μm) using a following gradient: the first 1 min.—linear gradient from 100 until 90% A, 1-4 min.—90-75% A, 4-5 min.—75-0% A, 5-9.5 min.—washing at 100% B, 9.5-10 min.—return to 100% A, and 10-12 min.—equilibration at 100% A, where A is 0.1% formic acid in water, and B is 0.1% formic acid in acetonitrile. Column temperature was set to 35° C., flow—0.25 ml/min. Samples were analyzed in positive ionization mode, using the following settings: capillary—2 kV, cone—26 eV, with two transitions 198.1>152.1 (collision—15 eV) and 198.1>181.1 (collision—10 eV) used for compound assignment. L-DOPA was identified by comparison with an L-DOPA commercial standard (Sigma-Aldrich), dissolved in DDW (5 μM) and injected with the same LC-MS conditions.

For relative quantification of betaxanthins in VIGS-silenced beet leaves by spectrophotometry, samples were extracted using the same procedure as detailed above, except 80% methanol was used instead of 80% ethanol. Extracts were diluted 4 fold in extraction solution, placed on a 96 well plate and analyzed with a Biotek Synergy HT Microplate Reader. Relative betaxanthin content of each sample was assessed by measuring absorption at 475 nm and subtracting the value of absorption at 600 nm.

For estimation of betacyanin content by spectrophotometry, the same extraction method was used as above. Samples were diluted in DDW to obtain solutions of O.D.535<2.0. Betacyanin quantification was based on absorption measurements at 535 nm and 600 nm, and calculated using a previously described method.

### Recombinant Expression in Yeast

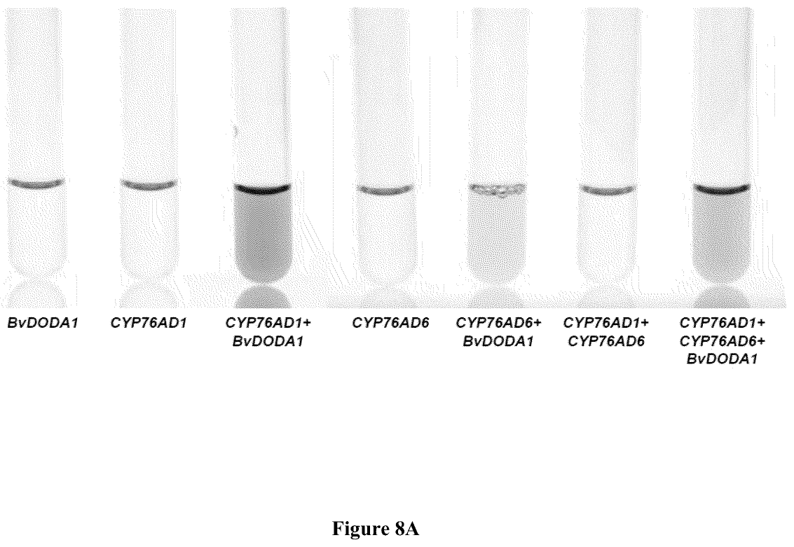

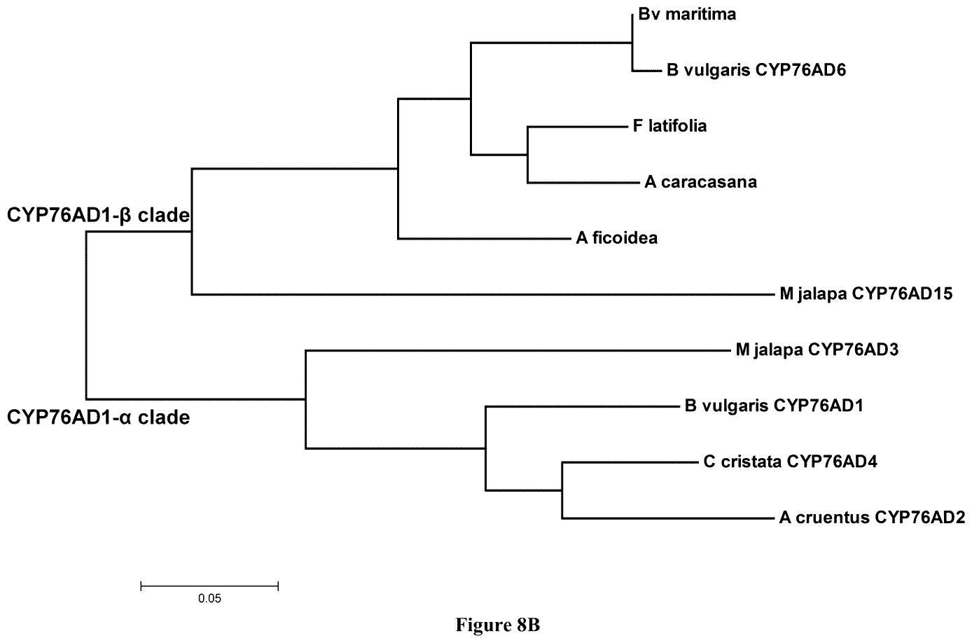

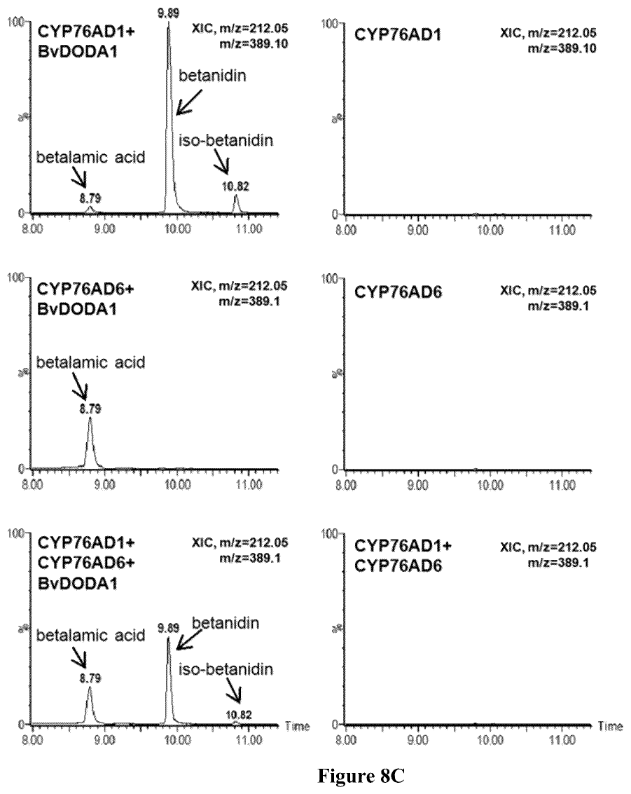

CYP76AD1, CYP76AD6 and BvDODA1 coding sequences were PCR-amplified and cloned to BP sites of a pDONR207 vector using Gateway cloning (Life technologies, Invitrogen) according to manufacturer's manual, and were next recombined to the LR sites of pAG423GAL-ccdB (for CYP76AD6), pAG425GAL (for BvDODA1), and pYES-DEST52 (for CYP76AD1, Invitrogen) vectors. In addition, the beta-glucoronidase (GUS) coding sequence was introduced to these vectors for control assays. The destination vectors were transformed using the PEG/LiAc method to S. cerevisiae strain BY4742, in sequential transformation. Each yeast clone was eventually transformed with the three destination vectors, expressing different combinations of CYP76AD1, CYP76AD6, BvDODA1 and GUS. Yeast strains were grown in standard SD medium overnight, containing 2% glucose and lacking histidine, leucine and uracil. The yeast were pelleted, re-suspended to O.D.600 1.0 in 2 ml SD media with 2 mM ascorbate, 1% raffinose and 2% galactose, lacking histidine, leucine and uracil, and grown overnight. Images in FIG. 8 are of media in which yeast were grown, following overnight galactose induction. For LC-MS analysis, 1 ml of liquid medium was dried in an evaporator and re-suspended in 100 μl of DDW.

### Bioinformatic Analyses

For co-expression analysis, contigs corresponding to DOPA 4,5-dioxygenase (MjDOD), cyclo-DOPA-5-O-glucosyltransferase (cDOPA5GT) and CYP76AD3 were separately used as ‘baits’. Genes co-expressing with either of the ‘baits’ were identified by using an R script to calculate Pearson correlation values, based on normalized count values of each gene across all 24 libraries.

For generation of the phylogenetic tree of CYP76AD1-like proteins, CYP76AD6 protein sequence was first used for a BLASTp query of the NCBI nr database. BLASTp top four hits with highest sequence identity and full protein sequence were selected for multiple sequence alignment, together with the CYP76AD1 protein and its previously described orthologs CYP76AD2, CYP76AD3 and CYP76AD4. Multiple sequence alignment was performed with Clustal Omega and processed into a neighbor-joining phylogenetic tree with MEGA software version 6.06. Bootstrapping of 100 iterations was used for validation. CYP76AD6 and CYP76AD1 pairwise alignment was done with Clustal Omega using default parameters and edited with GeneDoc software.

### Plant Transformation and Regeneration

Agrobacteria-mediated plant transformation and regeneration was done in tobacco leaf discs, eggplant, N. benthamiana, tomato, potato, Petunia, Nicotiana glauca according to methods known in the art. A protocol used for Solanum nigrum transformation is provided in hereinbelow. All plant species were transformed using agrobacteria GV3101 strain. Plant tissue culture was carried out in a climate rooms (22° C.; 70% humidity; 16/8 hours of light/dark, 2500 Lux light intensity).

Sequences of Genes Used in this Study

Sequences provided are contig sequences obtained from de-novo assembly of Beta vulgaris and Mirabilis jalapa transcriptome (RNA-seq) data. All sequences are presented in the 5′→−3′ direction.

>MjCYP76 from M. jalapa
(SEQ ID NO: 1)
CTTCAAATCCTCCCACGTATATATCAACAAAAACCAAAAACATCATCT
CATTACAATAATGGAGTATCATCTTCTCTTCCTTCTCCTCCTGCCGAT
CATCCTTATCTTCGTGTTCCTCAATCACAAGCCCAAGCAAACCAAACT
CGAACCACCAGGACCACGTCCATGGCCCATCATAGGCCACACCAACCT
CGTCGGCTCCAAACCTCATCAGTCCATTGCCAAACTAGCTCATATTTA
CGGTCCCATAATGTCACTTAAGCTAGGGAGAATCACTACTATTGTCAT
ATCTTCCCCTGAAGCAGCCAAAGACTTGTTCCTGAAACATGACCTTAC
TTTCTCAAGTAGGCAAGTTCCTCACGCTTCAACCGCTACAAACCATGA
CAAACTATCCATGGTTTGGCTCCCTGTATGTCCCAAATGGCGCTATCT
TAGAAAGATCGCGGCCATCCAACTATTCACCAACCAACAACTTGATGG
TGGTCAGGTACTAAGGCGTAAGAAAGTGGACGAGTTGATCCAATTTGT
GACTCGTTGTAGTAAACAAGGGCAAGTCATTGATATTGGAGAGGCTGT
TTTTATTACTACTCTTAACTTAATGTCAAATACCTTTTTTTCCAAGGA
TTTGTGTAGTTATGGTTTGGCCGAATCACGAGAGTTTAAGGATCTATT
TTGGGAGTTCATGAAAGTGCTAGCGAGTCCTAATGTTTCTGATTATTT
TCCATGGTTAAGATGGTTGGATTTGCAAGGCATCAAGAGAAGAAGCGA
GGGTTGTTATCGCAAGATGTTGGGTTTTTTTGGGGAGATTATTGATCA
GAGATTGAGAGATCCAATGTCATGTAAGAACGATGTATTGGACACTCT
ACTCAAACTTGTCGACCAAAAAGAGTTGAGCTTTGAGGATGTCAAACA
TATGCTTGTGGATTTGTTTGTTGCAGGGACGGATACAACTTCGAACAC
ATTGGAATGGACAATGGTAGAACTTTTACGCCACCCTAACATATTGAC
TAAAGCACAAACTGAACTCAGCCAAGTCATAGGCAAGGACAAGTTAGT
CCAAGAATCATGTATCACCAAGTTGCCATATCTTCAATCAATATTGAA
AGAAACATTCAGGTTGCACCCACCGACTCCTTTTTTACTTCCACACAA
AGCAATCGAGAACGTTGAACTATGCAACTATCACATACCTAAAGGTGC
TCAAGTTTGGGTGAATGTGTGGTCTATTGGCCGCGATCCCAACATTTG
GTCAAGCCCGGACTTATTTTCCCCAGAGAGATTTTTAGGTACCGACAT
TGATATAAAAGGTAACCATTTCGAGCTCATACCATTCGGAGCAGGAAG
AAGGATTTGCCCCGGGTTGTCACTAGCTTACAGGATGCTTCATTTGAT
TTTGGCCACTCTCCTTCATTCATTCAATTGGAACCTTCCTAATGACGT
ATGTCCGGAAAATATGGATATTGAAGAAAAATTTGGGATTACACTTCA
AAAGATCATGCCACTCAAAGTCATACCAAGCTCTAGGTGTCTGGATCA
CGACAACTAACTGGTGTTAATCTTTTACAACATATTGAATGATATATA
TCACGTTAAGGTACATGCGTCTAGATGCAGAAACTTGCATACGTTTCC
TTTTCAATAAAGTCTTGCATATGTATGGTTTATAGGGTGATGTAATTT
CTTGTTTGTTTTTACTTAAGTTTATAAGACATTTTTTTTTTTTTTTTA
TCGATGCTCTTACTCATTAAAATATTGGAACATATACAAAATTTTATA
TGTTTTAGATAAAATTATACAGGTTTTAATTTTAGGAACTTACCACTT
GTTCACACTGCGAGACTTAAATAGTCAAAATAGAAAATACACACTATT
AAAAAGTACTCACTATTTATCAAATTTAT.
>BvCYP76new from Beta vulgaris
(SEQ ID NO: 9)
CAGTCATTTAAAATCCTGTAAATTACTCACATACATAACCAAATTAAA
GTTGCCGCCGTACTGATTTCCAAAGGTCCTACATCTATCAATGGAGTA
TTACACCATAACGCTATTATTCATCATCTTCACAATATCAATATTCGG
TACAAAAATTTTGAGCAAGTCCAAACTTCCTCCAGGACCAACACCATG
GCCTATCATAGGCAACATCCTCGAGCTAGGCAAGTTACCTCATCAGGC
AGTTGACAAGCTCTCAAAAACCTATGGCCCTATATTATCTCTCAAACT
CGGAAGCATCACTACAATAGTAATATCATCCCCTGAAATCGTTAAAGA
AATGTTCCTCGAGCACGACTTAGCCCTTTCTAGTAGGCCTTCCCCGGA
TGCTTCGAGGGTGGGAAACCATAACAAATTTTCCATTGTTTGGCTCCC
AGTGTCCCCCAAATGGCGGGACCTTAGAAAAATTGCTACTATCCAATT
GTTTACTACTCAACGCCTAGATTCTAGCCAAGAACTACGGCAGATCAA
GGTGAACGAGCTGGTTGATTATGTACGACAGTGTTGTGAAAAGGGTCT
ACCTGTTGATGTTGGTAAAGCCGGGTTTACTACCACGTTAAATATGTT
GTCTAACACGTTTTTCTCCATGGATTTGGCTAGCCATGCTTCTTCGAA
TTCGCAAGAGTTCAAGGATCTTGTTTGGAGTCTTTTGGAAGAGGGTGC
AAAGCCTAATGTGTCGGATTTTTTCCCGATAGTTAGAGAATTGGATTT
GCAGGGAGTATCGAAAAATAGAAGGGTGCACATGAAGAAGCTAATGGG
AATTTTCGAGGAAATCATTGATGGAAGGTTGACAAAGTTAAAGGATGT
GAAGGATGATGTTCTAAGTACTTTACTCAAACTTGTTAAGGATGAAGA
ATTGAACCTTGACGACGTCAAACATATGCTCATGGATTTATTTTTAGC
GGGAACAGATACAACCTCTATCACATTGGAGTGGGCGATGACAGAATT
ACTTCGCAATCCAGAAAAGATGGAAAAGGTTCAAATTGAGTTGGACAA
AGTACTTGGCAAGGATAGTTCTCTGCAAGAGTCAATGATCTCAAAATT
ACCATATATTCAAGCAATAGTGAAAGAAACATTAAGATTACACCCACC
AACTCCTTTCTTGATTCCTCACAAAGCAGAAAAAGATGTATTGTTATG
CAACTATTTAGTGCCCAAAAACTCAATTATTTGGGTTAATTTATGGTC
GATCGCTCGTAGTCCAAGTGTGTGGCCAAATCCAGAATCATTTTCTCC
TGAAAGGTTTTTGGAGATGGAAATTGATATTAAAGGACGAGATTTCAA
ACTCATACCCTTTGGATCAGGAAGAAGAATGTGTCCTGGAATGCCACT
TGCTTATAGGATGACACATATGTTGTTGGCTACTCTTCTTCATTCATT
TAACTGGAAGTATGGTGAGGCAAGTCCAAAAGATATAGACATGAAAGA
AAAGTTTGGGTTGACGTTGCAAAAGGCTCAGCCACTTCAAGCAATTCC
AATTCCCAGATGATCAATCTCTTTTGAGGAGTTAGACGATTTCTTTGT
GTCAACCAACTAGTACTTATCGGAACATTTATTGTTAAATGTGTCTGT
TAGATGTACATATGATGTTGTTATACTAAGAAATAACGGTGTTTAAGT
GCAACATGGGACAAGAGGATTATGTAAAGTATCCTAGATTTTATTAGG
TGCTCCTACACCTATGTATTGGTGCATTTGCACCAAGTCGTCTGTAGA
AAACTTGAGACTTGGGAGGCAAGAACTTTACCTCTGTAAAAAAAACGC
GTTTTTTGCATTCTCCTTGTATCTTTTGCAAGAGATTGTACGCATTCA
CAAAACATCAAATACATTTGTGTTTACACAATAAGAATCGCCAACACT
GA.
>CYP82G1-like from M. jalapa
(SEQ ID NO: 36)
TACGTCTATTCTTGCCTCCCATTTCCTCCCTGCAAAAAGGAAGAAAAT
GGAAATACCCTTTAATTTGCAACCTATTTTTCTAATTATTGTACTATC
TACCTTCATTCTAACCCACCTAATAACAAAAAACACAAGAAAGTTCAA
CCAATGCAAATCAAAAACCAAGCCAATACCAAAACCACCTGGCTCACT
ACCCATAATAGGTCACTTGCACCTCCTAAGAGGTCATGGAACCACCCT
AGGTCGCACCCTCGGAGCCATGGCCGACAAGTCCGGACCCATTTTCGG
CCTCAAGCTAGGCCAACACAAGGCTATAGTCATTAGTAACTGGGAGAT
AGTCAAGGATTGTTTCACCAATAATGACATGAACCTTGCAACTAGACC
TAGCATGGCTATTAGCAAATATATTGGTTATGACTTTGCCTCTTTCTC
TATCTCACCTTATGGCCGATATTGGCGTGAGATTCGTAAGATCTCTAC
TGTTGAGCTCTTGTCAAACAAACGCCTTGAGAAAATGAAGCATGTTCG
AACTTTTGAGGTGAACTCTTGCCTTCAAATCTTCTATGAAAAATGTGT
CAAATCAGAGGATAATGAAAGTAATAAAGTAGCAAAAATTTGTCTTAA
AAAGTGGTTAGAATATATTGGTTTTAATATCGCACTATCTATGATAGT
TGGCAAGAGATTCTCCTACGACCAGTATTATAAAAAAAATACTGTTGC
ATCGAGATTTATGAAGGCAATAGAAAGAGCAACCCACTTAGCAGGACT
TCCTATACCCTCGGATTTTTTACCATGGGTTGAATGGATGGATTTGGT
GGGTTACATTGGTGCTATGAAGGAAACTCATAAGGAAATTGATGTGAT
TATTGGACATTGGCTTGATGAGCATATTCAAAAGAGGAAAGAGACGTT
AGAAAGGCGAGGTACAAGCATTGATGATAGTGACTTCATGGATGTAAT
GTTATCTAACATTACTGAACAATTTGCCAAGTCAACCTACTCTAAAAA
CACCATCATTAAAGCAACAGCTTTGACACTACTTCTAACAGGTTCAGA
AAGCACCTCAATCACACTAACATGGGCCATATCACTACTTCTAAACAA
CCCTACGGCCCAAAATTTGGCCCAACAAGAAATGGACAAACAAGTTGG
TAAACAAAGATGGGTTGAAGAATCTGACATCACCAATCTACCATACCT
CCAAGCCATCATAAAGGAAACTTTACGTTTGTATCCACCAAGCCCATT
AGCAGGCCCAAGAGTGGCCCAAGAAGACTGCCAAATCAATGGACATTG
TATCACAAAAGGAACCCGGGTGATTGTTAACATATGGAAGTTGCATCG
TGATCCGATGGTTTGGTCCGACCCAGATAAGTTCCGACCCGAAAGGTT
TATTGGGGAGCATAAGGATATTGATTTTAAGGGTCAATACTTTGGGTA
TATACCTTTTAGCTTGGGTAGGAGGGGTTGTCCTGGGATGAATTTTGG
GCTACAAGTGGTCCATTTGATGTTAGCTCGTTTGGTCCAAGGGTTCGA
TATTTGGGCTCAAAATGGTGGCCTAATTGATTTGAAGGAAGGGTTGGG
CTTGGCTTTGCCCAAGGCTGGTCCATTGGATGTCATTCTTGCACCACG
TTTGTCCAGTGAGCTTTATGGAGCTTTATCAAAGGTGAATGTTACTTA
TGGTCGGGCAATGAATGAACTAGCTTTGTATTTATAATTTTACAAATA
ATTTGTAAGATATGAACTGTTTATCTAGTCACTTTTCCGCGTCTTAGA
ATATCAATAAGCAAAAAGTTTTATGTAATAATATGTATTAGTGTGTTA
TTGTATCATGATTCGTCGGTATACTGAAAATATTTTACGTCTAGGAGA
ATGTGCACACATTTAATCTTATTTGCTTATAAGTATTATATACATAAA
ATTTTCTACTTTATAATTTAGGTATATTGAGCTTTCGACCAAAACCCA
AAAACTAAAGAAGTTTTTTTTAAAACATACAAGTTGGTATTGTTTAGA
TTTTTATTTATTTTTATTATCTCTTTTATTTGTACTTAGTGGGTGTGT
TAATAATGTAAAGAAGTATTAAATATTTATAGAGTTGGAAAAACCCAA
TATTTTACATTGGAAAAA.
>CYP78A9-like from M. jalapa
(SEQ ID NO: 37)
TACGTCTCTAACCTCCTTCTCCTCACCATCATCATCTTATTTCTCTCT
CTAACTTCCTATTTCTCTCTCTAGAATGGTCTCAACTAGTGACTCACT
CTGGCTTTTCACCTACTTAGCTTCAAAATATGATCATTTTACGACCCC
TAACTTTATTTATTCTTTGTTTTGTTTCGTAATATTTTTTATTTTGAT
TCATCTACTGTACTGGTCCCACCCAGGTGGTCCGGCATGGGGGAAGTA
TTCATGGACTCATCTCTCGGGATGGGCCGGGAAGCCCATCCCGGGCCC
AAGAAGTTGGCCCATTATAGGTGGTTTGGACCTGAAGATGGGACTGGC
TCATCGGAAGTTAGCCACAATGGCTGAGAAATACGGGACCATGGCTAA
GAGGCTCATGGCTTTTAGCCTAGGAGAAACACGCGTGATTGTCACGTG
CAATCCCGACGTGGCGAAAGAAATACTAAATAGCCCGGTTTTCTTGGA
CCGACCCGACCAGGAGTCGGCTTATGGGTTGATGTTTAACCGGTCTAT
AGGGTTCGCACCCTATGGGACCTACTGGCGAACCCTAAGGAAAATAAC
ATCAACCCATTTATTAAGCCCTAAACACGTAAAAGAATATGAGAACCT
ACGTAGGGAAATAATGGAACAAATGGTTGACTCGATATCGAACCAAGC
CGGACCGGTTCGGGTCCGGGAAATACTAAGGAGGGCTTCATTGGATAA
TATGATGGGGTCAGTGTTTGGTGGGCCCCACAAGGAGGTCGTTGAACA
GTTGATAGAAATGGTTGGTGAAGGGTATGACTTATTGGGTGTCCTTAA
TTTGGGGGACCATCTATCTTGGCTAGCCGAGTTTGACTTTCAAAAGGT
TCGGTTCAGGTGCTCTCAACTTGTTCCAAAAGTCAACCGGTTCGTTGG
TGAAATTATCGATGAACATCGGTCTAGTTCGGGTCAAATTATGAACCG
GGACTTTGTTGATGTTTTGTTGACTCTACCACAACATGAACAACTTTC
TCATTCTGACATGGTCGCTGTTCTTTGGGAGATGATATTTAGAGGAAC
AGATGCAACAGCAGTATTAATAGAATGGACATTAGCACGATTAGTAAT
TCATAAAGATATTCAATCAAAGGTCCAAAATGAGTTAGACCAAGTAGT
TGGAACATCACGAGCCGTTGAAGAGTCTGACCTATCATCACTGATATA
TCTAACGGCTGTGATTAAAGAAGTATTGCGGGTCCACCCACCTGGGCC
CCTCCTATCGTGGTCTCGCCTTTCAATTCAAGATACTACTATAGATGG
TTGTCATGTGCCAAAAGGTACTACTGCTATGGTTAACATGTGGGCTAT
TGCTAGAGATCCTAATGTGTGGGCCAACCCAGATGAATTTGACCCGGA
TAGATTTTTAATCGGTGGGTCCGAGTTTGAGTTCTCTGTTCTTGGGTC
GGATCTTAGACTTGCCCCTTTCGGGTCGGGTCGTAGGTCATGTCCTGG
GAAGGTCTTAGGTTTGACCACAGTCAGTTTTTGGGTTGCTTCACTCTT
GCATGAGTTTGAGTGGGTGACATCACCTAACGCTGACGTGGATTTGAG
TGAAGTGCTTAAGCTTTCGTGTGAGATGGCTCATCCTCTTACCGTGGA
AGCTAGGCCGCGACGTCATTAATTTTACAAAAGACGATATTCTCATAC
ACAGATTATTGTCAACTTTATCAATATATACGGGTGATAGTTTGACAT
TGTTATAGTATTCGGAAAAATATTTTCTAGAGATAAGTAAATTTATAA
TAATCTAAAAAACTGAATGTATAATTATATTAATTAATGTACTAATGT
TATTATAATGTTTATAAATGATTGTGAAATATATATGGTTAACATTAT
ATGTAATGAGAAATGGTTTGCTTGGCAAAAA.
>CYP86B1-like from M. jalapa
(SEQ ID NO: 38)
GCTAGCTGCCTTGTTCATTTCAAGAACAATATACACTAGTCAATCCAT
TTCAAATGGCCTACTTAGAAATTGTCATATTCTTCATTTTTCTCATCG
TAATTTGTTTTTCTTTTCGTGACAAAAATGGTCTCCCAACAAATTGGC
CGATTGTTGGGATGCTACCAGCTGTTCTAATCAATCTTCATAGAATTA
ATGACTATTTTGCTGAACTTGTTGCTAAATCAAATTTAACATTCTCAT
TTAAGGGTCCTTGGTTTAGCAACATGCGAATTTTAGCGACAGTTGATC
CAGCAAATGTGCACCATATAATGAGTAAGAATTTTAACAATTATCCTA
AGGGTGCTAAGTTTTACGACATCTTTGATATACTTGGAGATGGTATTT
TTAACACCGACTTTAATATATGGCAATACCATCGGAAAATGGCTCAGT
CATATATTGGTGACTCAAGATTTCAACAATTTTTGTTGAAGAAAGTTG
GGGAAAAGATTGAAGGTGGATTAATTCCTATCCTTGATCATGTTGCCA
TGCAAGGGTTACAAGTTGATTTACAAGATTTATTTGAAAGGTTTACTT
TTGATACTATTTGTTCACTTATTATGAACTATGATCCTATGTCTTTGT
CCATTGATTTTCCTGATGTTCCATCATCTAAGGCTCTAGATGTTGCTG
AAGAAGTCATACTTCTTCGTCATTTAGTACCTACAAGTGTTTGGAAGT
TTCAAAGATGGTTAGGTGTAGGGATGGAAAAGAAGCATAAGATAGCTT
GGCAAGTACTTGATGATTTCATTTATGAGTGCATATCAAGGAAAAGAG
AAGAGATGAGAGATAGTTTGTCCTATGAGAATAAGGACAATGAGATGG
GTGTTGATTTAATGACATTGTATATGAATGAAATCAAAAGTAATGAAC
TTGTCAAGGATGATCCTAACAAGTTTTTGAGGGATACTATTCTTAACT
TTTTTATTGCAGGTCGAGATACAACTAGTACTGCTTTATCGTGGTTTT
TCTATCTACTATCCAAGAATCCACAAGTGGTAGAAAAGATTAGACAAG
AGTTATCTTGGATCGTTTCACAAGAAAAAACTAAAAATTATGCTAATT
TGATTGATAAACTTGTTTATCTTCATGCCGCATTATGTGAAGCTTTAA
GATTATATCCTCCGGTGGTATTTGAAGCAAAGTCTCCGATTGAATCGG
ACACTTTACCAAGTGGTCATAAGATTGATCCTGACACACAAATAATCT
TAAATATGTACGCAATGGCGAGGATGAAAACAATATGGGGCGATGATT
GCGATCAATTTAAACCTGAGAGGTGGATATCATCAACAACAGGAAAGA
TTAAGCATGAACCTTCGTACAAGTTCTTGGCTTTTAATGCAGGACCAA
GAACTTGTGTAGGAAAAAATATGGCTTTTACTCAAATGAAAGCAATAG
CAATAGCTATATTACAAAACTATCATATACATGGTATTGATGGACAAG
TTATTGAACCTGATCTATCCATAATTCTCCATATGAAGAATGGATTCA
GAGTAACTGTTTCACCGTGTAACGTTTCTATATAACCATCGAGAGAAC
CAAGTTTCCTCGATCTCTTTTAAACATTGTAATGTTTAATTATCAACT
AAAAAATCGTATATTCTCTTCGTTTCATTTTTTTCTTTTTCTTTTTAT
CAAAGATCAAGGTAACGTCGGAAATAACCATATTGTTTTCTTTGGTTA
AATGAAAAGACAATGTAAGTTGTAAAAGTATGTAAGTGTAAAAATAAA
TTTAAAAAATTGTGTGAAAATAAGTTGTAATAAATAAAGAATAATGTT
GGTATTTTATTGTTTACTATTTTGATAAACAGAAAAAATAAAATAAAA
CTGTTTAAAAATAAAAGTGAGAAGAAAAAAAATAAAAGAAAAT.

Hypocotyl Transformation of Solanum nigrum

### Media

Liquid Jones (pH5.2)
Basal media (pH to 5.8 with NaOH before adding the agar)
MS with vitamins (cat M0222)=4.41 g/L
Sucrose 3%=30 g/L
Agarose (plant agar) 0.6%=6 g/L

TABLE 3
IND1 (first callus induction medium)

| | | | | | Amt. to add | Amt. to add |
|---|---|---|---|---|---|---|
| | Stock | | Final conc. | | to 1 L of | to 800 ml of |
| | solution | | in medium | | basal media | basal media |
| IAA | 0.2 | mg/ml | 0.02 | mg/L | 100 μl | 80 μl |
| BAP | 1 | g/L | 1 | mg/L | 1000 μl | 800 μl |

TABLE 4
IND2 (second callus induction medium)

| | | | | | Amt. to add | Amt. to add |
|---|---|---|---|---|---|---|
| | Stock | | Final conc. | | to 1 L of | to 800 ml of |
| | solution | | in medium | | basal media | basal media |
| IAA | 0.2 | mg/ml | 0.02 | mg/L | 100 μl | 80 μl |
| BAP | 1 | g/L | 1 | mg/L | 1000 μl | 800 μl |
| Kan* | 100 | mg/ml | 75 | mg/L | 750 μl | 600 μl |
| Tic | 100 | mg/ml | 100 | mg/L | 1000 μl | 800 μl |

*Make media without Kan for controls

TABLE 5
SHOOT (shoot induction medium)

| | | | | | Amt. to add | Amt. to add |
|---|---|---|---|---|---|---|
| | Stock | | Final conc. | | to 1 L of | to 800 ml of |
| | solution | | in medium | | basal media | basal media |
| BAP | 1 | g/L | 0.5 | mg/L | 500 μl | 400 μl |
| Kan* | 100 | mg/ml | 25 | mg/L | 250 μl | 200 μl |
| Tic | 100 | mg/ml | 100 | mg/L | 1000 μl | 800 μl |

*Make media without Kan for controls

TABLE 6
MAT (maturation medium)

| | | | Amt. to add | Amt. to add |
|---|---|---|---|---|
| | Stock | Final conc. | to 1 L of | to 800 ml of |
| | solution | in medium | basal media | basal media |
| Kan* | 100 mg/ml | 25 mg/L | 250 μl | 200 μl |
| Tic | 100 mg/ml | 100 mg/L | 1000 μl | 800 μl |

*Make media without Kan for controls

TABLE 7
ROOT (rooting medium in magenta)

| | | | |
|---|---|---|---|
| | | Amt. to add | Amt. to add |
| Stock | Final conc. | to 1 L of | to 800 ml of |
| solution | in medium | basal media | basal media |

| | |
|---|---|
| Kan* | 100 mg/ml |
| Tic | 100 mg/ml |
| IBA | 2 mg/ml |

*Make media without Kan for controls

### Procedure

Always work in a flow hood, close to a flame
Use forceps & scalpel sterilised with EtOH & flamed
Seal plates with parafilm before removing from the flow hood

### Prepare the Agrobacterium:

Pick a colony from a fresh transformation into Agrobacterium GV3101 & inoculate 7.5 ml LB buffer in a 50 ml falcon tube
Grow, shaking at 28° C. o/n
Add liquid Jones (pH5.2) up to a total volume of 25 ml

### Handling of the Explant:

Using seedlings that have expanded cotyledons, but no true leaves—remove individual seedlings using sterilised forceps & place onto a sterile Petri dish (approx. 30 hypocotyls per plate).

Cut the hypocotyl using a sterile scalpel, dipping the scalpel into the Agrobacterium mix before each incision.

For the control, dip the scalpel into liquid Jones before each incision.

Place all of the explants onto IND1 plates & place in the growth room in the dark for 3 days (wrap in foil).

### Subsequent Sub-Culturing:

Move the explants onto IND2 plates (spacing them out more ˜20 per plate) & place in the growth room with light/dark cycling
Check the plates every couple of days for contamination & transfer healthy explants to fresh media sooner if contamination is visible.

After 2-3 weeks, calli will form at the ends of the explants (where they were cut). Cut the calli away from the rest of the tissue & transfer to SHOOT media.

If calli have not formed—move to fresh IND2 media.

After 1-2 weeks, green shoots will start to develop from the calli. Transfer these calli to MAT media.

If shoots have not formed—move to fresh SHOOT media.

After 1-2 weeks, the shoots should be big enough to be cut away from the callus and transferred to ROOT media (in magentas).

If shoots are still small—move to fresh MAT media.

Move the plantlets to fresh ROOT media every 2-3 weeks until they have enough roots to move to soil.

### Example 2

### Transcriptome Profiling and Co-Expression Analysis in Mirabilis Jalapa Suggests the Involvement of a CYP76AD1 Subfamily Member in Betalain Biosynthesis

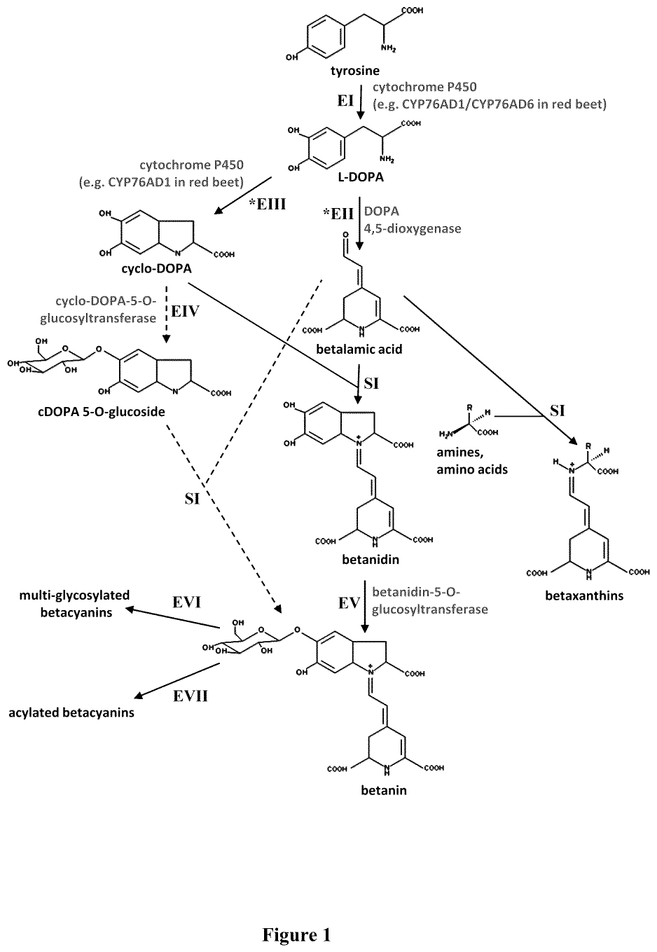

The primary goal of this study was to elucidate the first committed step in biosynthesis of betalains in plants, which commences with the aromatic amino acid tyrosine. Achieving this would close the gap in our current knowledge with respect to the plant enzymes and genes acting in the core betalain pathway (FIG. 1). Since genome and transcriptome sequence data from betalain-producing plants is currently very limited, we generated a comprehensive transcriptome dataset from the betalain pigmented Mirabilis jalapa (i.e. four o'clocks) plant. Libraries of 24 M. jalapa tissues were sequenced, including both betalain producing and non-producing ones. The selected set of tissues provided a temporal and spatial representation of betalain synthesis, as four different floral parts (i.e. petals, stamen, anthers and stigmas) were sampled in five developmental stages, each exhibiting increasing pigmentation during development (FIG. 2 A to F).

The transcriptome data was first analyzed by examination of gene expression patterns of the previously identified betalain-related genes in M. jalapa, namely DOPA 4,5-dioxygenase (MjDOD), cyclo-DOPA-5-O-glucosyltransferase (cDOPA5GT) and the cytochrome P450 CYP76AD3. These three genes were found to exhibit expression patterns which parallel pigment accumulation (i.e. increasing expression during flower development, and higher expression in red vs. green tissues) (FIG. 2G). Additional betalain related genes could thus potentially be found by co-expression analysis, searching for genes with expression patterns which highly correlate to those of MjDOD, cDOPA5GT or CYP76AD3.

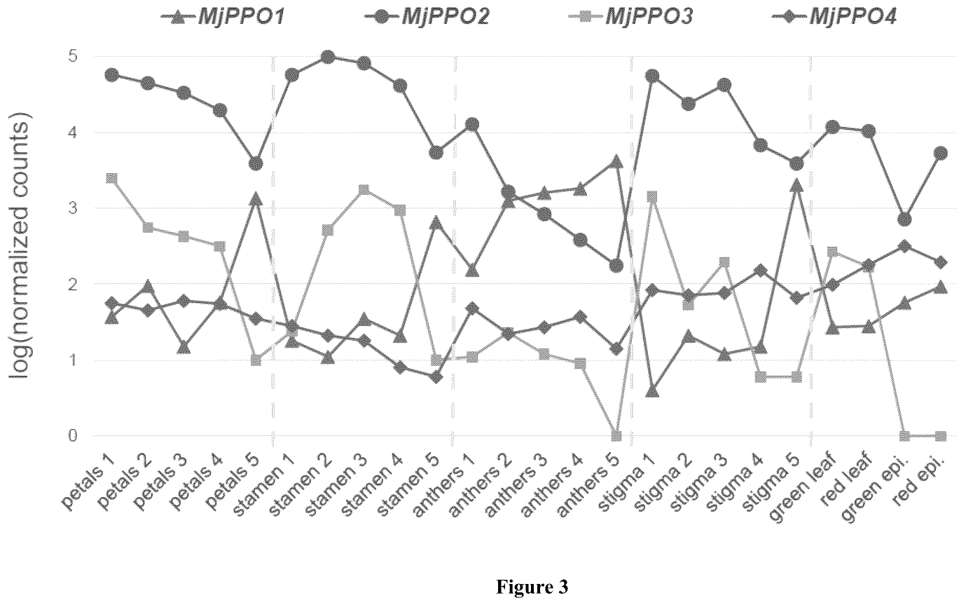

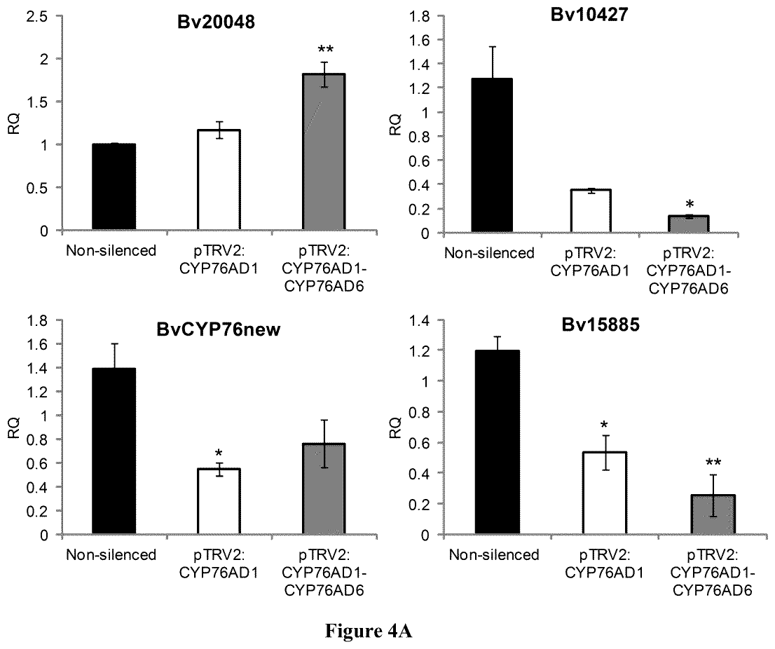

Tyrosine hydroxylation in betalain-producing plants is commonly thought to be catalyzed by a tyrosinase (polyphenol oxidase) enzyme. We therefore initially searched for a tyrosinase-encoding gene that is co-expressed with either MjDOD, cDOPA5GT or CYP76AD3, however none was found. A polyphenol oxidase expressed in a pattern which parallels betalain accumulation could neither be detected by manual examination of genes annotated as polyphenol oxidases in the M. jalapa dataset (FIG. 3). Alternatively, L-DOPA formation could in theory be catalyzed in M. jalapa by a cytochrome P450-type enzyme. Four cytochrome P450-encoding genes were found to be co-expressed with one or more of the known betalain related genes used as baits, namely CYP82G1-like (SEQ ID NO: 36), CYP78A9-like (SEQ ID NO: 37), CYP86B1-like (SEQ ID NO: 38) and CYP76AD1-like (SEQ ID NO: 1). The latter, hereinafter named MjCYP76, stood out as a promising candidate, exhibiting high expression values throughout the dataset, in a pattern which highly correlated with betalain accumulation (FIG. 2G).

### Example 3

CYP76AD1 and CYP76AD6 Co-Silencing Inhibits Betalain Production in Beta vulgaris

In order to examine whether MjCYP76 is indeed involved in betalain biosynthesis, a gene silencing assay was required. Since a robust and stable transformation procedure is not available for any betalain-producing plant we decided to apply Virus Induced Gene Silencing (VIGS), a widely used method for transient gene silencing in plants that is highly suitable for tracking pigmentation phenotypes. However, gene silencing with the VIGS method was previously shown to be particularly challenging in M. jalapa, most likely due to the inhibitory activity of the Mirabilis Antiviral Protein (MAP), and could only be achieved when the MAP gene is co-silenced with the gene of interest. Attempts for gene silencing in M. jalapa using VIGS were unsuccessful in our hands, including the attempted silencing of cDOPA5GT, which should result in partial loss of betalain pigmentation in the plant. We therefore proceeded to try and identify an MjCYP76 ortholog in a different betalain-producing plant species, in which gene silencing assays may be effectively implemented. An efficient VIGS method using vacuum infiltration was previously described in red beet, which was successfully used for silencing betalain related genes. Identification of an MjCYP76 ortholog in red beet could thus aid in assessment of the relevance of this gene to betalain biosynthesis.

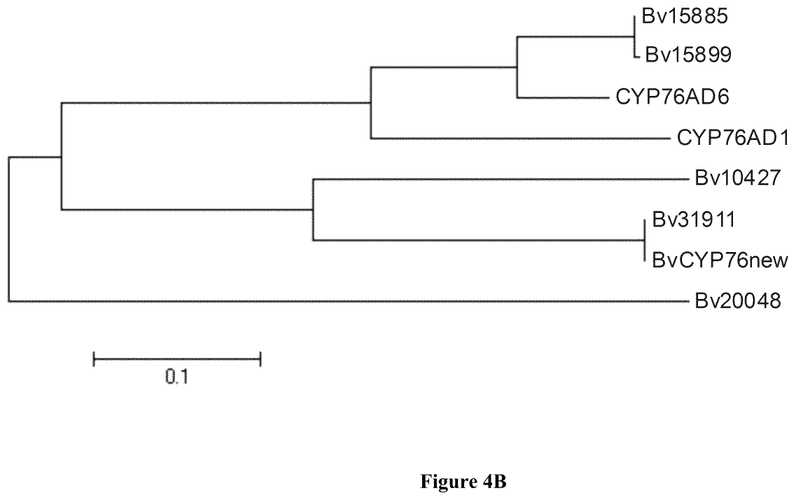

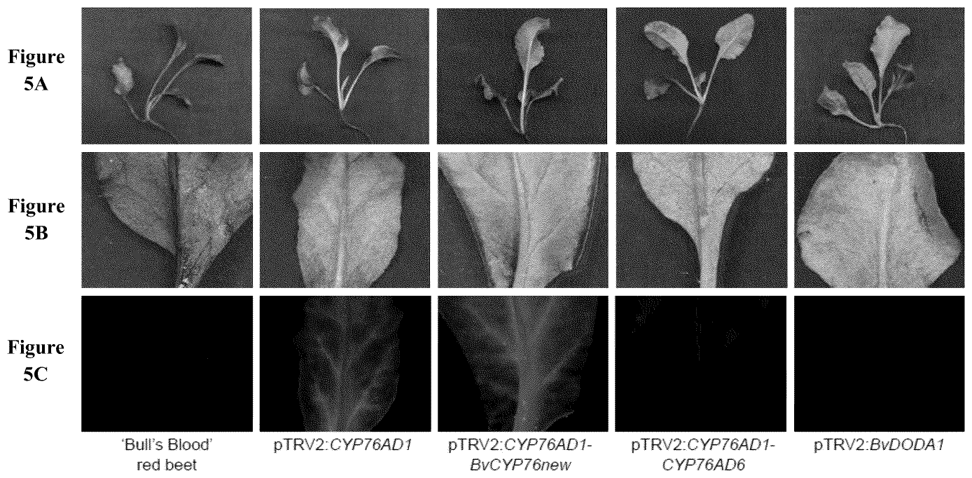

To this end, we sequenced the transcriptomes of hypocotyl tissues of three varieties of beet; a ‘golden beet’ variety which produces solely yellow betaxanthins, as well as a red beet and a ‘striped beet’ variety, both of which have red-pigmented hypocotyls. An MjCYP76 ortholog in beet, which would putatively play a key role in betalain biosynthesis, was expected to be highly expressed in all three tissues. Homologs were identified by tBLASTx querying of the beet trasncriptome dataset with the MjCYP76 coding sequence. Contigs representing eight different cytochrome P450 genes with high sequence homology to MjCYP76 (over 50% identity on the protein level) were found, including the previously characterized CYP76AD1 (FIG. 4A). The highest scoring gene, BvCYP76new, was selected as a potential candidate to be a functional ortholog of MjCYP76 in beet. An additional gene within this group, hereinafter named CYP76AD6, which holds a 60% identity with MjCYP76 on the nucleotide level and 53% on the amino acid level, exhibited markedly higher expression values compared to the other paralogs and was therefore also selected for subsequent analysis.

Fragments from BvCYP76new and CYP76AD6 were cloned into the VIGS vector pTRV2 and introduced into seedlings of the ‘Bull's Blood’ red beet variety, which produces plants with highly pigmented dark red leaves and is therefore particularly compatible for gene silencing assays for betalain related genes. Since the tyrosine hydroxylase reaction to form L-DOPA is essential for formation of all betalain compounds, including both red betacyanins and yellow betaxanthins, silencing of either BvCYP76new or CYP76AD6 was expected to produce plants exhibiting green patches, due to lack of betalain pigmentation. pTRV2:BvDODA1 and pTRV2:CYP76AD1 were also introduced to beet seedlings and served as positive controls for the assay. Silencing of BvCYP76new or CYP76AD6 did not result in any visible phenotype (data not shown), while the silencing of BvDODA1 and CYP76AD1 produced patches of green or yellow color, respectively, as was previously reported. One possible explanation for a lack of visible phenotype when silencing a gene putatively encoding the enzyme responsible for L-DOPA production, was that this enzyme may be redundant with another enzyme or enzymes that also catalyze the same reaction. One such candidate is CYP76AD1, which could possibly catalyze the tyrosine hydroxylation step in addition to its experimentally demonstrated activity of conversion of L-DOPA to cyclo-DOPA. CYP76AD1 would have to be acting redundantly with another enzyme, since the silencing of CYP76AD1 does not prevent the formation of betaxanthins, for which L-DOPA is an essential precursor (FIG. 1). This hypothesis could be examined by co-silencing of BvCYP76new or CYP76AD6 together with CYP76AD1.

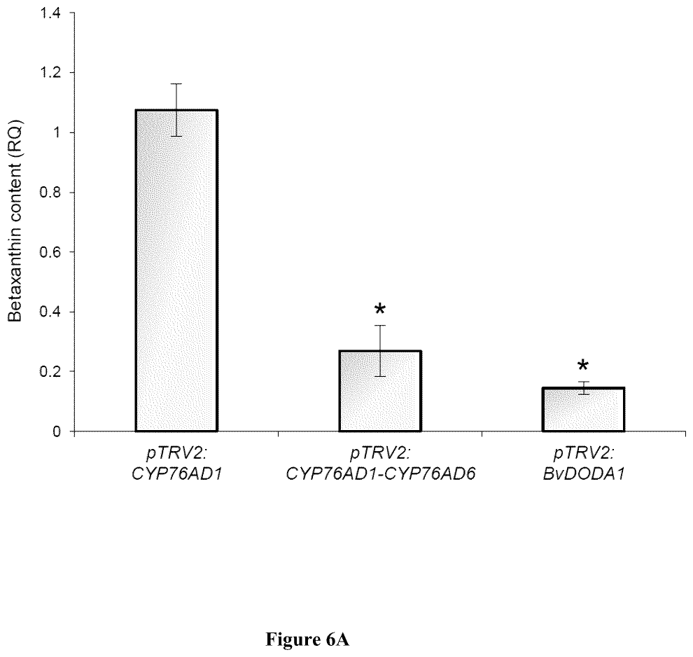

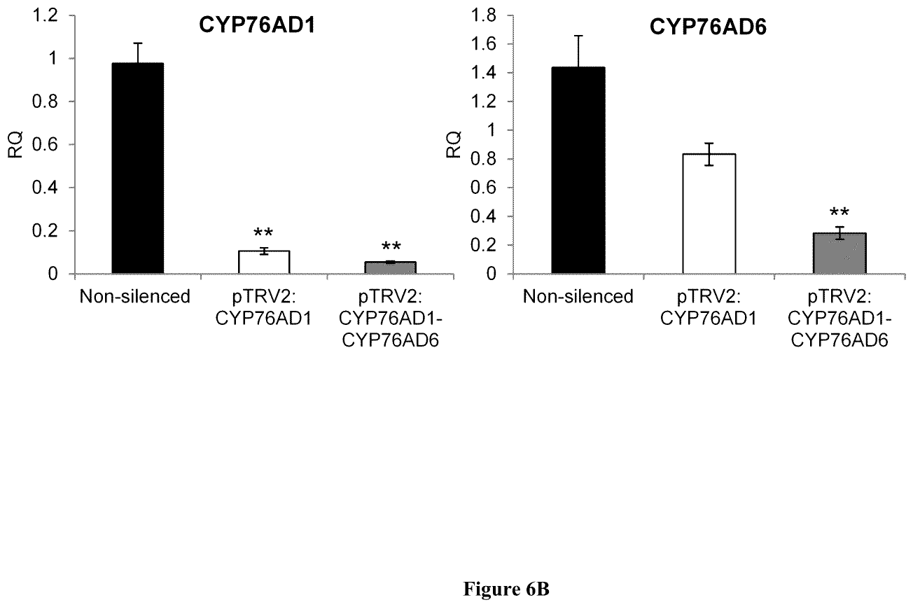

We therefore constructed pTRV2 vectors which harbor fragments of CYP76AD1 with either of the BvCYP76new or CYP76AD6 candidates, in tandem. A subsequent experiment was carried out for silencing of BvDODA1, CYP76AD1, BvCYP76new or CYP76AD6, as was done in the aforementioned VIGS experiment, in addition to co-silencing of CYP76AD1 with each of the CYP76 candidates. Plants in which CYP76AD1 and BvCYP76new were co-silenced produced yellow patches in leaves, similarly to plants in which only CYP76AD1 was silenced. However, co-silencing of CYP76AD1 and CYP76AD6 caused a clearly evident phenotype of green patches, which lack both betacyanins and betaxanthins, similarly to the phenotype obtained by BvDODA1 silencing (FIG. 5A, B). Differences in betaxanthin accumulation levels could also be observed by blue light imaging (FIG. 5C), under which betaxanthins typically exhibit strong fluorescence. Reduced betaxanthin accumulation in CYP76AD1-CYP76AD6 co-silenced tissue versus CYP76AD1 silenced tissue was further demonstrated by spectrophotometric analysis (FIG. 6A). These findings supported the notion of redundancy in the tyrosine hydroxylation step of betalain biosynthesis in red beet, by which this step may be catalyzed by either CYP76AD1 or CYP76AD6. Additionally, the fact that silencing of CYP76AD1 alone blocks production of betacyanins but not of betaxanthins, suggested that CYP76AD6 does not catalyze the conversion of L-DOPA to cyclo-DOPA, but only the formation of L-DOPA from tyrosine.

qRT-PCR analysis was performed in order to examine expression of CYP76AD1 and CYP76AD6 in plants infected with the pTRV2:CYP76AD1 or pTRV2:CYP76AD1-CYP76AD6 vectors. CYP76AD1 was found to be down-regulated upon infection with both vectors, while CYP76AD6 only showed down-regulation following infection with pTRV2:CYP76AD1-CYP76AD6 (FIG. 6B). Expression of four CYP76AD1 and CYP76AD6 paralogs was also examined. Three of the four paralogs examined showed down-regulation in tissues infected with at least one of the pTRV2:CYP76AD1 or pTRV2:CYP76AD1-CYP76AD6 vectors (FIG. 4A). Thus, the possible involvement of one or more of these paralogs in betalain biosynthesis cannot be ruled out at present. However, irrelevance of one of the three paralogs, BvCYP76new, to betalain biosynthesis may be inferred from results of the silencing experiments, as silencing of BvCYP76new alone caused no visible change in phenotype, and silencing of this gene together with CYP76AD1 produced the same phenotype as observed when CYP76AD1 was silenced alone.

### Example 4

Transient Expression of CYP76AD1 in N. benthamiana Enables Production of L-Dopa and Betacyanins

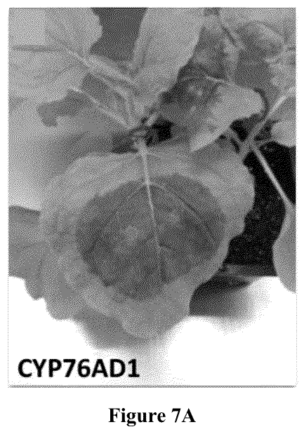

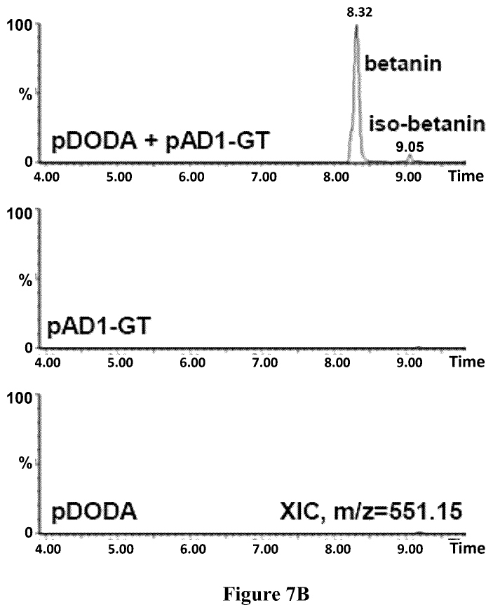

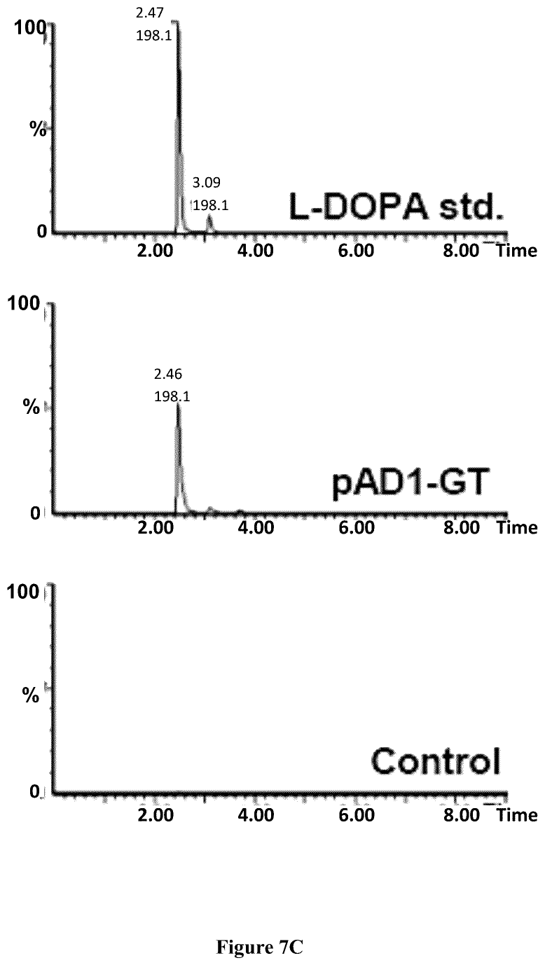

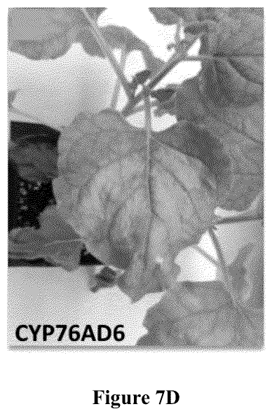

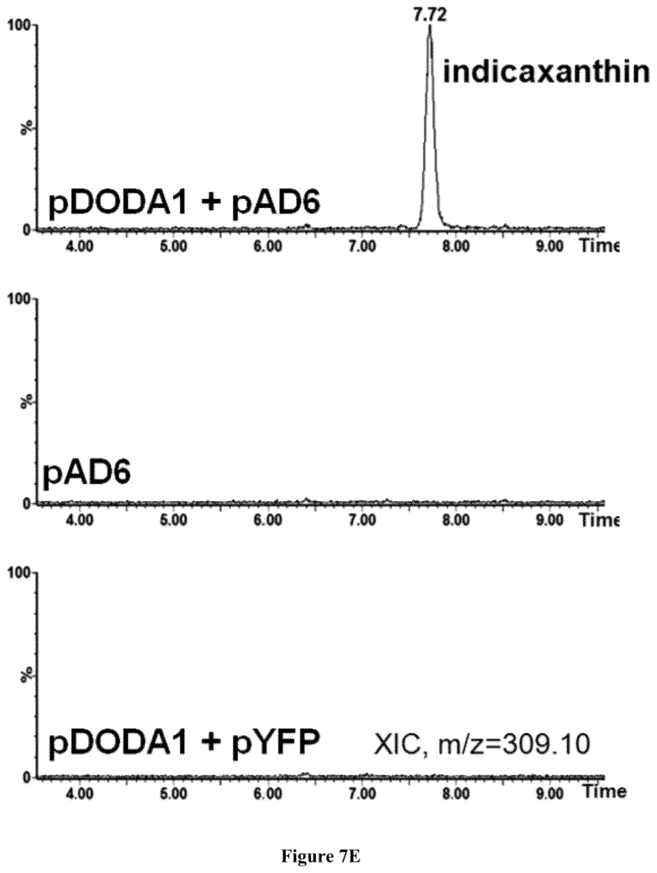

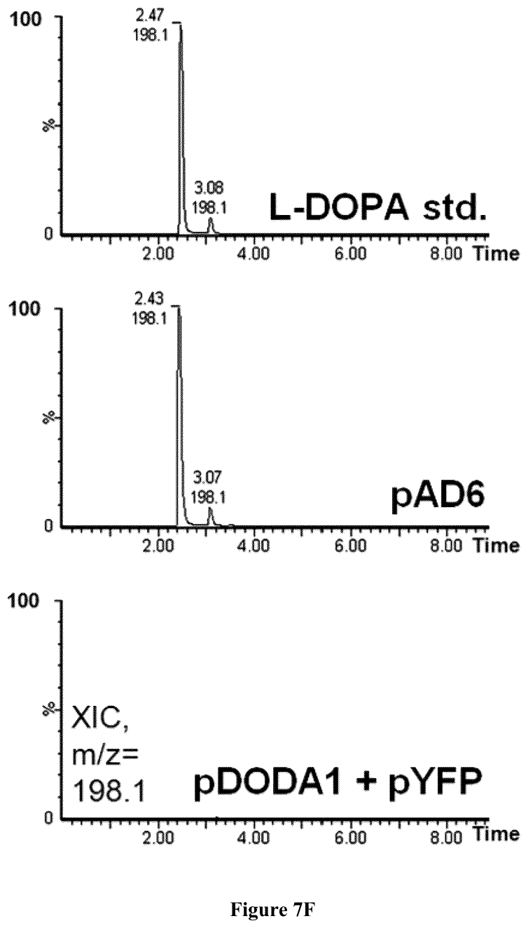

If CYP76AD1 indeed catalyzes both the formation of L-DOPA and its conversion to cyclo-DOPA, then heterologous expression of CYP76AD1 together with a DOD enzyme and a betalain related glucosyltransferase (e.g. cyclo-DOPA 5-O-glucosyltransferase or betanidin-5-O-glucosyltransferase) should be sufficient for biosynthesis of the widely common glucosylated betacyanin, betanin. To test this, we generated overexpression constructs for the beet BvDODA1 and CYP76AD1 genes and the M. jalapa gene cyclo-DOPA 5-O-glucosyltransferase (cDOPA5GT), to be used for transient, agroinfiltration-mediated overexpression in Nicotiana benthamiana leaves. Two vectors were made for this purpose; one for overexpression of BvDODA1 under the CaMV 35S promoter (pDODA) and another vector for the tandem expression of CYP76AD1 and cDOPA5GT, driven by the CaMV 35S and Arabidopsis ubiquitin-10 promoters, respectively (pAD1-GT). Co-infiltration of agrobacteria (Agrobacterium tumefaciens) harboring the pDODA and pAD1-GT vectors into N. benthamiana leaves caused the appearance of dark red pigmentation in infiltrated areas within 2-3 days post infiltration (FIG. 7A). Liquid chromatography-mass spectrometry (LC-MS) analysis revealed that the pigmented leaf tissue contained high levels of betanin, as well as the betanin isomer iso-betanin (FIG. 7B; Table 2). This result provided a first proof of concept for the possibility of engineering betalain production in-planta without substrate feeding. Additionally, the formation of betacyanins without the need for L-DOPA feeding or overexpression of a tyrosine hydroxylating enzyme (e.g. tyrosinase) indicated that CYP76AD1 catalyzes the formation of L-DOPA in addition to its previously known activity of converting L-DOPA to cyclo-DOPA.

However, the production of betalains may have alternatively been possible due to occurrence of L-DOPA in N. benthamiana leaves as a result of tyrosine hydroxylase activity by an endogenous enzyme. To examine whether L-DOPA production was enabled due to the expression of CYP76AD1, pAD1-GT was infiltrated into N. benthamiana leaves without pDODA. Introduction of a vector overexpressing YFP protein (pYFP) was used as a control for this experiment. LC-MS analysis confirmed the presence of L-DOPA in pAD1-GT infiltrated tissues but not in pYFP infiltrated tissues (FIG. 7C).

### Example 5

Transient Expression of CYP76AD6 in N. benthamiana Enables Production of L-DOPA and Betaxanthins

Tyrosine hydroxylase activity of CYP76AD6 was also assessed by transient overexpression in N. benthamiana leaves. An overexpression vector for CYP76AD6 under the CaMV 35S promoter (pAD6) was constructed and inserted to agrobacteria. Co-infiltration of pAD6 and pDODA resulted in the appearance of yellow pigmentation in infiltrated areas, visible within several days post infiltration. (FIG. 7D). LC-MS analysis of the yellow-pigmented tissue revealed the occurrence of one major betaxanthin compound, identified as indicaxanthin (proline-betaxanthin) (FIG. 7E). Two additional compounds were found to exhibit typical betaxanthin light absorption spectra, one of which was putatively identified as a dopaxanthin-hexoside, based on accurate mass and fragmentation (Table 2). Notably, glycosylated-betaxanthins do not occur, to the best of our knowledge, in natural betalain-producing species. The fact that co-infiltration of CYP76AD6 and BvDODA1 induced the production of betaxanthins but not of betacyanins coincides with the data obtained in the aforementioned gene silencing assays, confirming that CYP76AD6 catalyzes only the single step of L-DOPA formation and not its subsequent conversion to cyclo-DOPA. Agroinfiltration of the pAD6 vector alone resulted in production of L-DOPA in N. benthamiana leaves, verified by LC-MS (FIG. 7F).

### Example 6

### Recombinant Expression of CYP76AD1 and CYP76AD6 Allows Betalain Synthesis in Yeast

CYP76AD1 and CYP76AD6 activity was further evaluated by recombinant expression of each of the two enzymes in yeast, together with BvDODA1. To this end, full coding sequences of BvDODA1, CYP76AD1 and CYP76AD6 were cloned into galactose-inducible yeast overexpression vectors and transformed into Saccharomyces cerevisiae cells. Yeast were subsequently grown in standard SD media, with no L-DOPA supplementation. Similarly to the N. benthamiana transient expression assays discussed above, red-violet pigmentation was observed in media of yeast expressing CYP76AD1 and BvDODA1, while yellow pigmentation was evident in media of yeast expressing CYP76AD6 and BvDODA1. Expression of each of the cytochrome P450 enzymes without BvDODA1 or expression of BvDODA1 alone did not result in formation of red or yellow pigments. Additionally, expression of CYP76AD1, CYP76AD6, and BvDODA1 together, resulted in formation of orange pigmentation in the media (FIG. 8A). LC-MS analysis was used to verify the occurrence of betalains in the yellow, red-violet and orange-pigmented media (FIG. 7C; Table 2).

### Example 7

CYP76AD6 Belongs to a Phylogenetic Clade that was not Previously Associated with Betalain Biosynthesis

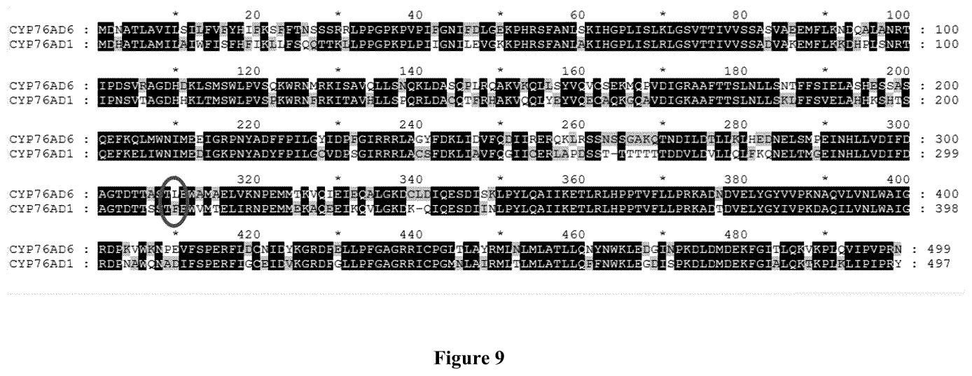

In a recently published study, large-scale phylogenetic analyses of Caryophyllales plants suggested the occurrence of several gene duplication events of the CYP76AD1 locus early in Caryophyllales evolutionary history. These events were suggested to give rise to three clades within the CYP76AD1 lineage, named CYP76AD1-α, CYP76AD1-β and CYP76AD1-γ. The inventors deduced that the CYP76AD1-α clade is directly associated with betalain biosynthesis as it includes, among others, the functionally characterized B. vulgaris CYP76AD1 gene and its M. jalapa ortholog CYP76AD3, which has also been implicated to play a similar role in betalain biosynthesis. However, nothing is currently known with respect to the function of genes belonging to the CYP76AD1-β and CYP76AD1-γ clades. Interestingly, the CYP76AD6 gene reported here [accession ‘Bv9_228610_ygeq.t1’ in the B. vulgaris genome] is positioned in the CYP76AD1-β clade, as demonstrated in the phylogenetic analyses presented by Brockington and co-workers, and further presented here in a maximum-likelihood phylogenetic tree (FIG. 8B). Pairwise alignment of the CYP76AD6 and CYP76AD1 protein sequences showed a 72% percent identity (FIG. 9). Their corresponding genes exhibit 70% sequence identity at the nucleotide level.

### Example 8

### Metabolic Engineering of Red Betalain Pigments in Transgenic Plants Requires Heterologous Expression of Three Biosynthetic Genes

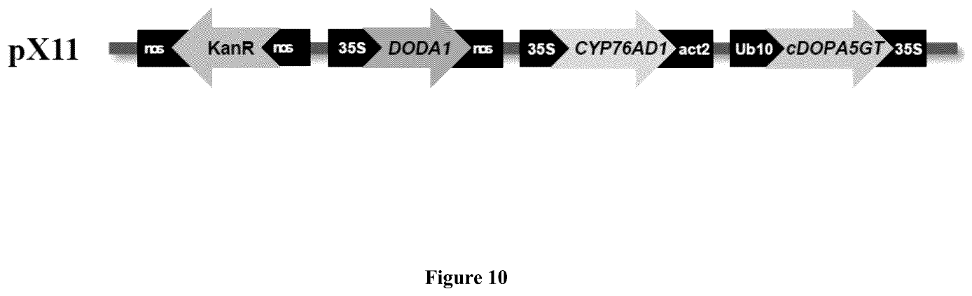

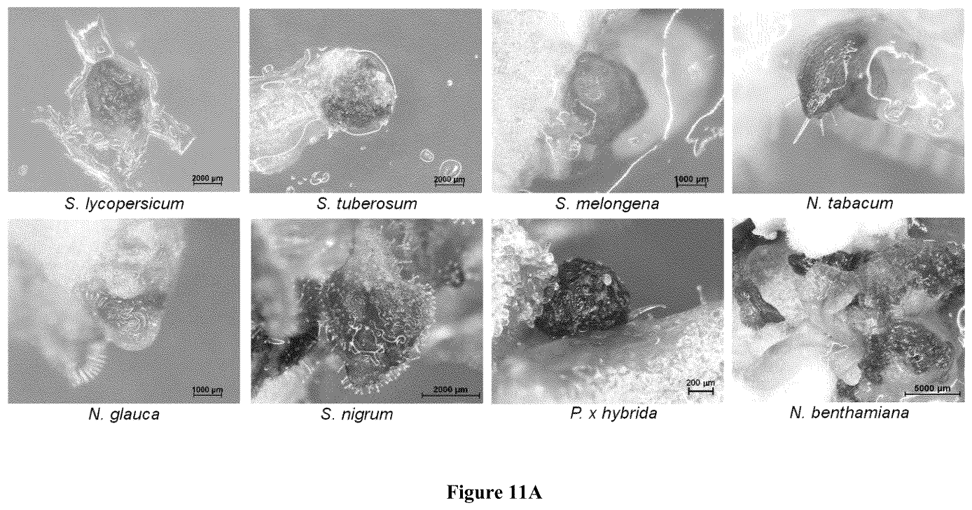

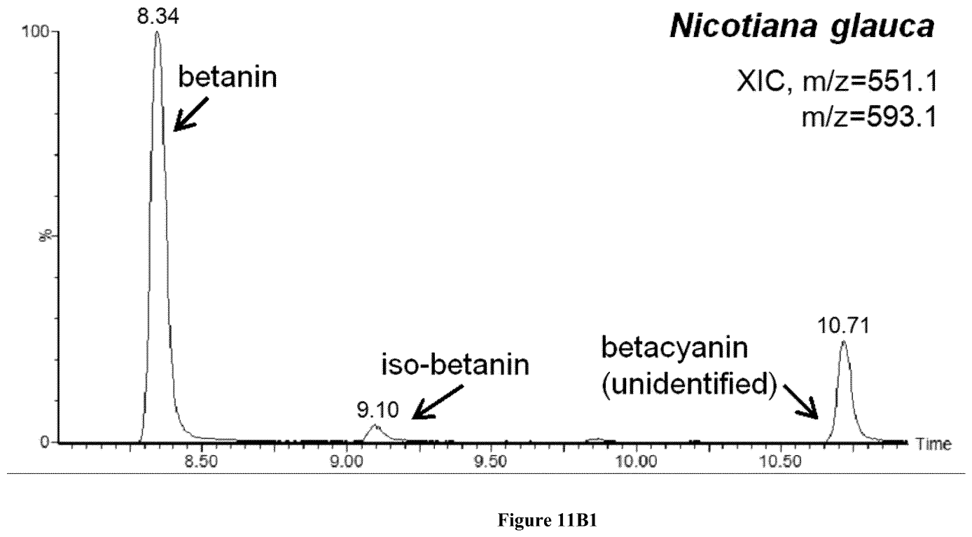

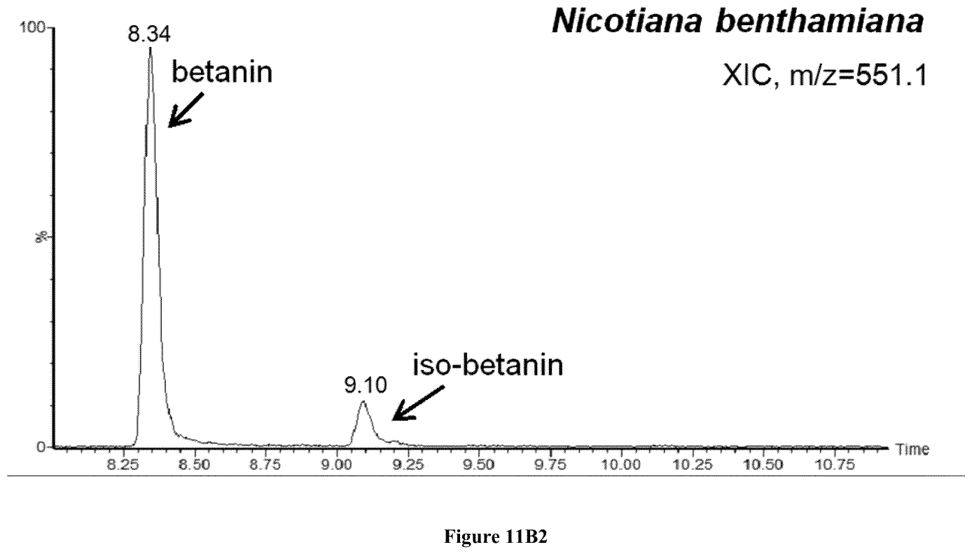

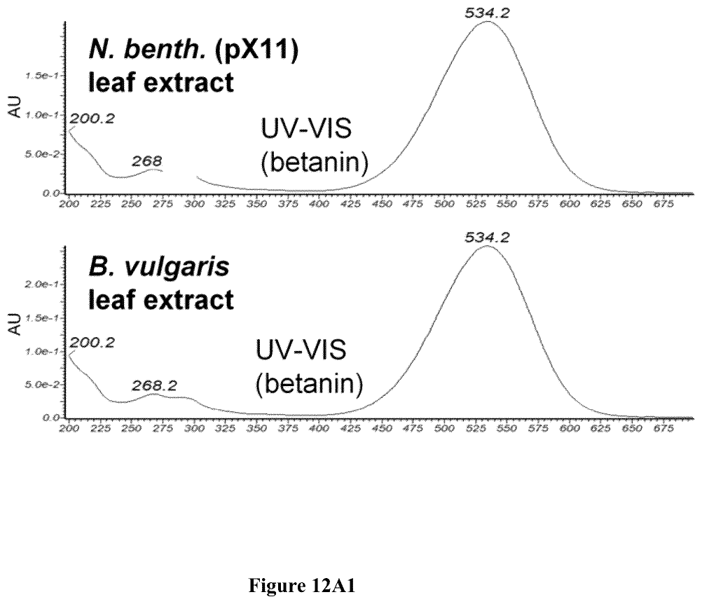

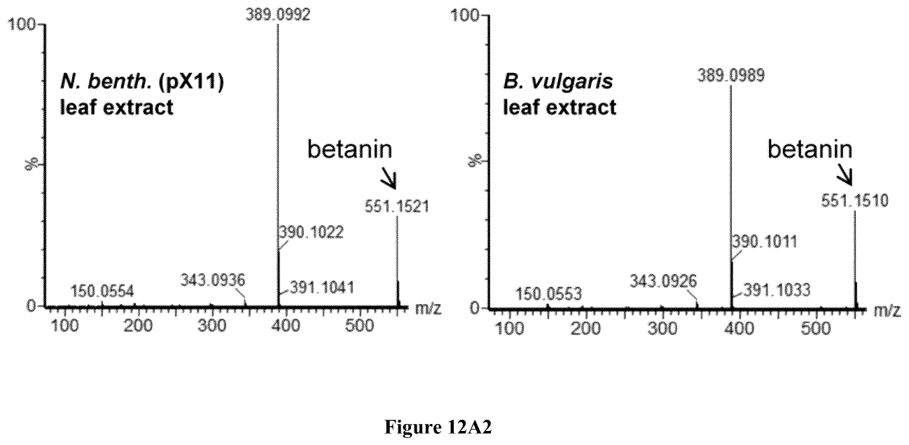

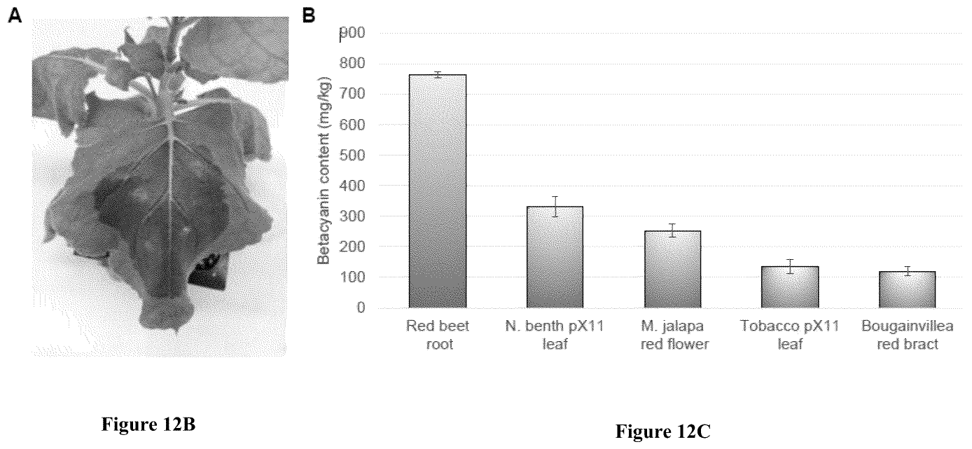

As discussed above, we were able to generate both red betacyanins and yellow betaxanthins through transient gene overexpression in N. benthamiana leaves. We subsequently attempted to engineer betalain production in naturally non-producing plant species through stable transformation. To this end, Goldenbraid cloning was used to generate a four-gene construct (termed hereafter pX11), in which CYP76AD1, BvDODA1 and cDOPA5GT are expressed in tandem and driven by constitutive promoters, in addition to a kanamycin resistance gene for transgene selection (FIG. 10). The pX11 vector was initially tested by agroinfiltration into N. benthamiana. Similarly to the co-infiltration of pDODA and pAD1-GT, introduction of pX11 resulted in red pigmentation of the infiltrated tissue (FIG. 12B). LC-MS analysis of pX11-infiltrated tissue was conducted in order to determine betalain composition. Peaks corresponding to betanin and iso-betanin were found, as previously observed by the co-infiltration of pDODA and pAD1-GT overexpression vectors (FIG. 11A). Also identified was the betaxanthin compound indicaxanthin (proline-betaxanthin), indicating some level of flux towards betaxanthin formation. Two additional metabolites were recognized as betacyanins, based on light absorption spectra and fragmentation, which included the nearly ubiquitous betacyanin fragment, betanidin (m/z=389; Table 2). The pX11-infiltrated leaf tissue appeared intensely red-pigmented, seemingly producing betacyanins in high quantities (FIG. 12B). Infiltrated tissue was therefore assessed for total betacyanin content by spectrophotometric analysis, and was found to contain an estimate of 330 mg kg-1, higher than red bracts of Bougainvillea glabra and red petals of M. jalapa, containing 120 mg kg-1 and 250 mg kg-1 betacyanins, respectively, and 2.3 fold lower than red beet root, which was estimated to contain 760 mg kg-1 (FIG. 12C).

The pX11 vector was next used for stable transformation of several plant species, including tobacco (Nicotiana tabacum), tomato (Solanum lycopersicum), potato (Solanum tuberosum), eggplant (Solanum melongena), tree tobacco (Nicotiana glauca), European black nightshade (Solanum nigrum), Petunia (Petunia x hybrida) and Nicotiana benthamiana. Explants of the different species were co-cultivated with agrobacteria and placed on selective media. In N. benthamiana, N. glauca, Petunia and tomato, small patches of red-violet pigmentation were visible on the explant surface within two days of co-cultivation with agrobacteria. In all species transformed, introduction of pX11 caused the formation of red-violet calli, which generally appeared within 1-2 weeks of culture (FIG. 11A). Pigmented N. benthamiana and N. glauca callus tissues were found to contain betanin and iso-betanin with LC-MS. Additionally, a novel unidentified betacyanin compound was found in the N. glauca callus (FIG. 10B, Table 2).

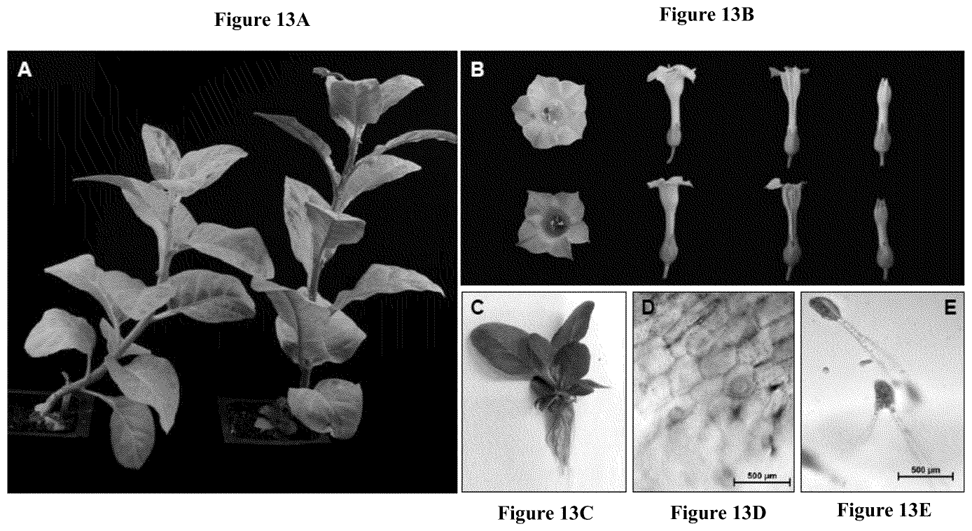

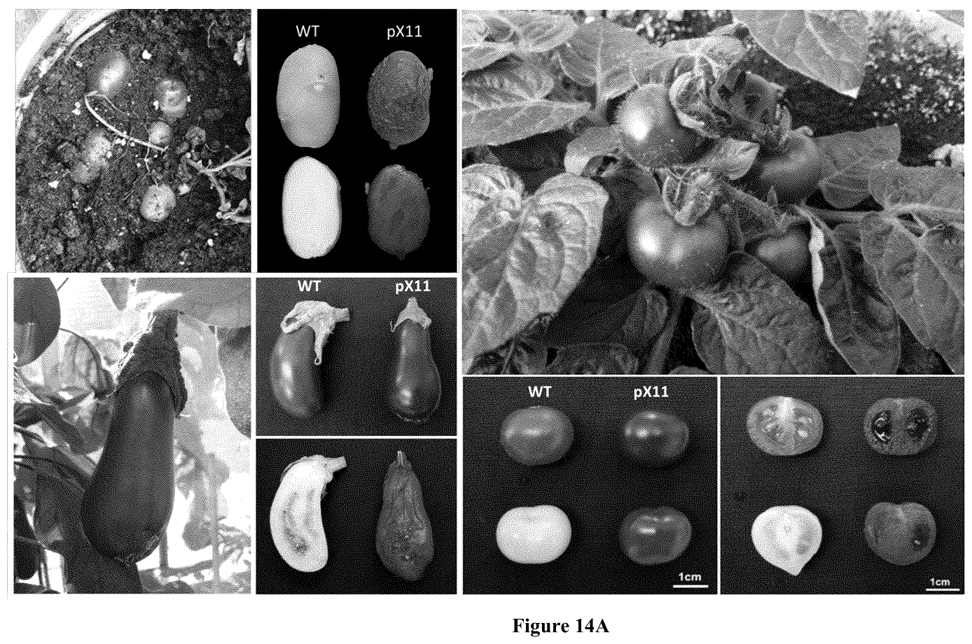

Tissue culture of pX11-transformed tobacco (Nicotiana tabacum L. cv. Samsun-NN) eventually led to the generation of mature, intensely red-pigmented plants that displayed betalain accumulation in all major plant organs including stems, leaves, roots, and flowers (FIG. 13). LC-MS analysis of transgenic pX11-tobacco leaves validated the occurrence of the betacyanins, betanin, and iso-betanin as well as the betaxanthin compound vulgaxanthin I (glutamine-betaxanthin) (Table 2). Betacyanin content of pX11-tobacco leaves was additionally measured spectrophotometrically to be 135 mg kg-1 (FIG. 12C).

### Example 9

### Discussion

### CYP76AD1 and CYP76AD6 Redundantly Catalyze Tyrosine Hydroxylation, the First Committed Step in Betalain Biosynthesis in Plants

In this study we have demonstrated the involvement of CYP76AD1 in tyrosine hydroxylation to L-DOPA in red beet, apart from its previously reported role in catalyzing the subsequent reaction leading to cyclo-DOPA formation. Furthermore, we identified an additional beet cytochrome P450 enzyme, CYP76AD6, which catalyzes the first hydroxylation step in redundancy with CYP76AD1 (FIG. 1). A CYP76AD6 ortholog, MjCYP76, was primarily identified in Mirabilis jalapa, exhibiting an expression pattern which parallels betalain accumulation. It is likely that MjCYP76 and the previously identified CYP76AD3, respectively, serve the same functions in M. jalapa as CYP76AD6 and CYP76AD1 do in red beet, although this remains to be conclusively verified by functional analyses, mostly gene silencing assays in M. jalapa.

CYP76AD6 involvement in the formation of L-DOPA was initially demonstrated by gene silencing assays; while silencing CYP76AD1 by itself inhibits formation of betacyanins but not betaxanthins, silencing CYP76AD1 and CYP76AD6 in parallel blocks the formation of both betalain groups. Silencing of CYP76AD6 alone produces no visible phenotype, since both L-DOPA and cyclo-DOPA formation continue to be catalyzed by CYP76AD1. This lack of visible phenotype may partially explain why CYP76AD6 and the tyrosine hydroxylation step remained last to be discovered within the core betalain biosynthesis pathway. While DOD and CYP76AD1-like genes could be identified in betalain-producing species by naturally occurring or artificially induced mutations that cause clearly visible phenotypes, CYP76AD6 could not, due do its functional redundancy with CYP76AD1. The suggested roles of CYP76AD1 and CYP76AD6 in betalain biosynthesis were further corroborated by recombinant expression in N. benthamiana and yeast cells, which led to the formation of betacyanins or betaxanthins, respectively, when coupled to BvDODA1 expression. Additionally, formation of L-DOPA in N. benthamiana was detected when either CYP76AD1 or CYP76AD6 were overexpressed without BvDODA1.

CYP76AD1 and CYP76AD6 belong to two separate clades within the CYP76AD1 subfamily, named CYP76AD1-α and CYP76AD1-β clades. It is conceivable that the two cytochrome P450 enzymes would share some degree of structural relatedness, as they redundantly catalyze an identical enzymatic reaction. Moreover, it was reported previously that cytochrome P450s belonging to a single subfamily catalyze subsequent steps in the same biosynthetic pathway. It is plausible that while the CYP76AD1-α clade may be comprised of enzymes which possess dual activity of L-DOPA and cyclo-DOPA formation, the CYP76AD1-β may include enzymes which catalyze only L-DOPA formation. However, functional analyses of corresponding genes from additional betalain-producing species will be needed to support this hypothesis.

Tyrosinase enzymes catalyze both the ortho-hydroxylation of monophenols and the oxidation of o-diphenols to o-quinones, thus directly converting tyrosine to the oxidized form of L-DOPA, dopaquinone. CYP76AD1, in a currently unknown reaction mechanism, also catalyzes both tyrosine hydroxylation and L-DOPA conversion to cyclo-DOPA. Our experimental results suggest that CYP76AD6 is unique in that it catalyzes the ortho-hydroxylation of tyrosine but not the conversion of L-DOPA to dopaquinone or cyclo-DOPA. The unique activity of the CYP76AD6 enzyme can be harnessed for high-scale production of L-DOPA that is highly advantageous and unexpected compared to current production methods. CYP76AD6 directly forms L-DOPA from the substrate tyrosine. The ubiquitous nature of tyrosine enables the possibility of expression of CYP76D6 in a variety of biological platforms including plants and microbes.

The discovery of a new class of tyrosine hydroxylating enzymes might also facilitate the identification of additional cytochrome P450 genes that may catalyze L-DOPA formation in biosynthesis of other L-DOPA derived metabolites. For example, the gene responsible for tyrosine hydroxylation in the biosynthesis of benzylisoquinoline alkaloids (BIAs) remains unknown to date, in an otherwise well-characterized pathway. It is possible that in BIA producing plants, L-DOPA is also catalyzed by a cytochrome P450 similar to CYP76AD1 and CYP76AD6.

Uncovering of the roles of CYP76AD1 and CYP76AD6 in L-DOPA formation advances our understanding of betalain biosynthesis in plants and essentially completes the identification of genes and enzymes that make the core betalain structures. Yet, the betalain pathway remains relatively poorly understood with regards to betalain structure modification through ‘decorating’ enzymes (e.g. glycosylating and acylating enzymes), subcellular transport and detoxification. Further characterization of the genes and regulatory factors involved in the biosynthesis of these pigments will hopefully be facilitated by the elucidation of the core pathway and through increasing availability of sequence data from betalain producing plants.

### Metabolic Engineering of Betalain Production in Plants

While anthocyanins and carotenoids are prevalent throughout the plant kingdom, betalains are pigments specific to the Caryophyllales plant order, in which they are produced in a phylogenetically ordered, mutually exclusive fashion with anthocyanins. Their enigmatic evolutionary history, as well as their nutritionally beneficial qualities and economical value as natural food colorants, have led to increasing interest in these pigments in recent years, including the prospect of synthesizing them in microorganisms and plants.

In this study, we have demonstrated for the first time the engineering of betalain production in plants, without substrate feeding, by both transient gene expression and stable transformation. This was made possible by identification of the additional tyrosine hydroxylase activity of CYP76AD1. Expression of CYP76AD1, in combination with BvDODA1 and cDOPA5GT, was therefore found to be sufficient for biosynthesis of betanin, without the need for an exogenous supply of L-DOPA. The use of the pX11 vector that includes the CYP76AD1, BvDODA1 and cDOPA5GT genes was first demonstrated by transient, agroinfiltrated-mediated expression in N. benthamiana, which resulted in the production of betalains in high quantity, visible within two days post infiltration. Attempts for the stable transformation of pX11 into plants and their subsequent cultivation in tissue culture, resulted in pigmentation of explants and the formation of red-violet calli and shoots in a variety of plant species within several days, demonstrating the potential use of betalains as visible genetic transformation markers. Stable transformation of tobacco plants with pX11 ultimately led to the generation of fully pigmented mature plants. LC-MS analysis of the betalain producing tobacco plants confirmed the occurrence of betanin as the major betalain produced.

The findings in this study therefore open the way for heterologous production of betalains in additional plant species, potentially resulting in the development of nutritionally enhanced food crops as well as new varieties of ornamentals. Apart from engineering red-pigmented betacyanin-producing plants, betalain production could principally be modified to be limited to betaxanthins by expressing CYP76AD6 instead of CYP76AD1, as exemplified by the transient expression experiments in N. benthamiana. Engineering of plants which exhibit both red and yellow pigmentation might also hypothetically be achieved by expressing both cytochrome P450s under different, non-constitutive promoter sequences (e.g. fruit-specific, flower-specific or inducible promoters). The engineered betalain producing plants will also serve as an exceptional genetic resource to examine the pigment-based attraction of pollinators to flowers and frugivores to fruit, as well as the role of pigments in assisting plant organs to resist abiotic and biotic cues.

### Example 10

### Materials and Methods (2)

### Generation of DNA Constructs

Gene sequences used in this study; cDOPA5GT (GenBank accession AB182643.1), BvDODA1 (accession HQ656027.1), CYP76AD1 (HQ656023.1). CYP76AD6 (KT962274), AroG175 (JC233128.1), AAH (HQ003815.1). CYP76AD15 sequence is provided herein. M. jalapa cDOPA5GT, CYP76AD15 and B. vulgaris CYP76AD1, BvDODA1, and CYP76AD6 transcripts were amplified from M. jalapa red petal and B. vulgaris red hypocotyl cDNA libraries. DNA constructs used for N. benthamiana agroinfiltration and for agrobacteria-mediated plant transformation were constructed with Goldenbraid cloning (Sarrion-Perdigones et al., 2013; GoldenBraid 2.0: A Comprehensive DNA Assembly Framework for Plant Synthetic Biology. Plant Physiology 162: 1618-1631). BvDODA1, CYP76AD1, CYP76AD6, CYP76AD15, cDOPA5GL AroG175 and AAH were initially cloned into a pUPD vector. pX11, pX11(E8), pX11(CHS), pX13, pDOPA3 and pDOPA4 are 3α1 vectors. pX12, pDOPA1 and pDOPA2 are 3Ω1 vectors. All vectors are based on a pCAMBIA backbone (Roberts et al., 1997; A Comprehensive Set of Modular Vectors for Advanced Manipulations and Efficient Transformation of Plants. In pCAMBIA Vector Release Manual Rockefeller Foundation Meeting of the International Program on Rice Biotechnology, September 15-19, Malacca, Malaysia).

### Plant Transformation and Regeneration

Agrobacteria-mediated plant transformation and regeneration was done according to the following methods; tobacco leaf discs (Horsch et al., 1985; Science 227: 1229-1231), eggplant (Van Eck and Snyder, 2006; Eggplant (Solanum melongena L.). In K Wang, ed, Agrobacterium Protocols. Springer, pp 439-448), tomato (McCormick, 1997; Transformation of tomato with Agrobacterium tumefaciens. In K Lindsey, ed, Plant tissue culture manual. Springer, pp 311-319), potato (Perl et al., 1992; Plant Molecular Biology 19: 815-823), and Petunia (Conner et al., 2009; Transformation and regeneration of Petunia. In T Gerats, J Strommer, eds, Petunia. Springer, pp 395-409). Transformation and cultivation of BY2 was done according to An, 1985 (Plant Physiology 79: 568-570). All plant species were transformed using agrobacteria GV3101 strain. Plant tissue culture was carried out in a climate rooms (22° C.; 70% humidity; 16/8 hours of light/dark, 2500 Lux light intensity).

Transient Expression in Nicotiana benthamiana

Transient gene expression assays in N. benthamiana for the BvDODA1 and CYP76AD15 genes and subsequent sampling for LC-MS analysis were performed as described hereinabove.

### Metabolite Extraction and Analysis

For LC-MS analyses, extraction of plant tissue from tomato fruit (flesh and peel), eggplant fruit (flesh), potato tuber (flesh), N. tabacum petals and N. benthamiana leaf was performed as previously described (Polturak et al., 2016). For extraction of BY2 cells, calli sampled from petri dishes (approx. 500 mg) were lysed by two freeze-thaw cycles with liquid nitrogen followed by disruption with metal beads. Cell-extract was centrifuged to remove cell debris and supernatant collected and filtered through 0.22 μm PVDF filters (Millipore) before analysis. LC-MS Analysis of L-DOPA and betalains was performed as previously described (Polturak et al., 2016). Relative quantification of L-DOPA in BY2 cells was determined by peak area, based on transition 198->152.

### Seed Germination Assays

Seeds of pX11 and wildtype (WT) tobacco plants were sown on petri dishes containing full Murashige-Skoog (MS) media (4.4 gr/liter) and 1% agar. For stress condition assays, the MS media additionally contained either 150 mM NaCl or 400 mM mannitol. Seeds were sown in three petri dishes, each containing 50 seeds per genotype. Sealed plates were kept in dark for 1 week and then moved to climate rooms (22° C.; 70% humidity; 16/8 hours of light/dark, 2500 Lux light intensity). Seed germination rates were examined for a period of 26 days.

### Plant Material and Growth Conditions

Mirabilis jalapa, N. tabacum, S. tuberosum, S. melongena and S. lycopersicum plants were soil-grown in a greenhouse with long-day light conditions (25° C.). Beta vulgaris and Nicotiana benthamiana plants were soil-grown in climate rooms (22° C.; 70% humidity; 18/6 hours of light/dark).

### Example 11

pX11 Expression in Tomato, Potato and Eggplant Results in Red-Pigmented Plants

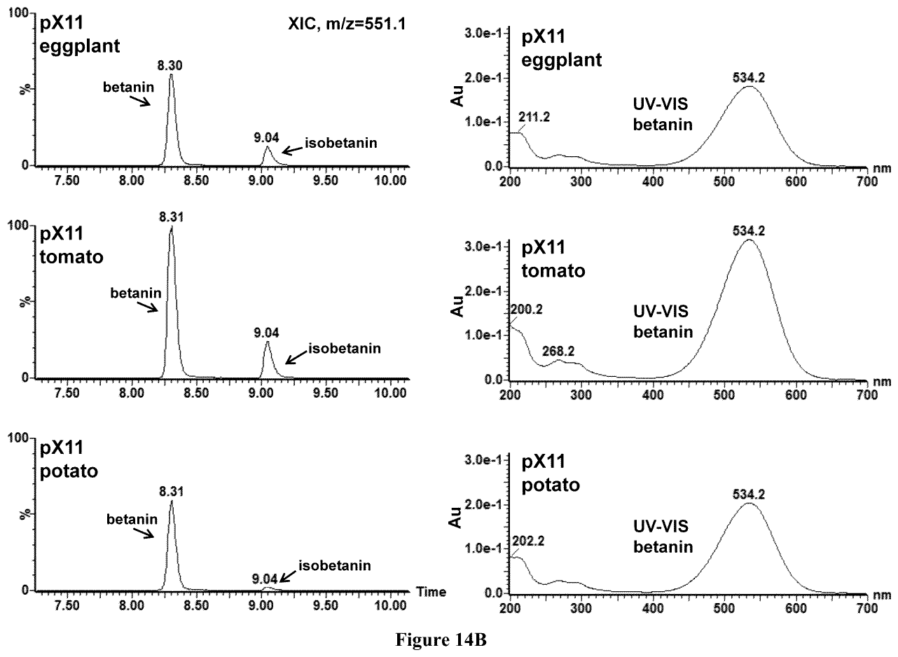

To inspect the feasibility of heterologous betalain production in food crops, the pX11 vector was introduced into tomato (Solanum lycopersicum var. MicroTom), potato (Solanum tuberosum var. Desiree) and eggplant (Solanum melongena line DR2) via agrobacteria-mediated stable transformation. Signs of red-violet pigmentation in transformed explants of all three species were observed within several days after co-cultivation with agrobacteria. As previously observed with pX11-expressing tobacco plants, transformed explants eventually evolved into entirely red-pigmented plants. Tomato and eggplant fruits, as well as potato tubers exhibited strong red-violet coloration (FIG. 14). LC-MS analysis of tomato fruit, eggplant fruit and potato tubers verified the occurrence of betalains, with betanin and isobetanin identified as the major occurring betacyanins (FIG. 14).

### Example 12

### Fruit-Specific Accumulation of Betalains in Tomato Plants

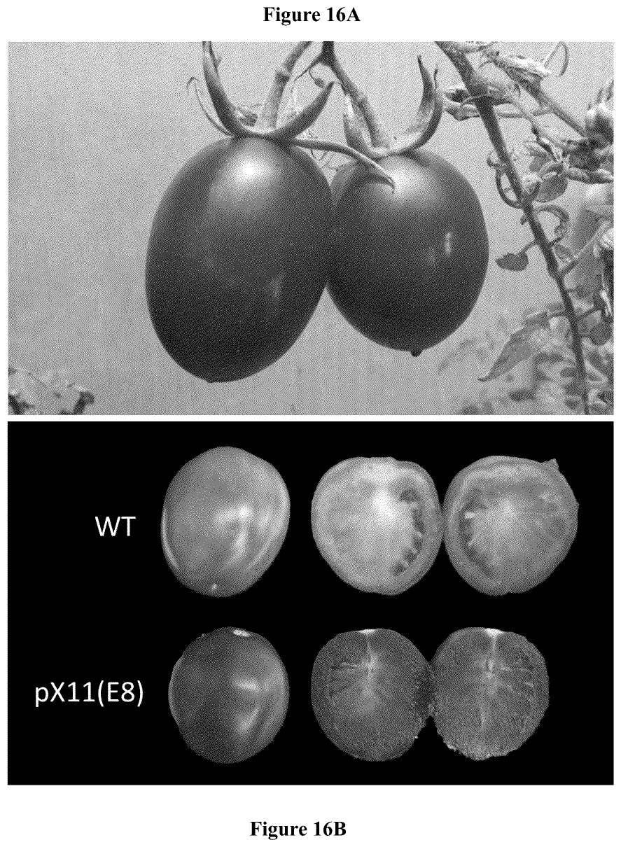

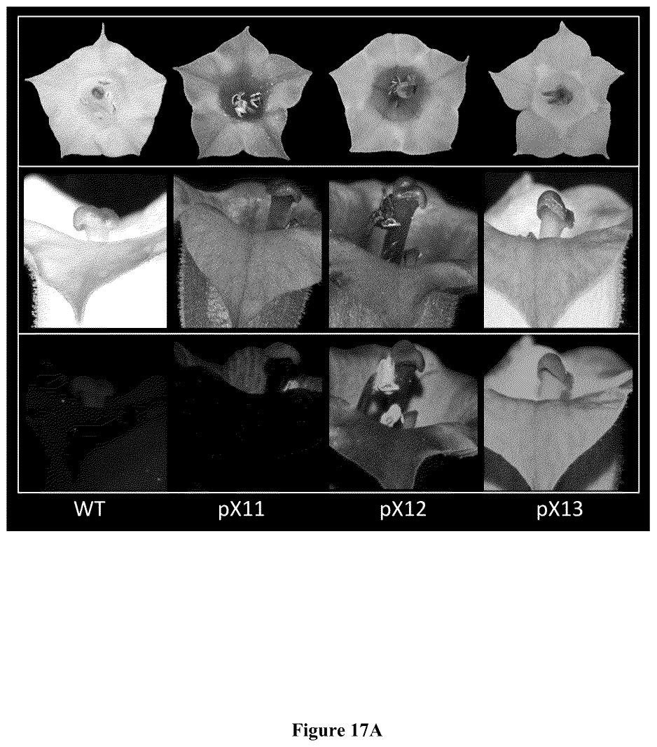

In all pX11-expressing plant species, betalain accumulation was visible in virtually all plant tissues and organs, owing to the constitutively-expressing promoter sequences used for gene expression, namely CaMV 35S for the BvDODA1 and CYP76AD1 genes, and the Arabidopsis Ubiquitin-10 promoter for cDOPA5GT. We initially hypothesized that constitutive production of nitrogen-containing compounds may lead to a significant metabolic burden on the transgenic plants. We therefore constructed a binary vector to be expressed in tomato, which would result in the restriction of pigment accumulation to ripening and ripe fruit. To this end, pX11 was modified to have CYP76AD1 driven by the fruit specific E8 promoter instead of CaMV 35S (FIG. 15). Transformation of the pX11(E8) vector to tomato var. M-82 indeed resulted in generation of plants which have betalain-pigmentation limited to ripe fruit (FIG. 16) albeit with lower betalain concentration than fruit of the pX11-expressing MicroTom tomatoes. Notably, pX11-expressing tomato, potato and eggplant plants did not eventually exhibit any apparent developmental phenotype or growth retardation.

### Example 13

### Flower-Specific Accumulation of Betalains in Petunias

In a similar approach, an additional version of the pX11 vector was constructed, in which CYP76AD1 is driven by the flower-specific Petunia x hybrida CHALCONE SYNTHASE (CHS) promoter instead of CaMV 35S (FIG. 15). Transformation of pX11(CHS) into Petunia x hybrida plants is expected to result in generation of plants that accumulate betalains only in petals, during flower maturation.

### Example 14

Expression of pX11, pX12 and pX13 in Tobacco Results in Differently-Colored Flowers

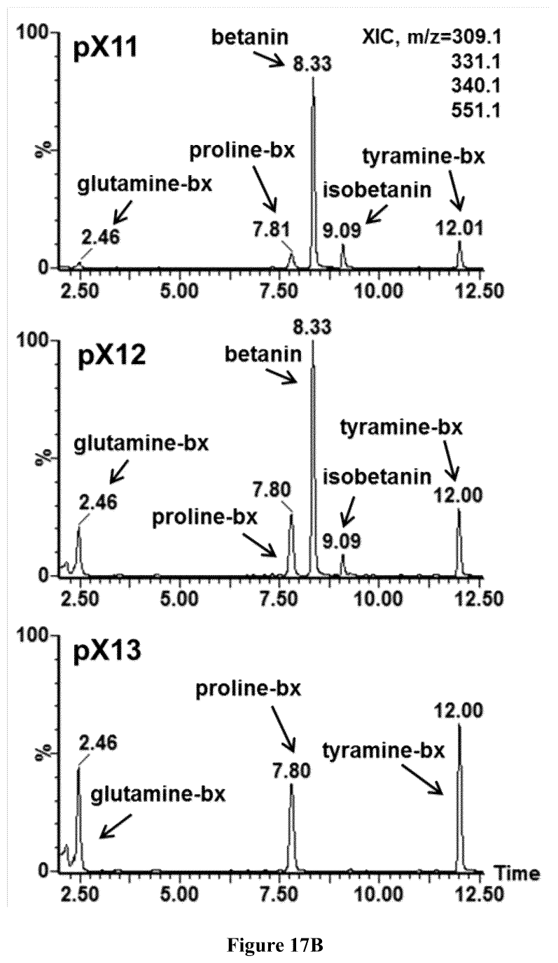

pX11-expressing plants displayed a predominantly red-violet color due to the accumulation of high amounts of betacyanins versus betaxanthins. pX11 incorporates the expression of CYP76AD1 that encodes an enzyme with dual-activity in betalain biosynthesis, catalyzing both hydroxylation of tyrosine to L-DOPA and conversion of L-DOPA to cyclo-DOPA. As described hereinabove and in Polturak et al. 2016 (New Phytol. 2016 April; 210(1):269-83, which is incorporated herein by reference in its entirety), a related enzyme in red beet, CYP76AD6, uniquely exhibits tyrosine 3-hydroxylation activity, to form L-DOPA. Since cyclo-DOPA derivatives are required for the formation of betacyanins, in-planta expression of CYP76AD6 instead of CYP76AD1 results in the formation of betaxanthins but not betacyanins, as was previously observed by transient expression in Nicotiana benthamiana. To explore the possibility of generating transgenic plants that accumulate only betaxanthin-type betalains, a binary vector was constructed for expression of BvDODA1 and CYP76AD6, hereinafter named pX13. An additional vector, pX12, was designed for expression of both cytochrome P450 genes CYP76AD1 and CYP76AD6, alongside BvDODA1 and cDOPA5GT (FIG. 15). pX12 and pX13 were next introduced into tobacco by stable transformation. While pX13-expressing tobacco plants produced only betaxanthins, resulting in formation of yellow-pigmented flowers, expression of pX12 in tobacco plants generated plants with flowers of an orange-pink hue (FIG. 17). LC-MS analysis of petals from pX11, pX12 and pX13 plants verified that the different colors observed in flowers of the three lines are the result of varying betacyanin/betaxanthin ratios; pX11 flower extracts predominantly contained betacyanins, pX13 extracts contained betaxanthins only, while pX12 extracts contained both groups of betalains, with a lower betacyanin/betaxanthin ratio than pX11 (FIG. 17). Differences in betaxanthin versus betacyanin accumulation could also be observed by blue light imaging, under which betaxanthins have a typical strong fluorescence (Gandia-Herrero et al., 2005). Thus, the flux towards biosynthesis of red-violet betacyanins or yellow betaxanthins can be manipulated via expression of CYP76AD1, CYP76AD6 or a combination of both.

### Example 15

pX11 and pX13 Expression in BY2 Cells

Biotechnological production of betalain pigments may provide new viable sources for their use as natural colorants for food, pharma and cosmetics industries. To date, most research has mainly been focused on development of Beta vulgaris hairy root culture or cell culture for betalain production.

Genetic engineering for heterologous betalain production enables the development of numerous new sources for these pigments. One viable source may be the culture of well-studied plant cell lines such as the tobacco BY2 or arabidopsis T87 lines. A previously reported attempt to use these lines for betalain production involved the expression of the Mirabilis jalapa DOD gene MjDOD together with a Shiitake mushroom tyrosinase gene. Production of betaxanthins in BY2 and T87 cells was demonstrated. However, within several weeks the cultured cells turned brown and stunted, most likely due to the toxic accumulation of dopaquinone and its derivatives, as a result of activity of the exogenous tyrosinase (Nakatsuka et al., 2013).

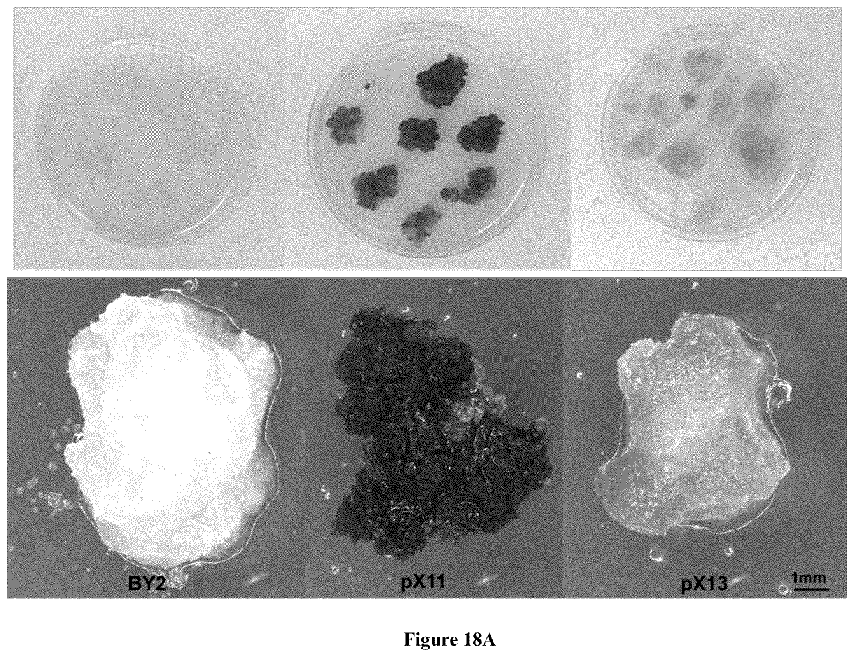

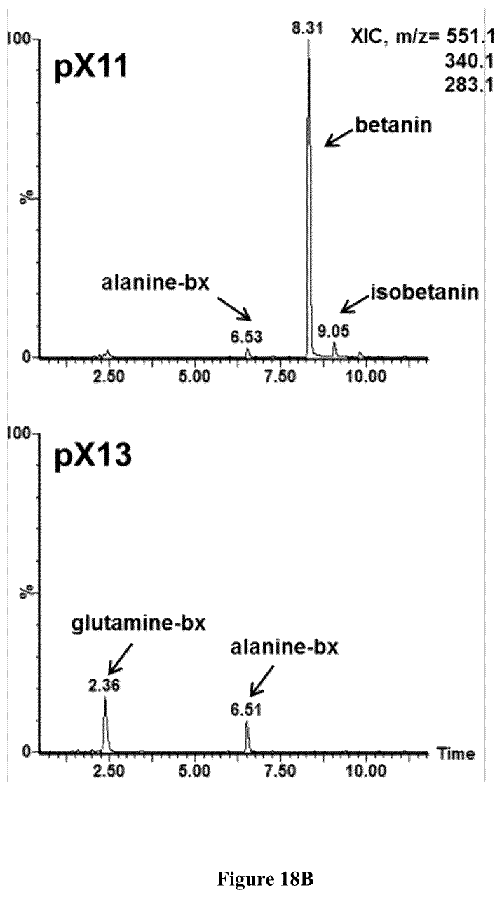

To explore the potential use of plant cell culture for betalain production through expression of betalain-related genes only, we expressed the pX11 and pX13 vectors in tobacco cell line BY2, resulting in generation of red-violet or yellow cells, respectively (FIG. 18). LC-MS analysis of cell extract of the pX11-expressing cells showed betanin and iso-betanin as the major betacyanins. Several betaxanthins were identified in both pX11 and pX13 cell lines, including glutamine-betaxanthin and alanine-betaxanthin (FIG. 18). Notably, pX11 and pX13 cell lines did not exhibit signs of browning or stunted growth as was previously reported for tyrosinase expressing calli (Nakatsuka et al., 2013).

### Example 16

### L-DOPA Production in Tobacco and BY2 Cells by Expression of CYP76AD6

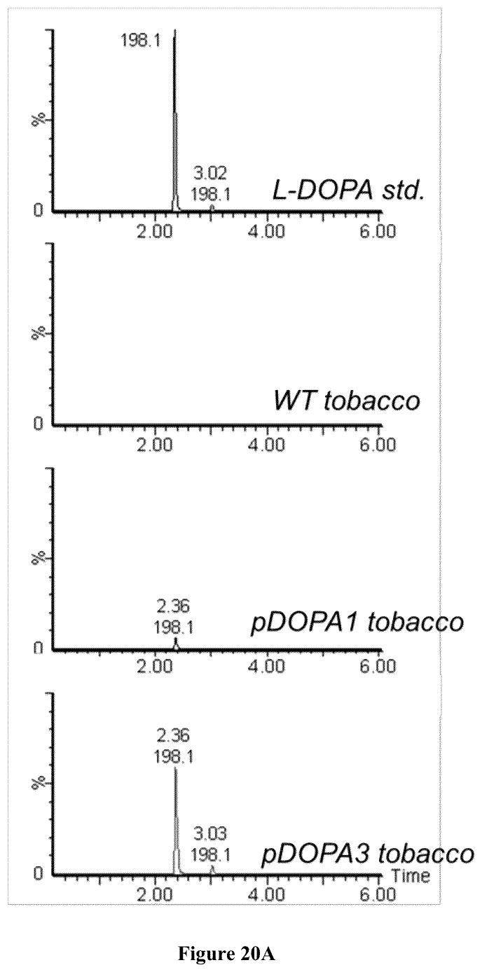

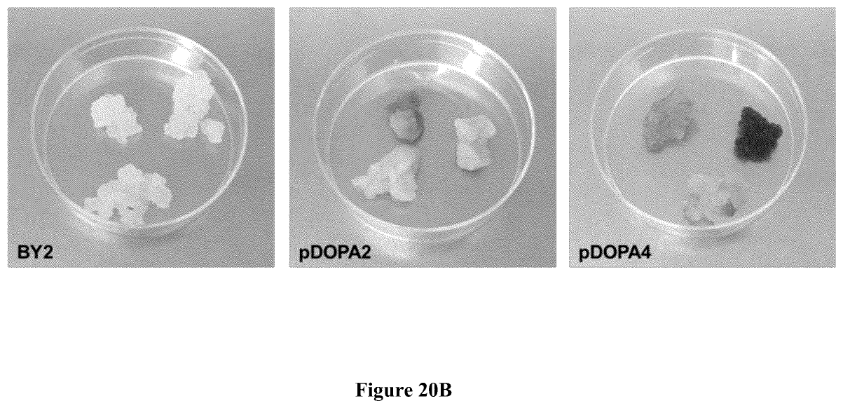

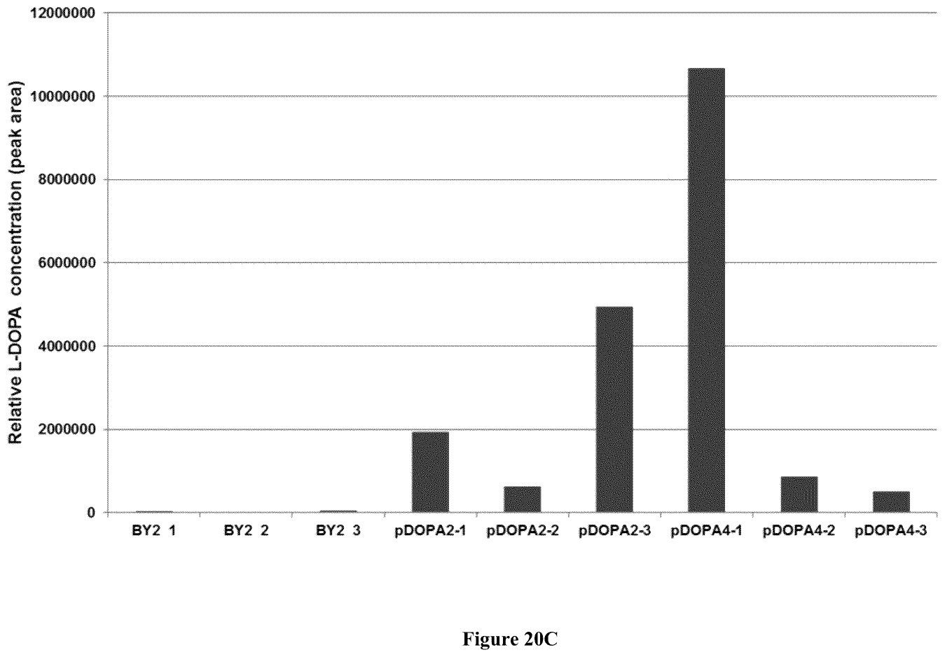

L-DOPA is an economically important compound that has been used for treatment of Parkinson's disease and is a precursor of high-value metabolites, including among others catecholeamines, benzilisoquinoline alkaloids and betalains. Current methods for industrial-scale production of L-DOPA show critical limitations. The tyrosine 3-hydroxylase activity of the CYP76AD6 enzyme may be implemented for production of L-DOPA that is advantageous compared to current production methods, by which L-DOPA would be directly formed from tyrosine. We explored the implementation of CYP76AD6 for L-DOPA production via expression in tobacco plants and in tobacco cell line BY2. To this end, four different vectors for expression of CYP76AD6 were constructed (FIG. 19); in one vector, CYP76AD6 is driven by the CaMV 35S promoter (hereinafter named pDOPA1). In another vector, pDOPA2, CYP76AD6 is driven by the Solanum lycopersicum ubiquitin 10 promoter (SlUb10). In vector pDOPA3, CYP76AD6 under 35S promoter is expressed together with two additional genes, AroG175 (Tzin et al., 2012) and aromatic amino acid hydroxylase (AAH) (Pribat et al., 2010), both of which are used in means of increasing tyrosine availability. In vector pDOPA4, CYP76AD6 under SlUb10 promoter is expressed together with AroG175 and AAH. All four vectors additionally express the neomycin phosphotransferase H(nptII) gene to confer kanamycin resistance for transformant selection. pDOPA1, pDOPA2, pDOPA3 and pDOPA4 were introduced into tobacco plants by agrobacteria-mediated stable transformation. LC-MS analysis of first generation (t0) plants confirmed that L-DOPA is produced in lines expressing the four different vectors. Analysis of pDOPA1 and pDOPA3 tobacco plants is presented in FIG. 20. The four pDOPA vectors were also introduced into BY2 cells lines. BY2 cells expressing pDOPA2 or pDOPA4 vectors exhibited varying degrees of gray to black pigmentation after several weeks of cultivation, likely due to formation of L-DOPA derivatives, possibly resulting from enzymatic or non-enzymatic oxidation of L-DOPA. LC-MS analysis of extracts obtained from pDOPA2 and pDOPA4-expressing calli showed occurrence of L-DOPA in varying concentrations (FIG. 20).

### Example 17

### Betalains Confer Plant Resistance to High Osmotic and High Salinity Stress Conditions

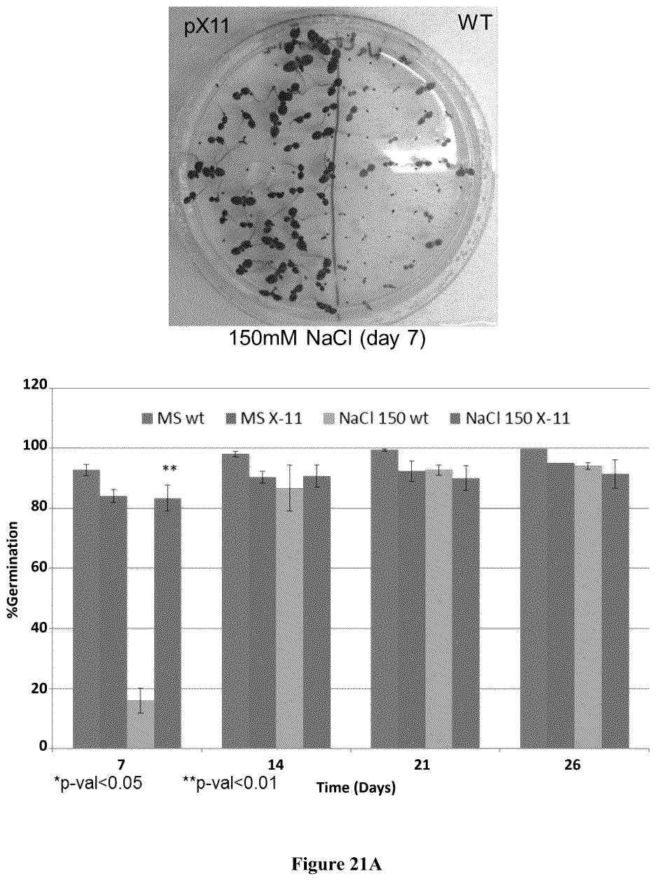

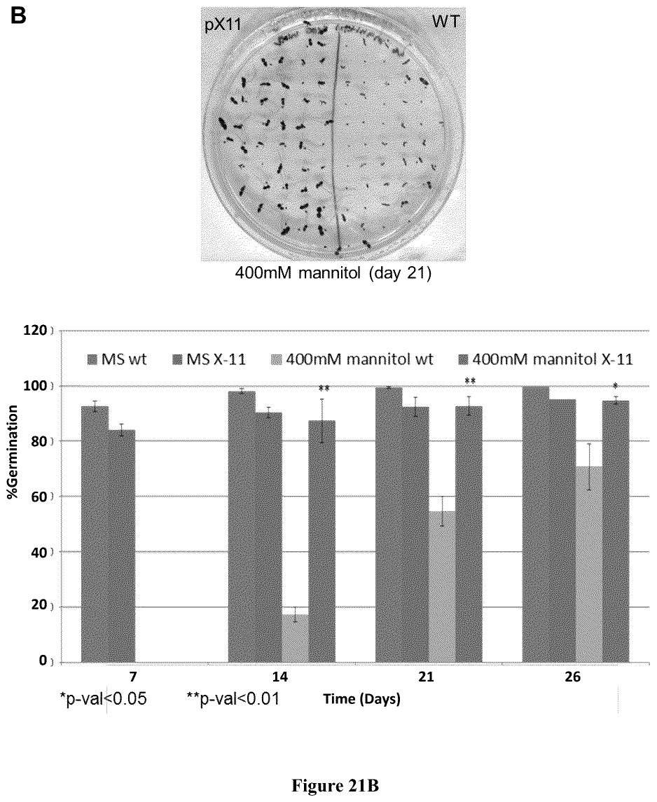

Considering the suggested roles of betalains in conferring resistance in plants to various abiotic stress factors such as drought, high salinity and excess light, enabling production of betalains in naturally non-producing plants may serve to increase their tolerance to abiotic stress factors. We therefore proceeded to examine this assumption via seed germination assays of betalain-producing tobacco plants. Seeds of pX11 and wildtype (WT) tobacco were placed on petri dishes containing Murashige-Skoog (MS) media supplemented with 400 mM mannitol or 150 mM NaCl, to mimic conditions of high osmotic stress or high salinity stress, respectively. Petri dishes containing MS media without additional supplementation were used as control. Germination rates of seeds sown on all plates were determined over a period of 26 days. While pX11 and WT tobacco seeds exhibited similar germination rates in the control MS media, pX11 seeds exhibited significantly higher germination versus WT seeds in plates containing 400 mM mannitol throughout the experiment time period. In plates containing 150 mM NaCl, pX11 seeds showed significantly higher germination rates after one week of incubation, but WT seeds obtained similar germination rates within two weeks and onwards (FIG. 21). Betalain-producing tobacco plants therefore exhibit increased tolerance to high osmotic and high salinity conditions compared with wildtype plants, having a higher germination rate in high osmotic stress conditions and faster germination in high salinity conditions.

### Example 18

Betalains Confer Resistance to Botrytis cinerea Infection in Tobacco Leaves

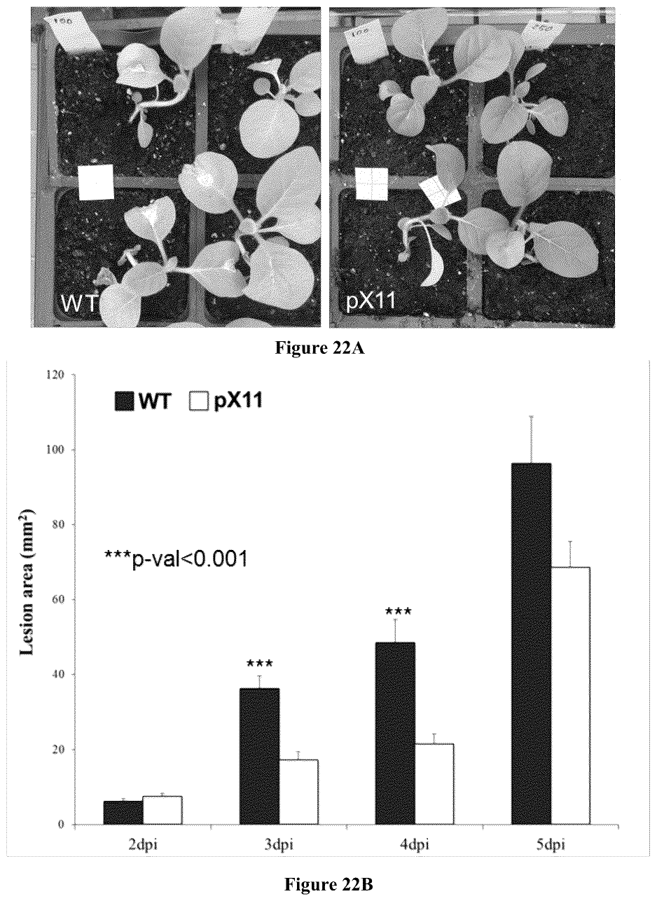

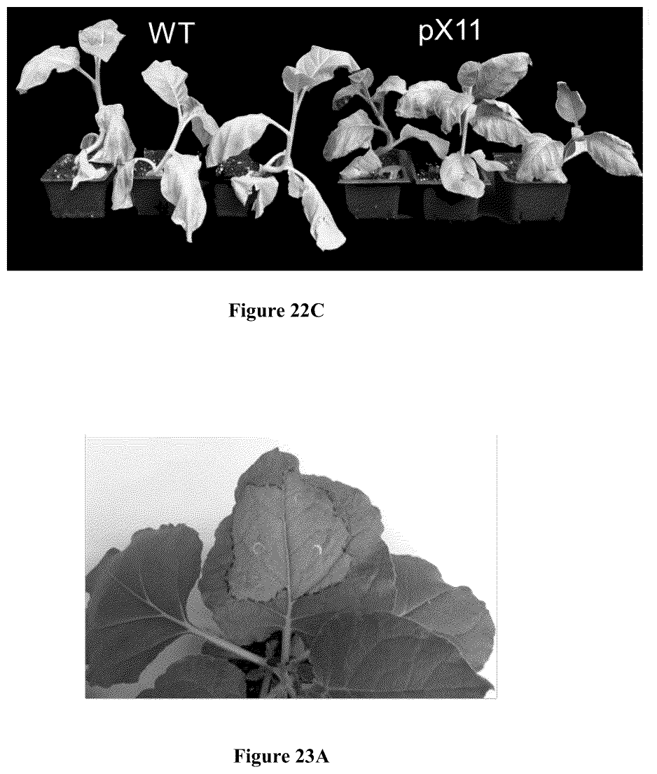

Betalains have previously been suggested to playa role in defense against pathogenic fungi, but evidence for antifungal activity of betalains in scientific literature is scarce. Heterologous betalain production in plants provides an excellent platform for studying antifungal activities of betalains in-planta, and may also be implemented for conferring resistance to phytopathogenic fungi in target crop species. To examine possible antifungal effects of betalains, wildtype and pX11 tobacco plants were subjected to leaf infection by gray mold fungus (Botrytis cinerea), a necrotrophic plant pathogen that is responsible for major losses in agricultural produce in both pre-harvest and post-harvest stages. Droplets of B. cinerea spore suspension in different concentration were applied on plant leaves, totaling approximately 100, 250 or 500 spores per plant. The degree of B. cinerea infection was then estimated by daily measurements of lesion size around the infection points. Leaves of pX11 tobacco exhibited significantly smaller lesion area upon infection compared to wildtype tobacco leaves (FIG. 22). Infected wildtype tobacco leaves also exhibited increased signs of necrosis compared to pX11 leaves within several days post infection (FIG. 22). Together, these results indicate increased resistance towards B. cinerea infection of betalain-producing plants.

### Example 19

CYP76AD15 is a Functional Ortholog of CYP76AD6 in Mirabilis jalapa

Considering the natural occurrence of additional betalain-producing plant species that consist of red or yellow-colored varieties, it is plausible that these species have a molecular mechanism similar to the one observed in red beet, where one cytochrome P450 enzyme catalyzes only tyrosine hydroxylation, while another catalyzes both tyrosine hydroxylation and cyclo-DOPA formation. It is also possible that the functional orthologs of CYP76AD1 and CYP76AD6 are found in the CYP76AD1-α and the CYP76AD1-β subclades, respectively. However, functional analyses of corresponding genes from additional betalain-producing species are needed to support this hypothesis.

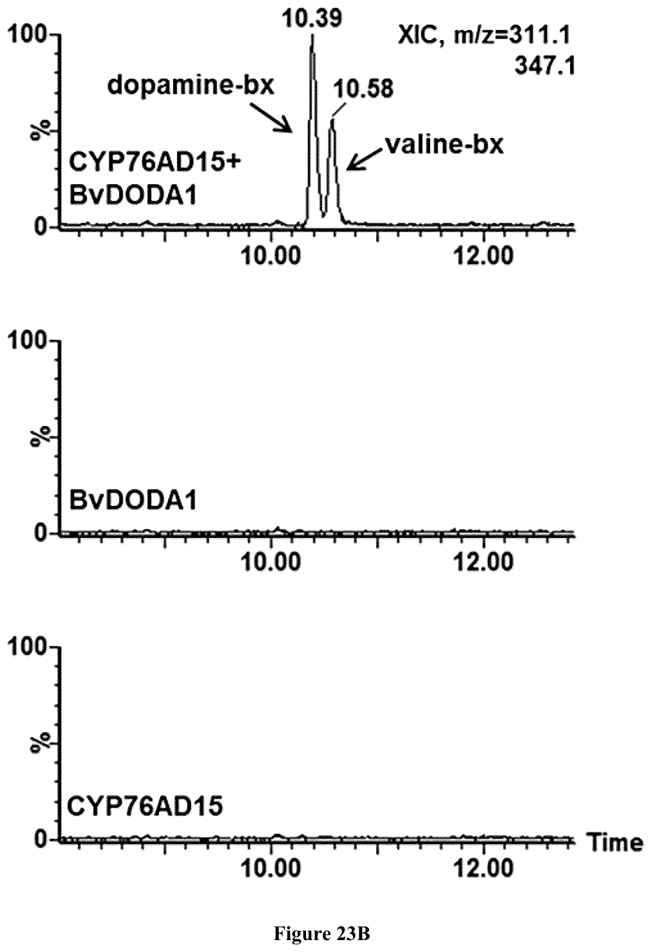

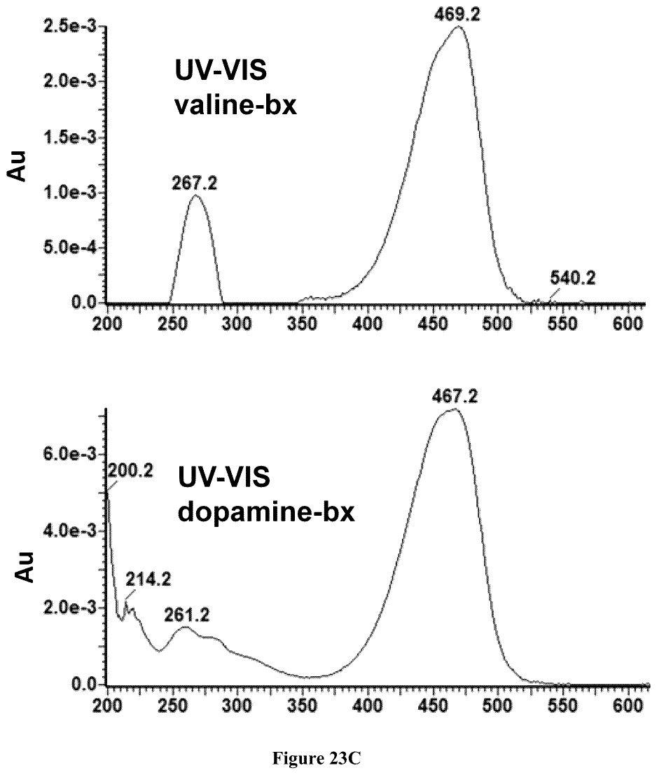

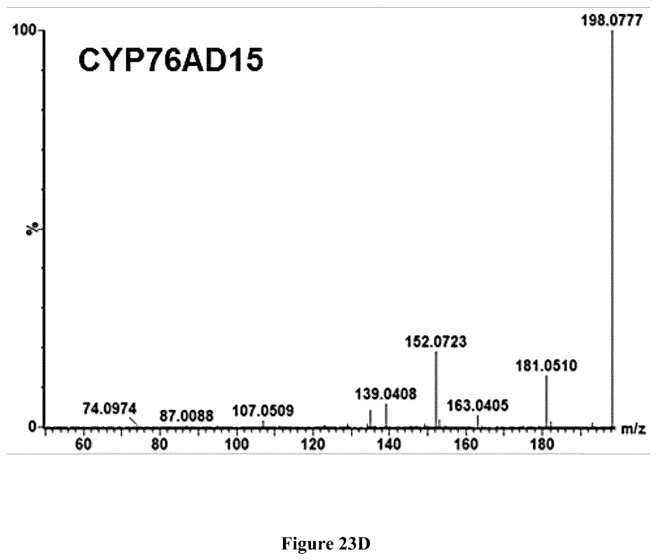

Functional orthologs of CYP76AD1 have previously been identified in several plants, including Mirabilis jalapa, where CYP76AD3 was identified based on sequence similarity to CYP76AD1 (Hatlestad et al., 2012). However, CYP76AD6 orthologs were not previously described. In order to assess the occurrence of a CYP76AD6 ortholog in M. jalapa, a tBLASTx query was performed in a previously obtained M. jalapa transcriptome dataset (Polturak et al., 2016), using the CYP76AD1 nucleotide sequence. This search resulted in the identification of several interrelated genes, including one gene with the highest sequence similarity to CYP76AD6, hereinafter named CYP76AD15. Interestingly, according to the data obtained in the M. jalapa transcriptome analysis, CYP76AD15 does not exhibit an expression pattern that parallels betalain accumulation. Instead, a generally constitutive expression pattern was observed for this gene. CYP76AD15 activity was tested by agroinfiltration assays in N. benthamiana. Similarly to CYP76AD6, CYP76AD15 was found to allow betaxanthin production in N. benthamiana leaves when co-infiltrated with BvDODA1, and L-DOPA production when infiltrated alone (FIG. 23). Occurrence of several betaxanthins was verified by LC-MS analysis, including dopamine-betaxanthin and valine-betaxanthin that were identified based on typical absorption patterns and MS/MS fragmentation. CYP76AD15 was therefore identified as a functional ortholog of CYP76AD6, catalyzing the formation of L-DOPA from tyrosine. Sequence analysis indicates that CYP76AD15 belongs to the CYP76AD1-β subclade together with CYP76AD6, while CYP76AD3 belongs in the CYP76AD1-α clade with CYP76AD1, thereby confirming the association of each of the subclades with a specific catalytic activity.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of producing L-DOPA from tyrosine, comprising introducing into a cell a heterologous polynucleotide that encodes a CYP76AD1-beta clade protein, wherein the heterologous polynucleotide is under the control of a promoter active in the cell, wherein the cell is selected from a plant cell, a bacterial cell and a fungal cell, and wherein said CYP76AD1-beta clade protein is a protein with at least 85% sequence identity to at least one of: SEQ ID NO: 10, SEQ ID NO: 11, SEQ ID NO: 12, SEQ ID NO: 13, SEQ ID NO: 18 and SEQ ID NO: 44, and that converts tyrosine to L-DOPA and does not convert L-DOPA to cyclo-DOPA, thereby producing betalains.

2. The method of claim 1, wherein the CYP76AD1-beta clade protein is selected from the group consisting of: SEQ ID NO: 10, SEQ ID NO: 11, SEQ ID NO: 12, SEQ ID NO: 13, SEQ ID NO: 18 and SEQ ID NO: 44.

3. The method of claim 1, wherein the CYP76AD1-beta clade protein is a protein with at least 85% sequence identity to SEQ ID NO: 18, and that converts tyrosine to L-DOPA and does not convert L-DOPA to cyclo-DOPA.

4. The method of claim 1, wherein the cell comprises tyrosine.

5. The method of claim 1, wherein the CYP76AD1-beta clade protein is a protein with at least 90% sequence identity to at least one of: SEQ ID NO: 10, SEQ ID NO: 11, SEQ ID NO: 12, SEQ ID NO: 13, SEQ ID NO: 18 and SEQ ID NO: 44, and that converts tyrosine to L-DOPA and does not convert L-DOPA to cyclo-DOPA.

6. The method of claim 5, wherein the CYP76AD1-beta clade protein is a protein with at least 90% sequence identity to SEQ ID NO: 18 or SEQ ID NO: 44, and that converts tyrosine to L-DOPA and does not convert L-DOPA to cyclo-DOPA.

7. The method of claim 6, wherein the CYP76AD1-beta clade protein is a protein with at least 90% sequence identity to SEQ ID NO: 18, and that converts tyrosine to L-DOPA and does not convert L-DOPA to cyclo-DOPA.

8. The method of claim 1, wherein the method is performed without provision of L-DOPA.

9. The method of claim 1, wherein the fungal cell is a yeast cell.

10. The method of claim 1, wherein the cell is a bacterial cell.

11. The method of claim 1, wherein the cell is a plant cell.

12. The method of claim 1, wherein the method is performed in culture.

13. The method of claim 1, wherein the cell is devoid of a heterologous polynucleotide that encodes a CYP76AD1-alpha clade protein, wherein the heterologous polynucleotide is under the control of a heterologous promoter active in the cell and wherein the CYP76AD1-alpha clade protein is SEQ ID NO: 14 or a protein with at least 90% sequence identity to SEQ ID NO: 14 and that converts L-DOPA to cyclo-DOPA.


SEQUENCE LISTING (86 sequences)

SEQ ID NO: 1 - DNA, 1901 bp, Mirabilis jalapa
  source 1..1901: mol_type = other DNA
cttcaaatcctcccacgtatatatcaacaaaaaccaaaaacatcatctcattacaataatggagtatcatcttctcttccttctcctcctgccgatcatccttatcttcgtgttcctcaatcacaagcccaagcaaaccaaactcgaaccaccaggaccacgtccatggcccatcataggccacaccaacctcgtcggctccaaacctcatcagtccattgccaaactagctcatatttacggtcccataatgtcacttaagctagggagaatcactactattgtcatatcttcccctgaagcagccaaagacttgttcctgaaacatgaccttactttctcaagtaggcaagttcctcacgcttcaaccgctacaaaccatgacaaactatccatggtttggctccctgtatgtcccaaatggcgctatcttagaaagatcgcggccatccaactattcaccaaccaacaacttgatggtggtcaggtactaaggcgtaagaaagtggacgagttgatccaatttgtgactcgttgtagtaaacaagggcaagtcattgatattggagaggctgtttttattactactcttaacttaatgtcaaatacctttttttccaaggatttgtgtagttatggtttggccgaatcacgagagtttaaggatctattttgggagttcatgaaagtgctagcgagtcctaatgtttctgattattttccatggttaagatggttggatttgcaaggcatcaagagaagaagcgagggttgttatcgcaagatgttgggtttttttggggagattattgatcagagattgagagatccaatgtcatgtaagaacgatgtattggacactctactcaaacttgtcgaccaaaaagagttgagctttgaggatgtcaaacatatgcttgtggatttgtttgttgcagggacggatacaacttcgaacacattggaatggacaatggtagaacttttacgccaccctaacatattgactaaagcacaaactgaactcagccaagtcataggcaaggacaagttagtccaagaatcatgtatcaccaagttgccatatcttcaatcaatattgaaagaaacattcaggttgcacccaccgactccttttttacttccacacaaagcaatcgagaacgttgaactatgcaactatcacatacctaaaggtgctcaagtttgggtgaatgtgtggtctattggccgcgatcccaacatttggtcaagcccggacttattttccccagagagatttttaggtaccgacattgatataaaaggtaaccatttcgagctcataccattcggagcaggaagaaggatttgccccgggttgtcactagcttacaggatgcttcatttgattttggccactctccttcattcattcaattggaaccttcctaatgacgtatgtccggaaaatatggatattgaagaaaaatttgggattacacttcaaaagatcatgccactcaaagtcataccaagctctaggtgtctggatcacgacaactaactggtgttaatcttttacaacatattgaatgatatatatcacgttaaggtacatgcgtctagatgcagaaacttgcatacgtttccttttcaataaagtcttgcatatgtatggtttatagggtgatgtaatttcttgtttgtttttacttaagtttataagacattttttttttttttttatcgatgctcttactcattaaaatattggaacatatacaaaattttatatgttttagataaaattatacaggttttaattttaggaacttaccacttgttcacactgcgagacttaaatagtcaaaatagaaaatacacactattaaaaagtactcactatttatcaaatttat

SEQ ID NO: 2 - DNA, 1663 bp, Mirabilis jalapa
  source 1..1663: mol_type = other DNA
cctcacctccataacaaagaaatgaccgccattaaaatgaacaccaatggtgaaggtgaaacacaacatatactaatgatacctttcatggcgcaagggcatttacgtcctttccttgagcttgctatgtttctatataaacgaagtcatgttatcattactcttcttactaccccgctcaatgcgggtttcctacgacatctccttcaccaccatagctattctagctcggggatcagaattgtcgagttacctttcaactcaaccaatcatggtcttccacctggcattgaaaacactgataaacttacactcccacttgtagtatcactttttcattcaaccatttctcttgaccctcaccttagagattatatttcccgccatttctcccctgcgcgccctcctctgtgtgtcatacatgatgtgttccttggttgggttgatcaagttgctaaagacgtgggctcaactggtgttgtttttactacgggtggcgcgtatggtacaagcgcatatgtgtccatttggaatgatctgcctcaccagaattactctgatgatcaagagtttccgcttcctggtttcccggagaatcataaattccgacgttctcaacttcatcggtttctgaggtatgccgatggatcagatgattggtcgaaatatttccaaccgcaattgaggcaatcaatgaagagttttggatggctatgtaattcagttgaggaaatcgaaacacttgggtttagtatcctcaggaactacactaaactacccatttggggtattggaccgttgatagcttcacctgtacaacattcatcatctgataataacagtactggtgccgagtttgttcaatggttgagcttgaaagaaccagattctgtattatacatctcatttggatcacagaacacaatttcaccaacccagatgatggaactagcagccggtttggagtcaagtgagaagccgtttttgtgggtgattcgagcaccatttgggttcgatatcaatgaggaaatgagaccagaatggctaccagagggattcgaggagcgaatgaaggtgaaaaaacaaggaaagttggtgtataagttgggaccacagttggagatacttaaccatgagtcaatcggagggttcttaactcattgtgggtggaattcgatccttgagtcacttcgagaaggtgtgcctatgttagggtggccattggcagccgaacaagcttataatttgaagtatttggaggacgaaatgggggttgcagtcgagttagcgaggggattggaaggagagataagtaaagagaaagtgaagagaattgtggagatgattttagagagaaatgaaggaagtaaaggatgggaaatgaaaaatagagcagtagaaatggggaagaaacttaaagacgctgtcaatgaggagaaggaactgaagggttcttctgttaaggcaatagatgatttcttagatgcggtcatgcaagctaagttggaaccttctcttcaataataagatcgatagtttatctcgtctcagagactcaaacaattgactactaggatgatcagaaagagagttatgttcagtgttctgttattatgcaagacttcaataataacagaaaaattcgtgattagattcataactt

SEQ ID NO: 3 - DNA, 1005 bp, Mirabilis jalapa
  source 1..1005: mol_type = other DNA
atgaaaggaacatactatataaatcatggtgatccactaatgtacttaaaaaaacatataaaactaaggcaattcttagaaggatggcaagaaaatgttgttattgaaaaaccaaagagtatacttatcatttctgctcattgggatactaatgtacctactgtcaactttgttgaacattgtgatactattcatgattttgatgactatcctgatcctttgtaccagatacaatatcgagcaccgggagcaccaaatttagcaaaaaaggtggaagagttactaaaagagtcaggaatggaatgtgagatagatacaaagagaggactcgatcatgcagcatggtttccactaatgtttatgtatcctgaagctaatattcctatttgtgagctctcagtccaaccaagcaaagatgggatccaccactataatgttgggaaagctctttctcctcttttgcaacaaggtgttctcatcattggctctggtggtactgttcatccttctgatgatactcctcattgtcccaatggtgttgctccttgggctattgagtttgataactggcttgaagatgctcttcttagtggaaggtatgaagatgtgaacaacttcaagaaattggcaccaaattgggagatatctcatccaggacaagaacatttgtaccctttgcatgtggcattaggggctgctggcaaaaatccaaagacacaacttattcatcgaagctgggctgccaatggtgtctttggatattccacctacaacttcactcccaccactcaaaaaactgattaaaacttcttttatatatctcctattcttgctttatttcttccttgttcaattgttttatttatgttgtaatattatgtaaaatttgcctaaattgggctaccggtgtattatactatgactaacatttgatatagttgctccgactactatgtcaaagctgtaaggtgtttgatcattgtgtaatcgaaaaatgttggattc

SEQ ID NO: 4 - DNA, 1680 bp, Mirabilis jalapa
  source 1..1680: mol_type = other DNA
aaagcatacaatgaccaccagcaatcattaaacaagagttaacactgtcactcatctaattttcttcaaaacactatttcttctctcatctctttaagtagttatttatttaattttgatcatcataatatggatttcttaacccttgtcatgatattatctattatcttcttcttttacaatctattaaaaatgaaattcactacccattcagatgcccaacttcctccaggacctaaaccaatgccaatattcggtaatatcttcgaactaggtgaaaaacctcaccgatcatttgctaacttagcaaaaacacacggaccactaatgtcgttaaggttaggaagtgtcaccaccattgtcgtctcctcggcagaggttgctaaagaaatgttccttaaaaatgaccagtcccttgcagatagaagtgtccctaattctgtaacggcaggtgaccatcacaagctaaccatgtcatggttgcctgtctctccaaaatggaaaaatttccgaaaaatcacagccgttcatttgctttctcctcaaaggcttgatgcttgccatgcacttagacatgccaaggtcaaacaactctatgaatatgttcaagagtgtgccctaaaaggagaagcagttgatattggcaaggccgcatttaccacttcacttaatctattatccaatttgttcttttcggttgagttagctaatcatacttctaatacttcccaagagtttaaacaacttatatgggatataatggaagatattggaaagcctaactatgctgattattttcctttacttaaatatgttgatccctctggcatacgacgtcgtttagcggctaattttgataaacttattgatgtatttcaaagctttatctccaaaaggttatcatctgcttattctagtgcaacaagtctcgatgatgtgcttgatgttcttctcaaactcctcaaggaaaaggagcttaatatgggtgagattaatcatcttcttgtggacatatttgatgcaggtacagacacaacatcaaatacattcgaatgggcaatggcagaactaatgaggaacccaataatgatgaaaagagctcaaaatgagatagcattagtattaggcaaagataatgccactattcaagaatctgatatagcaaacatgccttatctacaagcaattatcaaagaaacattacgtttacatcctccaactgtctttctattgccacgaaaagccattactaatgttaagctatatggttatattgtcccaaaaaatgcacagatacttgttaacttgtgggccattggtcgcgaccccaaggtctggaagaaccctaatgagttcttacctgataggtttttgaattctgatattgatgtcaaaggtcgagattttgggttgttgccttttggtgcagggagaaggatatgtcctggtatgaacttggcttataggatgttaactttgatgttggcaactcttcttcaatcttttgattggaagcttcctcatcggaatagtcctctggatttggatatggatgagaaatttggtatcgctttacaaaagactaaaccacttgagattattcctcttattaaatattgatgttttatatgttgtattattattgcaattgtttaaacatctttcttattattttggttg

SEQ ID NO: 5 - DNA, 749 bp, Mirabilis jalapa
  source 1..749: mol_type = other DNA
tcgatgtcaccaatttcccctatggacgtccaaaccacattcccaaagcaactagactcagtgattcgaaccaaggtggcaaggccagcaaaaaaaaggaaccaagaagagaagaagaaagaagaagagatacttgtgatagagttagaagagctaaaacaaaacgagtacgtcaaatttgatgtgtacataaatgatgattatgattacgagaataagaatgacggcgtgacgaggccaacggaaaaatatgcagggagtttcgtgagtttggcatcatcgggacagggaatggagagcaagaagagtgggttgagattggcgttgaccaagttgatcgatgaaattggtgctgatggtgatgacttcttagatgtggtgctagtggctaaagctgggaaaggtggtgttgttataaatgacgtcaagattgagttccttgcttgatttagttaagtattagtatggtagtggttgatgtattgccttttaatttgtgtacaagttacaactttatttgcaattcaccacttgtttttatttgtatatgagtttctggtttatctcaatatttagtcatggtgttactgttttatggtgtttcctcacgtatctaagaaaaaataacaatagatattggaattatcatgaattgtgcgcccatcactggtggtttgaaaaagaaagacaataaaaataagaaaaaattataaaaaaaattctataatatatttatttttttaaaaaaa

SEQ ID NO: 6 - DNA, 2176 bp, Mirabilis jalapa
  source 1..2176: mol_type = other DNA
catcccacttccattccgacaaccaactcaaccaccatcatcaaccctcctcacaacaacccccttctctctcacctttctccgatcaatcgccgccgttcattccgccacctttcacccaaactctccatccgaaacaaccaacaatccaccaacaacgaccctcttttcgacccatcaaaactcgaccgaagaaacatccttatcgggagtggtctcgccggcatctacggcggagccaccactctcggaggtaaccccgccctagctgctcccccgctccctgacgtcaaaacatgcaacctcacgtccacctccggtgggggccctgaggggctcgaatgttgcccaccctcattcagaaacatcgtagatttcaaccctcgaaactacgccggcccggcactccgggtcagacgtccggctcatgaagtcgcgaatgacccggagtacatggctaaatttaaagaagcaataagaagaatgagagcacttcctagatctgatccaagaaactttgtacaacaagctaatgttcattgtgcttattgtgatggctcataccgtcaagataaacacagtgacgtcgattacacggttcatttttcttggttattcttcccattccatagagtttacctctatttcttcgaacgtatcttggggaaattgatcgatgatccaactttcgcaatcccatactggaactgggatcatccagatggtatgagaatgccaactatttacacgaacccagattcaccattgtttgatgctcttagagatccggctcatcagccgcctagagttgcagatcttaactacaatgatgttgatactgatataagtccagagttgcagattgagtataacttaagagtgatgaattatcaaatgacaaggaataataaacctgagttgtttatgggtgcacctttgtaccctggtaatgctgctgaagagaagggtactggtgcacttgaaaatgttcctcataatttgcttcatgtttgggttggtgatcgtaatgacgatccatttcacaggaacatgggatccttcttctcggctggaagagacccaatattctacggtcaccatgcaaacgtagacagaatgtggtacatttggaataccaagcttagccatcaaaatttcactacaaaagattggctagacacttactttgttctctacgacgagaacaaacaagctgtcaggattcgaacttcagactgtctggatgttgaaaaacaactcggctacacctattcgaaccaagacttaccctggttagactcaacgactggaggaggagccattgaaccaaccccacccccaccatctggtgtggccgagttcccacaagttctagacaaggttatcagaaccagggtggctcgacccaagacctcaaggtcacaacaggaaaaggaagttgctacagaagtgctgaaagtgtctgtcaacattgataaagaagataattacattaagtttgatgtttttatcaatgatggtcaggattttgagaccggtggtcagactgatatcggtaagtcttatgcaggaagctttactagcgtccctcataagactagaaaaggtgctaggatggatatgaaccgccgggtttcacttcccttgactgagattttggagaatttgggtgctgatggtgatgattatattgatgttgctattgttcctaaggctggtttaggtaatgtcaccattactgctgttaaggttgattatattatttgattagtcactaatcaagtaattaataaattaaaattactaccatatataataattagttatgatgagtgtgttaatgtagtaagtggtattattattaagtgagtaaaataaaaagcgaccatgcattatgattcatgattgtgggagtcgtgtttcatgcatggtagtgatgggatgttatcgtatcttttggtgcgctttaaatctgtttccactttattattttttttttttccaagtatttccactttattgttatgacttgtcaaaagttggtattatataagtttgtcgttctatattaaaattattttttaatgtttttttttatttttacttttagtaaagttgttatcaaggacatgatatttttgaaggaatgaagacaaacattaatataactgtaagg

SEQ ID NO: 7 - DNA, 282 bp, Mirabilis jalapa
  source 1..282: mol_type = other DNA
caaacaagctgtcaggattcgaacctctgattgcctcgatgttgaaaaccaactcggctacacctatgaggaccaagatttgccatggttagactcatctgtcggaaaagacgcccctatacccaccaaaccccaaccatctgatgtggccgagttcccacaagtcctagacaaagttatccgtactagagtggctcgacccaaggccacaaggtccgagcaagaaaagcaggttgctgcagaagccctgaaagtatccgtcaacattgataaagaagataa

SEQ ID NO: 8 - DNA, 1016 bp, Mirabilis jalapa
  source 1..1016: mol_type = other DNA
acggaggcgcataaaaaaatggttgtattattttgctattgatatctgctgggaaaacaaggagtagtgtattcggattctcacatccccgaaatcgatcaacgtctttatttgggcggtttaaagcaaggaatatctgaggaattgctatcagtgactcatttgtgtgtcaaagcatatcaaccagctataggaggtgctaccgtgggtaacttgaattctttaggccccttttgaatgtcacgaaagagatgtgtccttattggtaaactgtttgatttctattgttgagaagtcaaaacctggacattagacaaccacttgtattatttgactgtttccgattaatcctactaatgacggtctctctcccttctcctataacgaccatcaacattcccgttgccactagaaacactaaaccactccctaagccattgatgctttatcaatcctgccaccctaccttgaaaatttcatgccaagcctcccacaacctcaaggtgaatccacaacgccgagatctcctaattgggctagccaccctatgtgggacccccaccttgtgtgtcccactagtctcagcagcaccagcagcagcaaccccttttccacagctgttggactccgtggttcggaccagggtggcaaggccagcaaaagaaagaagccaaagagagaaggatgatgaagaggaggtgcttgtggttagcttggagcaactaagggaagatgaatatgtcaaatttgatgtgtacatcaatgatgattatgactttgagaataagaatgatgggctgacaaggccaacaaaagaatatgcggggagttattctagcctgccatctccaaggcgactcatggagagcaagaagattgcgttgaagctggcgttgacacagctgattgatgacattggtgccgatagcgatgactatattgacgtggtgctggtgcctaaagcaggaaaaggtgctcttgttgtaaaaaatgtcaagattgagttccttgcttga

SEQ ID NO: 9 - DNA, 1922 bp, Beta vulgaris
  source 1..1922: mol_type = other DNA
cagtcatttaaaatcctgtaaattactcacatacataaccaaattaaagttgccgccgtactgatttccaaaggtcctacatctatcaatggagtattacaccataacgctattattcatcatcttcacaatatcaatattcggtacaaaaattttgagcaagtccaaacttcctccaggaccaacaccatggcctatcataggcaacatcctcgagctaggcaagttacctcatcaggcagttgacaagctctcaaaaacctatggccctatattatctctcaaactcggaagcatcactacaatagtaatatcatcccctgaaatcgttaaagaaatgttcctcgagcacgacttagccctttctagtaggccttccccggatgcttcgagggtgggaaaccataacaaattttccattgtttggctcccagtgtcccccaaatggcgggaccttagaaaaattgctactatccaattgtttactactcaacgcctagattctagccaagaactacggcagatcaaggtgaacgagctggttgattatgtacgacagtgttgtgaaaagggtctacctgttgatgttggtaaagccgggtttactaccacgttaaatatgttgtctaacacgtttttctccatggatttggctagccatgcttcttcgaattcgcaagagttcaaggatcttgtttggagtcttttggaagagggtgcaaagcctaatgtgtcggattttttcccgatagttagagaattggatttgcagggagtatcgaaaaatagaagggtgcacatgaagaagctaatgggaattttcgaggaaatcattgatggaaggttgacaaagttaaaggatgtgaaggatgatgttctaagtactttactcaaacttgttaaggatgaagaattgaaccttgacgacgtcaaacatatgctcatggatttatttttagcgggaacagatacaacctctatcacattggagtgggcgatgacagaattacttcgcaatccagaaaagatggaaaaggttcaaattgagttggacaaagtacttggcaaggatagttctctgcaagagtcaatgatctcaaaattaccatatattcaagcaatagtgaaagaaacattaagattacacccaccaactcctttcttgattcctcacaaagcagaaaaagatgtattgttatgcaactatttagtgcccaaaaactcaattatttgggttaatttatggtcgatcgctcgtagtccaagtgtgtggccaaatccagaatcattttctcctgaaaggtttttggagatggaaattgatattaaaggacgagatttcaaactcataccctttggatcaggaagaagaatgtgtcctggaatgccacttgcttataggatgacacatatgttgttggctactcttcttcattcatttaactggaagtatggtgaggcaagtccaaaagatatagacatgaaagaaaagtttgggttgacgttgcaaaaggctcagccacttcaagcaattccaattcccagatgatcaatctcttttgaggagttagacgatttctttgtgtcaaccaactagtacttatcggaacatttattgttaaatgtgtctgttagatgtacatatgatgttgttatactaagaaataacggtgtttaagtgcaacatgggacaagaggattatgtaaagtatcctagattttattaggtgctcctacacctatgtattggtgcatttgcaccaagtcgtctgtagaaaacttgagacttgggaggcaagaactttacctctgtaaaaaaaacgcgttttttgcattctccttgtatcttttgcaagagattgtacgcattcacaaaacatcaaatacatttgtgtttacacaataagaatcgccaacactga

SEQ ID NO: 10 - protein, 495 aa, Beta vulgaris
MDNATLAVILSILFVFYHIFKSFFTNSSSRRLPPGPKPVPIFGNIFDLGEKPHRSFANLSKIHGPLISLKLGSVTTIVVSSASVAEEMFLKNDQALANRTIPDSVRAGDHDKLSMSWLPVSQKWRNMRKISAVQLLSNQKLDASQPLRQAKVKQLLSYVQDCSKKMQPVDIGRAAFTTSLLLSNTFFSIELASHESSASQEFKQLMWNIMEEIGRPNYADFFPILGYIDPFGIRRRLAGYFDKLIDVFQDIIRERQKLRSSNSSGAKQTNDILDTLLKLHEDNELSMPEINHLLVDIFDAGTDTTASTLEWAMAELVKNPEMMTKVQNEIEEALGKDCLDIQESDISKLPYLQAIIKETLRLHPPTVFLLPRKADNDVELYGYVVPKNAQVLVNLWAIGRDPKVWKNPEVFSPERFLDCNIDYKGRDFELLPFGAGRRICPGLTLAYRMLNLMLATLLQNYNWKLEDGINPKDLDMDEKFGITLQKIKPLQVIPV

SEQ ID NO: 11 - protein, 499 aa, Froelichia latifolia
MDNTTLAVVLSVMFVLFHLLKTIFTNSSNNKLPPGPKRMPIFGNIFDLGEKPHRSFANFAKIHGPLISLKLGSVTTIVVSSASVAEEMFLKNDQALANRTIPDSVRAGDHDKLSMSWLPVSAKWRNLRKISAVQLLSNQRLDASQPLRQAKVKQLLAYVQNCSEKNQAVDIGRAAFTTSLNLLSNTFFSMELASHESSASQEFKQLMWNIMEEIGRPNYADFFPILGYIDPFGIRRRLAGYFDKLIDVFQDIIRERQKIRASNANGTKQTSDILDTLLKLYEDNELSMGEINHLLVDIFDAGTDTTASTLEWAMAELVKNPEMMVRAQNEIEEVLGKDCSNIQESDISKLPYLQAIIKESLRLHPPTVFLLPRKADVDVELYGYVVPKNAQVLVNLWAIGRDPKVWKNPEVFSPERFLECDIDYKGRDFELLPFGAGRRICPGLTLAYRMLNLMLATLLHNYNWKLGDDMDPKDLDMEEKFGITLQKIKPLQVIPVARK

SEQ ID NO: 12 - protein, 495 aa, Alternanthera caracasana
MDNTTLAVVLSIMFACFHLIKTIFSNSSNSKLPPGPKRLPIFGNIFDLGEKPHRSFANYAKLHGPLISLQLGSVMTVIVSSASVAEEMFLKNDQALANRTIPDSVRAGDHDKLSMSWLPVSPKWRNLRKISAVQLLSTQRLDASQPLRQAKVQQLLAYVQDCSKKGQPVDIGRAAFTTSLNLLSNTFFSMELASHESSASQEFKQLMWNIMEEIGRPNYADFFPILGYIDPFGIRRRLASYFDKLIAVFQDIIRERQKIRSSNVSGTKQTNDILDTLLKLHEENELSMGEINHLLVDIFDAGTDTTASTLEWAMAELVKNPYMMVKAQNEIEEALGKDCSNIQESDISKLPYLQAIIKESLRLHPPTVFLLPRKADHDVELYGYVVPKNAQVLVNLWAIGRDPKVWKNPEVFFPERFLGCDIDYKGRDFELLPFGAGRRICPGLTLAYRMLNLMLATLLHNYNWKLGDGMNPKDMDMEEKFGITLQKIKPLQVIP

SEQ ID NO: 13 - protein, 493 aa, Alternanthera ficoidea
MDNTTLALLLSGLFVFFHLIRSLVSHSSKSSKLPPGPKRMPIFGNIFDLGEKPHRSFANFSKIYGPLISLKLGSVTTVVVSSAEVAKEMFLKNDQALANRTIPDSVRAGDHDKLSMSWLPVSNKWRNLRKISAVQLLSTQRLDASQANRQAKVQQLLAYVQDCSKKGQPVDIGRAAFTTSLNLLSNTFFSIELASHESSASQEFKQLMWNIMEEIGRPNYADFFPILGYIDPFGIRRRLAAYFDKLIAVFQDIIQERQKIRSSGTKQTNDILDTLLNLYEENELSMGEINHLLVDIFDAGTDTTASTLEWAMAELVKNPEMMIRVQNEIEQAVGKDCPMIQESDISKLPYLQAIIKETLRLHPPTVFLLPRKADVDVELYGYTVPKDAQVLVNLWAIGRDPKVWKNSEVFSPERFLECDIDYKGRDFELLPFGAGRRICPGLTLAYRMLNLMMANFLYSFDWKLEGGMHPKDLDMDEKFGITLQKVKPLQVIP

SEQ ID NO: 14 - protein, 497 aa, Beta vulgaris
MDHATLAMILAIWFISFHFIKLLFSQQTTKLLPPGPKPLPIIGNILEVGKKPHRSFANLAKIHGPLISLRLGSVTTIVVSSADVAKEMFLKKDHPLSNRTIPNSVTAGDHHKLTMSWLPVSPKWRNFRKITAVHLLSPQRLDACQTFRHAKVQQLYEYVQECAQKGQAVDIGKAAFTTSLNLLSKLFFSVELAHHKSHTSQEFKELIWNIMEDIGKPNYADYFPILGCVDPSGIRRRLACSFDKLIAVFQGIICERLAPDSSTTTTTTTDDVLDVLLQLFKQNELTMGEINHLLVDIFDAGTDTTSSTFEWVMTELIRNPEMMEKAQEEIKQVLGKDKQIQESDIINLPYLQAIIKETLRLHPPTVFLLPRKADTDVELYGYIVPKDAQILVNLWAIGRDPNAWQNADIFSPERFIGCEIDVKGRDFGLLPFGAGRRICPGMNLAIRMLTLMLATLLQFFNWKLEGDISPKDLDMDEKFGIALQKTKPLKLIPIPRY

SEQ ID NO: 15 - protein, 496 aa, Amaranthus hybridus
  source 1..496: note = subsp. cruentus
MDNATLAMILTIWFISINFIKMFFYHQNTKLSLPPGPKPLPIIGNILEVGKKPHRSFANLAKIHGPLISLRLGSVTTIVVSSAEVAKEMFLKKDQPLSNRNVPNSVTAGDHHKLTMSWLPVSPKWRNFRKITAVHLLSPLRLDACQSLRHAKVQQLYQYVQECALKGQSVDIGKAAFTTSLNLLSKLFFSKELACHKSHESQELKQLIWNIMEDIGKPNYADYFPILGCIDPLGIRRRLAANFDKLISVFQTIISERLENDINSNATTNDVLDVLLQLYKQKELSMGEINHLLVDIFDAGTDTTSSTFEWVMAELIRNPKMMEKAQQEIHEVLGKDRQIQESDIIKLPYLQALIKETLRLHPPTVFLLPRKADMDVELYGYVVPKDAQILVNLWAIGRDSQVWEKPNVFLPERFLGSDVDVKGRDFGLLPFGAGKRICPGMNLAIRMLTLMLATLLQFFNWKLEDGMNPQDLDMDEKFGIALQKNKPLEIIPSLRH

SEQ ID NO: 16 - protein, 496 aa, Mirabilis jalapa
MDFLTLVMILSIIFFFYNLLKMKFTTHSDAQLPPGPKPMPIFGNIFELGEKPHRSFANLAKTHGPLMSLRLGSVTTIVVSSAEVAKEMFLKNDQSLADRSVPNSVTAGDHHKLTMSWLPVSPKWKNFRKITAVHLLSPQRLDACHALRHAKVKQLYEYVQECALKGEAVDIGKAAFTTSLNLLSNLFFSVELANHTSNTSQEFKQLIWDIMEDIGKPNYADYFPLLKYVDPSGIRRRLAANFDKLIDVFQSFISKRLSSAYSSATSLDDVLDVLLKLLKEKELNMGEINHLLVDIFDAGTDTTSNTFEWAMAELMRNPIMMKRAQNEIALVLGKDNATIQESDIANMPYLQAIIKETLRLHPPTVFLLPRKAITNVKLYGYIVPKNAQILVNLWAIGRDPKVWKNPNEFLPDRFLNSDIDVKGRDFGLLPFGAGRRICPGMNLAYRMLTLMLATLLQSFDWKLPHRNSPLDLDMDEKFGIALQKTKPLEIIPLIKY

SEQ ID NO: 17 - protein, 494 aa, Celosia cristata
MDNATLAMLLAIWFISFHFIKMLFTNQSTKLLPPGPKPLPIIGNILEVGKKPHRSFANLAKIHGPLISLKLGSVTTIVVSSAEVAKEMFLKKDQPLSNRTVPNSVTAGDHHKLTMSWLPVSPKWRNFRKITAVHLLSPLRLDACQSLRHAKVQQLFQYVQECAQKGQAVDIGKAAFTTSLNLLSKLFFSKELASHKSRESQEFKQLIWNIMEDIGKPNYADYFPILGCVDPSGIRRRLASNFDKLIEVFQCIIRQRLERNPSTPPTNDVLDVLLELYKQNELSMGEINHLLVDIFDAGTDTTSSTFEWVMAELIRNPEMMAKAQDEIEQVLGKDRQIQESDIIKLPYLQAIIKETLRLHPPTVFLLPRKADTDVELYGYIVPKDAQILVNLWAIGRDSQAWENPKVFSPDRFLGCEIDVKGRDFGLLPFGAGKRICPGMNLAIRMLTLMLATLLQFFNWKLQDGMSLEDLDMEEKFGIALQKTKPLRIIPVSRY

SEQ ID NO: 18 - protein, 499 aa, Beta vulgaris
MDNATLAVILSILFVFYHIFKSFFTNSSSRRLPPGPKPVPIFGNIFDLGEKPHRSFANLSKIHGPLISLKLGSVTTIVVSSASVAEEMFLKNDQALANRTIPDSVRAGDHDKLSMSWLPVSQKWRNMRKISAVQLLSNQKLDASQPLRQAKVKQLLSYVQVCSEKMQPVDIGRAAFTTSLNLLSNTFFSIELASHESSASQEFKQLMWNIMEEIGRPNYADFFPILGYIDPFGIRRRLAGYFDKLIDVFQDIIRERQKLRSSNSSGAKQTNDILDTLLKLHEDNELSMPEINHLLVDIFDAGTDTTASTLEWAMAELVKNPEMMTKVQIEIEQALGKDCLDIQESDISKLPYLQAIIKETLRLHPPTVFLLPRKADNDVELYGYVVPKNAQVLVNLWAIGRDPKVWKNPEVFSPERFLDCNIDYKGRDFELLPFGAGRRICPGLTLAYRMLNLMLATLLQNYNWKLEDGINPKDLDMDEKFGITLQKVKPLQVIPVPRN

SEQ ID NO: 19 - DNA, 16212 bp, synthetic construct
  misc_feature 1..16212: note = pX11 overexpression vector
  source 1..16212: mol_type = other DNA
cgctgtcatgagacgaattctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatccgatgagtaaataaacaaatacgcaaggggaacgcatgaaggttatcgctgtacttaaccagaaaggcgggtcaggcaagacgaccatcggaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatagatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacataacgaattcgtctcaggagagcgtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgacgcactccttctttactccaccatctcgtccttattgaaaacgtgggtagcaccaaaacgaatcaagtcgctggaactgaagttaccaatcacgctggatgatttgccagttggattaatcttgcctttccccgcatgaataatattgatgaatgcatgcgtgaggggtagttcgatgttggcaatagctgcaattgccgcgacatcctccaacgagcataattcttcagaaaaatagcgatgttccatgttgtcagggcatgcatgatgcacgttatgaggtgacggtgctaggcagtattccctcaaagtttcatagtcagtatcatattcatcattgcattcctgcaagagagaattgagacgcaatccacacgctgcggcaaccttccggcgttcgtggtctatttgctcttggacgttgcaaacgtaagtgttggatcggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcccggatccgggcggaaataggtaaagaagttgcggataaggtaattgccattgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccctccgtcaggagactagagccaagctgatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctgatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaataccacaacaaaacaaacaacaaacaacattacaatttactattctagtcgaaatgaaaatgatgaatggtgaagatgctaatgatcaaatgatcaaagaaagcttcttcataacacatggaaacccaatattaacagtagaagacacacatccattaagacctttctttgaaacttggagagagaaaatcttttctaagaaacctaaggcaattcttattatttctggtcattgggaaactgttaaacctactgttaatgctgtccatatcaatgatactatccatgattttgatgactatcctgctgctatgtaccagttcaagtatccagctcctggggaaccagaattggcaagaaaagtagaggaaattctgaaaaaatcgggtttcgaaacggcggaaactgatcaaaaacgtgggcttgatcatggtgcatgggtacctctaatgctaatgtatcctgaggctgatataccagtatgtcagctctcagttcagccgcatttagatggaacataccattacaacttaggacgagcattggctcccttgaaaaacgacggcgtattaatcattggttcaggaagtgcaactcaccctttggatgaaactcctcattattttgatggagttgcaccttgggcagctgcctttgattcatggcttcgtaaagctctcattaatggaaggtttgaagaagtgaatatatatgaaagcaaagcaccaaattggaaattagcacatcctttcccagaacatttttatccattgcatgttgttcttggcgctgctggtgaaaaatggaaggcagagcttattcatagcagttgggatcatggcaccttgtgtcatggctcctacaagttcacttcagcctaggcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcdcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggagactagagccaagctgatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctgatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaataccacaacaaaacaaacaacaaacaacattacaatttactattctagtcgaaatggatcatgcaacattagcaatgatactagccatttggttcatttcttttcatttcataaaattactttttagccaacaaactaccaaacttcttcctcctggtccaaaaccattgccaataattggtaacatcttagaagttggtaaaaaaccccatcgttcatttgctaatcttgctaaaattcacggccctttaatatcgttacgtctaggaagtgtaacaactattgttgtatcatcagcagatgtagctaaagaaatgttcttaaaaaaagaccaccctctttctaaccgtactattcctaattctgtcacggccggtgaccaccataaactcaccatgtcgtggttgcccgtttcgccgaaatggcggaattttcgtaagattacagccgtccatttgctttctcctcaaagacttgatgcttgccaaacctttcgccatgccaaggtgcaacagctttatgaatatgtacaagaatgtgcacaaaaaggccaagctgttgatattggcaaagctgcatttactacctcccttaatttgttatctaaactattcttttcagtggaattagcccaccataaatcacacacttctcaagagtttaaggaactaatatggaacattatggaagatattgggaaacctaattatgctgattattttcctattttaggctgtgttgatccatcaggtattcgtcgaagattagcatgtagttttgacaagttgattgcagtttttcagggtataatatgtgaaaggcttgcgcctgattcttcaactacaacaacaacgacgactgatgatgtgctagacgttcttcttcagctcttcaaacaaaatgagctcactatgggcgagattaaccatttgctcgtcgacatttttgatgctggaacagacactacatcaagtacttttgagtgggtcatgacagagttaattaggaatcctgaaatgatggaaaaggctcaagaagaaattaagcaagtattgggcaaagataaacaaattcaggaatcagacattattaacctaccttacttacaagccattatcaaagaaactttgcgactacatccaccaactgtatttcttttgcctcgtaaagccgacactgatgttgaactatatggttatattgtgcctaaagatgcacaaatacttgttaacttatgggctattggaagagatcctaatgcatggcaaaatgctgatattttttcgcccgaaagatttatagggtgtgaaaytgatgtcaaaggaagagattttggactcttaccttttggagccggaagaaggatatgtcctgggatgaatttggccattagaatgttaactttgatgctagctactttacttcaattcttcaattggaagcttgaaggagacataagtccaaaagacttagacatggatgagaaatttgggattgcgttacaaaagacaaagcctttaaaacttattccaatacctaggtattgataataagcttgctctcaagatcaaaggcttaaaaagctggggttttatgaatgggatcaaagtttctttttttcttttatatttgcttctccatttgtttgtttcatttccctttttgttttcgtttctatgatgcacttgtgtgtgacaaactctctgggtttttacttacgtctgcgtttcaaaaaaaaaaaccgctttcgttttgcgttttagtcccattgttttgtagctctgagtgatcgaattgatgcctctttattccttttgttccctataatttctttcaaaactcagaagaaaaaccttgaaactctttgcaatgttaatataagtattgtataagatttttattgatttggttattagtcttacttttgctacctccatcttcacttggaactgatattctgaatagttaaagcgttacatgtcttccattcacaaatgaacttaaactagcacaaagtcagatattttaagatcgcaccattttatataaccccaatcgtcaattctactgtttcaagttttacaccaaaacaattacgaggtgtatctattcgttactcttttcgcttattaatccttttacatgatgcaaaatataacaaattaatttcaaattgtcctgcacatttggtttatacatcttgattccaaaaagtcgctgtcaggaggtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagaatgaccgccattaaaatgaacaccaatggtgaaggtgaaacacaacatatactaatgatacctttcatggcgcaagggcatttacgtcctttccttgagcttgctatgtttctatataaacgaagtcattttatcattactcttcttactaccccgctcaatgcgggtttcctacgacatctccttcaccaccatagctattctagctcggggatcagaattgtcgagttacctttcaactcaaccaatcatggtcttccacctggcattgaaaacactgataaacttacactcccacttgtagtatcactttttcattcaaccatttctcttgaccctcaccttagagattatatttcccgccatttctcccctgcgcgccctcctctgtgtgtcatacatgatgtgttccttggttgggttgatcaagttgctaaagacgtgggctcaactggtgttgtttttactacgggtggcgcgtatggtacaagcgcatatgtgtccatttggaatgatctgcctcaccagaattactctgatgatcaagagtttccgcttcctggtttcccggagaatcataaattccgacgttctcaacttcatcggtttctgaggtatgccgatggatcagatgattggtcgaaatatttccaaccgcaattgaggcaatcaatgaagagttttggatggctatgtaattcagttgaggaaatcgaaacacttgggtttagtatcctcaggaactacactaaactacccatttggggtattggaccgttgatagcttcacctgtacaacattcatcatctgataataacagtactggtgccgagtttgttcaatggttgagcttgaaagaaccagattctgtattatacatctcatttggatcacagaacacaatttcaccaacccagatgatggaactagcagccggtttggagtcaagtgagaagccgtttttgtgggtgattcgagcaccatttgggttcgatatcaatgaggaaatgagrccagaatggctaccagagggattcgaggagcgaatgaaggtgaaaaaacaaggaaagttggtgtataagttgggaccacagttggagatacttaaccatgagtcaatcggagggttcttaactcattgtgggtggaattcgatccttgagtcacttcgagaaggtgtgcctatgttagggtggccattggcagccgaacaagcttataatttgaagtatttggaggacgaaatgggggttgcagtcgagttagcgaggggattggaaggagagataagtaaagagaaagtgaagagaattgtggagatgattttagagagaaatgaaggaagtaaaggatgggaaatgaaaaatagagcagtagaaatggggaagaaacttaaagacgctgtcaatgaggagaaggaactgaagggttcttctgttaaggcaatagatgatttcttagatgcggtcatgcaagctaagttggaaccttctcttcaataagcttcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacct

SEQ ID NO: 20 - DNA, 16919 bp, synthetic construct
  misc_feature 1..16919: note = pX11(E8)
  source 1..16919: mol_type = other DNA
ggaggggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaatggcaattaccttatccgcaacttctttacctatttccgcccggatccgggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgatccaacacttacgtttgcaacgtccaagagcaaatagaccacgaacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaacatcgaactacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctgtcaggagactagagccaagctgatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctgatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaataccacaacaaaacaaacaacaaacaacattacaatttactattctagtcgaaatgaaaatgatgaatggtgaagatgctaatgatcaaatgatcaaagaaagcttcttcataacacatggaaacccaatattaacagtagaagacacacatccattaagacctttctttgaaacttggagagagaaaatcttttctaagaaacctaaggcaattcttattatttctggtcattgggaaactgttaaacctactgttaatgctgtccatatcaatgatactatccatgattttgatgactatcctgctgctatgtaccagttcaagtatccagctcctggggaaccagaattggcaagaaaagtagaggaaattctgaaaaaatcgggtttcgaaacggcggaaactgatcaaaaacgtgggcttgatcatggtgcatgggtacctctaatgctaatgtatcctgaggctgatataccagtatgtcagctctcagttcagccgcatttagatggaacataccattacaacttaggacgagcattggctcccttgaaaaacgacggcgtattaatcattggttcaggaagtgcaactcaccctttggatgaaactcctcattattttgatggagttgcaccttgggcagctgcctttgattcatggcttcgtaaagctctcattaatggaaggtttgaagaagtgaatatatatgaaagcaaagcaccaaattggaaattagcacatcctttcccagaacatttttatccattgcatgttgttcttggcgctgctggtgaaaaatggaaggcagagcttattcatagcagttgggatcatggcaccttgtgtcatggctcctacaagttcacttcagcctaggcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcdcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggaggaattcatttttgacatccctaatgatattgttcacgtaattaagttttgtggaagtgagagagtccaattttgataagaaaagagtcagaaaacgtaatattttaaaagtctaaatctttctacaaataagagcaaatttatttattttttaatccaataaatattaatggaggacaaattcaattcacttggttgtaaaataaacttaaaccaataaccaaagaactaataaatctgaagtggaattattaaggataatgtacatagacaatgaagaaataataggttcgatgaattaataataattaaggatgttacaatcatcatgtgccaagtatatacacaatattctatgggatttataatttcgttacttcacttaacttttgcgtaaataaaacgaattatctgatattttataataaaacagttaattaagaaccatcatttttaacaacatagatatattatttctaatagtttaatgatacttttaaatcttttaaattttatgtttcttttagaaaataaaaattcaaaaaaattaaatatatttacaaaaactacaatcaaacacaacttcatatattaaaagcaaaatatattttgaaaatttcaagtgtcctaacaaataagacaagaggaaaatgtacgatgagagacataaagagaactaataattgaggagtcctataatatataataaagtttattagtaaacttaattattaaggactcctaaaatatatgataggagaaaatgaatggtgagagatattggaaaacttaataattaaggatattaaaatatatggtaaaagataggcaaagtatccattatccccttttaacttgaagtctacctaggcgcatgtgaaaggttgattttttgtcacgtcatatagctataacgtaaaaaaagaaagtaaaatttttaattttttttaatatatgacatattttaaacgaaatataggacaaaatgtaaatgaatagtaaaggaaacaaagattaatacttactttgtaagaatttaagataaatttaaaatttaatagatcaactttacgtctagaaagacccatatctagaaggaatttcacgaaatcggcccttattcaaaaataacttttaaataatgaattttaaattttaagaaataatatccaatgaataaatgacatgtagcattttacctaaatatttcaactattttaatccaatattaatttgttttattcccaacaatagaaagtcttgtgcagacatttaatctgacttttccagtactaaatattaattttctgaagattttcgggtttagtccacaagttttagtgagaagttttgctcaaaattttaggtgagaaggtttgatatttatcttttgttaaattaatttatctaggtgactattatttatttaagtagaaattcatatcattacttttgccaacttgtagtcataataggagtaggtgtatatgatgaaggaataaacaagttcagtgaagtgattaaaataaaatataatttaggtgtacatcaaataaaaaccttaaagtttagaaaggcaccgaataattttgcatagaagatattagtaaatttataaaaataaaagaaatgtagttgtcaagttgtcttcttttttttggataaaaatagcagttggcttatgtcattcttttacaacctccatgccacttgtccaattgttgacacttaactaattagtttgattcatgtatgaatactaaataattttttaggactgactcaaatatttttatattatcatagtaatatttatctaatttttaggaccacttattactaaataataaattaactactactatattattgttgtgaaacaacaacgttttggttgttatgatgaacactatatcagtatgaaaaattcaaaacgattagtataaattatattgaaaatttgatatttttctattcttaatcagacgtattgggtttcatattttaaaaagggactaaacttagaagagaagtttgtttgaaactacttttgtctctttcttgttcccatttctctcttagatttcaaaaagtgaactactttatctctttctttgttcacattttattttattctattataaatatggcatcctcatattgagatttttaatggatcatgcaacattagcaatgatactagccatttggttcatttcttttcatttcataaaattactttttagccaacaaactaccaaacttcttcctcctggtccaaaaccattgccaataattggtaacatcttagaagttggtaaaaaaccccatcgttcatttgctaatcttgctaaaattcacggccctttaatatcgttacgtctaggaagtgtaacaactattgttgtatcatcagcagatgtagctaaagaaatgttcttaaaaaaagaccaccctctttctaaccgtactattcctaattctgtcacggccggtgaccaccataaactcaccatgtcgtggttgcccgtttcgccgaaatggcggaattttcgtaagattacagccgtccatttgctttctcctcaaagacttgatgcttgccaaacctttcgccatgccaaggtgcaacagctttatgaatatgtacaagaatgtgcacaaaaaggccaagctgttgatattggcaaagctgcatttactacctcccttaatttgttatctaaactattcttttcagtggaattagcccaccataaatcacacacttctcaagagtttaaggaactaatatggaacattatggaagatattgggaaacctaattatgctgattattttcctattttaggctgtgttgatccatcaggtattcgtcgaagattagcatgtagttttgacaagttgattgcagtttttcagggtataatatgtgaaaggcttgcgcctgattcttcaactacaacaacaacgacgactgatgatgtgctagacgttcttcttcagctcttcaaacaaaatgagctcactatgggcgagattaaccatttgctcgtcgacatttttgatgctggaacagacactacatcaagtacttttgagtgggtcatgacagagttaattaggaatcctgaaatgatggaaaaggctcaagaagaaattaagcaagtattgggcaaagataaacaaattcaggaatcagacattattaacctaccttacttacaagccattatcaaagaaactttgcgactacatccaccaactgtatttcttttgcctcgtaaagccgacactgatgttgaactatatggttatattgtgcctaaagatgcacaaatacttgttaacttatgggctattggaagagatcctaatgcatggcaaaatgctgatattttttcgcccgaaagatttatagggtgtgaaaytgatgtcaaaggaagagattttggactcttaccttttggagccggaagaaggatatgtcctgggatgaatttggccattagaatgttaactttgatgctagctactttacttcaattcttcaattggaagcttgaaggagacataagtccaaaagacttagacatggatgagaaatttgggattgcgttacaaaagacaaagcctttaaaacttattccaatacctaggtattgataataagcttcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctcgctgtcaggaggtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagaatgaccgccattaaaatgaacaccaatggtgaaggtgaaacacaacatatactaatgatacctttcatggcgcaagggcatttacgtcctttccttgagcttgctatgtttctatataaacgaagtcattttatcattactcttcttactaccccgctcaatgcgggtttcctacgacatctccttcaccaccatagctattctagctcggggatcagaattgtcgagttacctttcaactcaaccaatcatggtcttccacctggcattgaaaacactgataaacttacactcccacttgtagtatcactttttcattcaaccatttctcttgaccctcaccttagagattatatttcccgccatttctcccctgcgcgccctcctctgtgtgtcatacatgatgtgttccttggttgggttgatcaagttgctaaagacgtgggctcaactggtgttgtttttactacgggtggcgcgtatggtacaagcgcatatgtgtccatttggaatgatctgcctcaccagaattactctgatgatcaagagtttccgcttcctggtttcccggagaatcataaattccgacgttctcaacttcatcggtttctgaggtatgccgatggatcagatgattggtcgaaatatttccaaccgcaattgaggcaatcaatgaagagttttggatggctatgtaattcagttgaggaaatcgaaacacttgggtttagtatcctcaggaactacactaaactacccatttggggtattggaccgttgatagcttcacctgtacaacattcatcatctgataataacagtactggtgccgagtttgttcaatggttgagcttgaaagaaccagattctgtattatacatctcatttggatcacagaacacaatttcaccaacccagatgatggaactagcagccggtttggagtcaagtgagaagccgtttttgtgggtgattcgagcaccatttgggttcgatatcaatgaggaaatgagrccagaatggctaccagagggattcgaggagcgaatgaaggtgaaaaaacaaggaaagttggtgtataagttgggaccacagttggagatacttaaccatgagtcaatcggagggttcttaactcattgtgggtggaattcgatccttgagtcacttcgagaaggtgtgcctatgttagggtggccattggcagccgaacaagcttataatttgaagtatttggaggacgaaatgggggttgcagtcgagttagcgaggggattggaaggagagataagtaaagagaaagtgaagagaattgtggagatgattttagagagaaatgaaggaagtaaaggatgggaaatgaaaaatagagcagtagaaatggggaagaaacttaaagacgctgtcaatgaggagaaggaactgaagggttcttctgttaaggcaatagatgatttcttagatgcggtcatgcaagctaagttggaaccttctcttcaataagcttcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctcgctgtcatgagacgaattctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatccgatgagtaaataaacaaatacgcaaggggaacgcatgaaggttatcgctgtacttaaccagaaaggcgggtcaggcaagacgaccatcggaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatagatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacataacgaattcgtctca

SEQ ID NO: 21 - DNA, 15191 bp, synthetic construct
  misc_feature 1..15191: note = pX11(CHS)
  source 1..15191: mol_type = other DNA
cgctgtcatgagacgaattctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatccgatgagtaaataaacaaatacgcaaggggaacgcatgaaggttatcgctgtacttaaccagaaaggcgggtcaggcaagacgaccatcggaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatagatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacataacgaattcgtctcaggaggggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaatggcaattaccttatccgcaacttctttacctatttccgcccggatccgggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgatccaacacttacgtttgcaacgtccaagagcaaatagaccacgaacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaacatcgaactacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctgtcaggagactagagccaagctgatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctgatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaataccacaacaaaacaaacaacaaacaacattacaatttactattctagtcgaaatgaaaatgatgaatggtgaagatgctaatgatcaaatgatcaaagaaagcttcttcataacacatggaaacccaatattaacagtagaagacacacatccattaagacctttctttgaaacttggagagagaaaatcttttctaagaaacctaaggcaattcttattatttctggtcattgggaaactgttaaacctactgttaatgctgtccatatcaatgatactatccatgattttgatgactatcctgctgctatgtaccagttcaagtatccagctcctggggaaccagaattggcaagaaaagtagaggaaattctgaaaaaatcgggtttcgaaacggcggaaactgatcaaaaacgtgggcttgatcatggtgcatgggtacctctaatgctaatgtatcctgaggctgatataccagtatgtcagctctcagttcagccgcatttagatggaacataccattacaacttaggacgagcattggctcccttgaaaaacgacggcgtattaatcattggttcaggaagtgcaactcaccctttggatgaaactcctcattattttgatggagttgcaccttgggcagctgcctttgattcatggcttcgtaaagctctcattaatggaaggtttgaagaagtgaatatatatgaaagcaaagcaccaaattggaaattagcacatcctttcccagaacatttttatccattgcatgttgttcttggcgctgctggtgaaaaatggaaggcagagcttattcatagcagttgggatcatggcaccttgtgtcatggctcctacaagttcacttcagcctaggcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcdcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggaggtgcactgtcaaattatgtcataaattctaatgagatgagtttatatagcatttacaaattcaaatacttaaaaaggatactacaatttggtattacgtgtcaaattcctgttcaaagctgatgctagaggtgacagaaatcatatgcaagaacgtcaaggccattcattttggttcaacagattgatgaaaaatctaagagcctttgatcttgagaagtaggtaaccattaatttgtgttgaaggtttgctacgaaaataaaaaggatgtcacgtgccatcaagttatagctacacgtgattactatctaccattctcctttagggttctcgtataaatactctacaatccccatgcaaaccataacacaatataaaagatacttacacttgtcacgtactacataaaaaaaaaaaaataccaaacttttttcaagcaaaaaatggatcatgcaacattagcaatgatactagccatttggttcatttcttttcatttcataaaattactttttagccaacaaactaccaaacttcttcctcctggtccaaaaccattgccaataattggtaacatcttagaagttggtaaaaaaccccatcgttcatttgctaatcttgctaaaattcacggccctttaatatcgttacgtctaggaagtgtaacaactattgttgtatcatcagcagatgtagctaaagaaatgttcttaaaaaaagaccaccctctttctaaccgtactattcctaattctgtcacggccggtgaccaccataaactcaccatgtcgtggttgcccgtttcgccgaaatggcggaattttcgtaagattacagccgtccatttgctttctcctcaaagacttgatgcttgccaaacctttcgccatgccaaggtgcaacagctttatgaatatgtacaagaatgtgcacaaaaaggccaagctgttgatattggcaaagctgcatttactacctcccttaatttgttatctaaactattcttttcagtggaattagcccaccataaatcacacacttctcaagagtttaaggaactaatatggaacattatggaagatattgggaaacctaattatgctgattattttcctattttaggctgtgttgatccatcaggtattcgtcgaagattagcatgtagttttgacaagttgattgcagtttttcagggtataatatgtgaaaggcttgcgcctgattcttcaactacaacaacaacgacgactgatgatgtgctagacgttcttcttcagctcttcaaacaaaatgagctcactatgggcgagattaaccatttgctcgtcgacatttttgatgctggaacagacactacatcaagtacttttgagtgggtcatgacagagttaattaggaatcctgaaatgatggaaaaggctcaagaagaaattaagcaagtattgggcaaagataaacaaattcaggaatcagacattattaacctaccttacttacaagccattatcaaagaaactttgcgactacatccaccaactgtatttcttttgcctcgtaaagccgacactgatgttgaactatatggttatattgtgcctaaagatgcacaaatacttgttaacttatgggctattggaagagatcctaatgcatggcaaaatgctgatattttttcgcccgaaagatttatagggtgtgaaaytgatgtcaaaggaagagattttggactcttaccttttggagccggaagaaggatatgtcctgggatgaatttggccattagaatgttaactttgatgctagctactttacttcaattcttcaattggaagcttgaaggagacataagtccaaaagacttagacatggatgagaaatttgggattgcgttacaaaagacaaagcctttaaaacttattccaatacctaggtattgataataagcttcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctcgctgtcaggaggtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagaatgaccgccattaaaatgaacaccaatggtgaaggtgaaacacaacatatactaatgatacctttcatggcgcaagggcatttacgtcctttccttgagcttgctatgtttctatataaacgaagtcattttatcattactcttcttactaccccgctcaatgcgggtttcctacgacatctccttcaccaccatagctattctagctcggggatcagaattgtcgagttacctttcaactcaaccaatcatggtcttccacctggcattgaaaacactgataaacttacactcccacttgtagtatcactttttcattcaaccatttctcttgaccctcaccttagagattatatttcccgccatttctcccctgcgcgccctcctctgtgtgtcatacatgatgtgttccttggttgggttgatcaagttgctaaagacgtgggctcaactggtgttgtttttactacgggtggcgcgtatggtacaagcgcatatgtgtccatttggaatgatctgcctcaccagaattactctgatgatcaagagtttccgcttcctggtttcccggagaatcataaattccgacgttctcaacttcatcggtttctgaggtatgccgatggatcagatgattggtcgaaatatttccaaccgcaattgaggcaatcaatgaagagttttggatggctatgtaattcagttgaggaaatcgaaacacttgggtttagtatcctcaggaactacactaaactacccatttggggtattggaccgttgatagcttcacctgtacaacattcatcatctgataataacagtactggtgccgagtttgttcaatggttgagcttgaaagaaccagattctgtattatacatctcatttggatcacagaacacaatttcaccaacccagatgatggaactagcagccggtttggagtcaagtgagaagccgtttttgtgggtgattcgagcaccatttgggttcgatatcaatgaggaaatgagrccagaatggctaccagagggattcgaggagcgaatgaaggtgaaaaaacaaggaaagttggtgtataagttgggaccacagttggagatacttaaccatgagtcaatcggagggttcttaactcattgtgggtggaattcgatccttgagtcacttcgagaaggtgtgcctatgttagggtggccattggcagccgaacaagcttataatttgaagtatttggaggacgaaatgggggttgcagtcgagttagcgaggggattggaaggagagataagtaaagagaaagtgaagagaattgtggagatgattttagagagaaatgaaggaagtaaaggatgggaaatgaaaaatagagcagtagaaatggggaagaaacttaaagacgctgtcaatgaggagaaggaactgaagggttcttctgttaaggcaatagatgatttcttagatgcggtcatgcaagctaagttggaaccttctcttcaataagcttcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacct

SEQ ID NO: 22 - DNA, 19745 bp, synthetic construct
  misc_feature 1..19745: note = pX12
  source 1..19745: mol_type = other DNA
cgctgtcatgagaccggatcctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatccgatgagtaaataaacaaatacgcaaggggaacgcatgaaggttatcgctgtacttaaccagaaaggcgggtcaggcaagacgaccatcggaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatagatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcaccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcataatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggtagcagcgaagtcgaggcatttctgtcctggctggaacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacataacggatccggtctcaggagagcgtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgacgcactccttctttactccaccatctcgtccttattgaaaacgtgggtagcaccaaaacgaatcaagtcgctggaactgaagttaccaatcacgctggatgatttgccagttggattaatcttgcctttccccgcatgaataatattgatgaatgcatgcgtgaggggtagttcgatgttggcaatagctgcaattgccgcgacatcctccaacgagcataattcttcagaaaaatagcgatgttccatgttgtcagggcatgcatgatgcacgttatgaggtgacggtgctaggcagtattccctcaaagtttcatagtcagtatcatattcatcattgcattcctgcaagagagaattgagacgcaatccacacgctgcggcaaccttccggcgttcgtggtctatttgctcttggacgttgcaaacgtaagtgttggatcggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcccggatccgggcggaaataggtaaagaagttgcggataaggtaattgccattgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccctccgtcaggagactagagccaagctgatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctgatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaataccacaacaaaacaaacaacaaacaacattacaatttactattctagtcgaaatgaaaatgatgaatggtgaagatgctaatgatcaaatgatcaaagaaagcttcttcataacacatggaaacccaatattaacagtagaagacacacatccattaagacctttctttgaaacttggagagagaaaatcttttctaagaaacctaaggcaattcttattatttctggtcattgggaaactgttaaacctactgttaatgctgtccatatcaatgatactatccatgattttgatgactatcctgctgctatgtaccagttcaagtatccagctcctggggaaccagaattggcaagaaaagtagaggaaattctgaaaaaatcgggtttcgaaacggcggaaactgatcaaaaacgtgggcttgatcatggtgcatgggtacctctaatgctaatgtatcctgaggctgatataccagtatgtcagctctcagttcagccgcatttagatggaacataccattacaacttaggacgagcattggctcccttgaaaaacgacggcgtattaatcattggttcaggaagtgcaactcaccctttggatgaaactcctcattattttgatggagttgcaccttgggcagctgcctttgattcatggcttcgtaaagctctcattaatggaaggtttgaagaagtgaatatatatgaaagcaaagcaccaaattggaaattagcacatcctttcccagaacatttttatccattgcatgttgttcttggcgctgctggtgaaaaatggaaggcagagcttattcatagcagttgggatcatggcaccttgtgtcatggctcctacaagttcacttcagcctaggcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcdcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggagactagagccaagctgatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctgatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaataccacaacaaaacaaacaacaaacaacattacaatttactattctagtcgaaatggatcatgcaacattagcaatgatactagccatttggttcatttcttttcatttcataaaattactttttagccaacaaactaccaaacttcttcctcctggtccaaaaccattgccaataattggtaacatcttagaagttggtaaaaaaccccatcgttcatttgctaatcttgctaaaattcacggccctttaatatcgttacgtctaggaagtgtaacaactattgttgtatcatcagcagatgtagctaaagaaatgttcttaaaaaaagaccaccctctttctaaccgtactattcctaattctgtcacggccggtgaccaccataaactcaccatgtcgtggttgcccgtttcgccgaaatggcggaattttcgtaagattacagccgtccatttgctttctcctcaaagacttgatgcttgccaaacctttcgccatgccaaggtgcaacagctttatgaatatgtacaagaatgtgcacaaaaaggccaagctgttgatattggcaaagctgcatttactacctcccttaatttgttatctaaactattcttttcagtggaattagcccaccataaatcacacacttctcaagagtttaaggaactaatatggaacattatggaagatattgggaaacctaattatgctgattattttcctattttaggctgtgttgatccatcaggtattcgtcgaagattagcatgtagttttgacaagttgattgcagtttttcagggtataatatgtgaaaggcttgcgcctgattcttcaactacaacaacaacgacgactgatgatgtgctagacgttcttcttcagctcttcaaacaaaatgagctcactatgggcgagattaaccatttgctcgtcgacatttttgatgctggaacagacactacatcaagtacttttgagtgggtcatgacagagttaattaggaatcctgaaatgatggaaaaggctcaagaagaaattaagcaagtattgggcaaagataaacaaattcaggaatcagacattattaacctaccttacttacaagccattatcaaagaaactttgcgactacatccaccaactgtatttcttttgcctcgtaaagccgacactgatgttgaactatatggttatattgtgcctaaagatgcacaaatacttgttaacttatgggctattggaagagatcctaatgcatggcaaaatgctgatattttttcgcccgaaagatttatagggtgtgaaaytgatgtcaaaggaagagattttggactcttaccttttggagccggaagaaggatatgtcctgggatgaatttggccattagaatgttaactttgatgctagctactttacttcaattcttcaattggaagcttgaaggagacataagtccaaaagacttagacatggatgagaaatttgggattgcgttacaaaagacaaagcctttaaaacttattccaatacctaggtattgataataagcttgctctcaagatcaaaggcttaaaaagctggggttttatgaatgggatcaaagtttctttttttcttttatatttgcttctccatttgtttgtttcatttccctttttgttttcgtttctatgatgcacttgtgtgtgacaaactctctgggtttttacttacgtctgcgtttcaaaaaaaaaaaccgctttcgttttgcgttttagtcccattgttttgtagctctgagtgatcgaattgatgcctctttattccttttgttccctataatttctttcaaaactcagaagaaaaaccttgaaactctttgcaatgttaatataagtattgtataagatttttattgatttggttattagtcttacttttgctacctccatcttcacttggaactgatattctgaatagttaaagcgttacatgtcttccattcacaaatgaacttaaactagcacaaagtcagatattttaagatcgcaccattttatataaccccaatcgtcaattctactgtttcaagttttacaccaaaacaattacgaggtgtatctattcgttactcttttcgcttattaatccttttacatgatgcaaaatataacaaattaatttcaaattgtcctgcacatttggtttatacatcttgattccaaaaagtcgctgtcaggaggtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagaatgaccgccattaaaatgaacaccaatggtgaaggtgaaacacaacatatactaatgatacctttcatggcgcaagggcatttacgtcctttccttgagcttgctatgtttctatataaacgaagtcattttatcattactcttcttactaccccgctcaatgcgggtttcctacgacatctccttcaccaccatagctattctagctcggggatcagaattgtcgagttacctttcaactcaaccaatcatggtcttccacctggcattgaaaacactgataaacttacactcccacttgtagtatcactttttcattcaaccatttctcttgaccctcaccttagagattatatttcccgccatttctcccctgcgcgccctcctctgtgtgtcatacatgatgtgttccttggttgggttgatcaagttgctaaagacgtgggctcaactggtgttgtttttactacgggtggcgcgtatggtacaagcgcatatgtgtccatttggaatgatctgcctcaccagaattactctgatgatcaagagtttccgcttcctggtttcccggagaatcataaattccgacgttctcaacttcatcggtttctgaggtatgccgatggatcagatgattggtcgaaatatttccaaccgcaattgaggcaatcaatgaagagttttggatggctatgtaattcagttgaggaaatcgaaacacttgggtttagtatcctcaggaactacactaaactacccatttggggtattggaccgttgatagcttcacctgtacaacattcatcatctgataataacagtactggtgccgagtttgttcaatggttgagcttgaaagaaccagattctgtattatacatctcatttggatcacagaacacaatttcaccaacccagatgatggaactagcagccggtttggagtcaagtgagaagccgtttttgtgggtgattcgagcaccatttgggttcgatatcaatgaggaaatgagrccagaatggctaccagagggattcgaggagcgaatgaaggtgaaaaaacaaggaaagttggtgtataagttgggaccacagttggagatacttaaccatgagtcaatcggagggttcttaactcattgtgggtggaattcgatccttgagtcacttcgagaaggtgtgcctatgttagggtggccattggcagccgaacaagcttataatttgaagtatttggaggacgaaatgggggttgcagtcgagttagcgaggggattggaaggagagataagtaaagagaaagtgaagagaattgtggagatgattttagagagaaatgaaggaagtaaaggatgggaaatgaaaaatagagcagtagaaatggggaagaaacttaaagacgctgtcaatgaggagaaggaactgaagggttcttctgttaaggcaatagatgatttcttagatgcggtcatgcaagctaagttggaaccttctcttcaataagcttcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctcgctgtcaggagactagagccaagctgatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctgatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaataccacaacaaaacaaacaacaaacaacattacaatttactattctagtcgaaatggataacgcaacacttgctgtgatcctttccattttgtttgtgttttaccacattttcaaatcctttttcaccaattcttcatctcgtaggcttcctcctggtcccaaacccgtgccaatttttggcaacattttcgatcttggcgaaaagcctcatcgatcttttgccaatctatctaaaattcacggccctttgattagcctaaagttaggaagtgtaacaactattgttgtttcctcggcctctgtggccgaggaaatgttccttaaaaatgaccaagcacttgctaaccgaaccattcctgactcggttagggctggtgaccacgacaaattatccatgtcgtggttgcctgtttcccaaaaatggagaaatatgagaaaaatctccgctgtccaattactctccaaccaaaaacttgatgctagtcaacctcttagacaagctaaggtgaaacaacttttatcatacgtacaagtttgttccgaaaaaatgcaacccgtcgatattggacgggccgcatttacaacgtcacttaatttattatcaaacacatttttctcaatcgaattagcaagtcatgaatctagtgcttcccaagagtttaaacaactcatgtggaatattatggaggaaattggaaggcctaattatgctgattttttccctattcttggttacattgatccctttggtataagacgtcgtttggctggttactttgataaactcattgatgttttccaagacattattcgtgaaagacaaaagcttcgatcttctaattcttccggcgcaaaacaaacaaatgacattcttgatactcttcttaaactccatgaagataatgagttgagtatgcctgaaattaatcaccttctcgtggatatctttgacgccggaacagacacaacagcaagcacattagaatgggcaatggccgaacttgtgaaaaacccggaaatgatgactaaagttcaaattgaaatcgaacaagctcttggaaaagattgcttagacatacaagaatccgacatctcaaaactaccttatttacaagccattataaaagaaacgttacgtttacaccctcctactgtgtttttgctgcctcgaaaggcagacaatgacgtagagttatatggctacgttgtaccaaagaatgctcaagtccttgtcaatctttgggcaattggtcgtgatccaaaggtatggaaaaatccggaagtattttctcctgaaaggtttttagattgcaatatcgattataaaggacgagatttcgaacttttaccctttggtgctggtagaaggatatgccctggacttactttggcatatagaatgttgaacttgatgttggctactcttcttcaaaactacaattggaaacttgaagatggtatcaatcctaaggatttagacatggatgagaaatttgggattacattgcaaaaggttaaacctcttcaagttattccagttcccagaaactaggcttgctctcaagatcaaaggcttaaaaagctggggttttatgaatgggatcaaagtttctttttttcttttatatttgcttctccatttgtttgtttcatttccctttttgttttcgtttctatgatgcacttgtgtgtgacaaactctctgggtttttacttacgtctgcgtttcaaaaaaaaaaaccgctttcgttttgcgttttagtcccattgttttgtagctctgagtgatcgaattgatgcctctttattccttttgttccctataatttctttcaaaactcagaagaaaaaccttgaaactctttgcaatgttaatataagtattgtataagatttttattgatttggttattagtcttacttttgctacctccatcttcacttggaactgatattctgaatagttaaagcgttacatgtcttccattcacaaatgaacttaaactagcacaaagtcagatattttaagatcgcaccattttatataaccccaatcgtcaattctactgtttcaagttttacaccaaaacaattacgaggtgtatctattcgttactcttttcgcttattaatccttttacatgatgcaaaatataacaaattaatttcaaattgtcctgcacatttggtttatacatcttgattccaaaaagt

SEQ ID NO: 23 - DNA, 13992 bp, synthetic construct
  misc_feature 1..13992: note = pX13
  source 1..13992: mol_type = other DNA
cgctgtcatgagacgaattctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatccgatgagtaaataaacaaatacgcaaggggaacgcatgaaggttatcgctgtacttaaccagaaaggcgggtcaggcaagacgaccatcggaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatagatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacataacgaattcgtctcaggagagcgtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgacgcactccttctttactccaccatctcgtccttattgaaaacgtgggtagcaccaaaacgaatcaagtcgctggaactgaagttaccaatcacgctggatgatttgccagttggattaatcttgcctttccccgcatgaataatattgatgaatgcatgcgtgaggggtagttcgatgttggcaatagctgcaattgccgcgacatcctccaacgagcataattcttcagaaaaatagcgatgttccatgttgtcagggcatgcatgatgcacgttatgaggtgacggtgctaggcagtattccctcaaagtttcatagtcagtatcatattcatcattgcattcctgcaagagagaattgagacgcaatccacacgctgcggcaaccttccggcgttcgtggtctatttgctcttggacgttgcaaacgtaagtgttggatcggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcccggatccgggcggaaataggtaaagaagttgcggataaggtaattgccattgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccctccgtcaggagactagagccaagctgatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctgatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaataccacaacaaaacaaacaacaaacaacattacaatttactattctagtcgaaatgaaaatgatgaatggtgaagatgctaatgatcaaatgatcaaagaaagcttcttcataacacatggaaacccaatattaacagtagaagacacacatccattaagacctttctttgaaacttggagagagaaaatcttttctaagaaacctaaggcaattcttattatttctggtcattgggaaactgttaaacctactgttaatgctgtccatatcaatgatactatccatgattttgatgactatcctgctgctatgtaccagttcaagtatccagctcctggggaaccagaattggcaagaaaagtagaggaaattctgaaaaaatcgggtttcgaaacggcggaaactgatcaaaaacgtgggcttgatcatggtgcatgggtacctctaatgctaatgtatcctgaggctgatataccagtatgtcagctctcagttcagccgcatttagatggaacataccattacaacttaggacgagcattggctcccttgaaaaacgacggcgtattaatcattggttcaggaagtgcaactcaccctttggatgaaactcctcattattttgatggagttgcaccttgggcagctgcctttgattcatggcttcgtaaagctctcattaatggaaggtttgaagaagtgaatatatatgaaagcaaagcaccaaattggaaattagcacatcctttcccagaacatttttatccattgcatgttgttcttggcgctgctggtgaaaaatggaaggcagagcttattcatagcagttgggatcatggcaccttgtgtcatggctcctacaagttcacttcagcctaggcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcdcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggagactagagccaagctgatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctgatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaataccacaacaaaacaaacaacaaacaacattacaatttactattctagtcgaaatggataacgcaacacttgctgtgatcctttccattttgtttgtgttttaccacattttcaaatcctttttcaccaattcttcatctcgtaggcttcctcctggtcccaaacccgtgccaatttttggcaacattttcgatcttggcgaaaagcctcatcgatcttttgccaatctatctaaaattcacggccctttgattagcctaaagttaggaagtgtaacaactattgttgtttcctcggcctctgtggccgaggaaatgttccttaaaaatgaccaagcacttgctaaccgaaccattcctgactcggttagggctggtgaccacgacaaattatccatgtcgtggttgcctgtttcccaaaaatggagaaatatgagaaaaatctccgctgtccaattactctccaaccaaaaacttgatgctagtcaacctcttagacaagctaaggtgaaacaacttttatcatacgtacaagtttgttccgaaaaaatgcaacccgtcgatattggacgggccgcatttacaacgtcacttaatttattatcaaacacatttttctcaatcgaattagcaagtcatgaatctagtgcttcccaagagtttaaacaactcatgtggaatattatggaggaaattggaaggcctaattatgctgattttttccctattcttggttacattgatccctttggtataagacgtcgtttggctggttactttgataaactcattgatgttttccaagacattattcgtgaaagacaaaagcttcgatcttctaattcttccggcgcaaaacaaacaaatgacattcttgatactcttcttaaactccatgaagataatgagttgagtatgcctgaaattaatcaccttctcgtggatatctttgacgccggaacagacacaacagcaagcacattagaatgggcaatggccgaacttgtgaaaaacccggaaatgatgactaaagttcaaattgaaatcgaacaagctcttggaaaagattgcttagacatacaagaatccgacatctcaaaactaccttatttacaagccattataaaagaaacgttacgtttacaccctcctactgtgtttttgctgcctcgaaaggcagacaatgacgtagagttatatggctacgttgtaccaaagaatgctcaagtccttgtcaatctttgggcaattggtcgtgatccaaaggtatggaaaaatccggaagtattttctcctgaaaggtttttagattgcaatatcgattataaaggacgagatttcgaacttttaccctttggtgctggtagaaggatatgccctggacttactttggcatatagaatgttgaacttgatgttggctactcttcttcaaaactacaattggaaacttgaagatggtatcaatcctaaggatttagacatggatgagaaatttgggattacattgcaaaaggttaaacctcttcaagttattccagttcccagaaactaggcttgctctcaagatcaaaggcttaaaaagctggggttttatgaatgggatcaaagtttctttttttcttttatatttgcttctccatttgtttgtttcatttccctttttgttttcgtttctatgatgcacttgtgtgtgacaaactctctgggtttttacttacgtctgcgtttcaaaaaaaaaaaccgctttcgttttgcgttttagtcccattgttttgtagctctgagtgatcgaattgatgcctctttattccttttgttccctataatttctttcaaaactcagaagaaaaaccttgaaactctttgcaatgttaatataagtattgtataagatttttattgatttggttattagtcttacttttgctacctccatcttcacttggaactgatattctgaatagttaaagcgttacatgtcttccattcacaaatgaacttaaactagcacaaagtcagatattttaagatcgcaccattttatataaccccaatcgtcaattctactgtttcaagttttacaccaaaacaattacgaggtgtatctattcgttactcttttcgcttattaatccttttacatgatgcaaaatataacaaattaatttcaaattgtcctgcacatttggtttatacatcttgattccaaaaagtcgctgtcaggagtctcaatggtaactttactctttatttaaccatacatttttttttatttttttcactttgttcttcatccactattgttctttgttcatcttgaacaaaagctccctccttctttgttcttcatccaccattgttcttcatcaatcattt

SEQ ID NO: 24 - DNA, 11797 bp, synthetic construct
  misc_feature 1..11797: note = pDOPA1 overexpression vector
  source 1..11797: mol_type = other DNA
cgctgtcatgagaccggatcctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatccgatgagtaaataaacaaatacgcaaggggaacgcatgaaggttatcgctgtacttaaccagaaaggcgggtcaggcaagacgaccatcggaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatagatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcaccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcataatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggtagcagcgaagtcgaggcatttctgtcctggctggaacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacataacggatccggtctcaggagagcgtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgacgcactccttctttactccaccatctcgtccttattgaaaacgtgggtagcaccaaaacgaatcaagtcgctggaactgaagttaccaatcacgctggatgatttgccagttggattaatcttgcctttccccgcatgaataatattgatgaatgcatgcgtgaggggtagttcgatgttggcaatagctgcaattgccgcgacatcctccaacgagcataattcttcagaaaaatagcgatgttccatgttgtcagggcatgcatgatgcacgttatgaggtgacggtgctaggcagtattccctcaaagtttcatagtcagtatcatattcatcattgcattcctgcaagagagaattgagacgcaatccacacgctgcggcaaccttccggcgttcgtggtctatttgctcttggacgttgcaaacgtaagtgttggatcggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcccggatccgggcggaaataggtaaagaagttgcggataaggtaattgccattgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccctccgtcaggagactagagccaagctgatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctgatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaataccacaacaaaacaaacaacaaacaacattacaatttactattctagtcgaaatggataacgcaacacttgctgtgatcctttccattttgtttgtgttttaccacattttcaaatcctttttcaccaattcttcatctcgtaggcttcctcctggtcccaaacccgtgccaatttttggcaacattttcgatcttggcgaaaagcctcatcgatcttttgccaatctatctaaaattcacggccctttgattagcctaaagttaggaagtgtaacaactattgttgtttcctcggcctctgtggccgaggaaatgttccttaaaaatgaccaagcacttgctaaccgaaccattcctgactcggttagggctggtgaccacgacaaattatccatgtcgtggttgcctgtttcccaaaaatggagaaatatgagaaaaatctccgctgtccaattactctccaaccaaaaacttgatgctagtcaacctcttagacaagctaaggtgaaacaacttttatcatacgtacaagtttgttccgaaaaaatgcaacccgtcgatattggacgggccgcatttacaacgtcacttaatttattatcaaacacatttttctcaatcgaattagcaagtcatgaatctagtgcttcccaagagtttaaacaactcatgtggaatattatggaggaaattggaaggcctaattatgctgattttttccctattcttggttacattgatccctttggtataagacgtcgtttggctggttactttgataaactcattgatgttttccaagacattattcgtgaaagacaaaagcttcgatcttctaattcttccggcgcaaaacaaacaaatgacattcttgatactcttcttaaactccatgaagataatgagttgagtatgcctgaaattaatcaccttctcgtggatatctttgacgccggaacagacacaacagcaagcacattagaatgggcaatggccgaacttgtgaaaaacccggaaatgatgactaaagttcaaattgaaatcgaacaagctcttggaaaagattgcttagacatacaagaatccgacatctcaaaactaccttatttacaagccattataaaagaaacgttacgtttacaccctcctactgtgtttttgctgcctcgaaaggcagacaatgacgtagagttatatggctacgttgtaccaaagaatgctcaagtccttgtcaatctttgggcaattggtcgtgatccaaaggtatggaaaaatccggaagtattttctcctgaaaggtttttagattgcaatatcgattataaaggacgagatttcgaacttttaccctttggtgctggtagaaggatatgccctggacttactttggcatatagaatgttgaacttgatgttggctactcttcttcaaaactacaattggaaacttgaagatggtatcaatcctaaggatttagacatggatgagaaatttgggattacattgcaaaaggttaaacctcttcaagttattccagttcccagaaactaggcttgctctcaagatcaaaggcttaaaaagctggggttttatgaatgggatcaaagtttctttttttcttttatatttgcttctccatttgtttgtttcatttccctttttgttttcgtttctatgatgcacttgtgtgtgacaaactctctgggtttttacttacgtctgcgtttcaaaaaaaaaaaccgctttcgttttgcgttttagtcccattgttttgtagctctgagtgatcgaattgatgcctctttattccttttgttccctataatttctttcaaaactcagaagaaaaaccttgaaactctttgcaatgttaatataagtattgtataagatttttattgatttggttattagtcttacttttgctacctccatcttcacttggaactgatattctgaatagttaaagcgttacatgtcttccattcacaaatgaacttaaactagcacaaagtcagatattttaagatcgcaccattttatataaccccaatcgtcaattctactgtttcaagttttacaccaaaacaattacgaggtgtatctattcgttactcttttcgcttattaatccttttacatgatgcaaaatataacaaattaatttcaaattgtcctgcacatttggtttatacatcttgattccaaaaagt

SEQ ID NO: 25 - DNA, 13626 bp, synthetic construct
  misc_feature 1..13626: note = pDOPA2 overexpression vector
  source 1..13626: mol_type = other DNA
cgctgtcatgagaccggatcctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatccgatgagtaaataaacaaatacgcaaggggaacgcatgaaggttatcgctgtacttaaccagaaaggcgggtcaggcaagacgaccatcggaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatagatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcaccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcataatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggtagcagcgaagtcgaggcatttctgtcctggctggaacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacataacggatccggtctcaggagagcgtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgacgcactccttctttactccaccatctcgtccttattgaaaacgtgggtagcaccaaaacgaatcaagtcgctggaactgaagttaccaatcacgctggatgatttgccagttggattaatcttgcctttccccgcatgaataatattgatgaatgcatgcgtgaggggtagttcgatgttggcaatagctgcaattgccgcgacatcctccaacgagcataattcttcagaaaaatagcgatgttccatgttgtcagggcatgcatgatgcacgttatgaggtgacggtgctaggcagtattccctcaaagtttcatagtcagtatcatattcatcattgcattcctgcaagagagaattgagacgcaatccacacgctgcggcaaccttccggcgttcgtggtctatttgctcttggacgttgcaaacgtaagtgttggatcggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcccggatccgggcggaaataggtaaagaagttgcggataaggtaattgccattgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccctccgtcaggaggtcaactaccccaatttaaattttatttgattaagatatttttatggacctactttataattaaaaatattttctatttgaaaaggaaggacaaaaatcatacaattttggtccaactactcctctctttttttttttggctttataaaaaaggaaagtgattagtaataaataattaaataatgaaaaaaggaggaaataaaattttcgaattaaaatgtaaaagagaaaaaggagagggagtaatcattgtttaactttatctaaagtaccccaattcgattttacatgtatatcaaattatacaaatattttattaaaatatagatattgaataattttattattcttgaacatgtaaataaaaattatctattatttcaatttttatataaactattatttgaaatctcaattatgattttttaatatcactttctatccatgataatttcagcttaaaaagttttgtcaataattacattaattttgttgatgaggatgacaagatttcggtcatcaattacatatacacaaattgaaatagtaagcaacttgattttttttctcataatgataatgacaaagacacgaaaagacaattcaatattcacattgatttatttttatatgataataattacaataataatattcttataaagaaagagatcaattttgactgatccaaaaatttatttatttttactataccaacgtcactaattatatctaataatgtaaaacaattcaatcttacttaaatattaatttgaaataaactatttttataacgaaattactaaatttatccaataacaaaaaggtcttaagaagacataaattctttttttgtaatgctcaaataaatttgagtaaaaaagaatgaaattgagtgatttttttttaatcataagaaaataaataattaatttcaatataataaaacagtaatataatttcataaatggaattcaatacttacctcttagatataaaaaataaatataaaaataaagtgtttctaataaacccgcaatttaaataaaatatttaatattttcaatcaaatttaaataattatattaaaatatcgtagaaaaagagcaatatataatacaagaaagaagatttaagtacaattatcaactattattatactctaattttgttatatttaatttcttacggttaaggtcatgttcacgataaactcaaaatacgctgtatgaggacatattttaaattttaaccaataataaaactaagttatttttagtatatttttttgtttaacgtgacttaatttttcttttctagaggagcgtgtaagtgtcaacctcattctcctaattttcccaaccacataaaaaaaaaataaaggtagcttttgcgtgttgatttggtacactacacgtcattattacacgtgttttcgtatgattggttaatccatgaggcggtttcctctagagtcggccataccatctataaaataaagctttctgcagctcattttttcatcttctatctgatttctattataatttctctgaattgccttcaaatttctctttcaaggttagaatttttctctattttttggtttttgtttgtttagattctgagtttagttaatcaggtgctgttaaagccctaaattttgagtttttttcggttgttttgatggaaaatacctaacaattgagttttttcatgttgttttgtcggagaatgcctacaattggagttcctttcgttgttttgatgagaaagcccctaatttgagtgtttttccgtcgatttgattttaaaggtttatattcgagtttttttcgtcggtttaatgagaaggcctaaaataggagtttttctggttgatttgactaaaaaagccatggaattttgtgtttttgatgtcgctttggttctcaaggcctaagatctgagtttctccggttgttttgatgaaaaagccctaaaattggagtttttatcttgtgttttaggttgttttaatccttataatttgagttttttcgttgttctgattgttgtttttatgaattttgcagaatggataacgcaacacttgctgtgatcctttccattttgtttgtgttttaccacattttcaaatcctttttcaccaattcttcatctcgtaggcttcctcctggtcccaaacccgtgccaatttttggcaacattttcgatcttggcgaaaagcctcatcgatcttttgccaatctatctaaaattcacggccctttgattagcctaaagttaggaagtgtaacaactattgttgtttcctcggcctctgtggccgaggaaatgttccttaaaaatgaccaagcacttgctaaccgaaccattcctgactcggttagggctggtgaccacgacaaattatccatgtcgtggttgcctgtttcccaaaaatggagaaatatgagaaaaatctccgctgtccaattactctccaaccaaaaacttgatgctagtcaacctcttagacaagctaaggtgaaacaacttttatcatacgtacaagtttgttccgaaaaaatgcaacccgtcgatattggacgggccgcatttacaacgtcacttaatttattatcaaacacatttttctcaatcgaattagcaagtcatgaatctagtgcttcccaagagtttaaacaactcatgtggaatattatggaggaaattggaaggcctaattatgctgattttttccctattcttggttacattgatccctttggtataagacgtcgtttggctggttactttgataaactcattgatgttttccaagacattattcgtgaaagacaaaagcttcgatcttctaattcttccggcgcaaaacaaacaaatgacattcttgatactcttcttaaactccatgaagataatgagttgagtatgcctgaaattaatcaccttctcgtggatatctttgacgccggaacagacacaacagcaagcacattagaatgggcaatggccgaacttgtgaaaaacccggaaatgatgactaaagttcaaattgaaatcgaacaagctcttggaaaagattgcttagacatacaagaatccgacatctcaaaactaccttatttacaagccattataaaagaaacgttacgtttacaccctcctactgtgtttttgctgcctcgaaaggcagacaatgacgtagagttatatggctacgttgtaccaaagaatgctcaagtccttgtcaatctttgggcaattggtcgtgatccaaaggtatggaaaaatccggaagtattttctcctgaaaggtttttagattgcaatatcgattataaaggacgagatttcgaacttttaccctttggtgctggtagaaggatatgccctggacttactttggcatatagaatgttgaacttgatgttggctactcttcttcaaaactacaattggaaacttgaagatggtatcaatcctaaggatttagacatggatgagaaatttgggattacattgcaaaaggttaaacctcttcaagttattccagttcccagaaactaggcttgttgtggttgtctggttgcgtctgttgcccgttgtctgttgcccattgtggtggttgtgtttgtatgatggtcgttaaggatcatcaatgtgttttcgctttttgttccattctgtttctcatttgtgaataataatggtatctttatgaatatgcagtttgtggtttcttttctgattgcagttctgagcattttgtttttgcttccgtttactataccacttacagtttgcactaatttagttgatatgcgagccatctgatgtttgatgattcaaatggcgtttatgtaactcgtacccgagtggatggagaagagctccattgccggtttgtttcatgggtggcggagggcaactcctgggaaggaacaaaagaaaaaccgtgatacgagttcatgggtgagagctccagcttgatcccttctctgtcgatcaaatttgaatttttggatcacggcaggctcacaagataatccaaagtaaaacataatgaatagtacttctcaatgatcacttatttttagcaaatcagcaattgtgcatgtcaaatgatttcggtgtaagagaaagagttgatgaatcaaaatatctgtagctggatcaagaatctgaggcagttgtatgtatcaatgatctttccgctacaatgatgttagctatccgagtcaaattgttgtagaattgcatacttcggcatcacattctggatgacataataaataggaagtcttcagatccctaaaaaattgagagctaataacattagtcctagatgtaactgggtgacaaccaagaaagagacatgcaaatactacttttgtttgaaggagcatccctggtttgacatattttttctgaatatcaaactttgaaactctacctagtctaatgtctaacgacagatcttactggtttaactgcagtgatatctactatcttttggaatgttttctccttcagttatacatcaagttccaagatgcaggtgtgcttgattgatgtacatggctgtgagaagtgcatcctgatgttcagatgatggttcattctaatgtcttttccttcaatcagttttctcagtctgacttagcttgtttcatctgcatgtttgaatgttcgtttactcatagtaattgcatttttgtagcagaacatatcattggtcatggtttcaactgtgcgcgagtcttatgcttattcaaactaggaaagcctccgtctagagggtacacgagttgttgctctgtgtgcgtcagtccatagtattaatcttgctagttgtagtatattgtttatgtggactcggaattcatcatatgctccttctttgcatcaagtaaggcaaggtaatgtatagaagctttttaactctttcatggaagctggcctttgccagcataccatccagaagatatcaaccctgcatcttggctgccg

SEQ ID NO: 26 - DNA, 16818 bp, synthetic construct
  misc_feature 1..16818: note = pDOPA3 overexpression vector
  source 1..16818: mol_type = other DNA
cgctgtcatgagacgaattctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatccgatgagtaaataaacaaatacgcaaggggaacgcatgaaggttatcgctgtacttaaccagaaaggcgggtcaggcaagacgaccatcggaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatagatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacataacgaattcgtctcaggagagcgtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgacgcactccttctttactccaccatctcgtccttattgaaaacgtgggtagcaccaaaacgaatcaagtcgctggaactgaagttaccaatcacgctggatgatttgccagttggattaatcttgcctttccccgcatgaataatattgatgaatgcatgcgtgaggggtagttcgatgttggcaatagctgcaattgccgcgacatcctccaacgagcataattcttcagaaaaatagcgatgttccatgttgtcagggcatgcatgatgcacgttatgaggtgacggtgctaggcagtattccctcaaagtttcatagtcagtatcatattcatcattgcattcctgcaagagagaattgagacgcaatccacacgctgcggcaaccttccggcgttcgtggtctatttgctcttggacgttgcaaacgtaagtgttggatcggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcccggatccgggcggaaataggtaaagaagttgcggataaggtaattgccattgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccctccgtcaggagactagagccaagctgatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctgatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaataccacaacaaaacaaacaacaaacaacattacaatttactattctagtcgaaatggataacgcaacacttgctgtgatcctttccattttgtttgtgttttaccacattttcaaatcctttttcaccaattcttcatctcgtaggcttcctcctggtcccaaacccgtgccaatttttggcaacattttcgatcttggcgaaaagcctcatcgatcttttgccaatctatctaaaattcacggccctttgattagcctaaagttaggaagtgtaacaactattgttgtttcctcggcctctgtggccgaggaaatgttccttaaaaatgaccaagcacttgctaaccgaaccattcctgactcggttagggctggtgaccacgacaaattatccatgtcgtggttgcctgtttcccaaaaatggagaaatatgagaaaaatctccgctgtccaattactctccaaccaaaaacttgatgctagtcaacctcttagacaagctaaggtgaaacaacttttatcatacgtacaagtttgttccgaaaaaatgcaacccgtcgatattggacgggccgcatttacaacgtcacttaatttattatcaaacacatttttctcaatcgaattagcaagtcatgaatctagtgcttcccaagagtttaaacaactcatgtggaatattatggaggaaattggaaggcctaattatgctgattttttccctattcttggttacattgatccctttggtataagacgtcgtttggctggttactttgataaactcattgatgttttccaagacattattcgtgaaagacaaaagcttcgatcttctaattcttccggcgcaaaacaaacaaatgacattcttgatactcttcttaaactccatgaagataatgagttgagtatgcctgaaattaatcaccttctcgtggatatctttgacgccggaacagacacaacagcaagcacattagaatgggcaatggccgaacttgtgaaaaacccggaaatgatgactaaagttcaaattgaaatcgaacaagctcttggaaaagattgcttagacatacaagaatccgacatctcaaaactaccttatttacaagccattataaaagaaacgttacgtttacaccctcctactgtgtttttgctgcctcgaaaggcagacaatgacgtagagttatatggctacgttgtaccaaagaatgctcaagtccttgtcaatctttgggcaattggtcgtgatccaaaggtatggaaaaatccggaagtattttctcctgaaaggtttttagattgcaatatcgattataaaggacgagatttcgaacttttaccctttggtgctggtagaaggatatgccctggacttactttggcatatagaatgttgaacttgatgttggctactcttcttcaaaactacaattggaaacttgaagatggtatcaatcctaaggatttagacatggatgagaaatttgggattacattgcaaaaggttaaacctcttcaagttattccagttcccagaaactaggcttgctctcaagatcaaaggcttaaaaagctggggttttatgaatgggatcaaagtttctttttttcttttatatttgcttctccatttgtttgtttcatttccctttttgttttcgtttctatgatgcacttgtgtgtgacaaactctctgggtttttacttacgtctgcgtttcaaaaaaaaaaaccgctttcgttttgcgttttagtcccattgttttgtagctctgagtgatcgaattgatgcctctttattccttttgttccctataatttctttcaaaactcagaagaaaaaccttgaaactctttgcaatgttaatataagtattgtataagatttttattgatttggttattagtcttacttttgctacctccatcttcacttggaactgatattctgaatagttaaagcgttacatgtcttccattcacaaatgaacttaaactagcacaaagtcagatattttaagatcgcaccattttatataaccccaatcgtcaattctactgtttcaagttttacaccaaaacaattacgaggtgtatctattcgttactcttttcgcttattaatccttttacatgatgcaaaatataacaaattaatttcaaattgtcctgcacatttggtttatacatcttgattccaaaaagtcgctgtcaggagatacatgagaattaagggagtcacggtatgacccccgccgatgacgcgggacaagccgttttacgtttggaactgacagaaccgcaacgttgaaggagccactgagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggcaatgatggctatggaagtgggttatcttcggcactcgacaacgatcaccaacggactgtgttgtaactgtgatccgaagccccgaggagcacgtcgtgtacagactaggctgcctggcaccctgtgtctcgtcaaggatacctttacctctagcaaagctaaactaaaaaagccgtcgcagagagaaatctttctaacaagtaggaagaggctgaatcagatacaagctgtttcaacggctgaaaaggagagagaggcagacaaaacatccactccacctattcccagtagcatccacgatatttcaaatggagatcacattcttggcttcggagccgatttgaccgaagatcatcctggctaccatgatcttgaatataagaggaggcgtagtcgtattgctgaccttgcgaagatacataaaataggtgaacccattccttgcgtggactacacatcggaagagattcgtgtgtggggtcatgtgttagatactctagtggacctctacccaactcatgcttgtaaggaatacctgaattgctacgagctgtttaacttcaagccaaactatattccacagctgcaagaactttcggaggtcctggaacggagcacaggatggcacatccgcccagtagctggactcctccaccctcgtgacttcctaaacggtctagccttccgcactttccattcaacccagtatgttcggcatggcagcaatcccatgtacacccccgagccagacatttgtcacgaggttttaggccacgtccccattctggctgatccggagtttgctgacttagcttgggccattggccaagcttctctgggtgcttcagagaaggatatctggcaccttacgaagctctactggtacactgtggagtttggcacagtaaaggaaggtaacgagattaaagcgttcggtgcaggccttctgtctagttttggggagctgaagcacatgcgggtggggacggatggattcatgcctgagttcgtggagcttgacccgtttaagaagatgcccaagatgagctacaaagacgggtaccagaagaggtattttctatgcgagagctttgcagatgcagcggcaaagcttagggcgtattccagaagcattctgaagcctgaggtgcagtccatcaagtttggagatacgccaatccggctctaagcttatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagcgctgtcaggagccgtacaaaattgattattgatgtcacaaatcaatcagagctaaattccaaaaaaaatatgatctttgaaacttgcatcaataactaacgattctaggaagagcccatttcgatttcaacaaaaagatgagagcttgattacatataaaatcaagagaaattaacgacattgaagagaattcgatctcgaaaccggaaacaaagacttataagtcgattagtctaattcaatgcagaagaaagaataccaggtttggccttcacgatggaaagcttcggatttggagccaagtctcataaacgccattgtggaagaaagtcttgagttggtggtaatgtaacagagtagtaagaacagagaagagagagagtgtgagatacatgaattgtcgggcaacaaaaatcctgaacatcttattttagcaaagagaaagagttccgagtctgtagcagaagagtgaggagaaatttaagctcttggacttgtgaattgttccgcctcttgaatacttcttcaatcctcatatattcttcttctatgttacctgaaaaccggcatttaatctcgcgggtttattccggttcaacattttttttgttttgagttattatctgggcttaataacgcaggcctgaaataaattcaaggcccaactgtttttttttttaagaagttgctgttaaaaaaaaaaaaagggaattaacaacaacaacaaaaaaagataaagaaaataataacaattactttaattgtagactaaaaaaacatagattttatcatgaaaaaaagagaaaagaaataaaaacttggatcaaaaaaaaaaacatacagatcttctaattattaacttttcttaaaaattaggtcctttttcccaacaattaggtttagagttttggaattaaaccaaaaagattgttctaaaaaatactcaaatttggtagataagtttccttattttaattagtcaatggtagatacttttttttcttttctttattagagtagattagaatcttttatgccaagtattgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcagaatggcttctatgatatcctcttcagctgtgactacagtcagccgtgcttctacggtgcaatcggccgcggtggctccattcggcggcctcaaatccatgactggattcccagttaagaaggtcaacactgacattacttccattacaagcaatggtggaagagtaaagtgcatgctgatgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcgatatgatcaccccacaatatctcgctgacctgatgagctggggcgcaattggcgcacgtaccaccgaatcgcaggtgcaccgcgaacaggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcggggaattcatcttttacccatacgatgttcctgactatgcgggctatccctatgacgtcccggactatgcaggatcctatccatatgacgttccagattacgctgctcagtaggcttatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataag

SEQ ID NO: 27 - DNA, 18647 bp, synthetic construct
  misc_feature 1..18647: note = pDOPA4 overexpression vector
  source 1..18647: mol_type = other DNA
cgctgtcatgagacgaattctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatccgatgagtaaataaacaaatacgcaaggggaacgcatgaaggttatcgctgtacttaaccagaaaggcgggtcaggcaagacgaccatcggaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatagatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacataacgaattcgtctcaggagagcgtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgacgcactccttctttactccaccatctcgtccttattgaaaacgtgggtagcaccaaaacgaatcaagtcgctggaactgaagttaccaatcacgctggatgatttgccagttggattaatcttgcctttccccgcatgaataatattgatgaatgcatgcgtgaggggtagttcgatgttggcaatagctgcaattgccgcgacatcctccaacgagcataattcttcagaaaaatagcgatgttccatgttgtcagggcatgcatgatgcacgttatgaggtgacggtgctaggcagtattccctcaaagtttcatagtcagtatcatattcatcattgcattcctgcaagagagaattgagacgcaatccacacgctgcggcaaccttccggcgttcgtggtctatttgctcttggacgttgcaaacgtaagtgttggatcggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcccggatccgggcggaaataggtaaagaagttgcggataaggtaattgccattgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccctccgtcaggaggtcaactaccccaatttaaattttatttgattaagatatttttatggacctactttataattaaaaatattttctatttgaaaaggaaggacaaaaatcatacaattttggtccaactactcctctctttttttttttggctttataaaaaaggaaagtgattagtaataaataattaaataatgaaaaaaggaggaaataaaattttcgaattaaaatgtaaaagagaaaaaggagagggagtaatcattgtttaactttatctaaagtaccccaattcgattttacatgtatatcaaattatacaaatattttattaaaatatagatattgaataattttattattcttgaacatgtaaataaaaattatctattatttcaatttttatataaactattatttgaaatctcaattatgattttttaatatcactttctatccatgataatttcagcttaaaaagttttgtcaataattacattaattttgttgatgaggatgacaagatttcggtcatcaattacatatacacaaattgaaatagtaagcaacttgattttttttctcataatgataatgacaaagacacgaaaagacaattcaatattcacattgatttatttttatatgataataattacaataataatattcttataaagaaagagatcaattttgactgatccaaaaatttatttatttttactataccaacgtcactaattatatctaataatgtaaaacaattcaatcttacttaaatattaatttgaaataaactatttttataacgaaattactaaatttatccaataacaaaaaggtcttaagaagacataaattctttttttgtaatgctcaaataaatttgagtaaaaaagaatgaaattgagtgatttttttttaatcataagaaaataaataattaatttcaatataataaaacagtaatataatttcataaatggaattcaatacttacctcttagatataaaaaataaatataaaaataaagtgtttctaataaacccgcaatttaaataaaatatttaatattttcaatcaaatttaaataattatattaaaatatcgtagaaaaagagcaatatataatacaagaaagaagatttaagtacaattatcaactattattatactctaattttgttatatttaatttcttacggttaaggtcatgttcacgataaactcaaaatacgctgtatgaggacatattttaaattttaaccaataataaaactaagttatttttagtatatttttttgtttaacgtgacttaatttttcttttctagaggagcgtgtaagtgtcaacctcattctcctaattttcccaaccacataaaaaaaaaataaaggtagcttttgcgtgttgatttggtacactacacgtcattattacacgtgttttcgtatgattggttaatccatgaggcggtttcctctagagtcggccataccatctataaaataaagctttctgcagctcattttttcatcttctatctgatttctattataatttctctgaattgccttcaaatttctctttcaaggttagaatttttctctattttttggtttttgtttgtttagattctgagtttagttaatcaggtgctgttaaagccctaaattttgagtttttttcggttgttttgatggaaaatacctaacaattgagttttttcatgttgttttgtcggagaatgcctacaattggagttcctttcgttgttttgatgagaaagcccctaatttgagtgtttttccgtcgatttgattttaaaggtttatattcgagtttttttcgtcggtttaatgagaaggcctaaaataggagtttttctggttgatttgactaaaaaagccatggaattttgtgtttttgatgtcgctttggttctcaaggcctaagatctgagtttctccggttgttttgatgaaaaagccctaaaattggagtttttatcttgtgttttaggttgttttaatccttataatttgagttttttcgttgttctgattgttgtttttatgaattttgcagaatggataacgcaacacttgctgtgatcctttccattttgtttgtgttttaccacattttcaaatcctttttcaccaattcttcatctcgtaggcttcctcctggtcccaaacccgtgccaatttttggcaacattttcgatcttggcgaaaagcctcatcgatcttttgccaatctatctaaaattcacggccctttgattagcctaaagttaggaagtgtaacaactattgttgtttcctcggcctctgtggccgaggaaatgttccttaaaaatgaccaagcacttgctaaccgaaccattcctgactcggttagggctggtgaccacgacaaattatccatgtcgtggttgcctgtttcccaaaaatggagaaatatgagaaaaatctccgctgtccaattactctccaaccaaaaacttgatgctagtcaacctcttagacaagctaaggtgaaacaacttttatcatacgtacaagtttgttccgaaaaaatgcaacccgtcgatattggacgggccgcatttacaacgtcacttaatttattatcaaacacatttttctcaatcgaattagcaagtcatgaatctagtgcttcccaagagtttaaacaactcatgtggaatattatggaggaaattggaaggcctaattatgctgattttttccctattcttggttacattgatccctttggtataagacgtcgtttggctggttactttgataaactcattgatgttttccaagacattattcgtgaaagacaaaagcttcgatcttctaattcttccggcgcaaaacaaacaaatgacattcttgatactcttcttaaactccatgaagataatgagttgagtatgcctgaaattaatcaccttctcgtggatatctttgacgccggaacagacacaacagcaagcacattagaatgggcaatggccgaacttgtgaaaaacccggaaatgatgactaaagttcaaattgaaatcgaacaagctcttggaaaagattgcttagacatacaagaatccgacatctcaaaactaccttatttacaagccattataaaagaaacgttacgtttacaccctcctactgtgtttttgctgcctcgaaaggcagacaatgacgtagagttatatggctacgttgtaccaaagaatgctcaagtccttgtcaatctttgggcaattggtcgtgatccaaaggtatggaaaaatccggaagtattttctcctgaaaggtttttagattgcaatatcgattataaaggacgagatttcgaacttttaccctttggtgctggtagaaggatatgccctggacttactttggcatatagaatgttgaacttgatgttggctactcttcttcaaaactacaattggaaacttgaagatggtatcaatcctaaggatttagacatggatgagaaatttgggattacattgcaaaaggttaaacctcttcaagttattccagttcccagaaactaggcttgttgtggttgtctggttgcgtctgttgcccgttgtctgttgcccattgtggtggttgtgtttgtatgatggtcgttaaggatcatcaatgtgttttcgctttttgttccattctgtttctcatttgtgaataataatggtatctttatgaatatgcagtttgtggtttcttttctgattgcagttctgagcattttgtttttgcttccgtttactataccacttacagtttgcactaatttagttgatatgcgagccatctgatgtttgatgattcaaatggcgtttatgtaactcgtacccgagtggatggagaagagctccattgccggtttgtttcatgggtggcggagggcaactcctgggaaggaacaaaagaaaaaccgtgatacgagttcatgggtgagagctccagcttgatcccttctctgtcgatcaaatttgaatttttggatcacggcaggctcacaagataatccaaagtaaaacataatgaatagtacttctcaatgatcacttatttttagcaaatcagcaattgtgcatgtcaaatgatttcggtgtaagagaaagagttgatgaatcaaaatatctgtagctggatcaagaatctgaggcagttgtatgtatcaatgatctttccgctacaatgatgttagctatccgagtcaaattgttgtagaattgcatacttcggcatcacattctggatgacataataaataggaagtcttcagatccctaaaaaattgagagctaataacattagtcctagatgtaactgggtgacaaccaagaaagagacatgcaaatactacttttgtttgaaggagcatccctggtttgacatattttttctgaatatcaaactttgaaactctacctagtctaatgtctaacgacagatcttactggtttaactgcagtgatatctactatcttttggaatgttttctccttcagttatacatcaagttccaagatgcaggtgtgcttgattgatgtacatggctgtgagaagtgcatcctgatgttcagatgatggttcattctaatgtcttttccttcaatcagttttctcagtctgacttagcttgtttcatctgcatgtttgaatgttcgtttactcatagtaattgcatttttgtagcagaacatatcattggtcatggtttcaactgtgcgcgagtcttatgcttattcaaactaggaaagcctccgtctagagggtacacgagttgttgctctgtgtgcgtcagtccatagtattaatcttgctagttgtagtatattgtttatgtggactcggaattcatcatatgctccttctttgcatcaagtaaggcaaggtaatgtatagaagctttttaactctttcatggaagctggcctttgccagcataccatccagaagatatcaaccctgcatcttggctgccgcgctgtcaggagatacatgagaattaagggagtcacggtatgacccccgccgatgacgcgggacaagccgttttacgtttggaactgacagaaccgcaacgttgaaggagccactgagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggcaatgatggctatggaagtgggttatcttcggcactcgacaacgatcaccaacggactgtgttgtaactgtgatccgaagccccgaggagcacgtcgtgtacagactaggctgcctggcaccctgtgtctcgtcaaggatacctttacctctagcaaagctaaactaaaaaagccgtcgcagagagaaatctttctaacaagtaggaagaggctgaatcagatacaagctgtttcaacggctgaaaaggagagagaggcagacaaaacatccactccacctattcccagtagcatccacgatatttcaaatggagatcacattcttggcttcggagccgatttgaccgaagatcatcctggctaccatgatcttgaatataagaggaggcgtagtcgtattgctgaccttgcgaagatacataaaataggtgaacccattccttgcgtggactacacatcggaagagattcgtgtgtggggtcatgtgttagatactctagtggacctctacccaactcatgcttgtaaggaatacctgaattgctacgagctgtttaacttcaagccaaactatattccacagctgcaagaactttcggaggtcctggaacggagcacaggatggcacatccgcccagtagctggactcctccaccctcgtgacttcctaaacggtctagccttccgcactttccattcaacccagtatgttcggcatggcagcaatcccatgtacacccccgagccagacatttgtcacgaggttttaggccacgtccccattctggctgatccggagtttgctgacttagcttgggccattggccaagcttctctgggtgcttcagagaaggatatctggcaccttacgaagctctactggtacactgtggagtttggcacagtaaaggaaggtaacgagattaaagcgttcggtgcaggccttctgtctagttttggggagctgaagcacatgcgggtggggacggatggattcatgcctgagttcgtggagcttgacccgtttaagaagatgcccaagatgagctacaaagacgggtaccagaagaggtattttctatgcgagagctttgcagatgcagcggcaaagcttagggcgtattccagaagcattctgaagcctgaggtgcagtccatcaagtttggagatacgccaatccggctctaagcttatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagcgctgtcaggagccgtacaaaattgattattgatgtcacaaatcaatcagagctaaattccaaaaaaaatatgatctttgaaacttgcatcaataactaacgattctaggaagagcccatttcgatttcaacaaaaagatgagagcttgattacatataaaatcaagagaaattaacgacattgaagagaattcgatctcgaaaccggaaacaaagacttataagtcgattagtctaattcaatgcagaagaaagaataccaggtttggccttcacgatggaaagcttcggatttggagccaagtctcataaacgccattgtggaagaaagtcttgagttggtggtaatgtaacagagtagtaagaacagagaagagagagagtgtgagatacatgaattgtcgggcaacaaaaatcctgaacatcttattttagcaaagagaaagagttccgagtctgtagcagaagagtgaggagaaatttaagctcttggacttgtgaattgttccgcctcttgaatacttcttcaatcctcatatattcttcttctatgttacctgaaaaccggcatttaatctcgcgggtttattccggttcaacattttttttgttttgagttattatctgggcttaataacgcaggcctgaaataaattcaaggcccaactgtttttttttttaagaagttgctgttaaaaaaaaaaaaagggaattaacaacaacaacaaaaaaagataaagaaaataataacaattactttaattgtagactaaaaaaacatagattttatcatgaaaaaaagagaaaagaaataaaaacttggatcaaaaaaaaaaacatacagatcttctaattattaacttttcttaaaaattaggtcctttttcccaacaattaggtttagagttttggaattaaaccaaaaagattgttctaaaaaatactcaaatttggtagataagtttccttattttaattagtcaatggtagatacttttttttcttttctttattagagtagattagaatcttttatgccaagtattgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcagaatggcttctatgatatcctcttcagctgtgactacagtcagccgtgcttctacggtgcaatcggccgcggtggctccattcggcggcctcaaatccatgactggattcccagttaagaaggtcaacactgacattacttccattacaagcaatggtggaagagtaaagtgcatgctgatgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcgatatgatcaccccacaatatctcgctgacctgatgagctggggcgcaattggcgcacgtaccaccgaatcgcaggtgcaccgcgaacaggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcggggaattcatcttttacccatacgatgttcctgactatgcgggctatccctatgacgtcccggactatgcaggatcctatccatatgacgttccagattacgctgctcagtaggcttatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataag

SEQ ID NO: 28 - DNA, 1050 bp, synthetic construct
  misc_feature 1..1050: note = Polynucleotide encoding E Coli AroG175 mutant
  source 1..1050: mol_type = other DNA
atgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcgatatgatcaccccacaatatctcgctgacctgatgagctggggcgcaattggcgcacgtaccaccgaatcgcaggtgcaccgcgaacaggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcggg

SEQ ID NO: 29 - DNA, 1185 bp, Physcomitrella patens
  source 1..1185: mol_type = other DNA
atggctatggaagtgggttatcttcggcactcgacaacgatcaccaacggactgtgttgtaactgtgatccgaagccccgaggagcacgtcgtgtacagactaggctgcctggcaccctgtgtctcgtcaaggatacctttacctctagcaaagctaaactaaaaaagccgtctcagagagaaatctttctaacaagtaggaagaggctgaatcagatacaagctgtttcaacggctgaaaaggagagagaggcagacaaaacatccactccacctattcccagtagcatccacgatatttcaaatggagaccacattcttggcttcggagccgatttgaccgaagatcatcctggctaccatgatcttgaatataagaggaggcgtagtcgtattgctgaccttgcgaagatacataaaataggtgaacccattccttgcgtggactacacatcggaagagattcgtgtgtggggtcatgtgttagatactctagtggacctctacccaactcatgcttgtaaggaatacctgaattgctacgagctgtttaacttcaagccaaactatattccacagctgcaagaactttcggaggtcctggaacggagcacaggatggcacatccgcccagtagctggactcctccaccctcgtgacttcctaaacggtctagccttccgcactttccattcaacccagtatgttcggcatggcagcaatcccatgtacacccccgagccagacatttgtcacgaggttttaggccacgtccccattctggctgatccggagtttgctgacttagcttgggccattggccaagcttctctgggtgcttcagagaaggatatctggcaccttacgaagctctactggtacactgtggagtttggcacagtaaaggaaggtaacgagattaaagcgttcggtgcaggccttctgtctagttttggggagctgaagcacatgcgggtggggacggatggattcatgcctgagttcgtggagcttgacccgtttaagaagatgcccaagatgagctacaaagacgggtaccagaagaggtattttctatgcgagagctttgcagatgcagcggcaaagcttagggcgtattccagaagcattctgaagcctgaggtgcagtccatcaagtttggagatacgccaatccggctctaa

SEQ ID NO: 30 - DNA, 1988 bp, Beta vulgaris
  source 1..1988: mol_type = other DNA
tcgatgtgttttcaagaagagcttgagcctacattctacaacaatatattgttcctctcttgcgcaattaagcccttaaacctgcaattatcttagaatttttgggtttctaatttattctctcctttaattagctagcttcttagattattcatatttttccagcattttcaaacaatggataacgcaacacttgctgtgatcctttccattttgtttgtgttttaccacattttcaaatcctttttcaccaattcttcatctcgtaggcttcctcctggtcccaaacccgtgccaatttttggcaacattttcgatcttggcgaaaagcctcatcgatcttttgccaatctatctaaaattcacggccctttgattagcctaaagttaggaagtgtaacaactattgttgtttcctcggcctctgtggccgaggaaatgttccttaaaaatgaccaagcacttgctaaccgaaccattcctgactcggttagggctggtgaccacgacaaattatccatgtcgtggttgcctgtttcccaaaaatggagaaatatgagaaaaatctccgctgtccaattactctccaaccaaaaacttgatgctagtcaacctcttagacaagctaaggtgaaacaacttttatcatacgtacaagtttgttccgaaaaaatgcaacccgtcgatattggacgggccgcatttacaacgtcacttaatttattatcaaacacatttttctcaatcgaattagcaagtcatgaatctagtgcttcccaagagtttaaacaactcatgtggaatattatggaggaaattggaaggcctaattatgctgattttttccctattcttggttacattgatccctttggtataagacgtcgtttggctggttactttgataaactcattgatgttttccaagacattattcgtgaaagacaaaagcttcgatcttctaattcttccggcgcaaaacaaacaaatgacattcttgatactcttcttaaactccatgaagataatgagttgagtatgcctgaaattaatcaccttctcgtggatatctttgacgccggaacagacacaacagcaagcacattagaatgggcgatggccgaacttgtgaaaaacccggaaatgatgactaaagttcaaattgaaatcgaacaagctcttggaaaagattgcttagacatacaagaatccgacatctcaaaactaccttatttacaagccattataaaagaaacgttacgtttacaccctcctactgtgtttttgctgcctcgaaaggcagacaatgacgtagagttatatggctacgttgtaccaaagaatgctcaagtccttgtcaatctttgggcaattggtcgtgatccaaaggtatggaaaaatccggaagtattttctcctgaaaggtttttagattgcaatatcgattataaaggacgagatttcgaacttttaccctttggtgctggtagaaggatatgccctggacttactttggcatatagaatgttgaacttgatgttggctactcttcttcaaaactacaattggaaacttgaagatggtatcaatcctaaggatttagacatggatgagaaatttgggattacattgcaaaaggttaaacctcttcaagttattccagttcccagaaactagctagtgggttgtgccgttgtgggctgtgggctgattgtgcacttttgtttcctctaattgttgttgcaaacttctctagaagggtattatcttttgtataaataaagcgaaagctacatgtcctattattaaattagtgtatactatattaagtaagtatgagcatatagtatccattattgtttttggttgaatcgtattaaactgataaatcaagtaaatgtctccaacctaagcattcaattattctgttagtcataacatatgtagagggtaagtatgaagaattaataacggcgtgatcatcctta

SEQ ID NO: 31 - DNA, 1500 bp, Beta vulgaris
  source 1..1500: mol_type = other DNA
atggataacgcaacacttgctgtgatcctttccattttgtttgtgttttaccacattttcaaatcctttttcaccaattcttcatctcgtaggcttcctcctggtcccaaacccgtgccaatttttggcaacattttcgatcttggcgaaaagcctcatcgatcttttgccaatctatctaaaattcacggccctttgattagcctaaagttaggaagtgtaacaactattgttgtttcctcggcctctgtggccgaggaaatgttccttaaaaatgaccaagcacttgctaaccgaaccattcctgactcggttagggctggtgaccacgacaaattatccatgtcgtggttgcctgtttcccaaaaatggagaaatatgagaaaaatctccgctgtccaattactctccaaccaaaaacttgatgctagtcaacctcttagacaagctaaggtgaaacaacttttatcatacgtacaagtttgttccgaaaaaatgcaacccgtcgatattggacgggccgcatttacaacgtcacttaatttattatcaaacacatttttctcaatcgaattagcaagtcatgaatctagtgcttcccaagagtttaaacaactcatgtggaatattatggaggaaattggaaggcctaattatgctgattttttccctattcttggttacattgatccctttggtataagacgtcgtttggctggttactttgataaactcattgatgttttccaagacattattcgtgaaagacaaaagcttcgatcttctaattcttccggcgcaaaacaaacaaatgacattcttgatactcttcttaaactccatgaagataatgagttgagtatgcctgaaattaatcaccttctcgtggatatctttgacgccggaacagacacaacagcaagcacattagaatgggcaatggccgaacttgtgaaaaacccggaaatgatgactaaagttcaaattgaaatcgaacaagctcttggaaaagattgcttagacatacaagaatccgacatctcaaaactaccttatttacaagccattataaaagaaacgttacgtttacaccctcctactgtgtttttgctgcctcgaaaggcagacaatgacgtagagttatatggctacgttgtaccaaagaatgctcaagtccttgtcaatctttgggcaattggtcgtgatccaaaggtatggaaaaatccggaagtattttctcctgaaaggtttttagattgcaatatcgattataaaggacgagatttcgaacttttaccctttggtgctggtagaaggatatgccctggacttactttggcatatagaatgttgaacttgatgttggctactcttcttcaaaactacaattggaaacttgaagatggtatcaatcctaaggatttagacatggatgagaaatttgggattacattgcaaaaggttaaacctcttcaagttattccagttcccagaaactag

SEQ ID NO: 32 - DNA, 2200 bp, Beta vulgaris
  source 1..2200: mol_type = other DNA
tactaatgtgcaaatgcataaatttaatttcagatactcttttttataaatattattatactgtgcattcttttaacccaaaaaaaaaactgtcttaatacaccatttaattcctttctctaggatccactatcacatgtagtatataaatataatcctatacagttcacgttatcaaacaccaaagcatcaaaagccttccacacttgtattattttggggtagttgatttgttagcgtgttatttgtgagatcatcatggatcatgcaacattagcaatgatactagccatttggttcatttcttttcatttcataaaattactttttagccaacaaactaccaaacttcttcctcctggtccaaaaccattgccaataattggtaacatcttagaagttggtaaaaaaccccatcgttcatttgctaatcttgctaaaattcacggccctttaatatcgttacgtctaggaagtgtaacaactattgttgtatcatcagcagatgtagctaaagaaatgttcttaaaaaaagaccaccctctttctaaccgtactattcctaattctgtcacggccggtgaccaccataaactcaccatgtcgtggttgcccgtttcgccgaaatggcggaattttcgtaagattacagccgtccatttgctttctcctcaaagacttgatgcttgccaaacctttcgccatgccaaggtgcaacagctttatgaatatgtacaagaatgtgcacaaaaaggccaagctgttgatattggcaaagctgcatttactacctcccttaatttgttatctaaactattcttttcagtggaattagcccaccataaatcacacacttctcaagagtttaaggaactaatatggaacattatggaagatattgggaaacctaattatgctgattattttcctattttaggctgtgttgatccatcaggtattcgtcgaagattagcatgtagttttgacaagttgattgcagtttttcagggtataatatgtgaaaggcttgcgcctgattcttcaactacaacaacaacgacgactgatgatgtgctagacgttcttcttcagctcttcaaacaaaatgagctcactatgggcgagattaaccatttgctcgtcgacatttttgatgctggaacagacactacatcaagtacttttgagtgggtcatgacagagttaattaggaatcctgaaatgatggaaaaggctcaagaagaaattaagcaagtattgggcaaagataaacaaattcaggaatcagacattattaacctaccttacttacaagccattatcaaagaaactttgcgactacatccaccaactgtatttcttttgcctcgtaaagccgacactgatgttgaactatatggttatattgtgcctaaagatgcacaaatacttgttaacttatgggctattggaagagatcctaatgcatggcaaaatgctgatattttttcgcccgaaagatttatagggtgtgaaattgatgtcaaaggaagagattttggactcttaccttttggagccggaagaaggatatgtcctgggatgaatttggccattagaatgttaactttgatgctagctactttacttcaattcttcaattggaagcttgaaggagacataagtccaaaagacttagacatggatgagaaatttgggattgcgttacaaaagacaaagcctttaaaacttattccaatacctaggtattgaaatttgttagacttacgtacaacaattattattgtttgtggttggttttgggttagcttcctgttcatgtttgtttgatgtgctcgattgaatttactacaaatcaacgaactccaattatatcttgtcaacttggagtaatggcttgggtttctcatctgtcatctcttttggttggctgtgcaagggagtgagcttgcagcttagcatccttcaagaagatttaattaataatatgtgtatgtgtatgtgtatgtgtggattgtatctcgtcattcattttgcttgttgtaatgattttagttaattattgtaattataaaggatttgatcctcctttgtgttgaaattatatatataatgtgtagcacgacaataaagtgacagataacttatataatcttattttctatgaaatgttacagacttgctttgtgttttaaaaaaa

SEQ ID NO: 33 - DNA, 1161 bp, Beta vulgaris
  source 1..1161: mol_type = other DNA
aaaacaagaagaaaacaaaacaaccttttatataactagaaagcaacaaaaaaaaaagaatgaaaatgatgaatggtgaagatgctaatgatcaaatgatcaaagaaagcttcttcataacacatggaaacccaatattaacagtagaagacacacatccattaagacctttctttgaaacttggagagagaaaatcttttctaagaaacctaaggcaattcttattatttctggtcattgggaaactgttaaacctactgttaatgctgtccatatcaatgatactatccatgattttgatgactatcctgctgctatgtaccagttcaagtatccagctcctggggaaccagaattggcaagaaaagtagaggaaattctgaaaaaatcgggtttcgaaacggcggaaactgatcaaaaacgtgggcttgatcatggtgcatgggtacctctaatgctaatgtatcctgaggctgatataccagtatgtcagctctcagttcagccgcatttagatggaacataccattacaacttaggacgagcattggctcccttgaaaaacgacggcgtattaatcattggttcaggaagtgcaactcaccctttggatgaaactcctcattattttgatggagttgcaccttgggcagctgcctttgattcatggcttcgtaaagctctcattaatggaaggtttgaagaagtgaatatatatgaaagcaaagcaccaaattggaaattagcacatcctttcccagaacatttttatccattgcatgttgttcttggcgctgctggtgaaaaatggaaggcagagcttattcatagcagttgggatcatggcaccttgtgtcatggctcctacaagttcacttcagcctagttttacttttaaacactatgtctacagtctatgttattgtatttggtaatttggtatgtggtgttgtttttgttttattcttttgagttattgtacttggtatttggtgttgttttcatttggtgcataattcttttagtctatatattgctattcttttgaatgaggaataaatcgcgagctcgatgtaatagtttgttttattgtttcaaatctatcattatatatatatatatattattaaaaaaaaattattgtgttactcgcaaaaaaa

SEQ ID NO: 34 - protein, 499 aa, Beta vulgaris
MDNATLAVILSILFVFYHIFKSFFTNSSSRRLPPGPKPVPIFGNIFDLGEKPHRSFANLSKIHGPLISLKLGSVTTIVVSSASVAEEMFLKNDQALANRTIPDSVRAGDHDKLSMSWLPVSQKWRNMRKISAVQLLSNQKLDASQPLRQTKVKQLLSYVQDCSKKMQPVDIGRAAFTTSLNLLSNTFFSIELASHESSASQEFKQLMWNIMEEIGRPNYADFFPILGYIDPFGIRRRLAGYFDKLIDVFQDIIRERQKLRSSNSSGAKQTNDILDTLLKLHEDNELSMPEINHLLVDIFDAGTDTTASTLEWAMAELVKNPEMMTKVQIEIEQALGKDCLDIQESDISKLPYLQGIIKETLRLHPPTVFLLPRKADNDVELYGYVVPKNAQVLVNLWAIGRDPKVWKNPEVFSPERFLDCNIDYKGRDFELLPFGAGRRICPGLTLAYRMLNLMLATLLQNYNWKLEDGINPKDLDMDEKFGITLQKVKPLQVIPVPRN

SEQ ID NO: 35 - DNA, 1799 bp, Mirabilis jalapa
  source 1..1799: mol_type = other DNA
tttgtctattattggctcaaaatccctctctctatctttttgtaaaagaaaatagttgttcaacttagggaattattgatatttcattatggaaaacacaatgttaggtgttatcctagcaaccattttcctcacttttcacataatgaagatgttatttagtccttccaaggttaaactacccccgggtccgagaccattgccaattattggtaatattctcgagcttggggataaaccacatcgttcttttgcaaaccttgcgaaaattcacggtcctttagttactttgaaactcgggagtgtaaccactattgtggtttcctcttctgaagttgctaaagaaatgtttttgaaaaatgaccaacctttggcaaatcgtaccatacctgactcagtaagagcaggtaaccatgacaaactatcaatgtcgtggttgcctgtatcacccaaatggcgaaatcttagaaagatttcagccgtccaattgctctcaactcaacgacttgatgcaagtcaagctcatagacaagctaaaatcaaacaacttattgagtacgtaaaaaaatgcagtaaaatcggccaatacgtcgatattggccaagttgcattcactacatcacttaatttactatcaaacacattcttttcaaaagaactagcatcatttgattcagataatgcacaagagttcaaacaactaatgtggtgcattatggaagaaattggtaggcctaattatgccgattattttcctatcttgggttatgtcgatccattcggtgctagacgtcgactttctcgttacttcgatcaactaattgaagtatttcaagtgattattcgtgagagacttacacatgataataatattgtgggtaataacaatgatgttttagctacgttgctcgatctttataaacaaaacgagttaactatggatgaaatcaaccatttactagtggacatttttgatgctggtacggatacaacagcaagtacactagaatgggcaatgtcagagctcataaaaaatccacacataatggccaaagctcaagaggaggtccggcgagccaccatgtctcacggcggagctacggtggcggaaatacaagaatcggatatcaataatcttccatacatacaatctattattaaagaaacacttcgtttacacccaccaactgtgtttttacttcctagaaaagctgacgtggatgtccaattattcggctatgtggtccccaaaaatgctcaagtcctagtcaatttatgggccattggtcgtgacccaaatgtgtggcccgacccggaagtttttagtcccgaaagatttatggattgtgagattgatgtcaagggtcgtgattttgagctattgcctttcggggcgggtcgtcggatttgtccgggattgtctttggcttatcggatgcttaatttgatgttggctaatatggtacattcttttgattggaaattacccggtgttgaaaatggatccgggtcggaaatggatagtttggatatggatgagaaatttggtatcactttgcaaaaggttcaaccccttaaggttattcctgtctcaaggaaatagatttttggaagatcgatggttgggagataatcatattgtttttaattattgttggtgtattttattgttattattggggacaattttataataaaagatataaataagttcctaattgtgatttatattgtgaattttctagttaatataaatacatatgaatatttatgatgaaaaaaaaaaaaaaaaaaaaagg

SEQ ID NO: 36 - DNA, 2130 bp, Mirabilis jalapa
  source 1..2130: mol_type = other DNA
tacgtctattcttgcctcccatttcctccctgcaaaaaggaagaaaatggaaataccctttaatttgcaacctatttttctaattattgtactatctaccttcattctaacccacctaataacaaaaaacacaagaaagttcaaccaatgcaaatcaaaaaccaagccaataccaaaaccacctggctcactacccataataggtcacttgcacctcctaagaggtcatggaaccaccctaggtcgcaccctcggagccatggccgacaagtccggacccattttcggcctcaagctaggccaacacaaggctatagtcattagtaactgggagatagtcaaggattgtttcaccaataatgacatgaaccttgcaactagacctagcatggctattagcaaatatattggttatgactttgcctctttctctatctcaccttatggccgatattggcgtgagattcgtaagatctctactgttgagctcttgtcaaacaaacgccttgagaaaatgaagcatgttcgaacttttgaggtgaactcttgccttcaaatcttctatgaaaaatgtgtcaaatcagaggataatgaaagtaataaagtagcaaaaatttgtcttaaaaagtggttagaatatattggttttaatatcgcactatctatgatagttggcaagagattctcctacgaccagtattataaaaaaaatactgttgcatcgagatttatgaaggcaatagaaagagcaacccacttagcaggacttcctataccctcggattttttaccatgggttgaatggatggatttggtgggttacattggtgctatgaaggaaactcataaggaaattgatgtgattattggacattggcttgatgagcatattcaaaagaggaaagagacgttagaaaggcgaggtacaagcattgatgatagtgacttcatggatgtaatgttatctaacattactgaacaatttgccaagtcaacctactctaaaaacaccatcattaaagcaacagctttgacactacttctaacaggttcagaaagcacctcaatcacactaacatgggccatatcactacttctaaacaaccctacggcccaaaatttggcccaacaagaaatggacaaacaagttggtaaacaaagatgggttgaagaatctgacatcaccaatctaccatacctccaagccatcataaaggaaactttacgtttgtatccaccaagcccattagcaggcccaagagtggcccaagaagactgccaaatcaatggacattgtatcacaaaaggaacccgggtgattgttaacatatggaagttgcatcgtgatccgatggtttggtccgacccagataagttccgacccgaaaggtttattggggagcataaggatattgattttaagggtcaatactttgggtatataccttttagcttgggtaggaggggttgtcctgggatgaattttgggctacaagtggtccatttgatgttagctcgtttggtccaagggttcgatatttgggctcaaaatggtggcctaattgatttgaaggaagggttgggcttggctttgcccaaggctggtccattggatgtcattcttgcaccacgtttgtccagtgagctttatggagctttatcaaaggtgaatgttacttatggtcgggcaatgaatgaactagctttgtatttataattttacaaataatttgtaagatatgaactgtttatctagtcacttttccgcgtcttagaatatcaataagcaaaaagttttatgtaataatatgtattagtgtgttattgtatcatgattcgtcggtatactgaaaatattttacgtctaggagaatgtgcacacatttaatcttatttgcttataagtattatatacataaaattttctactttataatttaggtatattgagctttcgaccaaaacccaaaaactaaagaagttttttttaaaacatacaagttggtattgtttagatttttatttatttttattatctcttttatttgtacttagtgggtgtgttaataatgtaaagaagtattaaatatttatagagttggaaaaacccaatattttacattggaaaaa

SEQ ID NO: 37 - DNA, 1903 bp, Mirabilis jalapa
  source 1..1903: mol_type = other DNA
tacgtctctaacctccttctcctcaccatcatcatcttatttctctctctaacttcctatttctctctctagaatggtctcaactagtgactcactctggcttttcacctacttagcttcaaaatatgatcattttacgacccctaactttatttattctttgttttgtttcgtaatattttttattttgattcatctactgtactggtcccacccaggtggtccggcatgggggaagtattcatggactcatctctcgggatgggccgggaagcccatcccgggcccaagaagttggcccattataggtggtttggacctgaagatgggactggctcatcggaagttagccacaatggctgagaaatacgggaccatggctaagaggctcatggcttttagcctaggagaaacacgcgtgattgtcacgtgcaatcccgacgtggcgaaagaaatactaaatagcccggttttcttggaccgacccgaccaggagtcggcttatgggttgatgtttaaccggtctatagggttcgcaccctatgggacctactggcgaaccctaaggaaaataacatcaacccatttattaagccctaaacacgtaaaagaatatgagaacctacgtagggaaataatggaacaaatggttgactcgatatcgaaccaagccggaccggttcgggtccgggaaatactaaggagggcttcattggataatatgatggggtcagtgtttggtgggccccacaaggaggtcgttgaacagttgatagaaatggttggtgaagggtatgacttattgggtgtccttaatttgggggaccatctatcttggctagccgagtttgactttcaaaaggttcggttcaggtgctctcaacttgttccaaaagtcaaccggttcgttggtgaaattatcgatgaacatcggtctagttcgggtcaaattatgaaccgggactttgttgatgttttgttgactctaccacaacatgaacaactttctcattctgacatggtcgctgttctttgggagatgatatttagaggaacagatgcaacagcagtattaatagaatggacattagcacgattagtaattcataaagatattcaatcaaaggtccaaaatgagttagaccaagtagttggaacatcacgagccgttgaagagtctgacctatcatcactgatatatctaacggctgtgattaaagaagtattgcgggtccacccacctgggcccctcctatcgtggtctcgcctttcaattcaagatactactatagatggttgtcatgtgccaaaaggtactactgctatggttaacatgtgggctattgctagagatcctaatgtgtgggccaacccagatgaatttgacccggatagatttttaatcggtgggtccgagtttgagttctctgttcttgggtcggatcttagacttgcccctttcgggtcgggtcgtaggtcatgtcctgggaaggtcttaggtttgaccacagtcagtttttgggttgcttcactcttgcatgagtttgagtgggtgacatcacctaacgctgacgtggatttgagtgaagtgcttaagctttcgtgtgagatggctcatcctcttaccgtggaagctaggccgcgacgtcattaattttacaaaagacgatattctcatacacagattattgtcaactttatcaatatatacgggtgatagtttgacattgttatagtattcggaaaaatattttctagagataagtaaatttataataatctaaaaaactgaatgtataattatattaattaatgtactaatgttattataatgtttataaatgattgtgaaatatatatggttaacattatatgtaatgagaaatggtttgcttggcaaaaa

SEQ ID NO: 38 - DNA, 1915 bp, Mirabilis jalapa
  source 1..1915: mol_type = other DNA
gctagctgccttgttcatttcaagaacaatatacactagtcaatccatttcaaatggcctacttagaaattgtcatattcttcatttttctcatcgtaatttgtttttcttttcgtgacaaaaatggtctcccaacaaattggccgattgttgggatgctaccagctgttctaatcaatcttcatagaattaatgactattttgctgaacttgttgctaaatcaaatttaacattctcatttaagggtccttggtttagcaacatgcgaattttagcgacagttgatccagcaaatgtgcaccatataatgagtaagaattttaacaattatcctaagggtgctaagttttacgacatctttgatatacttggagatggtatttttaacaccgactttaatatatggcaataccatcggaaaatggctcagtcatatattggtgactcaagatttcaacaatttttgttgaagaaagttggggaaaagattgaaggtggattaattcctatccttgatcatgttgccatgcaagggttacaagttgatttacaagatttatttgaaaggtttacttttgatactatttgttcacttattatgaactatgatcctatgtctttgtccattgattttcctgatgttccatcatctaaggctctagatgttgctgaagaagtcatacttcttcgtcatttagtacctacaagtgtttggaagtttcaaagatggttaggtgtagggatggaaaagaagcataagatagcttggcaagtacttgatgatttcatttatgagtgcatatcaaggaaaagagaagagatgagagatagtttgtcctatgagaataaggacaatgagatgggtgttgatttaatgacattgtatatgaatgaaatcaaaagtaatgaacttgtcaaggatgatcctaacaagtttttgagggatactattcttaacttttttattgcaggtcgagatacaactagtactgctttatcgtggtttttctatctactatccaagaatccacaagtggtagaaaagattagacaagagttatcttggatcgtttcacaagaaaaaactaaaaattatgctaatttgattgataaacttgtttatcttcatgccgcattatgtgaagctttaagattatatcctccggtggtatttgaagcaaagtctccgattgaatcggacactttaccaagtggtcataagattgatcctgacacacaaataatcttaaatatgtacgcaatggcgaggatgaaaacaatatggggcgatgattgcgatcaatttaaacctgagaggtggatatcatcaacaacaggaaagattaagcatgaaccttcgtacaagttcttggcttttaatgcaggaccaagaacttgtgtaggaaaaaatatggcttttactcaaatgaaagcaatagcaatagctatattacaaaactatcatatacatggtattgatggacaagttattgaacctgatctatccataattctccatatgaagaatggattcagagtaactgtttcaccgtgtaacgtttctatataaccatcgagagaaccaagtttcctcgatctcttttaaacattgtaatgtttaattatcaactaaaaaatcgtatattctcttcgtttcatttttttctttttctttttatcaaagatcaaggtaacgtcggaaataaccatattgttttctttggttaaatgaaaagacaatgtaagttgtaaaagtatgtaagtgtaaaaataaatttaaaaaattgtgtgaaaataagttgtaataaataaagaataatgttggtattttattgtttactattttgataaacagaaaaaataaaataaaactgtttaaaaataaaagtgagaagaaaaaaaataaaagaaaat

SEQ ID NO: 39 - DNA, 817 bp, Portulaca grandiflora
  source 1..817: mol_type = other DNA
atgggtgttgggaaggaagtgtcgtttaaggagagtttcttcttgtctcatgggaatccagccatgttggcggatgaatcctttatagcgaggaacttccttttgggatggaaaaagaatgtgtttcctgtgaaacccaagtccattttagtagtctctgctcactgggagactgatgtcccttgtgtatctgctggtcagtaccctaatgttatctacgatttcactgaagtccctgcttccatgtttcagatgaagtacccagctccagggtgcccaaagctggcaaaaagagtgcaggaactgctgatagcgggaggtttcaagagcgcgaagctagatgaggagcggggtttcgaccacagctcatgggtgccactgagcatgatgtgcccggaggcggacatcccggtgtgccagctttcggtgcagcctgggcttgacgcgacccaccacttcaacgtggggcgagcgttggccccactcaaaggggaaggtgtcctcttcattggctccggtggggctgtccacccttctgatgacacccctcattggttcgatggtgttgctccctgggctgctgagtttgatcaatggcttgaggatgctctcctagaaggaaggtacgaagatgtgaataactatcaaacaaaagcaccagaagggtggaagctagcacatccaattccagaacattttctaccattgcatgtagccatgggtgcaggtggtgagaaatcaaaggcagagcttatttatcgtacttgggatcatggtactcttggctacgcctcttacaagttcacttccatatgat

SEQ ID NO: 40 - DNA, 1078 bp, Bougainvillea glabra
  source 1..1078: mol_type = other DNA
atgggtggggagaaaaaaatgaagggaacatactatatagcacatggtgatccaattatgtacatcaataaatcaataaaacttaggcactttttagaagagtggaaagagaatgtggttatggagaaacctatatgtattcttgtgatttcagctcattgggatactgatgttcccactgttaaccttgttgaacattgtgataccatccatgattttgatgactatcctgatcccttgtaccagataaagtatccagctcctggggctccaaaattggcaatgaaagtgcaagagttattgaaaggtggaggattcaaatgtgaagtggatacaaagcgtggacttgaccatgctgcatggtttccactcatgttgatgtatcctgaagctgatattccaatttgtgagctctcagttcaaaccaacaaggatgggacccaccactacaaccttggaaaagcactttctcctctcttgaatgatgatgttctcatcattggctctggtggagctgttcatccctcagatgatactcctcattgtcctaatggtgttgctccttgggctttgcagtttgacaattggcttgaagatgctcttctcagtggaaggtatgaagatgtgaaagaattcaaaaaaatggcaccaaattgggagatatcacatccagggcaagagcacttgtaccctttgcatgtggctcttggggctgcaggcaacaacgtaaagacagagcttattcatcaaacttgggctgccaatggcgtctttggatattcctcctacaagttcacatccacttgaacaataaattaaatattcatctttacacatttcaataattcccttcttgtgtttgctttttctggtattaaatttgtttgtcatttttttttagagtcgacagttgagggtctttttaaaacaattttttttatctttatggcgagagtgacatccttgtaaaattcatttattatggtgtatttggatactagtatttcatttgagatgcatgaattatgttaaattcactgtttgaataacaataaatttataaatttgaatttgactc

SEQ ID NO: 41 - protein, 228 aa, Amanita muscaria
MVPSFVVYSSWVNGRQRYIRQAFASILFYIIRDTTLSFPSHTTMSTKPETDLQTVLDSEIKEWHFHIYFHQNNAAEHQAALELRDAVLRLRQDGAFVAVPLFRVNMDPMGPHPVGSYEIWVPSETFASVFSYLCMNRGRLSILVHPLTREELRDHEIRNAWIGPSFPLNLANLPIKSDEIPLQYPSLKLGYSSTAHKMSLEERRKLGDDIEAVLRGEKEAARAPHRDA

SEQ ID NO: 42 - protein, 275 aa, Beta vulgaris
MKMMNGEDANDQMIKESFFITHGNPILTVEDTHPLRPFFETWREKIFSKKPKAILIISGHWETVKPTVNAVHINDTIHDFDDYPAAMYQFKYPAPGEPELARKVEEILKKSGFETAETDQKRGLDHGAWVPLMLMYPEADIPVCQLSVQPHLDGTYHYNLGRALAPLKNDGVLIIGSGSATHPLDETPHYFDGVAPWAAAFDSWLRKALINGRFEEVNIYESKAPNWKLAHPFPEHFYPLHVVLGAAGEKWKAELIHSSWDHGTLCHGSYKFTSA

SEQ ID NO: 43 - protein, 500 aa, Mirabilis jalapa
MTAIKMNTNGEGETQHILMIPFMAQGHLRPFLELAMFLYKRSHVIITLLTTPLNAGFLRHLLHHHSYSSSGIRIVELPFNSTNHGLPPGIENTDKLTLPLVVSLFHSTISLDPHLRDYISRHFSPARPPLCVIHDVFLGWVDQVAKDVGSTGVVFTTGGAYGTSAYVSIWNDLPHQNYSDDQEFPLPGFPENHKFRRSQLHRFLRYADGSDDWSKYFQPQLRQSMKSFGWLCNSVEEIETLGFSILRNYTKLPIWGIGPLIASPVQHSSSDNNSTGAEFVQWLSLKEPDSVLYISFGSQNTISPTQMMELAAGLESSEKPFLWVIRAPFGFDINEEMRPEWLPEGFEERMKVKKQGKLVYKLGPQLEILNHESIGGFLTHCGWNSILESLREGVPMLGWPLAAEQAYNLKYLEDEMGVAVELARGLEGEISKEKVKRIVEMILERNEGSKGWEMKNRAVEMGKKLKDAVNEEKELKGSSVKAIDDFLDAVMQAKLEPSLQ

SEQ ID NO: 44 - protein, 504 aa, Mirabilis jalapa
MENTMLGVILATIFLTFHIMKMLFSPSKVKLPPGPRPLPIIGNILELGDKPHRSFANLAKIHGPLVTLKLGSVTTIVVSSSEVAKEMFLKNDQPLANRTIPDSVRAGNHDKLSMSWLPVSPKWRNLRKISAVQLLSTQRLDASQAHRQAKIKQLIEYVKKCSKIGQYVDIGQVAFTTSLNLLSNTFFSKELASFDSDNAQEFKQLMWCIMEEIGRPNYADYFPILGYVDPFGARRRLSRYFDQLIEVFQVIIRERLTHDNNIVGNNNDVLATLLDLYKQNELTMDEINHLLVDIFDAGTDTTASTLEWAMSELIKNPHIMAKAQEEVRRATMSHGGATVAEIQESDINNLPYIQSIIKETLRLHPPTVFLLPRKADVDVQLFGYVVPKNAQVLVNLWAIGRDPNVWPDPEVFSPERFMDCEIDVKGRDFELLPFGAGRRICPGLSLAYRMLNLMLANMVHSFDWKLPGVENGSGSEMDSLDMDEKFGITLQKVQPLKVIPVSRK

SEQ ID NO: 45 - DNA, 24 bp, synthetic construct
  misc_feature 1..24: note = BvDODA1 Fwd + XhoI
  source 1..24: mol_type = other DNA
tgtgctagacgttcttcttcagct

SEQ ID NO: 46 - DNA, 20 bp, synthetic construct
  misc_feature 1..20: note = BvDODA1 Rev + SacI
  source 1..20: mol_type = other DNA
aaaatgtcgacgagcaaatg

SEQ ID NO: 47 - DNA, 22 bp, synthetic construct
  misc_feature 1..22: note = CYP76AD1 Fwd + XhoI
  source 1..22: mol_type = other DNA
cttcatctcgtaggcttcctcc

SEQ ID NO: 48 - DNA, 20 bp, synthetic construct
  misc_feature 1..20: note = CYP76AD1 Rev + SacI
  source 1..20: mol_type = other DNA
gatgaggcttttcgccaaga

SEQ ID NO: 49 - DNA, 20 bp, synthetic construct
  misc_feature 1..20: note = CYP76AD1 Fwd + BamHI
  source 1..20: mol_type = other DNA
cctgtatcggctaaatggcg

SEQ ID NO: 50 - DNA, 21 bp, synthetic construct
  misc_feature 1..21: note = CYP76AD1 Rev + EcoRI
  source 1..21: mol_type = other DNA
caattgcacagcggagatttt

SEQ ID NO: 51 - DNA, 20 bp, synthetic construct
  misc_feature 1..20: note = BvCYP76new Fwd + XhoI
  source 1..20: mol_type = other DNA
ttgatgctgtttgcgcagtc

SEQ ID NO: 52 - DNA, 20 bp, synthetic construct
  misc_feature 1..20: note = BvCYP76new Rev + SacI
  source 1..20: mol_type = other DNA
tgcgaagatttcgccatttt

SEQ ID NO: 53 - DNA, 23 bp, synthetic construct
  misc_feature 1..23: note = CYP76AD6 Fwd + XhoI
  source 1..23: mol_type = other DNA
tggtgctgattttcgtcgtagag

SEQ ID NO: 54 - DNA, 19 bp, synthetic construct
  misc_feature 1..19: note = CYP76AD6 Rev + SacI
  source 1..19: mol_type = other DNA
tggcaccacccttcaagtg

SEQ ID NO: 55 - DNA, 20 bp, synthetic construct
  misc_feature 1..20: note = BvDODA1 Fwd
  source 1..20: mol_type = other DNA
taaagctccctcctggtcca

SEQ ID NO: 56 - DNA, 20 bp, synthetic construct
  misc_feature 1..20: note = BvDODA1 Rev
  source 1..20: mol_type = other DNA
ccgcagaacgatgaggtttg

SEQ ID NO: 57 - DNA, 23 bp, synthetic construct
  misc_feature 1..23: note = CYP76AD1 Fwd
  source 1..23: mol_type = other DNA
tcaaactcggaagcatcactaca

SEQ ID NO: 58 - DNA, 20 bp, synthetic construct
  misc_feature 1..20: note = CYP76AD1 Rev
  source 1..20: mol_type = other DNA
gggctaagtcgtgctcgagg

SEQ ID NO: 59 - DNA, 35 bp, synthetic construct
  misc_feature 1..35: note = CYP76AD6 Fwd1
  source 1..35: mol_type = other DNA
aaaaactcgaggttaaacctactgttaatgctgtc

SEQ ID NO: 60 - DNA, 31 bp, synthetic construct
  misc_feature 1..31: note = CYP76AD6 Rev1
  source 1..31: mol_type = other DNA
aaaaagagctcgctgcccaaggtgcaactcc

SEQ ID NO: 61 - DNA, 34 bp, synthetic construct
  misc_feature 1..34: note = CYP76AD6 Fwd2
  source 1..34: mol_type = other DNA
aaaaactcgaggctaatcttgctaaaattcacgg

SEQ ID NO: 62 - DNA, 33 bp, synthetic construct
  misc_feature 1..33: note = CYP76AD6 Rev2
  source 1..33: mol_type = other DNA
aaaaagagctcttatggtgggctaattccactg

SEQ ID NO: 63 - DNA, 34 bp, synthetic construct
  misc_feature 1..34: note = cDOPA5GT Fwd1
  source 1..34: mol_type = other DNA
aaaaaggatccgctaatcttgctaaaattcacgg

SEQ ID NO: 64 - DNA, 33 bp, synthetic construct
  misc_feature 1..33: note = cDOPA5GT Rev1
  source 1..33: mol_type = other DNA
aaaaagaattcttatggtgggctaattccactg

SEQ ID NO: 65 - DNA, 32 bp, synthetic construct
  misc_feature 1..32: note = cDOPA5GT Fwd2
  source 1..32: mol_type = other DNA
aaaaactcgagctgcaagagtcaatgatctca

SEQ ID NO: 66 - DNA, 33 bp, synthetic construct
  misc_feature 1..33: note = cDOPA5GT Rev2
  source 1..33: mol_type = other DNA
aaaaagagctctcatctgggaattggaattgct

SEQ ID NO: 67 - DNA, 32 bp, synthetic construct
  misc_feature 1..32: note = BvDODA1 Fwd
  source 1..32: mol_type = other DNA
aaaaactcgagcacaacagcaagcacattaga

SEQ ID NO: 68 - DNA, 33 bp, synthetic construct
  misc_feature 1..33: note = BvDODA1 Rev
  source 1..33: mol_type = other DNA
aaaaagagctcagtccagggcatatccttctac

SEQ ID NO: 69 - DNA, 41 bp, synthetic construct
  misc_feature 1..41: note = CYP76AD1 Fwd
  source 1..41: mol_type = other DNA
gcgccgtctcgctcgaatgaaaatgatgaatggtgaagatg

SEQ ID NO: 70 - DNA, 40 bp, synthetic construct
  misc_feature 1..40: note = CYP76AD1 Rev
  source 1..40: mol_type = other DNA
gcgccgtctcgctcgaagcctaggctgaagtgaacttgta

SEQ ID NO: 71 - DNA, 40 bp, synthetic construct
  misc_feature 1..40: note = CYP76AD6 Fwd
  source 1..40: mol_type = other DNA
gcgccgtctcgctcgaatggatcatgcaacattagcaatg

SEQ ID NO: 72 - DNA, 43 bp, synthetic construct
  misc_feature 1..43: note = CYP76AD6 Rev
  source 1..43: mol_type = other DNA
gcgccgtctcgctcgaagctcaatacctaggtattggaataag

SEQ ID NO: 73 - DNA, 37 bp, synthetic construct
  misc_feature 1..37: note = Synthetic
  source 1..37: mol_type = other DNA
gcgccgtctcgctcgaatggataacgcaacacttgct

SEQ ID NO: 74 - DNA, 32 bp, synthetic construct
  misc_feature 1..32: note = Synthetic
  source 1..32: mol_type = other DNA
gcgccgtctcgttgcccattctaatgtgcttg

SEQ ID NO: 75 - DNA, 31 bp, synthetic construct
  misc_feature 1..31: note = Synthetic
  source 1..31: mol_type = other DNA
gcgccgtctcggcaatggccgaacttgtgaa

SEQ ID NO: 76 - DNA, 40 bp, synthetic construct
  misc_feature 1..40: note = Synthetic
  source 1..40: mol_type = other DNA
gcgccgtctcgctcgaagcctagtttctgggaactggaat

SEQ ID NO: 77 - DNA, 39 bp, synthetic construct
  misc_feature 1..39: note = Synthetic
  source 1..39: mol_type = other DNA
gcgccgtctcgctcgaatgaccgccattaaaatgaacac

SEQ ID NO: 78 - DNA, 33 bp, synthetic construct
  misc_feature 1..33: note = Synthetic
  source 1..33: mol_type = other DNA
gcgccgtctcggcctcatttcctcattgatatc

SEQ ID NO: 79 - DNA, 31 bp, synthetic construct
  misc_feature 1..31: note = Synthetic
  source 1..31: mol_type = other DNA
gcgccgtctcgaggccagaatggctaccaga

SEQ ID NO: 80 - DNA, 43 bp, synthetic construct
  misc_feature 1..43: note = Synthetic
  source 1..43: mol_type = other DNA
gcgccgtctcgctcgaagcttattgaagagaaggttccaactt

SEQ ID NO: 81 - DNA, 55 bp, synthetic construct
  misc_feature 1..55: note = Synthetic
  source 1..55: mol_type = other DNA
ggggacaagtttgtacaaaaaagcaggcttcatgaaaatgatgaatggtgaagat

SEQ ID NO: 82 - DNA, 55 bp, synthetic construct
  misc_feature 1..55: note = Synthetic
  source 1..55: mol_type = other DNA
ggggaccactttgtacaagaaagctgggtcctaggctgaagtgaacttgtaggag

SEQ ID NO: 83 - DNA, 53 bp, synthetic construct
  misc_feature 1..53: note = Synthetic
  source 1..53: mol_type = other DNA
ggggacaagtttgtacaaaaaagcaggcttcatggatcatgcaacattagcaa

SEQ ID NO: 84 - DNA, 57 bp, synthetic construct
  misc_feature 1..57: note = Synthetic
  source 1..57: mol_type = other DNA
ggggaccactttgtacaagaaagctgggtctcaatacctaggtattggaataagttt

SEQ ID NO: 85 - DNA, 59 bp, synthetic construct
  misc_feature 1..59: note = Synthetic
  source 1..59: mol_type = other DNA
ggggacaagtttgtacaaaaaagcaggcttcatggataacgcaacacttgctgtgatcc

SEQ ID NO: 86 - DNA, 62 bp, synthetic construct
  misc_feature 1..62: note = Synthetic
  source 1..62: mol_type = other DNA
ggggaccactttgtacaagaaagctgggtcctagtttctgggaactggaataacttgaagag